(19) United States
(12) Patent Application Publication
(10) Pub. No.: US 2011/0229525 A1
(43) Pub. Date: Sep. 22, 2011

(54) MODULATION OF CYTOKINE SIGNALING

(75) Inventors: Jack Jacek Hawiger, Nashville, TN (US); Antonio Digiandomenico, Clarksburg, MD (US)

(73) Assignee: Vanderbilt University, Nashville, TN (US)

(21) Appl. No.: 13/047,495

(22) Filed: Mar. 14, 2011

Related U.S. Application Data
(60) Provisional application No. 61/313,240, filed on Mar. 12, 2010.

(51) Int. Cl. A61K 38/02; C07K 19/00; C07H 21/00; C12N 5/071; A61P 37/02; A61P 29/00; A61P 9/00; A61P 35/00; A61P 3/10; A61P 25/00

(52) U.S. Cl. 424/278.1; 530/350; 536/23.4; 514/21.92; 435/375; 514/19.2; 514/6.9; 514/15.4; 514/19.3; 514/17.7; 514/20.8; 514/18.7; 514/18.9

(57) ABSTRACT

Cell penetrating suppressor of cytokine signaling (CP-SOCS) molecules engineered to be resistant to intracellular degradation are discussed. Methods of treating diseases associated with cytokine signaling include one or more CP-SOCS degradation resistant molecules.

### CROSS-REFERENCE TO RELATED APPLICATIONS

This application claims priority to U.S. provisional application No. 61/313,240, filed Mar. 12, 2010, which is incorporated herein by reference in its entirety.

### STATEMENT REGARDING FEDERALLY SPONSORED RESEARCH OR DEVELOPMENT

This disclosure was made with United States government support under grant number HL069452, and training grants F31-GM077030, and T32-AI007281 awarded by the United States Public Health Service National Institutes of Health. The United States government has certain rights in the disclosure.

### SEQUENCE LISTINGS

The sequence listing, containing the file named 20004_{—}0029_sequence_listing_ST25.txt which comprises the DNA and polypeptide sequences described herein was created on Mar. 11, 2011, and is hereby incorporated by reference in its entirety.

### FIELD

Embodiments of the disclosure provide compositions and methods for modulating cytokine signaling in vivo or in vitro. In particular, a cytokine modulator comprises a degradation resistant cell penetrating suppressor of cytokine signaling (SOCS).

### BACKGROUND

Inflammation constitutes a fundamental mechanism of diseases caused by microbial, autoimmune, and metabolic factors. These inducers evoke production of cytokines, chemokines, and other mediators of the host immune and inflammatory response. The inflammatory response depends on tightly regulated intracellular signal transduction by stress-responsive transcription factors as positive effectors of proinflammatory signaling in the nucleus (Hawiger, J. (2001) Immunol. Res. 23, 99-109). The genome can respond physiologically to proinflammatory cues by expressing a set of repressors that extinguish inflammation when the homeostatic balance is not excessively tipped in favor of proinflammatory agonists (e.g., IL-1, IL-6, TNF-α, and IFN-γ). Overproduction of these agonists contributes to runaway systemic inflammation dubbed “cytokine storm” that underlies life-threatening sepsis. Moreover, they mediate chronic tissue injury in inflammatory bowel disease, rheumatoid arthritis, multiple sclerosis, and other autoimmune diseases (Dinarello, C. A. (2000) Chest 118, 503-508; Opal, S. M., and DePalo, V. A. (2000) Chest 117, 1162-1172). To counteract the deleterious action of proinflammatory cytokines and chemokines, a set of extracellular anti-inflammatory molecules including TGF-β, IL-10, and IL-1R antagonist are produced. In addition, an intracellular negative feedback system has evolved to limit the duration and strength of proinflammatory signaling. This system is comprised of intracellular inhibitory proteins such as an inhibitory member of the Interleukin 1-Receptor Associated Kinase (IRAK)-M family, inhibitors of transcription factor NF-kβ (Ikβ), proteins that inhibit activated STAT (PIAS), suppressors of cytokine signaling (SOCS), and ubiquitin-modifying enzyme A20 (Alexander, W. S., and Hilton, D. J. (2004) Annu. Rev. Immunol. 22, 503-529; Liew, F. Y., Xu, D., Brint, E. K., and O'Neill, L. A. (2005) Nat. Rev. Immunol. 5, 446-458; Rakesh, K., and Agrawal, D. K. (2005) Biochem. Pharmacol. 70, 649-657; Coornaert, B., Carpentier, I., and Beyaert, R. (2009) J. Biol. Chem. 284, 8217-8221). While SH2-containing inositol 5 phosphatases (SHIP and SHIP 1) counteract signaling events based on tyrosine phosphorylation, SOCS proteins prevent cytokine receptor signaling by binding to the cytoplasmic tail of cytokine receptors and/or catalytic sites on JAK kinases.

### SUMMARY

This Summary is provided to present a synopsis of the disclosure with a brief description of the nature and substance of the disclosure. It is submitted with the understanding that it will not be used to interpret or limit the scope or meaning of the claims.

Certain embodiments of the disclosure pertain to a recombinant polypeptide comprising a suppressor of cytokine signaling (SOCS) polypeptide. Still further, the embodiments of the disclosure contemplate a recombinant polypeptide comprising a SOCS polypeptide and a cell penetrating domain, wherein the suppressor of cytokine signaling (SOCS) polypeptide lacks: (i) a functional C-terminal SOCS box, (ii) a functional PEST domain or motif, or combinations thereof.

In certain embodiments of the disclosure wherein the polypeptide lacks a functional C-terminal SOCS box, the loss of function may comprise one or more mutations, substitutions, deletions or combinations thereof rendering the C-terminal SOCS box non-functional. In certain embodiments, the one or more mutations, substitutions, deletions or combinations thereof rendering the C-terminal SOCS box non-functional may be within the c-terminal SOCS box. In alternate embodiments, the C-terminal SOCS box is deleted.

In certain embodiments of the disclosure wherein the polypeptide lacks a functional PEST domain or motif, the loss of function may comprise one or more mutations, substitutions, deletions or combinations thereof rendering the PEST domain non-functional. In certain embodiments, the one or more mutations, substitutions, deletions or combinations thereof rendering the PEST domain non-functional may be within the PEST domain. In alternate embodiments, the PEST domain is deleted.

In certain embodiments wherein the polypeptide comprising a SOCS polypeptide is contemplated, the SOCS polypeptide is selected from the group consisting of SOCS 1, SOCS 2, SOCS 3, SOCS 4, SOCS 5, SOCS 6, SOCS 7, variants mutants, analogs, fragments, species or combinations thereof. In specific embodiments wherein the polypeptide comprising a SOCS polypeptide is contemplated, the SOCS peptide is SOCS 3.

Certain other embodiments of the disclosure pertain to an isolated nucleic acid encoding a recombinant polypeptide comprising a suppressor of cytokine signaling (SOCS) polypeptide and a cell penetrating domain, wherein the suppressor of cytokine signaling (SOCS) polypeptide lacks: (i) a functional C-terminal SOCS box, (ii) PEST domain, or combinations thereof.

In certain embodiments of the disclosure wherein the nucleic acid encodes a recombinant polypeptide comprising a functional C-terminal SOCS box, the loss of function may comprise one or more mutations, substitutions, deletions or combinations thereof within the nucleic acid rendering the C-terminal SOCS box non-functional. In certain embodiments, the one or more mutations, substitutions, deletions or combinations thereof within the nucleic acid rendering the C-terminal SOCS box non-functional may be within area of the nucleic acid encoding the c-terminal SOCS box. In alternate embodiments, the nucleic acid does not encode the C-terminal SOCS box.

In certain embodiments of the disclosure wherein the nucleic acid encodes a recombinant polypeptide lacking a functional PEST domain, the loss of function may comprise one or more mutations, substitutions, deletions or combinations thereof within the nucleic acid rendering the PEST domain non-functional. In certain embodiments, the one or more mutations, substitutions, deletions or combinations thereof within the nucleic acid rendering the PEST domain non-functional may be within area of the nucleic acid encoding the PEST domain. In alternate embodiments, the nucleic acid does not encode the PEST domain.

In certain embodiments wherein the nucleic acid encoding a polypeptide comprising a SOCS polypeptide is contemplated, the nucleic acid may encode a SOCS peptide selected from the group consisting of SOCS 1, SOCS 2, SOCS 3, SOCS 4, SOCS 5, SOCS 6, SOCS 7, variants mutants, analogs, fragments, species or combinations thereof. In specific embodiments the nucleic acid encodes a polypeptide comprising a SOCS peptide, the SOCS peptide is SOCS 3.

Still further, certain other embodiments concern a pharmaceutical composition comprising a nucleic acid expressing a recombinant polypeptide or a recombinant polypeptide, the isolated nucleic acid or recombinant polypeptide comprising a suppressor of cytokine signaling (SOCS) polypeptide and a cell penetrating domain, wherein the suppressor of cytokine signaling (SOCS) polypeptide lacks: (i) a functional C-terminal SOCS box, (ii) PEST domain or motif, or combinations thereof.

Certain other embodiments relate to a method of increasing half-life (t_{1/2}) of a suppressor of cytokine signaling (SOCS) polypeptides in vitro or in vivo, comprising: engineering a recombinant polypeptide or an isolated nucleic acid encoding a polypeptide comprising a suppressor of cytokine signaling (SOCS) polypeptide and a cell penetrating domain, wherein the suppressor of cytokine signaling (SOCS) polypeptide lacks: (i) a functional C-terminal SOCS box, (ii) PEST domain, or combinations thereof; administering the isolated nucleic acid or recombinant polypeptide to a cell or patient and, increasing half-life (t_{1/2}) of a suppressor of cytokine signaling (SOCS) polypeptides in vitro or in vivo.

Certain embodiments of the disclosure pertain to a method of modulating cytokine signaling in vitro or in vivo, comprising: administering to a patient, an effective amount of a recombinant polypeptide or an isolated nucleic acid encoding a polypeptide comprising a suppressor of cytokine signaling (SOCS) polypeptide and a cell penetrating domain, wherein the suppressor of cytokine signaling (SOCS) polypeptide lacks: (i) a functional C-terminal SOCS box, (ii) PEST domain, or combinations thereof; administering the isolated nucleic acid or recombinant polypeptide to a cell or patient; and, modulating cytokine signaling in vitro or in vivo.

Other embodiments of the disclosure relate to a method of treating a disease or disorder in a patient, associated with cytokine signaling, comprising: administering to a patient in need thereof, a therapeutically effective amount of a cytokine modulator in a pharmaceutical composition; and, treating the disease or disorder in the patient.

In embodiments related to a method of treating a disease or a disorder by administering a cytokine modulator, the cytokine modulator may comprise a recombinant polypeptide having a suppressor of cytokine signaling (SOCS) polypeptide and a cell penetrating domain, wherein the suppressor of cytokine signaling (SOCS) polypeptide lacks: (i) a functional C-terminal SOCS box, (ii) PEST domain, or combinations thereof.

The method of claim 15, wherein a cytokine modulator comprises a nucleic acid encoding for a polypeptide having a suppressor of cytokine signaling (SOCS) polypeptide and a cell penetrating domain, wherein the suppressor of cytokine signaling (SOCS) polypeptide lacks: (i) a functional C-terminal SOCS box, (ii) PEST domain, or combinations thereof.

In embodiments related to a method of treating a disease or a disorder by administering a cytokine modulator, the cytokine modulator may comprise a cell expressing a polypeptide comprising a suppressor of cytokine signaling (SOCS) protein and a cell penetrating domain, wherein the suppressor of cytokine signaling (SOCS) protein lacks: (i) a functional C-terminal SOCS box, (ii) PEST domain, or combinations thereof.

In embodiments related to a method of treating a disease or a disorder by administering a cytokine modulator, a disease associated with cytokine signaling comprises: autoimmune diseases or disorders, cardiovascular diseases or disorders, neurological diseases or disorders, neuroinflammatory diseases or disorders, inflammatory eye disorder, inflammatory skin disorders, cancer, neurodegenerative diseases or disorders, inflammatory diseases or disorders, liver, pancreas or kidney diseases or disorders, inflammatory disorders of placenta and amnion, diabetes, apoptosis, or aberrant cell proliferation.

Certain other embodiments of the disclosure relate to a method of modulating an immune response comprising: administering to a patient in need thereof, a therapeutically effective amount of a cytokine modulator in a pharmaceutical composition; and, modulating an immune response.

In embodiments of the disclosure related to modulating an immune response, the cytokine modulator may comprise a recombinant polypeptide having a suppressor of cytokine signaling (SOCS) polypeptide and a cell penetrating domain, wherein the suppressor of cytokine signaling (SOCS) polypeptide lacks: (i) a functional C-terminal SOCS box, (ii) PEST domain, or combinations thereof.

In other embodiments of the disclosure related to modulating an immune response, the cytokine modulator may comprise a nucleic acid encoding for a polypeptide having a suppressor of cytokine signaling (SOCS) polypeptide and a cell penetrating domain, wherein the suppressor of cytokine signaling (SOCS) polypeptide lacks: (i) a functional C-terminal SOCS box, (ii) PEST domain, or combinations thereof.

In other embodiments of the disclosure related to modulating an immune response, the cytokine modulator may comprise a cell expressing a polypeptide comprising a suppressor of cytokine signaling (SOCS) polypeptide and a cell penetrating domain, wherein the suppressor of cytokine signaling (SOCS) polypeptide lacks: (i) a functional C-terminal SOCS box, (ii) PEST domain, or combinations thereof.

Other embodiments of the disclosure concern a method of protecting a cell in vivo or in vitro from apoptosis, comprising: contacting a cell in vitro or in vivo with a therapeutically effective amount of a cytokine modulator in a pharmaceutical composition; and, of protecting the cell in vivo or ex vivo from apoptosis.

In embodiments of the disclosure where a method of protecting a cell in vivo or in vitro from apoptosis is concerned, the cytokine modulator may comprise a recombinant polypeptide having a suppressor of cytokine signaling (SOCS) polypeptide and a cell penetrating domain, wherein the suppressor of cytokine signaling (SOCS) protein lacks: (i) a functional C-terminal SOCS box, (ii) PEST domain, or combinations thereof.

In other embodiments of the disclosure where a method of protecting a cell in vivo or in vitro from apoptosis is concerned, the cytokine modulator may comprise a nucleic acid encoding for a polypeptide having a suppressor of cytokine signaling (SOCS) polypeptide and a cell penetrating domain, wherein the suppressor of cytokine signaling (SOCS) protein lacks: (i) a functional C-terminal SOCS box, (ii) PEST domain, or combinations thereof.

In other embodiments of the disclosure where a method of protecting a cell in vivo or in vitro from apoptosis is concerned, the cytokine modulator may comprise a cell expressing a polypeptide comprising a suppressor of cytokine signaling (SOCS) polypeptide and a cell penetrating domain, wherein the suppressor of cytokine signaling (SOCS) polypeptide lacks: (i) a functional C-terminal SOCS box, (ii) PEST domain, or combinations thereof.

### BRIEF DESCRIPTION OF THE DRAWINGS

FIGS. 1A-1B show the design of recombinant CP-SOCS3 proteins for bacterial expression and affinity purification. FIG. 1A shows a schematic representation of full-length wild-type SOCS3, showing the different functional domains of the protein: KIR (Kinase Inhibitory Region), SH2 domain, PEST motif, and SOCS box; Non-CP-SOCS3, the non-cell penetrating SOCS3 that lacks the MTM, but contains an N-terminal 6X-His Tag (white); CP-SOCS3, cell-penetrating full-length SOCS3 with a 12 amino acid MTM (red) at the NH2-terminal, and 6X-His Tag (white); CP-SOCS3ΔSB, cell-penetrating SOCS3 deletion mutant lacking the C-terminal SOCS box, but possessing the MTM (red) and 6X-His Tag (white) at the N-terminus. FIG. 1B: Immunoblot displaying expressed and purified non-CP-SOCS3 (26.6 kDa), CP-SOCS3 (27.9 kDa), and CP-SOCS3ΔSB (23.4 kDa).

FIGS. 2A-2D show endogenous SOCS3 turnover in RAW macrophages stimulated with proinflammatory agonists. RAW 264.7 cells were stimulated with 100 Units/ml IFN-γ and 250 ng/ml LPS for 4 h to induce SOCS3 expression (t=0). After the treatments indicated below, samples were collected at 0, 0.5, 1, 1.5, 2, 4, & 6 h. SOCS3 protein levels were quantified by immunoblotting (IB) using infrared Odyssey Li-Cor system software. FIG. 2A: RAW cells incubated without (squares) or with (circles) 15 μg/ml cycloheximide. FIG. 2B: RAW cells incubated without (squares) or with (diamonds) 15 μg/ml cycloheximide and 1 μM epoxomicin. FIG. 2C: RAW cells incubated without (squares) or with (triangles) 15 μg/ml cycloheximide plus 40 μg/ml calpeptin. FIG. 2D: RAW cells incubated without (squares) or with (inverted triangle) all three inhibitors: 15 μg/ml cycloheximide, 40 μg/ml calpeptin, and 1 μM epoxomicin. Values shown are the mean±S.E. of n=3 (in A&B) or n=4 (in C&D) independent experiments.

FIGS. 3A-3L show the intracellular delivery of recombinant SOCS3 proteins. Fluorescence confocal laser scanning microscopy of proteinase K-treated and non-fixed RAW macrophages shows intracellular localization of FITC-labeled CP-SOCS3 proteins (green). FIGS. 3A-3C: Confocal images of RAW cells incubated with FITC alone. FIG. 3A: FITC image: no fluorescent signal observed. FIG. 3B: Differential interference contrast (DIC) image of the RAW cells depicted above. FIG. 3C: Merged view of DIC and FITC images. FIGS. 3D-3F: Confocal images of RAW cells incubated with FITC-labeled nonCP-SOCS3. FIG. 3D: FITC image—no fluorescent signal detected. FIG. 3E: DIC image of the RAW cells depicted above. FIG. 3F: Merged view of DIC and FITC images. FIGS. 3G-3H: Confocal images of RAW cells incubated with FITC-labeled CP-SOCS3. FIG. 3G: FITC image—strong fluorescence throughout the cytoplasm. FIG. 3H: DIC image of RAW cells depicted above. FIG. 3I: Merged view of DIC and FITC images showing localization of FITC-labeled CP-SOCS3 throughout the cytoplasm of the RAW cells. FIGS. 3J-3I: Confocal images of RAW cells incubated with FITC-labeled CP-SOCS3ΔSB. FIG. 3J: FITC image—strong fluorescence throughout the cytoplasm. FIG. 3K: DIC image of RAW cells depicted above. FIG. 3L: Merged view of DIC and FITC images showing localization of FITC-labeled HMS3Δsb throughout the cytoplasm. All images are representative of multiple unfixed cells from three independent experiments.

FIGS. 4A-4I: Intracellular delivery of CP-SOCS3 bypasses endosomal membrane compartment. Fluorescence confocal laser scanning microscopy of RAW macrophages incubated with FITC-labeled recombinant proteins (green), and the endosomal marker FM-595 (red). FIGS. 4A-4C: RAW cells incubated with FITC-labeled non-CP-SOCS3 (green) and FM-595 (red). FIG. 4A: FITC image—no fluorescent signal detected. FIG. 4B: FM-595 only—endosomes detected throughout the cell. FIG. 4C: Merged view of FITC and FM-595 images. FIGS. 4D-4F: Confocal images of RAW cells incubated with FITC-labeled CP-SOCS3 and FM-595. FIG. 4D: FITC image—fluorescent signal throughout the cytoplasm FIG. 4E: FM-595 only—endosomes detected throughout the cell. FIG. 4F: Merged view of FITC and FM-595 images—no overlapping green and red fluorescent signals. FIGS. 4G-4I: Confocal images of RAW cells incubated with FITC-labeled CP-SOCS3ΔSB and FM-595. FIG. 4G: FITC images—fluorescent signal throughout the cytoplasm. FIG. 4H: FM-595 only—endosomes detected throughout the cell. FIG. 4I: Merged view of FITC and FM-595 images—no overlapping green and red fluorescent signals. All images are representative of multiple unfixed cells from three independent experiments.

FIGS. 5A and 5B: Proteasomal inhibitor extends the half-life of CP-SOCS3 and deletion of the SOCS box dramatically improves the intracellular stability of CP-SOCS3. FIG. 5A: Stimulated RAW macrophages were treated for 1 h with CP-SOCS3 in the presence (inverted triangle) or absence (squares) of 1 μM epoxomicin. Half-life was determined by immunoblot analysis of samples collected at 0, 0.5, 2, 4, 6, 12, & 24 hours. FIG. 5B: Stimulated RAW macrophages were treated for 1 hour with CP-SOCS3ΔSB in the absence (open squares) or presence (solid circles) of 1 μM epoxomicin. Half-life was determined by immunoblot analysis of samples collected at 0, 0.5, 2, 4, 6, 12, & 24 hours. Values are the mean±S.E. of three independent experiments (n=3).

FIGS. 6A-6E: CP-SOCS3ΔSB inhibits STAT1 phosphorylation and displays prolonged anti-inflammatory activity associated with intracellular persistence as compared to full-length CP-SOCS3 in AMJ2.C8 macrophage cell line. The cells were treated for 1 h with CP-SOCS3, or CP-SOCS3ΔSB. FIGS. 6A, 6B. Six hours or 24 h following protein treatment, cells were stimulated with 100 Units/ml IFN-γ and 0.5 μg/ml LPS for 6 h. Supernatants were collected before treatment (t=0 h) and after the 6 h stimulation, at 12 and 30 h, respectively. Samples analyzed for inflammatory cytokine/chemokine production by CBA. FIG. 6A: TNF-α (pg/mL). FIG. 6B: MCP-1 (pg/ml). After 1 hour pre-treatment of macrophages with cell-penetrating proteins, cells were stimulated with 100 Units/ml IFN-γ and 0.2 μg/ml LPS for 15 minutes. Cells were harvested with 1×CBA lysis buffer and analyzed for phosphorylated STAT1 levels by CBA. FIG. 6C. pSTAT1 (Units/ml). FIG. 6D: Immunoblotting results of CP-SOCS3 or CP-SOCS3ΔSB protein levels in cells after 6 h stimulation, at 12 and 30 h. FIG. 6E: Immunoblotting results of pSTAT1 in AMJ2.C8 macrophages treated with CP-SOCS3 or CP-SOCS3ΔSB for 1 hour and stimulated for 15 minutes. Values are the mean±S.E. of n=4 (FIGS. 6A, 6B, 6D) or n=3 (FIGS. 6C, 6E) independent experiments.

FIG. 7A-7E: CP-SOCS3ΔSB displays prolonged anti-inflammatory activity and intracellular persistence in primary macrophages. Bone marrow-derived macrophages (BMDM) obtained from C3H/HeJ mice were treated for 1 h with 10 μM CP-SOCS3, or 10 μM CP-SOCS3ΔSB. FIGS. 7A, 7B, 7D: Six hours or 24 h following protein treatment, cells were stimulated with 100 Units/ml IFN-γ and 0.5 μg/ml LPS for 6 h. Supernatants were collected before treatment (t=0 h) and after the 6 h stimulation, at 12 and 30 h, respectively. Samples analyzed for inflammatory cytokine/chemokine production by CBA. A. TNF-α (pg/ml). FIG. 7B. MCP-1 (pg/ml). FIG. 7C: After 1 hour pre-treatment of macrophages with cell-penetrating proteins, cells were stimulated with 100 Units/ml IFN-γ and 0.2 μg/ml LPS for 15 minutes. Cells were harvested with 1×CBA lysis buffer and analyzed for phosphorylated STAT1 by CBA. FIG. 7C: pSTAT1 (Units/ml). FIG. 7D: Immunoblots of CP-SOCS3 or CP-SOCS3ΔSB protein levels in cells after 6 h stimulation, at 12 and 30 h. FIG. 7E. Immunoblots of pSTAT1 levels in BMDM treated with CP-SOCS3 or CP-SOCS3ΔSB for 1 hour and stimulated for 15 minutes. Values are the mean±S.E. of n=4 (in FIGS. 7A, 7B, 7D) or (in FIG. 7C) n=3 independent experiments.

### DETAILED DESCRIPTION

The present disclosure is described with reference to the attached figures, wherein like reference numerals are used throughout the figures to designate similar or equivalent elements. The figures are not drawn to scale and they are provided merely to illustrate the instant disclosure. Several aspects of the disclosure are described below with reference to example applications for illustration. It should be understood that numerous specific details, relationships, and methods are set forth to provide a full understanding of the disclosure. One having ordinary skill in the relevant art, however, will readily recognize that the disclosure can be practiced without one or more of the specific details or with other methods. The present disclosure is not limited by the illustrated ordering of acts or events, as some acts may occur in different orders and/or concurrently with other acts or events. Furthermore, not all illustrated acts or events are required to implement a methodology in accordance with the present disclosure.

Unless otherwise defined, all terms (including technical and scientific terms) used herein have the same meaning as commonly understood by one of ordinary skill in the art to which this disclosure belongs. It will be further understood that terms, such as those defined in commonly used dictionaries, should be interpreted as having a meaning that is consistent with their meaning in the context of the relevant art and will not be interpreted in an idealized or overly formal sense unless expressly so defined herein.

All genes, gene names, and gene products disclosed herein are intended to correspond to homologs from any species for which the compositions and methods disclosed herein are applicable. Thus, the terms include, but are not limited to genes and gene products from humans and mice. It is understood that when a gene or gene product from a particular species is disclosed, this disclosure is intended to be exemplary only, and is not to be interpreted as a limitation unless the context in which it appears clearly indicates. Thus, for example, for the genes disclosed herein, which in some embodiments relate to mammalian nucleic acid and amino acid sequences are intended to encompass homologous and/or orthologous genes and gene products from other animals including, but not limited to other mammals, fish, amphibians, reptiles, and birds. In preferred embodiments, the genes or nucleic acid sequences are human.

### DEFINITIONS

The terminology used herein is for the purpose of describing particular embodiments only and is not intended to be limiting of the disclosure. As used herein, the singular forms “a”, “an” and “the” are intended to include the plural forms as well, unless the context clearly indicates otherwise. Furthermore, to the extent that the terms “including”, “includes”, “having”, “has”, “with”, or variants thereof are used in either the detailed description and/or the claims, such terms are intended to be inclusive in a manner similar to the term “comprising.”

The term “about” or “approximately” means within an acceptable error range for the particular value as determined by one of ordinary skill in the art, which will depend in part on how the value is measured or determined, i.e., the limitations of the measurement system. For example, “about” can mean within 1 or more than 1 standard deviation, per the practice in the art. Alternatively, “about” can mean a range of up to 20%, preferably up to 10%, more preferably up to 5%, and more preferably still up to 1% of a given value. Alternatively, particularly with respect to biological systems or processes, the term can mean within an order of magnitude, preferably within 5-fold, and more preferably within 2-fold, of a value. Where particular values are described in the application and claims, unless otherwise stated the term “about” meaning within an acceptable error range for the particular value should be assumed.

As used herein, the term “safe and effective amount” or “therapeutic amount” refers to the quantity of a component which is sufficient to yield a desired therapeutic response without undue adverse side effects (such as toxicity, irritation, or allergic response) commensurate with a reasonable benefit/risk ratio when used in the manner of this disclosure. By “therapeutically effective amount” is meant an amount of a compound of the present disclosure effective to yield the desired therapeutic response. The specific safe and effective amount or therapeutically effective amount will vary with such factors as the particular condition being treated, the physical condition of the patient, the type of mammal or animal being treated, the duration of the treatment, the nature of concurrent therapy (if any), and the specific formulations employed and the structure of the compounds or its derivatives.

As used herein, “modulation” means either an increase (stimulation) or a decrease (inhibition) in the expression, in vivo amounts of a gene. This includes any amounts in vivo, functions and the like as compared to normal controls. The term includes, for example, increased, enhanced, increased, agonized, promoted, decreased, reduced, suppressed blocked, or antagonized. Modulation can increase activity or amounts more than 1-fold, 2-fold, 3-fold, 5-fold, 10-fold, 100-fold, etc., over baseline values. Modulation can also decrease its activity or amounts below baseline values.

The term “variant,” when used in the context of a polynucleotide sequence, may encompass a polynucleotide sequence related to a wild type gene. This definition may also include, for example, “allelic,” “splice,” “species,” or “polymorphic” variants. A splice variant may have significant identity to a reference molecule, but will generally have a greater or lesser number of polynucleotides due to alternate splicing of exons during mRNA processing. The corresponding polypeptide may possess additional functional domains or an absence of domains. Species variants are polynucleotide sequences that vary from one species to another. Of particular utility in the disclosure are variants of wild type gene products. Variants may result from at least one mutation in the nucleic acid sequence and may result in altered mRNAs or in polypeptides whose structure or function may or may not be altered. Any given natural or recombinant gene may have none, one, or many allelic forms. Common mutational changes that give rise to variants are generally ascribed to natural deletions, additions, or substitutions of nucleotides. Each of these types of changes may occur alone, or in combination with the others, one or more times in a given sequence.

The resulting polypeptides generally will have significant amino acid identity relative to each other. A polymorphic variant is a variation in the polynucleotide sequence of a particular gene between individuals of a given species. Polymorphic variants also may encompass “single nucleotide polymorphisms” (SNPs) or single base mutations in which the polynucleotide sequence varies by one base. The presence of SNPs may be indicative of, for example, a certain population with a propensity for a disease state, that is susceptibility versus resistance.

Derivative polynucleotides include nucleic acids subjected to chemical modification, for example, replacement of hydrogen by an alkyl, acyl, or amino group. Derivatives, e.g., derivative oligonucleotides, may comprise non-naturally-occurring portions, such as altered sugar moieties or inter-sugar linkages. Exemplary among these are phosphorothioate and other sulfur containing species which are known in the art. Derivative nucleic acids may also contain labels, including radionucleotides, enzymes, fluorescent agents, chemiluminescent agents, chromogenic agents, substrates, cofactors, inhibitors, magnetic particles, and the like.

A “derivative” polypeptide or peptide is one that is modified, for example, by glycosylation, pegylation, phosphorylation, sulfation, reduction/alkylation, acylation, chemical coupling, or mild formalin treatment. A derivative may also be modified to contain a detectable label, either directly or indirectly, including, but not limited to, a radioisotope, fluorescent, and enzyme label.

As used herein, the term “fragment or segment”, as applied to a nucleic acid sequence, gene or polypeptide, will ordinarily be at least about 5 contiguous nucleic acid bases (for nucleic acid sequence or gene) or amino acids (for polypeptides), typically at least about 10 contiguous nucleic acid bases or amino acids, more typically at least about 20 contiguous nucleic acid bases or amino acids, usually at least about 30 contiguous nucleic acid bases or amino acids, preferably at least about 40 contiguous nucleic acid bases or amino acids, more preferably at least about 50 contiguous nucleic acid bases or amino acids, and even more preferably at least about 60 to 80 or more contiguous nucleic acid bases or amino acids in length. “Overlapping fragments” as used herein, refer to contiguous nucleic acid or peptide fragments which begin at the amino terminal end of a nucleic acid or protein and end at the carboxy terminal end of the nucleic acid or protein. Each nucleic acid or peptide fragment has at least about one contiguous nucleic acid or amino acid position in common with the next nucleic acid, protein or peptide fragment, more preferably at least about three contiguous nucleic acid bases or amino acid positions in common, most preferably at least about ten contiguous nucleic acid bases amino acid positions in common.

“Patient” or “subject” refers to mammals and includes human and veterinary subjects.

As used herein the phrase “diagnostic” means identifying the presence or nature of a pathologic condition. Diagnostic methods differ in their sensitivity and specificity. The “sensitivity” of a diagnostic assay is the percentage of diseased individuals who test positive (percent of “true positives”). Diseased individuals not detected by the assay are “false negatives.” Subjects who are not diseased and who test negative in the assay are termed “true negatives.” The “specificity” of a diagnostic assay is 1 minus the false positive rate, where the “false positive” rate is defined as the proportion of those without the disease who test positive. While a particular diagnostic method may not provide a definitive diagnosis of a condition, it suffices if the method provides a positive indication that aids in diagnosis.

As used herein the phrase “diagnosing” refers to classifying a disease or a symptom, determining a severity of the disease, monitoring disease progression, forecasting an outcome of a disease and/or prospects of recovery. The term “detecting” may also optionally encompass any of the above. Diagnosis of a disease according to the present disclosure can be effected by determining a level of a polynucleotide or a polypeptide of the present disclosure in a biological sample obtained from the subject, wherein the level determined can be correlated with predisposition to, or presence or absence of the disease. It should be noted that a “biological sample obtained from the subject” may also optionally comprise a sample that has not been physically removed from the subject, as described in greater detail below.

The term “sample” is meant to be interpreted in its broadest sense. A “sample” refers to a biological sample, such as, for example; one or more cells, tissues, or fluids (including, without limitation, plasma, serum, whole blood, cerebrospinal fluid, lymph, tears, urine, saliva, milk, pus, and tissue exudates and secretions) isolated from an individual or from cell culture constituents, as well as samples obtained from, for example, a laboratory procedure. A biological sample may comprise chromosomes isolated from cells (e.g., a spread of metaphase chromosomes), organelles or membranes isolated from cells, whole cells or tissues, nucleic acid such as genomic DNA in solution or bound to a solid support such as for Southern analysis, RNA in solution or bound to a solid support such as for Northern analysis, cDNA in solution or bound to a solid support, oligonucleotides in solution or bound to a solid support, polypeptides or peptides in solution or bound to a solid support, a tissue, a tissue print and the like.

“Treating” or “treatment” of a state, disorder or condition includes: (1) Preventing or delaying the appearance of clinical or sub-clinical symptoms of the state, disorder or condition developing in a mammal that may be afflicted with or predisposed to the state, disorder or condition but does not yet experience or display clinical or subclinical symptoms of the state, disorder or condition; or (2) Inhibiting the state, disorder or condition, i.e., arresting, reducing or delaying the development of the disease or a relapse thereof (in case of maintenance treatment) or at least one clinical or sub-clinical symptom thereof; or (3) Relieving the disease, i.e., causing regression of the state, disorder or condition or at least one of its clinical or sub-clinical symptoms. The benefit to a subject to be treated is either statistically significant or at least perceptible to the patient or to the physician.

A “prophylactically effective amount” refers to an amount effective, at dosages and for periods of time necessary, to achieve the desired prophylactic result. Typically, since a prophylactic dose is used in subjects prior to or at an earlier stage of disease, the prophylactically effective amount will be less than the therapeutically effective amount and prevents or is protective against the disease or infection.

### Compositions

SOCS proteins are encoded by immediate early genes and they influence the extent and outcome of proinflammatory cytokine signaling (Alexander, W. S., and Hilton, D. J. (2004) Annu. Rev. Immunol. 22, 503-529). The SOCS family is composed of eight cytoplasmic SH2 domain—containing proteins: SOCS1 to SOCS7 and cytokine-inducible SH2 (CIS). These physiologic suppressors uniquely disrupt proinflammatory signaling by either inhibiting the activity of JAK kinases or interacting with ligand-occupied cytokine receptors (Nicholson, S. E., et al. (1999) EMBO J. 18, 375-385). In addition, SOCS proteins contain a C-terminal SOCS box that associates with elongins B/C and cullin to form a ubiquitin E3 ligase that targets SOCS proteins and their signaling complexes for proteasomal degradation (Kamura, T., et al. (1998) Genes Dev. 12, 3872-3881). Several members of the SOCS family, including SOCS1 and SOCS3, contain a proline, glutamine, serine, threonine (PEST) motif, which targets proteins for rapid intracellular proteolysis by calpain proteases (Babon, J. J., et al. (2006) Mol. Cell. 22, 205-216). Among the SOCS family members, SOCS1 and SOCS3 are the best characterized in terms of their abilities to regulate proinflammatory cytokine signaling. Although structurally similar to SOCS1, SOCS3 does not inhibit cytokine signaling by binding directly to JAK, rather it inhibits JAK only in the presence of gp130 (Kubo, M., Hanada, T. and Yoshimura, A. (2003) Nat. Immunol. 4, 1169-1176).

Using a new technology platform, these two physiologic inhibitors of inflammation and apoptosis for intracellular delivery in vivo (Jo, D., Liu, D., Yao, S., Collins, R. D., and Hawiger, J. (2005) Nat. Med. 11, 892-898; DiGiandomenico, A., Wylezinski, L. S., and Hawiger, J. (2009) Sci. Signal. 2, ra37). Functional studies demonstrate that cell-penetrating (CP) forms of SOCS1 and SOCS3 potently inhibit the JAK/STAT signaling pathway in cultured cells and CP-SOCS3 suppresses inflammation and protects vital organs from failure in mice challenged with the superantigenic staphylococcal enterotoxin B or endotoxic lipopolysaccharide.

When fluorescently tagged CP-SOCS3 was administered in vivo its striking persistence in blood leukocytes, lymphocytes, and spleen cells it was noted (see, also, for example, the Examples section which follows). These findings further led to the investigation, in the intracellular turnover of CP-SOCS3 as compared to endogenous SOCS3 induced by proinflammatory agonists, shown in the Examples section which follows. The role of the PEST motif and SOCS box in the turnover of CP-SOCS3 and its endogenous counterpart was also investigated. In brief, the results indicate a remarkable half-life prolongation for CP-SOCS3 as compared to endogenous wild-type SOCS3 and provide compelling evidence that protein degradation motifs play an important role in the turnover of full-length SOCS3. Moreover, deletion of the SOCS box, which controls proteasomal degradation, led to a much longer-acting (t1/2=29 h) suppressor of proinflammatory agonists-induced cytokine and chemokine production.

In a preferred embodiment, a recombinant polypeptide comprises a suppressor of cytokine signaling (SOCS) protein and a cell penetrating motif, wherein the suppressor of cytokine signaling (SOCS) protein lacks: (i) a functional C-terminal SOCS box, (ii) PEST domain or motif, or combinations thereof.

In another preferred embodiment, the SOCS box comprises one or more mutations, deletions or combinations thereof, which would result in the loss of functional activity of the SOCS box. In alternative embodiments, the SOCS box is deleted.

In another preferred embodiment, the PEST domain or motif comprises one or more mutations, substitutions, deletions, or combinations thereof. Preferably the PEST domain or motif is deleted from the recombinant polypeptide. As used herein, “PEST motif” refers to a region of a polypeptide rich in the amino acids proline (P); glutamic acid (E); serine (S); or threonine (T) that is associated with rapidly degraded proteins.

In another preferred embodiment, the SOCS protein or peptide is selected from the group consisting of SOCS 1, SOCS 2, SOCS 3, SOCS 4, SOCS 5, SOCS 6, SOCS 7, variants, mutants, analogs, fragments, species or combinations thereof. However, any SOCS protein, such as SOCS-1, SOCS-2, SOCS-3, SOCS-4, SOCS-5, SOCS-6, or SOCS-7 (or fragment thereof), from any species, in any combination, can be used as the source of the SOCS sequence. The SOCS protein(s) used can be selected based on the purpose to be accomplished by importing the molecule into the selected cell. In some embodiments, the SOCS protein or peptide comprises sequences from other SOCS proteins or peptides, either encoded by nucleic sequences or synthesized. In other embodiments, the SOCS nucleic acid sequence or peptide sequences contain peptide or nucleic acid sequences from other molecules as long as they do not affect the function and activity of the SOCS molecule. For example, the cell penetrating (CP) sequence. Such nucleic acid sequences can be referred to as “cell-penetrating SOCS nucleic acids.” In certain embodiments, the cell penetrating peptides or amino acid sequences are those of SEQ ID NO: 1, SEQ ID NO: 2 or SEQ ID NO: 3. Also disclosed are vectors and cells comprising the cell-penetrating SOCS nucleic acids. The SOCS sequence can comprise a SOCS protein.

In certain embodiments, the SOCS peptides may be encoded by nucleic acid sequences. For example, a SOCS polypeptide encoding nucleic acid may be a human nucleic acid capable of expressing a human SOCS polypeptide. In such embodiments, SEQ ID NO: 11 may correspond to a nucleic acid expressing human SOCS1 polypeptide SEQ ID NO: 10. SEQ ID NO: 13 may correspond to a nucleic acid expressing human SOCS2 polypeptide SEQ ID NO: 12. SEQ ID NO: 15 may correspond to a nucleic acid expressing human SOCS3 polypeptide SEQ ID NO: 14. SEQ ID NO: 17 may correspond to a nucleic acid expressing human SOCS4 polypeptide SEQ ID NO: 16. SEQ ID NO: 19 may correspond to a nucleic acid expressing human SOCS5 polypeptide SEQ ID NO: 18. SEQ ID NO: 21 may correspond to a nucleic acid expressing human SOCS6 polypeptide SEQ ID NO: 20. SEQ ID: NO 23 may correspond to a nucleic acid expressing human SOCS7 polypeptide SEQ ID NO: 22.

The SOCS sequence can also be defined functionally. Cytokine signaling induces the expression of SOCS proteins through the JAK-STAT signaling pathway. The induced SOCS proteins block the interaction of STATs with receptors by steric hindrance or competition via SH2-domain-mediated binding to JAKs and cytokine receptors; or inhibit the catalytic activity of JAKs though binding via the KIR and SH2 region. Therefore, “SOCS sequence” as used herein can also be defined as being any amino acid sequence capable of functioning as a suppressor of cytokine signaling. Such suppression can be defined as a 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% suppression of cytokine signaling. This suppression can be measured by measuring expansion of lymphoid progenitors, STAT5 phosphorylation, or expression of TNF-α, IL-6, and other cytokines. Examples of measuring suppression can be found, for example, in both herein incorporated by reference in their entirety for their teaching regarding measuring suppression of intracellular signaling induced by cytokines and growth factors. Alternatively, full-length SOCS proteins or their fragments can contain one or more mutated residues rendering them dominant negative inhibitors of endogenous SOCS proteins. Such inhibitors can prevent SOCS proteins from extinguishing physiologic signaling evoked by growth factors and hormones (examples include reversal of anemia during chronic infection or reversal of insulin and leptin resistance in metabolic syndrome that characterizes type II diabetes).

In a preferred embodiment, the SOCS protein or peptide is SOCS 3.

In another preferred embodiment, an isolated nucleic acid encodes the recombinant SOCS polypeptides comprising a suppressor of cytokine signaling (SOCS) peptide and a cell penetrating domain, wherein the suppressor of cytokine signaling (SOCS) peptide lacks: (i) a functional C-terminal SOCS box, (ii) PEST motif, or combinations thereof.

In another preferred embodiment, a composition comprises an isolated cell expressing a polypeptide comprising a suppressor of cytokine signaling (SOCS) protein and a cell penetrating domain, wherein the suppressor of cytokine signaling (SOCS) protein lacks: (i) a functional C-terminal SOCS box, (ii) PEST motif or combinations thereof.

In addition to the known functional SOCS variants, derivatives of the SOCS proteins can also function in the disclosed methods and compositions. Protein variants and derivatives are well understood to those of skill in the art and can involve amino acid sequence modifications. For example, amino acid sequence modifications typically fall into one or more of three classes: substitutional, insertional or deletional variants. Insertions include amino and/or carboxyl terminal fusions as well as intrasequence insertions of single or multiple amino acid residues. Insertions ordinarily will be smaller insertions than those of amino or carboxyl terminal fusions, for example, on the order of one to four residues. Deletions are characterized by the removal of one or more amino acid residues from the protein sequence. These variants ordinarily are prepared by site specific mutagenesis of nucleotides in the DNA encoding the protein, thereby producing DNA encoding the variant, and thereafter expressing the DNA in recombinant cell culture. Techniques for making substitution mutations at predetermined sites in DNA having a known sequence are well known, for example M13 primer mutagenesis and PCR mutagenesis. Amino acid substitutions are typically of single residues, but can occur at a number of different locations at once; insertions usually will be on the order of about from 1 to 10 amino acid residues; and deletions will range about from 1 to 30 residues. Deletions or insertions preferably are made in adjacent pairs, i.e. a deletion of 2 residues or insertion of 2 residues. Substitutions, deletions, insertions or any combination thereof can be combined to arrive at a final construct. Substitutional variants are those in which at least one residue has been removed and a different residue inserted in its place.

Substantial changes in function can be made by selecting substitutions that differ more significantly in their effect on maintaining (a) the structure of the polypeptide backbone in the area of the substitution, for example as a sheet or helical conformation, (b) the charge or hydrophobicity of the molecule at the target site or (c) the bulk of the side chain. The substitutions which in general are expected to produce the greatest changes in the protein properties will be those in which (a) a hydrophilic residue, e.g. seryl or threonyl, is substituted for (or by) a hydrophobic residue, e.g. leucyl, isoleucyl, phenylalanyl, valyl or alanyl; (b) a cysteine or proline is substituted for (or by) any other residue; (c) a residue having an electropositive side chain, e.g., lysyl, arginyl, or histidyl, is substituted for (or by) an electronegative residue, e.g., glutamyl or aspartyl; or (d) a residue having a bulky side chain, e.g., phenylalanine, is substituted for (or by) one not having a side chain, e.g., glycine, in this case, (e) by increasing the number of sites for sulfation and/or glycosylation.

The replacement of one amino acid residue with another that is biologically and/or chemically similar is known to those skilled in the art as a conservative substitution. For example, a conservative substitution would be replacing one hydrophobic residue for another, or one polar residue for another. The substitutions include combinations such as, for example, Gly, Ala; Val, Ile, Leu; Asp, Glu; Asn, Gln; Ser, Thr; Lys, Arg; and Phe, Tyr. Such conservatively substituted variations of each explicitly disclosed sequence are included within the mosaic polypeptides provided herein.

Substitutional or deletional mutagenesis can be employed to insert sites for N-glycosylation (Asn-X-Thr/Ser) or O-glycosylation (Ser or Thr). Deletions of cysteine or other labile residues also can be desirable. Deletions or substitutions of potential proteolysis sites, e.g. Arg, is accomplished for example by deleting one of the basic residues or substituting one by glutaminyl or histidyl residues.

Certain post-translational derivatizations are the result of the action of recombinant host cells on the expressed polypeptide. Glutaminyl and asparaginyl residues are frequently post-translationally deamidated to the corresponding glutamyl and asparyl residues. Alternatively, these residues are deamidated under mildly acidic conditions. Other post-translational modifications include hydroxylation of proline and lysine, phosphorylation of hydroxyl groups of seryl or threonyl residues, methylation of the o-amino groups of lysine, arginine, and histidine side chains (T. E. Creighton, Proteins: Structure and Molecular Properties, W. H. Freeman & Co., San Francisco pp 79-86 [1983]), acetylation of the N-terminal amine and, in some instances, amidation of the C-terminal carboxyl.

It is understood that one way to define the variants and derivatives of the disclosed proteins herein is through defining the variants and derivatives in terms of homology/identity to specific known sequences. For example, SOCS variants can have at least 40% or 45% or 50% or 55% or 60% or 65% 70% or 75% or 80% or 85% or 90% or 95% homology to the stated sequence. Those of skill in the art readily understand how to determine the homology of two proteins.

Another way of calculating homology can be performed by published algorithms. Optimal alignment of sequences for comparison can be conducted by the local homology algorithm of Smith and Waterman Adv. Appl. Math. 2: 482 (1981), by the homology alignment algorithm of Needleman and Wunsch, J. Mol. Biol. 48: 443 (1970), by the search for similarity method of Pearson and Lipman, Proc. Natl. Acad. Sci. U.S.A. 85: 2444 (1988), by computerized implementations of these algorithms (GAP, BESTFIT, FASTA, and TFASTA in the Wisconsin Genetics Software Package, Genetics Computer Group, 575 Science Dr., Madison, Wis.), or by inspection.

As this specification discusses various proteins and protein sequences it is understood that the nucleic acids that can encode those protein sequences are also disclosed. This would include all degenerate sequences related to a specific protein sequence, i.e. all nucleic acids having a sequence that encodes one particular protein sequence as well as all nucleic acids, including degenerate nucleic acids, encoding the disclosed variants and derivatives of the protein sequences. Thus, while each particular nucleic acid sequence may not be written out herein, it is understood that each and every sequence is in fact disclosed and described herein through the disclosed protein sequence.

It is understood that there are numerous amino acid and peptide analogs which can be incorporated into the disclosed compositions. These amino acids can readily be incorporated into polypeptide chains by charging tRNA molecules with the amino acid of choice and engineering genetic constructs that utilize, for example, amber codons, to insert the analog amino acid into a peptide chain in a site specific way (Thorson et al., Methods in Mol. Biol. 77:43-73 (1991), Zoller, Current Opinion in Biotechnology, 3:348-354 (1992); Ibba, Biotechnology & Genetic Engineering Reviews 13:197-216 (1995), Cahill et al., TIBS, 14 (10):400-403 (1989) all of which are herein incorporated by reference at least for material related to amino acid analogs).

Molecules can be produced that resemble peptides, but which are not connected via a natural peptide linkage. For example, linkages for amino acids or amino acid analogs can include CH₂NH—, —CH₂S—, —CH₂—CH₂, —CH═CH— (cis and trans), —COCH₂—, —CH(OH)CH₂—, and —CHH₂SO— (These and others can be found in Spatola, A. F. in Chemistry and Biochemistry of Amino Acids, Peptides, and Proteins, B. Weinstein, eds., Marcel Dekker, New York, p. 267 (1983); Spatola, A. F., Vega Data (March 1983), Vol. 1, Issue 3, Peptide Backbone Modifications (general review); Morley, Trends Pharm Sci (1980) pp. 463-468; Hudson, D. et al., Int J Pept Prot Res 14:177-185 (1979) (—CH₂NH—, CH₂CH₂—); Spatola et al. Life Sci 38:1243-1249 (1986) (—CHH₂—S); Hann J. Chem. Soc Perkin Trans. 1307-314 (1982) (—CH—CH—, cis and trans); Almquist et al. J. Med. Chem. 23:1392-1398 (1980) (—COCH₂—); Jennings-White et al. Tetrahedron Lett 23:2533 (1982) (—COCH₂—); Szelke et al. European Appln, EP 45665 CA (1982): 97:39405 (1982) (—CH(OH)CH₂—); Holladay et al. Tetrahedron. Lett 24:4401-4404 (1983) (—C(OH)CH₂—); and Hruby Life Sci 31:189-199 (1982) (—CH₂—S—); each of which is incorporated herein by reference. A particularly preferred non-peptide linkage is —CH₂NH—. It is understood that peptide analogs can have more than one atom between the bond atoms, such as β-alanine, γ-aminobutyric acid, and the like.

Amino acid analogs and analogs and peptide analogs often have enhanced or desirable properties, such as, more economical production, greater chemical stability, enhanced pharmacological properties (half-life, absorption, potency, efficacy, etc.), altered specificity (e.g., a broad-spectrum of biological activities), reduced antigenicity, and others.

D-amino acids can be used to generate more stable peptides, because D amino acids are not recognized by peptidases and such. Systematic substitution of one or more amino acids of a consensus sequence with a D-amino acid of the same type (e.g., D-lysine in place of L-lysine) can be used to generate more stable peptides. Cysteine residues can be used to cyclize or attach two or more peptides together. This can be beneficial to constrain peptides into particular conformations. (Rizo and Gierasch Ann. Rev. Biochem. 61:387 (1992), incorporated herein by reference).

### Prevention and Treatment Using SOCS Compositions

In a preferred embodiment, the SOCS molecules are administered to patients suffering from diseases or disorders associated with abnormal signaling of cytokines or preventing diseases or disorders associated with the abnormal signaling of cytokines. The “abnormal” signaling means that some or all cytokines are continuously active resulting in immune and other cells being continuously acted upon producing deviations in the cellular responses. The term “abnormal” also is applied to those cases where some or all cytokines are not being active and their effects on other cells deviates from a normal cellular activity. Since the cytokines are key in regulating the immune response, such diseases or disorders associated with cytokine signaling, comprise without limitation: autoimmune diseases or disorders, cardiovascular diseases or disorders, neurological diseases or disorders, neuroinflammatory diseases or disorders, inflammatory eye disorder, inflammatory skin disorders, cancer including leukemia and lymphoma, neurodegenerative diseases or disorders, inflammatory diseases or disorders, liver diseases or disorders, pancreas or kidney diseases or disorders, diabetes, inflammatory disorders of placenta and amnion that contribute to loss of pregnancy or prematurity, other diseases or disorders mediated by inflammation, foreign antigens (e.g. virus, bacteria, etc) apoptosis, or aberrant proliferation.

In another preferred embodiment, the pharmaceutical composition comprises a recombinant polypeptide having a suppressor of cytokine signaling (SOCS) protein and a cell penetrating domain, wherein the suppressor of cytokine signaling (SOCS) peptide lacks: (i) a functional C-terminal SOCS box, (ii) PEST domain or motif, or combinations thereof. The SOCS box further comprises one or more mutations, substitutions, deletions or combinations thereof. In another embodiment, the C-terminal SOCS box is deleted. In another embodiment, the PEST domain or motif comprises one or more mutations, substitutions, deletions, or combinations thereof. In another embodiment, the PEST domain is deleted.

In preferred embodiments, the SOCS protein is selected from the group consisting of SOCS 1, SOCS 2, SOCS 3, SOCS 4, SOCS 5, SOCS 6, SOCS 7, variants mutants, analogs, fragments, species or combinations thereof.

In another preferred embodiment, the SOCS comprises a nucleic acid which encodes the SOCS protein and or the CP protein.

In another preferred embodiment, a method of preventing or treating a disease or disorder associated with cytokine signaling comprises increasing the half-life (t_{1/2}) of a suppressor of cytokine signaling (SOCS) peptides in vitro or in vivo, comprising a recombinant polypeptide of a suppressor of cytokine signaling (SOCS) peptide and a cell penetrating domain, wherein the suppressor of cytokine signaling (SOCS) peptide lacks: (i) a functional C-terminal SOCS box, (ii) PEST domain, or combinations thereof.

In another preferred embodiment, a method of modulating cytokine signaling in vivo, comprises administering to a patient, an effective amount of a recombinant protein a suppressor of cytokine signaling (SOCS) peptides in vitro or in vivo, comprising a recombinant polypeptide comprising a suppressor of cytokine signaling (SOCS) peptide and a cell penetrating domain, wherein the suppressor of cytokine signaling (SOCS) peptide lacks: (i) a functional C-terminal SOCS box, (ii) PEST domain, or combinations thereof.

In another preferred embodiment, a method of modulating cytokine signaling in vivo, comprises administering to a patient, an effective amount of a recombinant protein a suppressor of cytokine signaling (SOCS) peptides in vitro or in vivo, comprising an isolated nucleic acid expressing a recombinant polypeptide comprising a suppressor of cytokine signaling (SOCS) peptide and a cell penetrating domain, wherein the suppressor of cytokine signaling (SOCS) peptide lacks: (i) a functional C-terminal SOCS box, (ii) PEST domain, or combinations thereof.

In another preferred embodiment, a method of modulating an immune response comprises administering to a patient in need thereof, a therapeutically effective amount of a cytokine modulator in a pharmaceutical composition. Preferably, a cytokine modulator comprises a recombinant polypeptide having a suppressor of cytokine signaling (SOCS) protein and a cell penetrating domain, wherein the suppressor of cytokine signaling (SOCS) protein lacks: (i) a functional C-terminal SOCS box, (ii) PEST domain, or combinations thereof.

In another preferred embodiment, a cytokine modulator comprises a nucleic acid encoding for a polypeptide having a suppressor of cytokine signaling (SOCS) protein and a cell penetrating domain, wherein the suppressor of cytokine signaling (SOCS) protein lacks: (i) a functional C-terminal SOCS box, (ii) PEST domain, or combinations thereof.

In another preferred embodiment, a cytokine modulator comprises a cell expressing a polypeptide comprising a suppressor of cytokine signaling (SOCS) protein and a cell penetrating domain, wherein the suppressor of cytokine signaling (SOCS) protein lacks: (i) a functional C-terminal SOCS box, (ii) PEST domain, or combinations thereof.

In preferred embodiments, the compositions can be administered as the recombinant protein, a nucleic acid expressing the recombinant protein, an isolated cell expressing the recombinant protein. The cells can be autologous, heterologous, syngeneic, haplotyped matched, cell lines, stem cells and the like.

In another preferred embodiment, a method of protecting a cell in vivo or in vitro from undergoing apoptosis, comprises contacting a cell in vitro or in vivo with a therapeutically effective amount of a cytokine modulator in a pharmaceutical composition.

### Administration of Compositions

Delivery of a therapeutic SOCS polypeptide or polynucleotide to appropriate cells can be effected ex vivo, in situ, or in vivo by use of any suitable approach known in the art. For example, for in vivo therapy, a nucleic acid encoding the desired SOCS molecule, either alone or in conjunction with a vector, liposome, or precipitate may be injected directly into the subject, and in some embodiments, may be injected at the site where the expression of the specific binding agent or antibody compound is desired. For ex vivo treatment, the subject's cells are removed, the nucleic acid is introduced into these cells, and the modified cells are returned to the subject either directly or, for example, encapsulated within porous membranes which are implanted into the patient. See, e.g. U.S. Pat. Nos. 4,892,538 and 5,283,187.

There are a variety of techniques available for introducing nucleic acids into viable cells. The techniques vary depending upon whether the nucleic acid is transferred into cultured cells in vitro, or in vivo in the cells of the intended host. Techniques suitable for the transfer of nucleic acid into mammalian cells in vitro include the use of liposomes, electroporation, microinjection, cell fusion, chemical treatments, DEAE-dextran, and calcium phosphate precipitation. Other in vivo nucleic acid transfer techniques include transfection with viral vectors (such as adenovirus, Herpes simplex I virus, adeno-associated virus or retrovirus) and lipid-based systems. The nucleic acid and transfection agent are optionally associated with a microparticle. Exemplary transfection agents include calcium phosphate or calcium chloride co-precipitation, DEAE-dextran-mediated transfection, quaternary ammonium amphiphile DOTMA ((dioleoyloxypropyl)trimethylammonium bromide, commercialized as Lipofectin by GIBCO-BRL)) (Feigner et al, (1987) Proc. Natl. Acad. Sci. USA 84, 7413-7417; Malone et al. (1989) Proc. Natl. Acad. Sci. USA 86 6077-6081); lipophilic glutamate diesters with pendent trimethylammonium heads (Ito et al. (1990) Biochem. Biophys. Acta 1023, 124-132); the metabolizable parent lipids such as the cationic lipid dioctadecylamido glycylspermine (DOGS, Transfectam, Promega) and dipalmitoylphosphatidyl ethanolamylspermine (DPPES) (J. P. Behr (1986) Tetrahedron Lett. 27, 5861-5864; J. P. Behr et al. (1989) Proc. Natl. Acad. Sci. USA 86, 6982-6986); metabolizable quaternary ammonium salts (DOTB, N-(1-[2,3-dioleoyloxy]propyl)-N,N,N-trimethylammonium methylsulfate (DOTAP) (Boehringer Mannheim), polyethyleneimine (PEI), dioleoyl esters, ChoTB, ChoSC, DOSC) (Leventis et al. (1990) Biochim. Inter. 22, 235-241); 3beta[N—(N′,N′-dimethylaminoethane)-carbamoyl]cholesterol (DC-Chol), dioleoylphosphatidyl ethanolamine (DOPE)/3β[N—(N′,N′-dimethylaminoethane)-carbamoyl]cholesterolDC-Chol in one to one mixtures (Gao et al., (1991) Biochim. Biophys. Acta 1065, 8-14), spermine, spermidine, lipopolyamines (Behr et al., Bioconjugate Chem, 1994, 5: 382-389), lipophilic polylysines (LPLL) (Zhou et al., (1991) Biochim. Biophys. Acta 939, 8-18), [[(1,1,3,3-tetramethylbutyl)cre-soxy]ethoxy]ethyl]dimethylbenzylammonium hydroxide (DEBDA hydroxide) with excess phosphatidylcholine/cholesterol (Ballas et al., (1988) Biochim. Biophys. Acta 939, 8-18), cetyltrimethylammonium bromide (CTAB)/DOPE mixtures (Pinnaduwage et al, (1989) Biochim. Biophys. Acta 985, 33-37), lipophilic diester of glutamic acid (TMAG) with DOPE, CTAB, DEBDA, didodecylammonium bromide (DDAB), and stearylamine in admixture with phosphatidylethanolamine (Rose et al., (1991) Biotechniques 10, 520-525), DDAB/DOPE (TransfectACE, GIBCO BRL), and oligogalactose bearing lipids. Exemplary transfection enhancer agents that increase the efficiency of transfer include, for example, DEAE-dextran, polybrene, lysosome-disruptive peptide (Ohmori N I et al, Biochem Biophys Res Commun Jun. 27, 1997; 23 5 (3):726-9), chondroitan-based proteoglycans, sulfated proteoglycans, polyethylenimine, polylysine (Pollard H et al. J Biol Chem, 1998 273 (13):7507-11), integrin-binding peptide, linear dextran nonasaccharide, glycerol, cholesteryl groups tethered at the 3′-terminal internucleoside link of an oligonucleotide (Letsinger, R. L. 1989 Proc Natl Acad Sci USA 86: (17):6553-6), lysophosphatide, lysophosphatidylcholine, lysophosphatidylethanolamine, and 1-oleoyl lysophosphatidylcholine.

In some situations it may be desirable to deliver the nucleic acid with an agent that directs the nucleic acid-containing vector to target cells. Such “targeting” molecules include antibodies specific for a cell-surface membrane protein on the target cell, or a ligand for a receptor on the target cell. Where liposomes are employed, proteins which bind to a cell-surface membrane protein associated with endocytosis may be used for targeting and/or to facilitate uptake. Examples of such proteins include capsid proteins and fragments thereof tropic for a particular cell type, antibodies for proteins which undergo internalization in cycling, and proteins that target intracellular localization and enhance intracellular half-life. In other embodiments, receptor-mediated endocytosis can be used. Such methods are described, for example, in Wu et al., 1987 or Wagner et al., 1990. For review of the currently known gene marking and gene therapy protocols, see Anderson 1992. See also WO 93/25673 and the references cited therein. For additional reviews of gene therapy technology, see Friedmann, Science, 244: 1275-1281 (1989); Anderson, Nature, supplement to vol. 392, no 6679, pp. 25-30 (1998); and Miller, Nature, 357: 455-460 (1992).

The compositions or agents identified by the methods described herein may be administered to animals including human beings in any suitable formulation. For example, the compositions for modulating cytokine signaling may be formulated in pharmaceutically acceptable carriers or diluents such as physiological saline or a buffered salt solution. Suitable carriers and diluents can be selected on the basis of mode and route of administration and standard pharmaceutical practice. A description of exemplary pharmaceutically acceptable carriers and diluents, as well as pharmaceutical formulations, can be found in Remington's Pharmaceutical Sciences, a standard text in this field, and in USP/NF. Other substances may be added to the compositions to stabilize and/or preserve the compositions.

The compositions of the disclosure may be administered to animals by any conventional technique. The compositions may be administered directly to a target site by, for example, surgical delivery to an internal or external target site, or by catheter to a site accessible by a blood vessel. Other methods of delivery, e.g., liposomal delivery or diffusion from a device impregnated with the composition, are known in the art. The compositions may be administered in a single bolus, multiple injections, or by continuous infusion (e.g., intravenously). For parenteral administration, the compositions are preferably formulated in a sterilized pyrogen-free form.

The compounds can be administered with one or more therapies. The chemotherapeutic agents may be administered under a metronomic regimen. As used herein, “metronomic” therapy refers to the administration of continuous low-doses of a therapeutic agent.

Dosage, toxicity and therapeutic efficacy of such compounds can be determined by standard pharmaceutical procedures in cell cultures or experimental animals, e.g., for determining the LD₅₀ (the dose lethal to 50% of the population) and the ED₅₀ (the dose therapeutically effective in 50% of the population). The dose ratio between toxic and therapeutic effects is the therapeutic index and it can be expressed as the ratio LD₅₀/ED₅₀. Compounds that exhibit high therapeutic indices are preferred. While compounds that exhibit toxic side effects may be used, care should be taken to design a delivery system that targets such compounds to the site of affected tissue in order to minimize potential damage to uninfected cells and, thereby, reduce side effects.

The data obtained from the cell culture assays and animal studies can be used in formulating a range of dosage for use in humans. The dosage of such compounds lies preferably within a range of circulating concentrations that include the ED₅₀ with little or no toxicity. The dosage may vary within this range depending upon the dosage form employed and the route of administration utilized. For any compound used in the method of the disclosure, the therapeutically effective dose can be estimated initially from cell culture assays. A dose may be formulated in animal models to achieve a circulating plasma concentration range that includes the IC₅₀ (i.e., the concentration of the test compound which achieves a half-maximal inhibition of symptoms) as determined in cell culture. Such information can be used to more accurately determine useful doses in humans. Levels in plasma may be measured, for example, by high performance liquid chromatography.

As defined herein, a therapeutically effective amount of a compound (i.e., an effective dosage) means an amount sufficient to produce a therapeutically (e.g., clinically) desirable result. The compositions can be administered one from one or more times per day to one or more times per week; including once every other day. The skilled artisan will appreciate that certain factors can influence the dosage and timing required to effectively treat a subject, including but not limited to the severity of the disease or disorder, previous treatments, the general health and/or age of the subject, and other diseases present. Moreover, treatment of a subject with a therapeutically effective amount of the compounds of the disclosure can include a single treatment or a series of treatments.

### Formulations

While it is possible for a composition to be administered alone, it is preferable to present it as a pharmaceutical formulation. The active ingredient may comprise, for topical administration, from 0.001% to 10% w/w, e.g., from 1% to 2% by weight of the formulation, although it may comprise as much as 10% w/w but preferably not in excess of 5% w/w and more preferably from 0.1% to 1% w/w of the formulation. The topical formulations of the present disclosure, comprise an active ingredient together with one or more acceptable carrier(s) therefor and optionally any other therapeutic ingredients(s). The carrier(s) must be “acceptable” in the sense of being compatible with the other ingredients of the formulation and not deleterious to the recipient thereof.

Formulations suitable for topical administration include liquid or semi-liquid preparations suitable for penetration through the skin to the site of where treatment is required, such as liniments, lotions, creams, ointments or pastes, and drops suitable for administration to the eye, ear, or nose. Drops according to the present disclosure may comprise sterile aqueous or oily solutions or suspensions and may be prepared by dissolving the active ingredient in a suitable aqueous solution of a bactericidal and/or fungicidal agent and/or any other suitable preservative, and preferably including a surface active agent. The resulting solution may then be clarified and sterilized by filtration and transferred to the container by an aseptic technique. Examples of bactericidal and fungicidal agents suitable for inclusion in the drops are phenylmercuric nitrate or acetate (0.002%), benzalkonium chloride (0.01%) and chlorhexidine acetate (0.01%). Suitable solvents for the preparation of an oily solution include glycerol, diluted alcohol and propylene glycol.

Lotions according to the present disclosure include those suitable for application to the skin or eye. An eye lotion may comprise a sterile aqueous solution optionally containing a bactericide and may be prepared by methods similar to those for the preparation of drops. Lotions or liniments for application to the skin may also include an agent to hasten drying and to cool the skin, such as an alcohol or acetone, and/or a moisturizer such as glycerol or an oil such as castor oil or arachis oil.

Creams, ointments or pastes according to the present disclosure are semi-solid formulations of the active ingredient for external application. They may be made by mixing the active ingredient in finely-divided or powdered form, alone or in solution or suspension in an aqueous or non-aqueous fluid, with the aid of suitable machinery, with a greasy or non-greasy basis. The basis may comprise hydrocarbons such as hard, soft or liquid paraffin, glycerol, beeswax, a metallic soap; a mucilage; an oil of natural origin such as almond, corn, arachis, castor or olive oil; wool fat or its derivatives, or a fatty acid such as stearic or oleic acid together with an alcohol such as propylene glycol or macrogels. The formulation may incorporate any suitable surface active agent such as an anionic, cationic or non-ionic surface active such as sorbitan esters or polyoxyethylene derivatives thereof. Suspending agents such as natural gums, cellulose derivatives or inorganic materials such as silicaceous silicas, and other ingredients such as lanolin, may also be included.

While various embodiments of the present disclosure have been described above, it should be understood that they have been presented by way of example only, and not limitation. Numerous changes to the disclosed embodiments can be made in accordance with the disclosure herein without departing from the spirit or scope of the disclosure. Thus, the breadth and scope of the present disclosure should not be limited by any of the above described embodiments.

Embodiments of the disclosure may be practiced without the theoretical aspects presented. Moreover, the theoretical aspects are presented with the understanding that Applicants do not seek to be bound by the theory presented. It will be appreciated that those skilled in the art, upon consideration of this disclosure, may make modifications and improvements within the spirit and scope of the disclosure. The following non-limiting examples are illustrative of the disclosure.

All documents mentioned herein are incorporated herein by reference. All publications and patent documents cited in this application are incorporated by reference for all purposes to the same extent as if each individual publication or patent document were so individually denoted. By their citation of various references in this document, Applicants do not admit any particular reference is “prior art” to their disclosure.

### EXAMPLES

The following examples are included to demonstrate preferred embodiments of the invention. It should be appreciated by those of skill in the art that the techniques disclosed in the examples which follow represent techniques discovered by the inventors to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit or scope of the invention. The following Examples are offered by way of illustration and not by way of limitation.

### Example 1

### Extended Anti-Inflammatory Action of a Degradation-Resistant Mutant of Cell-Penetrating Suppressor of Cytokine Signaling

### Experimental Procedures

Cell Culture—RAW 264.7 macrophages (a murine peritoneal macrophage cell line), and AMJ2.C8 macrophages (a murine alveolar macrophage cell line) were cultured in DMEM (Mediatech) supplemented with 10% heat-inactivated Fetal Bovine Serum (FBS) (Atlanta Biologicals), and 100 Units/ml Penicillin/100 μg/ml Streptomycin (Mediatech) under standard conditions. Primary bone marrow-derived macrophages (BMDM) were prepared as follows: Female 8 week old C3H/HeJ mice were sacrificed and their femurs removed with the hip and knee joints intact. Femurs were sterilized by rinsing in 70% ethanol and the hip and knee joints removed. Bone marrow cells were collected by inserting a 27 gauge needle into the open end of bone and flushing the marrow with 10 ml DMEM. The cell suspension was filtered through a 70 μm nylon membrane and pelleted by centrifugation. Cells were resuspended in DMEM supplemented with 10% FBS, 10 mM HEPES (Mediatech), 50 Units/ml Penicillin/50 μg/ml Streptomycin and 20% L-cell conditioned media (LCM) to direct differentiation of naïve bone marrow cells to macrophages. On day 3 of culture, fresh medium was replaced. On day 7, the purity of the macrophage culture was determined to be ≧95% as measured by FACS analysis gating on macrophage specific cell surface markers. BMDM were then plated and used in the experiments as indicated.

SOCS3 plasmid constructs—Full-length murine SOCS3 was provided by M. Shong, at Chungnam National University in Korea. The hydrophobic Membrane Translocating Motif (MTM) was derived from a hydrophobic region of the signal sequence of Fibroblast Growth Factor 4 (Hawiger, J. (1999) Curr. Opin. Chem. Biol. 3, 89-94). The MTM and/or 6× Histidine (His) Tag were added to SOCS3 using standard PCR conditions. The following primers were used to engineer full-length and SOCS3 deletion mutant constructs: CP-SOCS3ΔSB (reverse)—5′ GA GGA TTC TTA GGA GGA GAG AGG TCG GCT CAG TAC CAG C 3′ (SEQ ID NO: 4); CP-SOCS3ΔSB (forward)—5′ CG GGA TCC GCC ATG GCC CAT CAT CAC CAT CAC CAT AAT GCC CAT ACC GGT GCA GCT GTG CTT CTC CCT GTG C 3′ (SEQ ID NO: 5); CP-SOCS3 (reverse)—5′ GA GAA TTC TTA AAG TGG AGC ATC ATA CTG ATC CAG G 3′ (SEQ ID NO: 6); CP-SOCS3 (forward)—5′ CG GGA TCC GCC ATG GCC CAT CAT CAC CAT CAC CAT AAT GCC CAT ACC GGT GCA GCT GTG CTT CTC CCT GTG C 3′ (SEQ ID NO: 7); non CP-SOCS3 (reverse)—5′ GA GAA TTC TTA AAG TGG AGC ATC ATA CTG ATC CAG G 3′ (SEQ ID NO: 8); non CP-SOCS3 (forward)—5′ CG GGA TCC GCC ATG GCC CAT CAT CAC CAT CAC CAT AAT GCC CAT ACC GGT ATG GTC ACC CAC AGC AAG TTT CCC G 3′ (SEQ ID NO: 9).

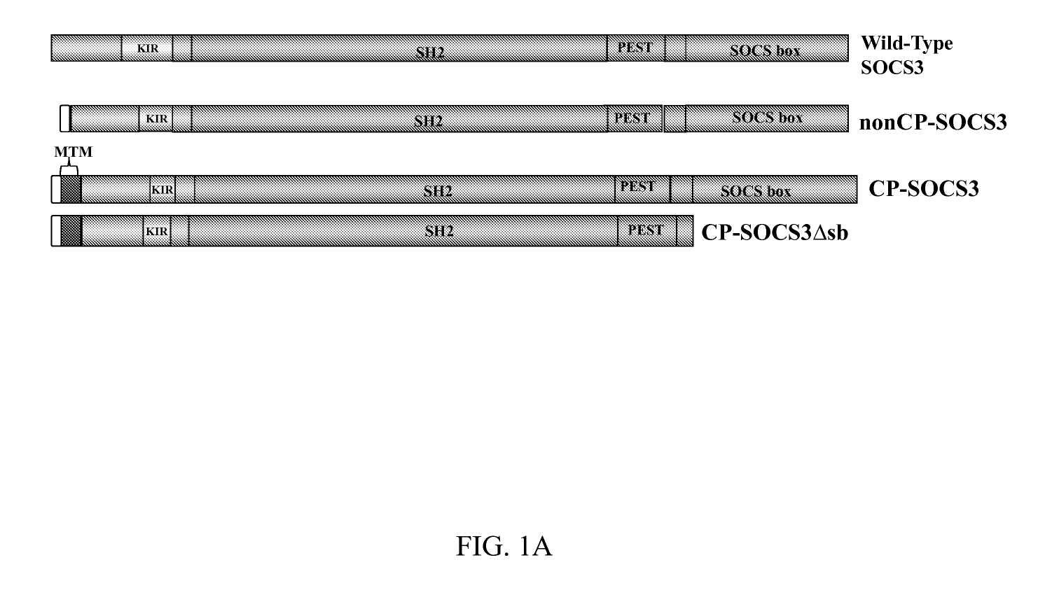

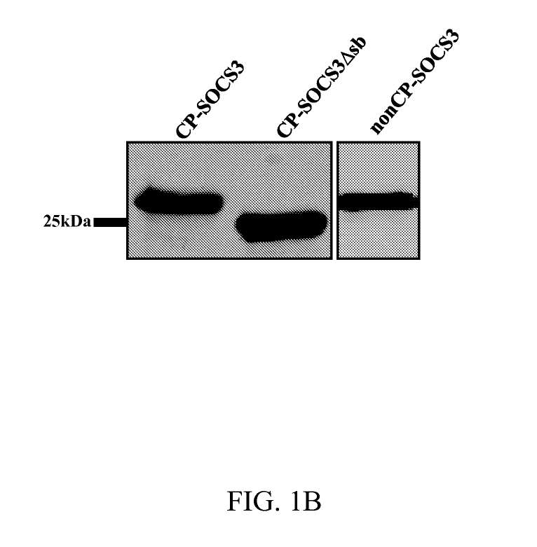

Production of Recombinant Proteins—Non-CP-SOCS3, CP-SOCS3, and CP-SOCS3ΔSB constructs were cloned into the pET21a (+) vector using standard engineering techniques. Plasmid constructs were then transformed into BL21 (DE3) RP E. coli cells and positive clones identified and verified by DNA sequencing. Positive clones were grown up in liquid Luria Broth cultures, containing ampicillin. Expression of proteins was induced by incubating bacterial cells for 3 h with 0.5 mM IPTG. After 3 h, cells were collected by centrifugation, cell pellet weighed, and resuspended in 5 ml per gram weight of resuspension buffer (100 mM sodium phosphate—monobasic, 10 mM Tris base, 8M Urea, pH 8.0). Bacterial cells were lysed by sonication and recombinant SOCS3 proteins were purified with histidine affinity column by FPLC (AKTA Purifyer, GE Healthcare, Piscataway, N.J.) using Ni-NTA resin (Qiagen). The protein was then refolded through a 2-step dialysis to remove denaturant (DiGiandomenico, A., Wylezinski, L. S., and Hawiger, J. (2009) Sci. Signal. 2, ra37). The final diluent was DMEM supplemented with penicillin/streptomycin (concentrations listed above). Identification of purified proteins was done by western blot analysis (FIG. 1B). Protein concentration of CP-SOCS3 and CP-SOCS3ΔSB was determined by the Bradford Assay. Proteins were stored at −40° C. (long term), or at 4° C. (short term) until used in assays.

Half-life Determination—The half-life of endogenous SOCS3 was determined as follows: RAW cells were stimulated with 100 Units/ml IFN-γ (EMD Biosciences) and 250 ng/ml LPS (Sigma) for 4 h to induce SOCS3 expression. This 4 h post-stimulation time point represents t=0 h, from which cells were harvested following the indicated treatments. In the designated experiments, cells were treated with 15 μg/ml cycloheximide (Sigma) 4 h after LPS/IFN-γ stimulation, 1 μM Epoxomicin (Sigma) 2 h after LPS/IFN-γ stimulation, and 40 μg/ml Calpeptin (VWR) was added 3.5 h post-stimulation. Following treatment, cells were harvested at the indicated time points, and lysed with 1×CBA lysis buffer (BD Biosciences). Samples were then heated at 100° C. for 20 min, and centrifuged to clear lysates of cellular debris. Supernatants were then snap frozen and stored at −40° C. until immunoblotting was performed. The half-life of recombinant full-length CP-SOCS3 or CP-SOCS3ΔSB proteins was determined as follows: RAW cells were stimulated for 1 h with 100 Units/ml IFN-γ and 250 ng/ml LPS, and during the same time interval, cells were treated with 1 μM of CP-SOCS3 or CP-SOCS3ΔSB while incubating at 37° C. Cells were rinsed 3 times with PBS (Mediatech) warmed to 37° C., and treated with 10 μg/ml of proteinase K (Sigma) for 15 min to degrade any non-internalized protein attached to the outside of the cell. Cells were rinsed again 3 times with warm PBS, and incubated with DMEM supplemented with FBS and penicillin/streptomycin (see above). At the indicated time intervals, cells were harvested, lysed, and prepared for immunoblotting, as described above. The medium was also collected, snap frozen at −40° C., and later analyzed by immunoblot for SOCS3 protein.

Immunoblotting—Lysates collected from half-life experiments, cytokine/chemokine assays, and STAT1 phosphorylation assays were mixed with 6×SDS loading buffer and boiled at 100° C. for 5 min. Samples were resolved on 12% SDS-PAGE, and transferred to nitrocellulose membrane. Membranes were probed with rabbit anti-SOCS3 (Ab-cam), which recognizes a C-terminal epitope of SOCS3, or rabbit anti-6×His Tag antibody (Rockland), or mouse anti-pSTAT1 (Y701) antibody (BD Biosciences) and mouse anti-β-actin (Ab-cam) according to manufacturer's protocol. Bands were developed using the following secondary antibodies: donkey anti-rabbit IR Dye 800 (LI-COR biosciences), and donkey anti-mouse IR Dye 680 (LI-COR biosciences). Probing was performed according to the individual manufacturer's protocol. Bands were visualized using Licor's Odyssey Infrared Imaging System. SOCS3 and 6×His Tag protein bands were normalized against the levels of expressed β-actin. Quantification and analysis of bands were performed using Odyssey software (version 3.0).

Cytokine and Chemokine Analysis—Cultured primary cells (BMDM) or established cell line (AMJ2.C8 macrophages), that were plated the previous day with 1×106 cells/well in a 96-well plate, were pre-treated for 1 h with 10 μM of the indicated cell-penetrating protein (CP-SOCS3 or CP-SOCS3ΔSB). Medium containing added protein was then removed and replaced with fresh DMEM supplemented with DMEM supplemented with FBS, HEPES, penicillin/streptomycin and LCM (for BMDM) or FBS and penicillin/streptomycin (for AMJ2.C8 macrophages). At 6 h or 24 h after CP-SOCS3 or CP-SOCS3ΔSB treatment, cells were stimulated with 100 Units/ml IFN-γ and 500 ng/ml LPS for 6 h. Supernatants (50 μl) were collected before CP-proteins or diluent were added (0 h), and at the end of the 6 h activation with proinflammatory agonists. This means that test samples were analyzed at 12 h and 30 h, following CP-protein treatment. Samples were assayed for the presence of inflammatory cytokines/chemokine using the Mouse Inflammatory Cytokine Bead Array (CBA) Kit (BD Biosciences). Cytokine analysis was performed according to manufacturer's protocol and flow cytometry was performed using BD FACS Calibur. Data acquisition and analysis were done using BD Pro Cell Quest Software and BD 6-bead analysis software. Cells were also harvested at the end of the stimulation period (12 h & 30 h), and lysates were immunoblotted to determine the level of cell-penetrating proteins remaining in the cell. Lysates were prepared for immunoblotting as described above. Additionally, at the end of the stimulation period, cell viability was ≧95% after staining cells with fluorescein and ethidium bromide to detect live and dead cells.

STAT 1 Phosphorylation Assay—BMDM or AMJ2.C8 macrophages, (plated the previous day with 5×106 cells/well in a 12-well plate) were pre-treated for 1 h with 10 μM of CP-SOCS3 or 10 μM CP-SOCS3ΔSB. Medium containing the added protein was removed and replaced with DMEM supplemented with FBS, HEPES, penicillin/streptomycin and LCM (for BMDM) or DMEM supplemented with FBS and penicillin/streptomycin (for AMJ2.C8 macrophages). Cells were then stimulated with 100 Units/ml IFN-γ and 0.2 μg/ml LPS for 15 minutes. Cells were lysed with 1×CBA lysis buffer. Lysates were assayed to determine the levels of phosphorylated STAT1 using the Phospho Stat1 (Y701) Flex Set Cytometric Bead Array (BD Biosciences) according to the manufacturer's protocol. Flow cytometry was performed using BD FACS Calibur, and data acquisition and analysis was performed using BD Pro Cell Quest Software and BD 4-Bead analysis software. BMDM lysates were also subjected to Western blot analysis to verify phosphorylated STAT1 levels. Lysates were prepared for immunoblotting as described above. At the end of the stimulation period, cell viability was ≧95% after staining cells with fluorescein and ethidium bromide to detect live and dead cells.

FITC Labeling of Proteins—Recombinant SOCS3 proteins were labeled with FITC (Fluorescein isothiocyanate) (Pierce) as previously reported (Jo, D., Liu, D., Yao, S., Collins, R. D., and Hawiger, J. (2005) Nat. Med. 11, 892-898), and briefly described here. Approximately 1 mg of CP-SOCS3 or CP-SOCS3ΔSB was added to 0.5 ml conjugation buffer (0.4M Carbonate, 0.1M Bicarbonate at final concentration, pH 9.0). FITC was dissolved into dimethylformamide (DMF) to a final concentration of 30 mg/ml. A 2-fold excess of FITC solution was added to the cell-penetrating protein/conjugation buffer, and mixture was gently stirred for 1 hour at room temperature in the dark. FITC-CP-protein solution was additionally incubated at 37° C. in the dark to ensure labeling. After labeling, proteins were dialyzed in the dark against DMEM (no FBS or penicillin/streptomycin supplement) for 2-4 h to remove excess dye. The relative fluorescence of the FITC-labeled proteins was determined using a Fusion Universal Microplate Analyzer (Perkin Elmer Lifesciences) at 485 nm excitation, 535 nm emission, and 20 nm band pass. Protein solutions with equivalent fluorescence units were used in all experiments. A solution of FITC only was used as a control for labeling. FITC-labeled proteins were stored at 4° C. until added to RAW cells for intracellular delivery and subcellular localization experiments.

Protease Accessibility Assay & Confocal Microscopy—RAW cells were plated at 1×105 cells in MAT-TEK 35 mm plate with a #1.5 coverglass, and incubated overnight at 37° C. The following day, media was replaced with DMEM (no FBS supplement), and ˜1 μM of FITC-labeled recombinant proteins, or FITC only (based on equivalent fluorescence) was added to cells, and incubated at 37° C. for 1 h. Cells were gently rinsed three times with warm PBS, and treated for 15 min at 37° C. with 10 μg/ml proteinase K to degrade any protein that had not been internalized into cells. Cells were again rinsed three times with PBS, and cold fresh media supplemented with FBS was placed on the cells. RAWs were kept cold until microscopy could be completed. In experiments to determine intracellular localization of recombinant FITC-labeled SOCS3 proteins, cells were additionally treated with 5 μM of FM595 (Invitrogen), a fluorescent marker for endosomal/plasma membrane. Cells were labeled for 5 min at 25° C. following proteinase K treatment to remove non-internalized FITC-labeled CP-SOCS3, CP-SOCS3ΔSB and non-CP-SOCS3 (control). Cells were then rinsed three times with cold PBS, and incubated with DMEM (supplemented with 10% FBS), and kept on ice. Microscopy examination was completed immediately after labeling. Confocal microscopy was performed using a Zeiss LSM 310 META inverted confocal microscope, and results were analyzed with Zeiss LSM Image Browser (Version 4.2.0.121).

Statistical Analysis—Experimental data was graphed using GraphPad Prism 4 (Version 4.03) software. Two-way ANOVA was used to determine the significance of difference between groups of data. Data are expressed as mean±standard error (S.E.).

### Results and Discussion

The outcome of inflammation depends on the genome-orchestrated balance between proinflammatory mediators and anti-inflammatory suppressors. SOCS3 inhibits pro-inflammatory signaling at the level of the JAK/STAT pathway (Alexander, W. S., and Hilton, D. J. (2004) Annu. Rev. Immunol. 22, 503-529). However, excessive pro-inflammatory signaling can overwhelm this protective mechanism, leading to SOCS3 degradation via the Ubiquitin-proteosome pathway, depletion of intracellular SOCS3 stores, and attendant pathological consequences. In this regard, cell-penetrating (CP) forms of wild-type SOCS3 that are persistently expressed in primary, immunocompetent cells and that function as potent anti-inflammatory suppressors in vivo, were engineered. The possibility of further reinforcing the intracellular pool of SOCS3 and extending its anti-inflammatory potential by engineering a degradation-resistant form of the protein was further investigated. The development and characterization of this mutant led to experimental proof of its significantly prolonged inhibition of proinflammatory signaling in an inflammation-relevant cell type.

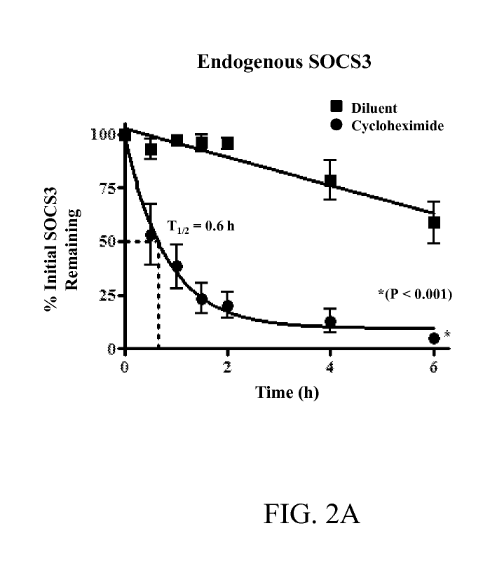

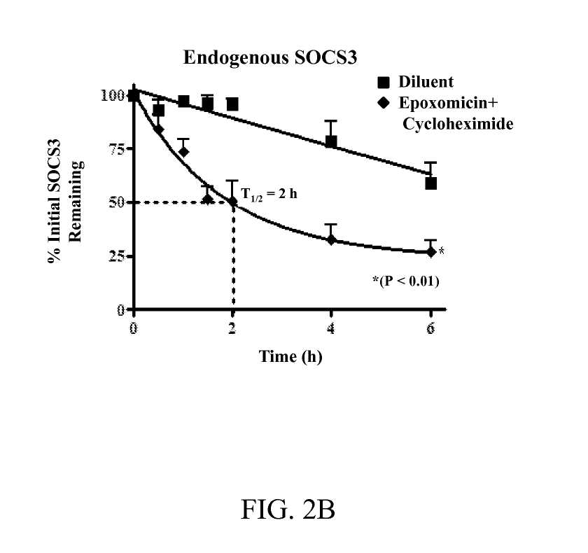

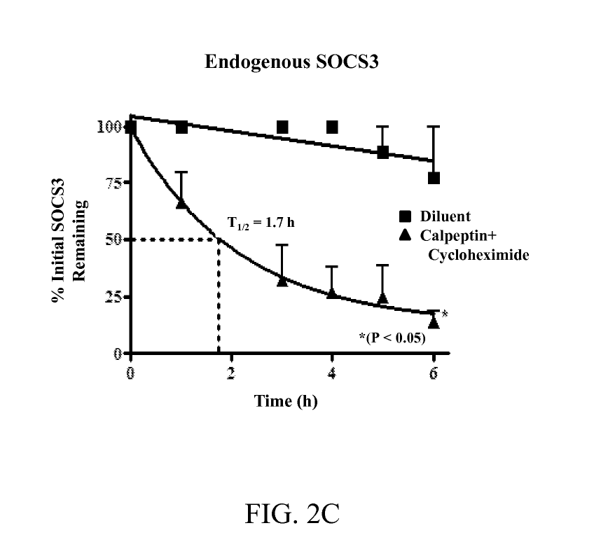

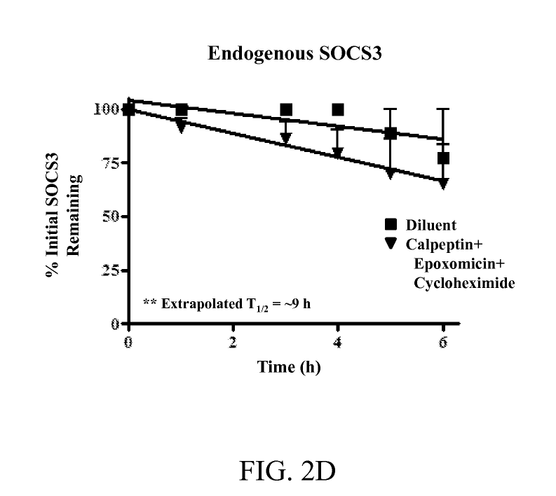

Endogenous SOCS3 is rapidly degraded in stimulated RAW macrophages—Previous protein turnover studies of transfected SOCS3 indicated a t1/2 of 1.6 h when the protein is over-expressed in monkey COS cells. To determine the t1/2 of endogenously expressed SOCS3 in the murine peritoneal macrophage cell line, RAW 264.7, conditions for quantitative measurement of its expression upon stimulation with proinflammatory agonists was established. RAW macrophages were stimulated for 4 h with LPS and IFN-γ to induce SOCS3 protein that was readily measured by quantitative immunoblotting using the infrared Odyssey system. At this time point, cycloheximide (15 μg/ml) was added to inhibit de novo protein synthesis and the cells were sampled at specified time intervals to determine SOCS3 protein levels. Endogenous SOCS3 was rapidly degraded as documented by its t1/2 of 39 min or 0.7 h (FIG. 2A). This rate of SOCS3 turnover is similar to that observed in murine pro-B cell line Ba/F3 but faster than that of ectopically expressed SOCS3 in COS cells. Rapid turnover of SOCS3 depends on its two protein degradation motifs, a C-terminal SOCS box and a PEST motif (FIG. 1). The proteosome-independent degradation mechanism is based on recognition and cleavage of PEST sequences by calpain proteases. In turn, the SOCS box functions as a platform for E3 ligase formed by elongin B/C and cullin 5 to target the SOCS3 protein for ubiquitin-mediated proteosome degradation (Babon, J. J., et al. (2008) J. Mol. Biol. 381, 928-940; Babon, J. J., et al. (2009) J. Mol. Biol. 387, 162-174). SOCS box-dependent proteasomal degradation can be attenuated by the irreversible inhibitor epoxomicin. Therefore, endogenous SOCS3 turnover was analyzed in RAW macrophages treated with inhibitors of proteosome- and calpain-based proteolysis to determine the role of these degradative pathways in the stability of endogenous SOCS3. Treatment with epoxomicin extended the t1/2 of SOCS3 from 0.7 h to 2 h (FIG. 2B). When cells were treated with calpeptin to inhibit the activity of calpain proteases that recognize the PEST motif, the rate of SOCS3 turnover was also increased from 0.7 h to 1.7 h (FIG. 2C). Combined treatment of RAW macrophages with both inhibitors extended the t1/2 over 10-fold for endogenous SOCS3 to ˜9 h (FIG. 2D). Taken together, these results indicate that inhibitors of proteosomes and of calpain act synergistically to inhibit degradation of endogenous SOCS3 mediated by its SOCS box and PEST motif.

Intracellular delivery and turnover of recombinant cell-penetrating SOCS3—The rapid turnover of endogenous SOCS3 in macrophages exceeds that of the previously reported value for ectopically expressed SOCS3 in a COS cell line (Siewert, E., et al. (1999) Eur. J. Biochem. 265, 251-257). Although the forced expression of genes that encode intracellular signal transducers and their regulators has provided valuable information about the mechanism of intracellular action of these molecules, this method is subject to variable efficiency of transfection and an inability to control the abundance of the expressed proteins. In contrast, intracellular delivery of physiologic proteins based on the attachment of a cell-penetrating (CP) membrane translocating motif (MTM) to a recombinant intracellular anti-inflammatory protein allows its controlled delivery in terms of time and concentration to analyze and inhibit signal transduction. Recombinant CP-SOCS3 inhibits the JAK/STAT pathway and prevents cytokine-mediated lethal inflammation and apoptosis of the liver (Jo, D., Liu, D., Yao, S., Collins, R. D., and Hawiger, J. (2005) Nat. Med. 11, 892-898). It was hypothesized that CP-SOCS3 may have an extended t1/2 relative to endogenous SOCS3, as FITC-labeled CP-SOCS3 persists for 8 h in blood leukocytes, lymphocytes, and spleen cells in vivo. To investigate this possibility, full-length CP-SOCS3 and a deletion mutant in which the SOCS box (amino acids 185-225) had been deleted (FIG. 1A), were engineered. This mutant, CP-SOCS3ΔSB, was comprised of amino acids 1-184 of murine SOCS3, including the discovered PEST motif (Babon, J. J. (2006) Mol. Cell. 22, 205-216). A 12 amino acid membrane translocating motif (MTM) was added at the NH2-terminal end of the recombinant protein, which enabled recombinant SOCS3 to cross the plasma membrane in cultured cells and in vivo (FIG. 1A).

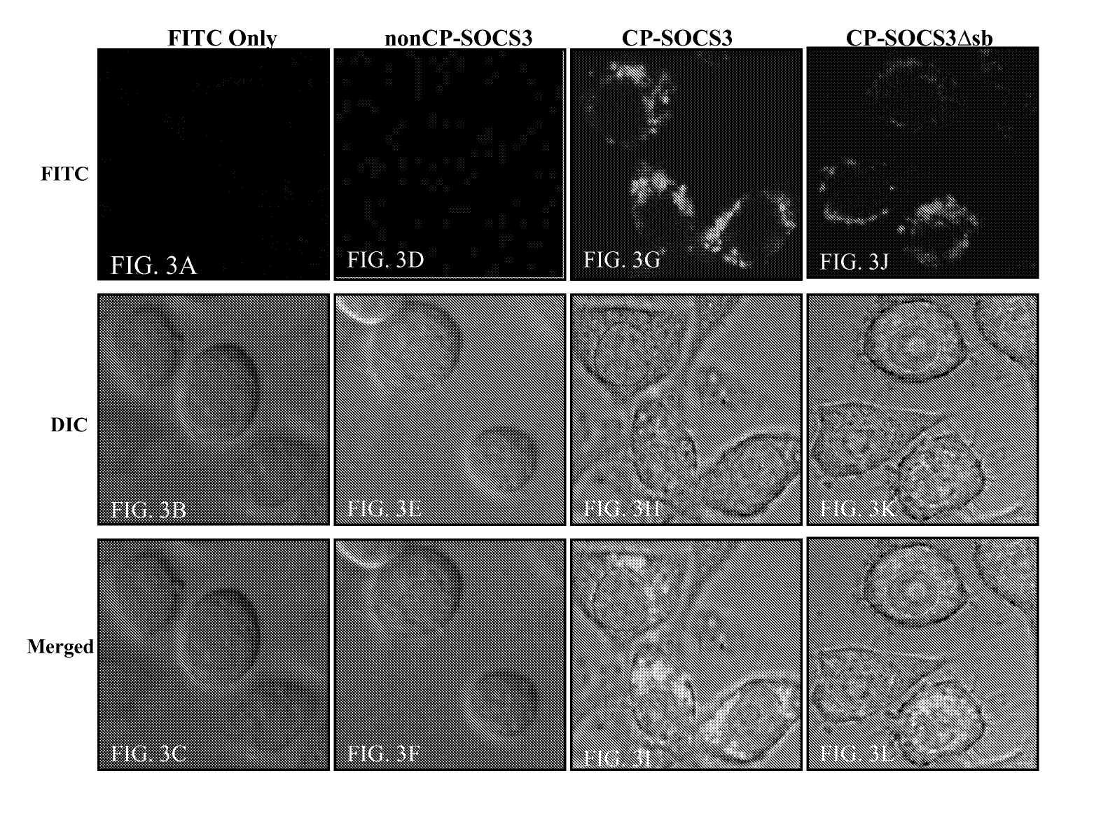

Cellular uptake of full-length CP-SOCS3, and CP-SOCS3ΔSB proteins labeled with FITC was analyzed using the protease accessibility assay. In this assay, FITC-labeled purified recombinant proteins are added to RAW macrophages for 1 h to allow entry of proteins into the cells. After washing cells to remove extracellular pools of FITC-labeled proteins, proteinase K is added to the media to degrade any membrane-bound proteins that had not been translocated into cells. Proteinase K-treated cells were analyzed directly (without fixation) by confocal microscopy to visualize fluorescent signals indicative of internalized proteins (FIG. 3). As a control, SOCS3 lacking the MTM (nonCP-SOCS3) (FIG. 1A), was not detected in the cells following the protease accessibility assay (FIGS. 3D-3F). In striking contrast, full-length CP-SOCS and CP-SOCS3ΔSB deletion mutant produced strong fluorescent signals in RAW macrophages (FIGS. 3G-3I, and 3J-3L). Significantly, the fluorescent signal was detected in the cytoplasm and not in the nuclei of RAW macrophages indicating that deleting the SOCS box did not alter intracellular distribution of CP-SOCS3ΔSB.

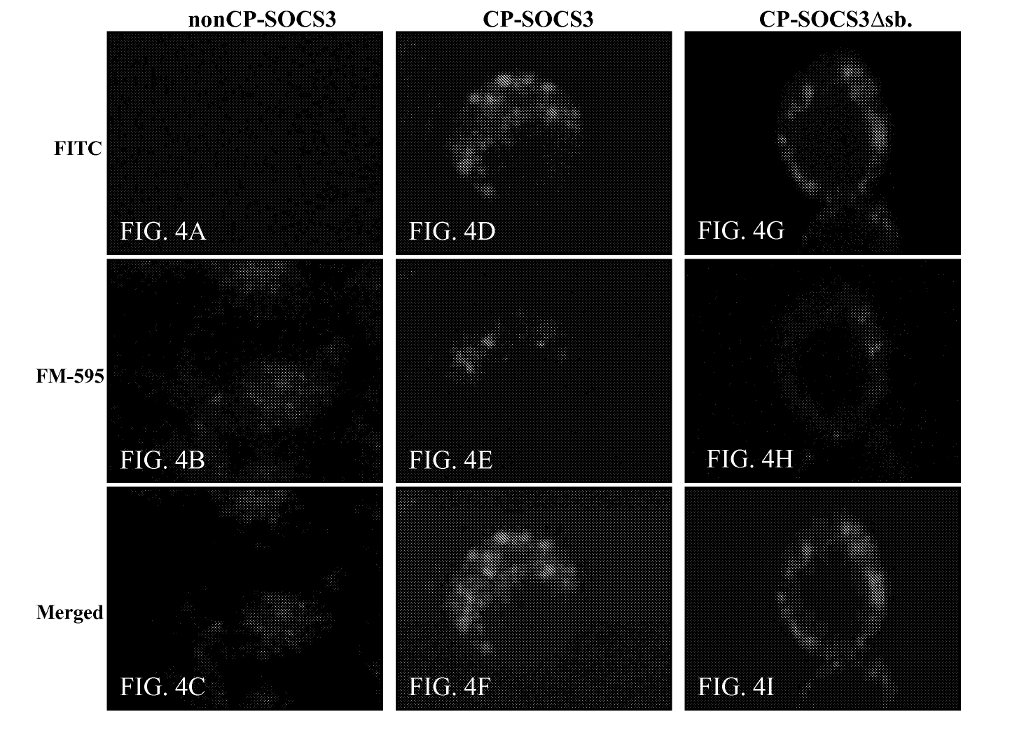

The mechanism of intracellular delivery of short peptides (Mr 2,800), was shown as an endocytosis-independent process of crossing the plasma membrane mediated by hydrophobic MTM (Veach, R. A., Liu, D., Yao, S., Chen, Y., Liu, X. Y., Downs, S., and Hawiger, J. (2004) J. Biol. Chem. 279, 11425-11431). In particular, the helical hair-pin design of the MTM allows for its insertion directly into the plasma membrane, and the “looping-unlooping” of the hairpin allows for the movement of attached peptides through the phospholipid bilayer to the interior of the cell. However, it is plausible that a larger “cargo”, such as that of SOCS3 proteins (Mr 27,000), may induce uptake through the endosomal pathway. Therefore, to address the potential role of endocytosis in the intracellular delivery of CP-SOCS3, its subcellular distribution was analyzed as compared to CP-SOCS3ΔSB in RAW macrophages that had also been labeled with FM-595, an endosomal/plasma membrane marker. Confocal microscope analysis revealed that the FITC-labeled recombinant cell-penetrating SOCS3 proteins (CP-SOCS3 and CP-SOCS3ΔSB), and the endosomal marker FM-595 exhibited distinct distribution throughout the cytoplasm, and did not appear to co-localize with one another (FIG. 4). Interestingly, both CP-SOCS3 and CP-SOCS3ΔSB displayed a punctuate pattern of dispersal throughout the cytoplasm of RAW macrophages, possibly suggesting that these proteins form aggregates with itself or with signaling complexes in the cytosol. This is of potential significance because aggregates of CP-SOCS3 or CP-SOCS3ΔSB could much more efficiently sequester target proteins in the cytosol thereby interfering with pro-inflammatory signaling pathways (i.e., JAK/STAT pathway). Consistent with initial results shown in FIG. 3, FITC-labeled nonCP-SOCS3 did not enter the cell, and only the endosomal marker signal was detected in these samples (FIGS. 4A-4C). These results strongly evidence that CP-SOCS3 and CP-SOCS3ΔSB are delivered to the cytoplasm of RAW macrophages by crossing the plasma membrane independently of the endosomal pathway, thereby avoiding its influence on intracellular turnover of recombinant SOCS3 proteins.

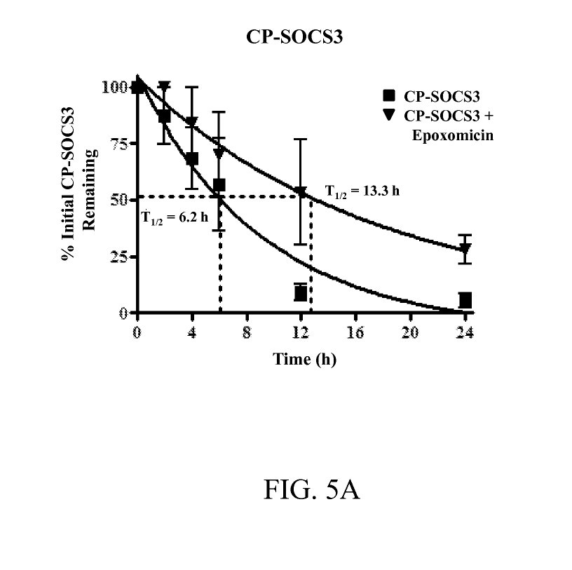

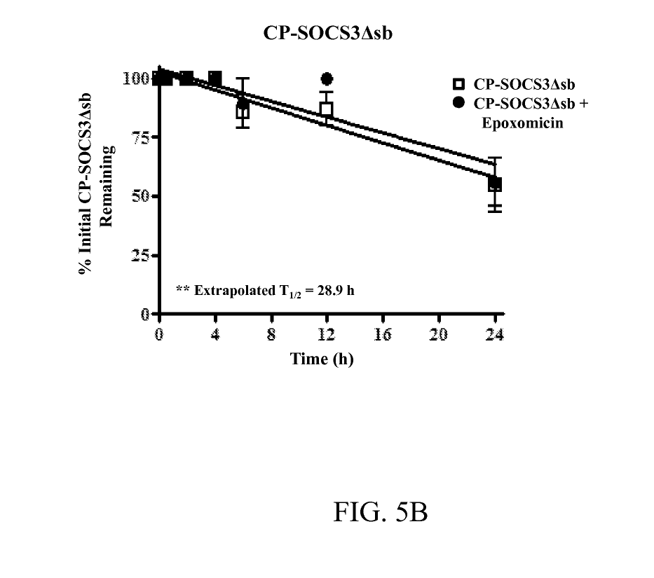

Having established intracellular delivery of recombinant CP-SOCS3 and CP-SOCS3ΔSB that appears to bypass endocytic pathway, their t1/2 in RAW cells was determined under the same conditions as employed in the t1/2 measurements of the endogenous SOCS3 (see above). Proinflammatory agonist-stimulated RAW macrophages were pulsed with CP-SOCS3 or CP-SOCS3ΔSB (1 μM final concentration), and samples collected at regular intervals were analyzed by quantitative immunoblotting. Since these experiments were performed in a stimulated cell line known to express SOCS3 under the same conditions (FIG. 2), blots were probed with an anti-6×His Tag antibody to distinguish recombinant SOCS3 proteins from endogenous SOCS3. As a control, samples from RAW macrophages that did not receive recombinant cell-penetrating protein treatment, but were stimulated with IFN-γ and LPS were probed with the anti-6×His-Tag antibody. No band corresponding to the molecular weight of SOCS3 was detected. It was found that CP-SOCS3 had a t1/2 of 6.2 h (FIG. 5A) as compared to the much shorter t1/2 of 0.7 h for endogenous SOCS3. Hence, in the absence of protease inhibitors, recombinant CP-SOCS3 displays a significantly extended half-life. These results help explain the previous in vivo observations in which FITC-labeled CP-SOCS3 was detectable in the blood leukocytes and lymphocytes and spleen cells of mice 8 h after intraperitoneal administration. The t1/2 of CP-SOCS3 is approximately 9 times longer than that of endogenous SOCS3 (0.7 hours), but is similar to that of endogenous SOCS3 in RAW macrophages treated with inhibitors of proteolysis mediated by calpain and proteasomes (FIG. 2D). Under the experimental conditions employed in these experiments, CP-SOCS3 appears to be more resistant to these two intracellular protein degradation mechanisms than endogenous SOCS3. Moreover, the t1/2 of CP-SOCS3 is extended to 13.3 hours, when the proteasomal pathway of proteolysis is inhibited with epoxomicin (FIG. 5A). This result indicates that CP-SOCS3 turnover is in part regulated by the proteasomal pathway of degradation.

The role of the proteasomal pathway in CP-SOCS3 degradation was further explored by analysis of the CP-SOCS3 mutant, CP-SOCS3ΔSB, in which the SOCS box was deleted (FIG. 5B). The t1/2 of CP-SOCS3ΔSB was extended to ˜29 hours, a remarkable 58-fold increase in stability relative to endogenous SOCS3. In comparison, NH2-truncated SOCS3 that lacked Lys-6 displayed only a 4-fold gain in stability following retroviral transduction of pro-B lymphocyte Ba/F3 cell line. Importantly, the t1/2 of CP-SOCS3ΔSB remained virtually unchanged in the presence of epoxomicin (FIG. 5B), thereby providing additional proof that CP-SOCS3ΔSB is resistant to proteasomal degradation.

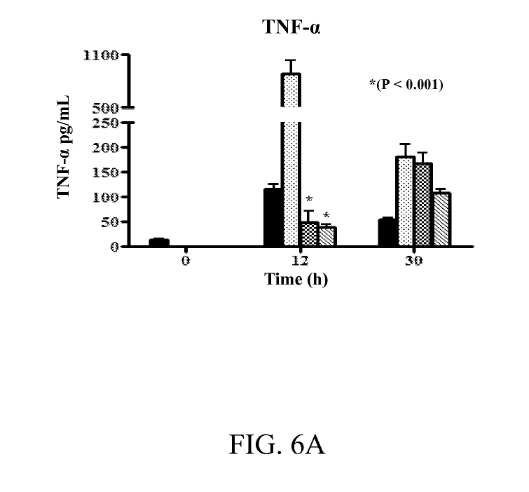

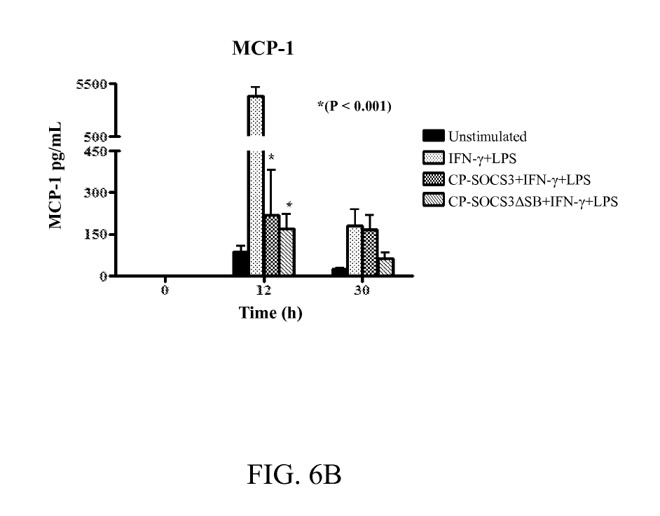

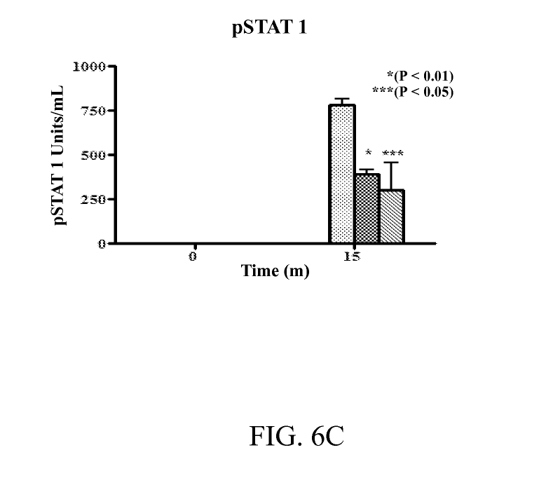

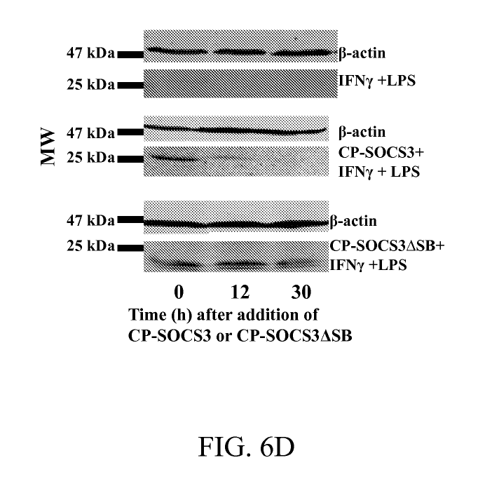

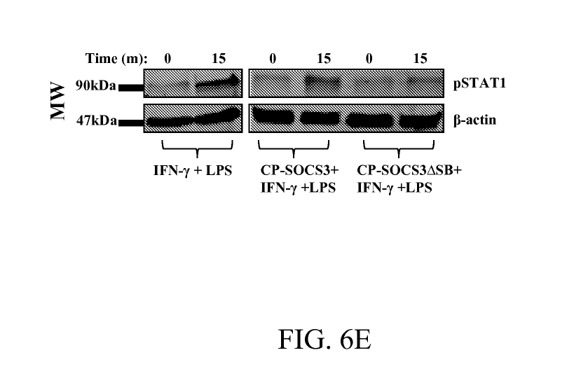

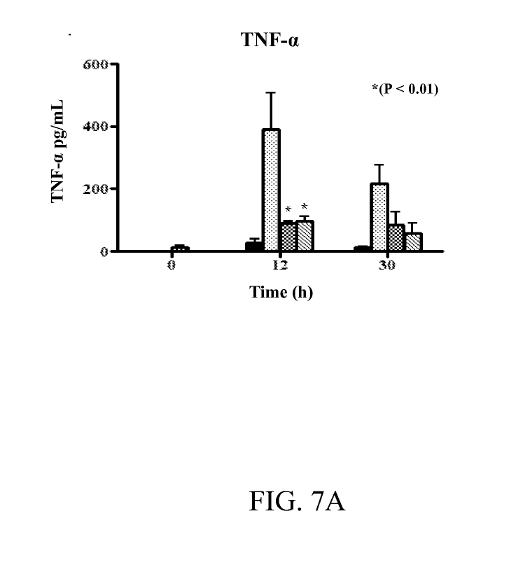

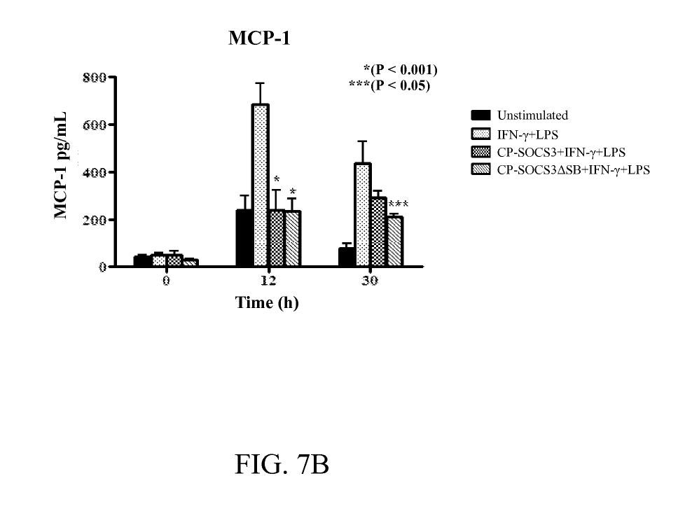

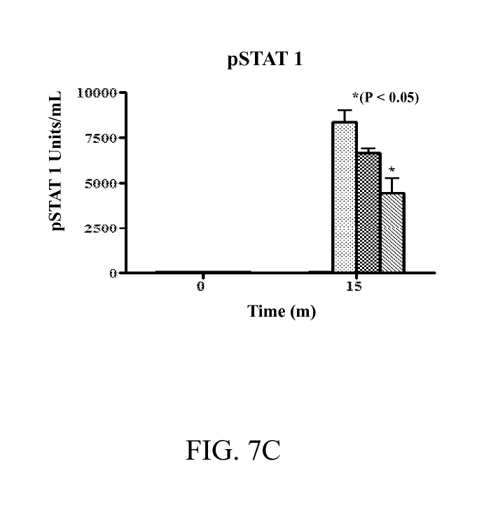

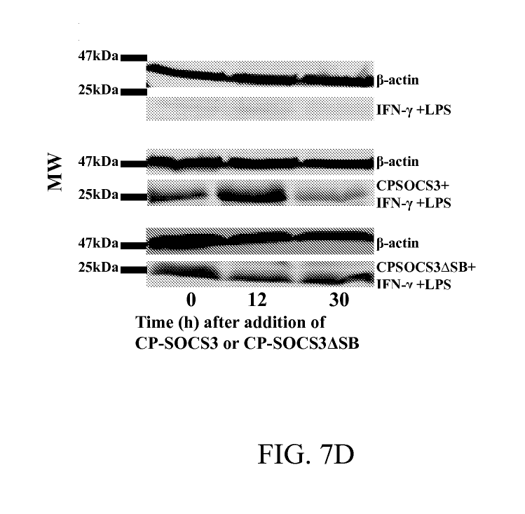

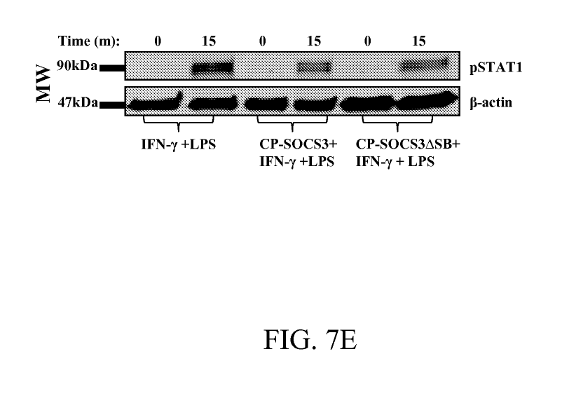

Deletion of the SOCS Box Extends Cytokine/Chemokine Suppression Mediated by CP-SOCS3—The SOCS box acts as an independent recognition motif for binding of Elongin B/C and Cullin 5 to form a functional E3 ubiquitin ligase scaffold that targets signaling complexes formed by a variety of cytokines and their cognate receptors for proteasomal degradation (Babon, J. J., et al. (2008) J. Mol. Biol. 381, 928-940). As such, it was reasoned that the decreased turnover rate of CP-SOCS3ΔSB might significantly affect its capacity to suppress intracellular signaling. Therefore, it was assessed whether deletion of the SOCS box would change the inhibitory activity of CP-SOCS3ΔSB mutant upon intracellular delivery. To that end, AMJ2.C8 macrophages were treated with either CP-SOCS3 or CP-SOCS3ΔSB for 1 h; cells were then rinsed and replaced with fresh media. At 6 h or 24 h following cell-penetrating protein treatment, cells were stimulated with IFN-γ and LPS for 6 h (=12 h and 30 h after CP-protein treatment, respectively), and samples were analyzed for inflammatory cytokine and chemokine production. As shown in FIG. 6, both CP-SOCS3 and CP-SOCS3ΔSB inhibit proinflammatory agonists-induced production of the cytokine TNF-α, and the chemokine, MCP-1 at 12 h post cell-penetrating protein treatment (FIGS. 6A, 6B, 6C). In contrast, at 30 h, only CP-SOCS3ΔSB maintained its inhibitory activity whereas the CP-SOCS3 anti-inflammatory effect was negligible. These functional results are consistent with the persistence of CP-SOCS3ΔSB in AMJ2.C8 macrophages at 30 h, as detected by immunoblotting (FIG. 6D). These experiments were extended to bone marrow-derived macrophages (BMDM) obtained from C3H/HeJ mice. These freshly obtained primary cells were treated with CP-SOCS3 or CP-SOCS3ΔSB for 1 hour, followed by 6 h stimulation with IFN-γ and LPS at 6 h and 24 h post cell-penetrating protein treatment (see above). It was found that both CP-SOCS3 and CP-SOCS3ΔSB significantly reduced the production of TNF-α, and MCP-1 (FIGS. 7A-7C) at 12 h post cell-penetrating protein treatment. At 30 h post-protein treatment, both proteins CP-SOCS3 and CP-SOCS3ΔSB suppressed the production of these inflammatory mediators as compared with untreated (no cell-penetrating protein) controls, although CP-SOCS3ΔSB was slightly more effective than CP-SOCS3 (FIGS. 7A, 7B). Immunoblot analysis confirmed that CP-SOCS3ΔSB also persists for 30 h in BMDM, a fact that supports the functional data (FIG. 7D). Taken together, these results demonstrated that CP-SOCS3ΔSB, which has a significantly increased t1/2, also retains its inhibitory function following intracellular delivery, as evidenced by the reduced production of TNF-α and MCP-1.

CP-SOCS3ΔSB Exerts Anti-Inflammatory Activity by Reducing STAT1 Phosphorylation—It has been firmly established that SOCS3 regulates the JAK/STAT pathway by binding to both activated JAK kinase, and/or the cytoplasmic domain of the phosphorylated gp130 receptor, which inhibits docking and subsequent activation of STAT proteins (Kiu, H., et al. (2009) Growth Fact. 27, 384-393). SOCS3, induced by TLR4 stimulation, indirectly regulates this signaling pathway by modulating LPS-induced signaling pathway, including signals transduced through the JAK/STAT pathway (Dimitriou, I. D., et al. (2008) Immunol. Rev. 224, 265-283). Moreover, SOCS3 deficiency in cells causes a significant increase in STAT1 phosphorylation and an IFN-γ-like gene response. Full-length CP-SOCS3 can inhibit STAT1 phosphorylation in AMJ2.C8 macrophages. Since IFN-γ and LPS were used to stimulate inflammatory conditions in macrophages, it was ascertained whether the attenuation in cytokine and chemokine production evidenced above is due to CP-SOCS3ΔSB-mediated inhibition of STAT1 phosphorylation. Therefore, AMJ2.C8 macrophages were treated with either CP-SOCS3 or CP-SOCS3ΔSB for 1 h. After replacing the medium, the macrophages were stimulated with IFN-γ and LPS for 15 min to induce STAT1 phosphorylation. The cells were harvested and lysates were assayed for phosphorylated STAT1 using a flow cytometric bead-based assay. It was determined that CP-SOCS3 or CP-SOCS3ΔSB reduced STAT1 phosphorylation in AMJ2.C8 macrophages (FIG. 6E). Western blot analysis of the lysates verified lower levels of phosphorylated STAT1 in CP-SOCS3 and CP-SOCS3ΔSB treated samples as compared to untreated controls (FIG. 6E). Thus, the lack of SOCS box in CP-SOCS3ΔSB did not impede STAT1 phosphorylation in IFN-γ and LPS-stimulated cells.

This analysis was extended to primary macrophages. BMDM were treated with CP-SOCS3 or CP-SOCS3ΔSB and stimulated with IFN-γ and LPS according to the same protocol as outlined above. These primary cells displayed heightened responsiveness to IFN-γ and LPS as attested by the higher level of STAT1 phosphorylation. Nevertheless, STAT1 phosphorylation was reduced in CP-SOCS3- or CP-SOCS3ΔSB-treated BMDM (FIG. 7D). These results were verified by immunoblot analysis of phosphorylated STAT1 (FIG. 7F). Cumulatively, these functional analysis demonstrates that SOCS box deletion mutant, CP-SOCS3ΔSB, functions through a similar mechanism as a full-length CP-SOCS3. Both inhibit STAT1 phosphorylation through the interaction of their centrally-located SH2 domain with the cytokine receptor and/or JAK kinase. It is apparent that SOCS box in CP-SOCS3 is dispensable for its cytokine signaling suppressing function while its intracellular turnover is greatly reduced.

Overall, these results identify key mechanisms that play a role in intracellular turnover of endogenous SOCS3 and recombinant CP-SOCS3. In addition, a SOCS box-deleted form of CP-SOCS3 was developed and characterized, that has a greatly extended t1/2 life, which prolongs its ability to suppress proinflammatory cytokine signaling. The extended anti-inflammatory activity of CP-SOCS3ΔSB supports a SOCS box-independent mechanism of cytokine signaling suppression. Furthermore, the unexpectedly extended t1/2 of CP-SOCS3 suggests that addition of the MTM to recombinant CP-SOCS3ΔSB may provide a protective “shield”, against intracellular protein degradation mediated by the PEST domain and possibly other putative protein degradation sites in SOCS3.

In summary, a SOCS box-deleted form of CP-SOCS3 was engineered that suppresses proinflammatory cytokine signaling much more effectively than its wild-type counterpart. Deletion of the SOCS box from CP-SOCS3 greatly extends the half-life of CP-SOCS3, whereas endogenous wild-type SOCS3 is rapidly degraded following its induction with proinflammatory agonists in macrophages. This increased stability, coupled with the capacity for rapid, intracellular delivery, renders the SOCS3 mutant an attractive candidate for protein therapeutic approaches to suppress pathologic inflammation. Further in vivo studies of long-acting CP-SOC3 forms in relevant models of acute and chronic inflammation will expand our understanding of the global role of SOCS3 in modulating signals generated by a variety of proinflammatory agonists in multiple organ system. In principle, the results presented here may also be applicable to the conversion of other conditionally-labile suppressors into more stable, persistently-acting forms for use in intracellular therapy.

### Example 2

### Intracellular Delivery of a Cell-Penetrating SOCS1 that Targets IFN-γ Signaling

### Materials and Methods

Cell culture: The murine alveolar macrophage cell line AMJ2.C8 was obtained from the American Type Culture Collection (Manassas, Va.; TIB-71) and cells were cultured in Dulbecco's modified Eagle's medium (DMEM) (Mediatech, Inc., Manassas, Va.) supplemented with 5% fetal bovine serum (FBS), 10 mM Hepes, penicillin (100 U/ml), and streptomycin (100 mg/ml) at 37° C. in 5% CO2 in humid air. Cell viability was >80% before use in all experiments. HEK 293T cells were maintained in DMEM supplemented with 10% FBS, penicillin (100 U/ml), and streptomycin (100 mg/ml) at 37° C. in 5% CO2 in humid air. HEK 293F cells were maintained in FreeStyle 293 medium supplemented with G418 (25 mg/ml; Invitrogen, Carlsbad, Calif.) at 37° C. in 8% CO2 in humid air. HEK 293-6E cells stably expressing Epstein-Barr virus (EBV) nuclear antigen I were provided by Y. Durocher (National Research Council, Canada) and maintained in FreeStyle 293 protein expression medium supplemented with G418 (25 mg/ml) at 37° C. in 5% CO2 in humid air.

Isolation and culture of BMDMs: For each preparation, bone marrow from C3H/HeJ mice was prepared by flushing mouse femurs and tibias with ice-cold DMEM supplemented with L-glutamine. Bone marrow cells were pooled, passed through a 25 5/8-gauge needle, and filtered through a 70-mm cell strainer. Pooled cells (1×106 cells/ml) were suspended in DMEM supplemented with 10% FBS, 10 mM Hepes, penicillin (100 U/ml), streptomycin (100 mg/ml), and 20% L929 conditioned medium followed by plating on 150-mm bacterial Petri dishes. Cells were incubated at 37° C. in 5% CO2 in humid air. Every 3 days, non-adherent cells were removed, cells were washed, and culture medium was replaced. Cells were used in experiments after 10 days of culture for up to 2 weeks after maturation. When analyzed by flow cytometry, 95% of the adherent cells were MAC3+, CD3−, and B220−. The viability of BMDMs was >80% before use in all experiments.

Preparation of plasmids encoding cell-penetrating SOCS1 for the production of recombinant proteins in E. coli: Full-length SOCS1 murine complementary DNA (cDNA) was provided by M. Shong (Chungnam National University, Korea). Polymerase chain reaction (PCR) primers encoding an MTM composed of 12 amino acid residues from a signal sequence hydrophobic region of FGF4 and an Nde I site sequence at the 5′ or 3′ ends of Socs1 were engineered (Integrated DNA Technologies, Coralville, Iowa) and used to amplify the sequence of Socs1. PCR products were gel-purified (Qiagen, Valencia, Calif.) and cloned into pCR-TOPO-2.1 according to the manufacturer's specifications and were used to transform chemically competent E. coli JM109 cells (Invitrogen). The 5′ or 3′ MTM-containing Socs1 DNA was subsequently cloned into pET28a (EMD Chemicals, Inc., Darmstadt, Germany) and propagated in E. coli DH5a (Invitrogen). The pET28a constructs containing MTM at the 5′ or the 3′ end of the Socs1 sequence were transferred to E. coli BL21 expression vectors (Stratagene, La Jolla, Calif.) for determination of the abundance of SOCS1 proteins after induction with isopropyl β-D-1-thiogalactopyranoside (IPTG). SOCS 1 DNA without the MTM was constructed as a control. The truncated forms of SOCS1, lacking the PEST motif and SOCS box, were constructed by PCR mutagenesis and produced in BL21 expression strains of E. coli.

Preparation of plasmids encoding cell-penetrating SOCS1 plasmids for the production of recombinant proteins in human cells: DNAs encoding non-CP-SOCS1 and CP-SOCS1 were subcloned into the mammalian expression vector pTT5, which were then used to transfect HEK 293-6E cells. PCR primers were constructed that encompassed a Kozak translation initiation sequence with an ATG initiation codon in front of a 6× histidine tag and the MTM sequence. Primers contained Eco RI and Bam HI restriction site sequences to facilitate subcloning into the mammalian expression vector pTT5, which harbors the EBVoriP in the vector backbone. HEK 293-6E cells produce substantially more protein when the EBVoriP is present in the vector backbone. Non-CP-SOCS1 was constructed similarly except for lacking the MTM sequence.

Production, purification, and reconstitution of recombinant SOCS1 proteins: The production of recombinant SOCS1 proteins in E. coli BL21 cells was induced with 0.1 to 0.5 mM IPTG and proteins were expressed as insoluble IBs. IBs were purified with the Bugbuster Protein Extraction Reagent (EMD Chemicals, Inc., Darmstadt, Germany) according to the manufacturer's protocol. Alternatively, IBs were prepared with a protocol adapted in our laboratory. Briefly, pelleted bacteria were suspended in IB buffer [20 mM tris-HCl (pH 7.5), 10 mM EDTA, 1% Triton X-100, and 0.3 M NaCl] followed by the addition of lysozyme (1.0 mg/ml) and sonication. IBs were purified by repeated centrifugation and sonication, passed through a 0.45-mm syringe filter, and solubilized in solubilization buffer A [6 M guanidine hydrochloride (GuHCl), 100 mM NaH2PO4, and 10 mM tris-HCl (pH 8.0)] followed by gravity nickel-nitrilotriacetic (Qiagen) liquid chromatography. E. coli-derived proteins used for cytokine experiments were purified with histidine affinity columns by FPLC (AKTA Purifyer, GE Healthcare, Piscataway, N.J.). Briefly, proteins were bound to histidine columns in buffer A, washed extensively with buffer B [6MGuHCl, 100 mM NaH2PO4, and 10 mM tris-HCl (pH 6.0)] and eluted with buffer C [6 M GuHCl, 100 mM NaH2PO4, and 10 mM tris-HCl (pH 4.0)]. For recombinant proteins produced in HEK 293-6E cells, pTT5 vectors containing either non-CP SOCS1 or CP-SOCS1 DNA were propagated in E. coli DH5a followed by plasmid purification by cesium chloride gradient. Transient transfection of HEK 293-6E cells with pTT5 vectors was performed by complexing DNA with linear polyethyleneimine (PEI) (Polysciences, Warrington, Calif.) from a stock solution of 1 mg/ml. Briefly, DNA (1 mg) and PEI (2 mg) per 106 cells (total ˜108 cells used per transfection) were suspended in Opti-MEMI (Invitrogen), prewarmed to 37° C., and allowed to incubate for 30 min at room temperature before being added to cells. Protein expression was allowed to proceed for 72 hours, with shaking at 125 rpm, in tissue culture flasks at 37° C. in 5% CO2 in humid air. Cells were harvested by centrifugation and suspended in buffer A, passed through a 0.2-mm filter, and purified by FPLC with a dual-step histidine purification method. Briefly, HEK-produced SOCS1 proteins were initially purified with a HisTrap FF Crude column (GE Healthcare, Piscataway, N.J.) as described above, except that elution was performed under a 50-ml pH gradient from pH 6.0 to pH 4.0 after extensive washing with buffer B. Fractions containing SOCS1 proteins with a minimal number of contaminating proteins were pooled and purified again over a HisTrap HP column under similar conditions as for the crude column. With this method, the purity of SOCS1 proteins was consistently greater than 90% as quantified by the Odyssey Infrared Imaging System (LI-COR, Inc., Lincoln, Nebr.). Refolding buffer conditions for each protein were established with a matrix-assisted protein refolding kit (Pierce Biotechnology, Rockford, Ill.). Proteins (at 100 mg/ml) were dialyzed against refolding buffer consisting of 50 mM tris-HCl, 150 mM NaCl, 0.8 mM KCl, 1.0 mM EDTA, 0.55M GuHCl, 0.1M NDSB201, 0.44M L-arginine, and 1 mM oxidized and reduced glutathione (pH 8.0) overnight at 4° C. E. coli-produced proteins were then exhaustively dialyzed against post-refolding buffer consisting of DMEM supplemented with 0.3 M L-arginine, 2.5 mM polyethylene glycol (PEG) 3350, and 1% penicillin and streptomycin.

HEK 293-6E-produced protein was dialyzed against DMEM supplemented with 100 mML-arginine and 1% penicillin and streptomycin. The presence of L-arginine in the post refolding buffers was required to maintain protein stability, whereas PEG 3350 was used to minimize protein precipitation after a single freeze-thaw cycle. After dialysis, E. coli-produced protein solutions were passed through a 0.45-mm syringe filter and concentrated by Millipore Ultrafiltration Devices (Millipore, Billerica, Mass.). Concentrated proteins were used immediately for experiments, whereas non concentrated proteins were stored at −80° C. Any contaminating LPS in recombinant proteins was analyzed by the Limulus assay (Pyrosate, East Falmouth, Mass.) and averaged about 1.0 ng per microgram of E. coli-produced protein but was not detectable for proteins produced in HEK 293-6E cells.

Immunoprecipitations and Western blotting analysis: To determine whether cell-penetrating proteins could cross the cell membrane, BMDMs from C3H/HeJ mice or AMJ2.C8 macrophages were treated with equimolar concentrations of non-CP-SOCS1 (0.75 mg) and CP-SOCS1 (0.78 mg) or diluent alone for 1 hour at 37° C. Pelleted cells were washed with ice-cold DMEM containing 150 mM L-arginine (DMEM+LA) and treated with proteinase K (5 mg/ml) for 10 min at 37° C. to remove proteins attached to the cell surface, followed by two additional washes in ice cold DMEM+LA. Pelleted cells were treated with lysis buffer [20 mM Hepes (pH 7.0), 2% NP-40, 50 mM KCl, 0.1 mM EDTA, and 2 mM MgCl2] supplemented with protease inhibitors (Sigma-Aldrich, St. Louis, Mo.) followed by passage thorough a 25 5/8-gauge syringe needle. Lysates were cleared by centrifugation at 9000 g for 15 min at 4° C. followed by preclearing of the supernatant with protein G-Sepharose beads for 30 min at 4° C. Lysates containing non-CP-SOCS1 or CP-SOCS1 were immunoprecipitated with a monoclonal antibody specific for SOCS1 (5.0 mg, US Biological, Swampscott, Mass.) overnight at 4° C. followed by incubation with protein G-Sepharose beads for 2 hours at 4° C. Where indicated, nonspecific immunoglobulin G1 antibodies (Zymed Laboratories, San Francisco, Calif.) were used as an immunoprecipitation control in CP-SOCS1-treated cells. Beads were washed three times with lysis buffer, followed by the elution of antibody complexes during incubation of beads in 2×SDS sample buffer at 100° C. for 5 min. Samples were resolved by SDS-polyacrylamide gel electrophoresis (SDS-PAGE), transferred by Western blotting to nitrocellulose membranes, and analyzed with goat polyclonal antibodies against SOCS1 (Abcam Inc., Cambridge, Mass.). Western blots were developed with fluorescently labeled secondary antibodies and visualized with the Odyssey Infrared Imaging System (LI-COR).

Analysis of protein complexes associated with intracellular CP-SOCS1: Co-immunoprecipitation analyses was used to identify cellular proteins targeted by CP-SOCS1. Cells pulsed with non-CP-SOCS1 or CP-SOCS1 proteins were subjected to procedures identical to those described in the preceding section with the exception of the lysis buffer, which consisted of 20 mM Hepes (pH 7.4), 150 mM NaCl, 1.5 mM MgCl2, 0.1 mM EDTA, 0.1% NP-40, 10% glycerol, and protease and phosphatase inhibitors (Sigma-Aldrich). Antibodies used for co-immunoprecipitations included monoclonal anti-SOCS1, anti-STAT1 (BD Transduction Laboratories, San Jose, Calif.), and anti-JAK2 (Chemicon Inc., Temecula, Calif.). Co-immunoprecipitation samples were subjected to SDS-PAGE and Western blot analysis with anti-STAT1 phosphorylated at Tyr701 (BD Biosciences), SOCS1 (polyclonal) (Abcam), or JAK2 phosphorylated at Tyr1007 and Tyr1008 (Chemicon).

Analysis of STAT1 phosphorylation: BMDMs derived from C3H/HeJ mice or AMJ2.C8 cells were treated with different concentrations of non-CP-SOCS1 or CP-SOCS1 and analyzed for the extent of STAT1 phosphorylation. Cells (3.0×106 total cells) were suspended in medium containing the individual proteins for 1 hour followed by the addition of IFN-γ (10 to 30 U/ml; EMD Chemicals, Inc., Darmstadt, Germany) and LPS (100 ng/ml, Sigma-Aldrich). To analyze the time course of the function of CP-SOCS1, AMJ2.C8 cells were incubated with protein for 1 hour after which the protein was removed and the cells were suspended in SOCS1-free DMEM+5% FBS (time 0). Cells were stimulated with IFN-g (2 U/ml) starting at time 0 and at the subsequent time points. Analysis of the phosphorylation of STAT1 under conditions in which the SOCS1 proteins were expressed by transfection was performed in HEK 293F cells. Cells were transfected with the plasmids pTT5, pTT5-non-CP-SOCS1, or pTT5-CP-SOCS1 with 293fectin (Invitrogen) according to the manufacturer's specifications. After overnight incubation, 3×106 cells were analyzed for the extent of STAT1 phosphorylation after incubation with IFN-γ for 15 min at 37° C. and 5% CO2. For all experiments, total cell lysates were standardized for protein concentration by the method of Bradford, and the abundance of phosphorylated STAT1 was quantified by cytometric bead array (CBA, BD Biosciences) according to the manufacturer's protocol or by Western blotting analysis with an antibody specific for phosphorylated STAT1.

Analysis of the production of proinflammatory cytokines and chemokines: The ability of non-CP-SOCS1 and CP-SOCS1 to inhibit IFN-g-induced production of cytokines and chemokines in cultured BMDMs from C3H/HeJ mice and in AMJ2.C8 cells was analyzed. Immediately before addition to cells, E. coli-produced protein solutions were diluted threefold, resulting in a final DMEM buffer supplemented with 100 mM L-arginine, 0.8 mM PEG 3350, 10% FBS, and 1% penicillin and streptomycin. Cells (4.0×105) were incubated with the appropriate protein (4.0 mM) for 30 min followed by the addition of IFN-g (100 U/ml) without removal of the SOCS1 proteins. Supernatants were sampled 24 hours after the addition of agonist and analyzed by the MILLIPLEX mouse cytokine-chemokine kit (Millipore, St. Charles, Mo.) according to the manufacturer's specifications. HEK 293-6E-produced proteins were concentrated after dialysis, diluted twofold with DMEM containing 10% FBS and 1% penicillin and streptomycin (resulting in a final DMEM buffer with 50 mM L-arginine) and used immediately in experiments. Cells (4.0×105) were incubated with the appropriate protein (˜2.0 mM) for 60 min followed by the addition of IFN-g (30 or 100 U/ml) without removal of the SOCS1 proteins. Supernatants were sampled between 24 and 48 hours after the addition of agonist and analyzed as described above. For analysis of the effects of SOCS1 proteins expressed by transfection on IFN-γ-induced production of cytokines under conditions of forced expression, HEK 293T cells were used. Cells were transfected with the plasmids pTT5, pTT5-non-CP-SOCS1, or pTT5-CP-SOCS1 by 293fectin as described earlier. After overnight incubation of 1.5×105 transfected cells in a 24-well plate, cells were stimulated for 24 hours with human IFN-γ (10 or 100 U/ml) in the presence of IL-1β (0.1 ng/ml), followed by sampling of the supernatants. Supernatant fractions were analyzed by CBA with a human chemokine kit (BD Biosciences).

### Results

Engineering of a recombinant, cell-penetrating SOCS1 protein in Escherichia coli: A CP form of SOCS3 produced in the E. coli expression system is effective in reducing inflammation and apoptosis in vivo. However, SOCS1 has greater anti-inflammatory capabilities than does SOCS3, which is manifested in SOCS1 primarily targeting STAT1, whereas SOCS3 targets STAT3. Especially relevant is the phenotype of mice deficient in Socs1, which includes rampant inflammation of multiple organs mediated by endogenous IFN-γ, while the expression of SOCS 3 is maintained. A series of recombinant CP and non-CP forms of murine SOCS1 were designed in an attempt to target the IFN-γ-induced signaling pathway. Deletion mutants of CP-SOCS1 were constructed that lacked either the proline, glutamic acid, serine, threonine (PEST) motif or both the PEST motif and the SOCS box, to establish whether these motifs were dispensable for the anti-inflammatory activity of CP-SOCS1. All proteins contained a polyhistidine tag to facilitate their purification by metal-affinity chromatography. Cell-penetrating forms of SOCS1 contained a physiologic MTM derived from the hydrophobic signal sequence region of human fibroblast growth factor 4 (FGF4), which enables attached cargo to cross the plasma membrane. Recombinant mouse non-CP-SOCS1 and CP-SOCS1 proteins (containing an N- or C-terminal MTM) expressed as inclusion bodies (IBs) from E. coli were purified and reconstituted, and their purities and yields were similar. The presence of contaminating LPS in recombinant proteins was analyzed by the Limulus assay, which usually reveals the presence of LPS at concentrations of 1 ng per microgram of recombinant protein. Therefore, for these experiments, LPS-hyporesponsive AMJ2.C8 macrophages (34) or bone marrow-derived macrophages (BMDMs) obtained from LPS-hypo-responsive C3H/HeJ mice were used to mitigate the potential effect of contamination of recombinant proteins by residual LPS.

Intracellular delivery of CP-SOCS1: The abilities of CP-SOCS1 and non-CP-SOCS1 proteins to traverse the cell membrane of LPS-hyporesponsive AMJ2.C8 macrophages were analyzed. This experiment was based on a protease-accessibility assay and on the immunoprecipitation of internalized SOCS1. Cultured cells were treated with non-CP-SOCS1 or CP-SOCS1 proteins for 1 hour. Subsequently, the broad-range protease, proteinase K, was applied to remove SOCS1 proteins from the cell surface, thereby preventing contamination of the cell lysates used in the subsequent analysis by SOCS1. Cells treated with the non-CP-SOCS1 protein and cells treated with diluent as negative controls were used. An isotype-matched antibody for cells treated with CP-SOCS1 provided an additional control for these experiments. Lysates of cells treated with CP-SOCS1 and controls for immunoprecipitation with the indicated antibodies were prepared. Endogenous SOCS1 was not detected in diluent-treated cells, consistent with previous reports that SOCS1 is undetectable unless induced by proinflammatory agonists. In contrast, an immunoreactive band consistent with the size of CP-SOCS1 was immunoprecipitated from lysates of cells treated with CP-SOCS1 by an antibody against SOCS1 (anti-SOCS1). That this band was detected in samples treated with proteinase K indicated the intracellular location of CP-SOCS1 because it was not accessible to protease activity. The intracellular concentration of CP-SOCS1 in AMJ2.C8 cells, based on packed cell volume, was 11.6 nM.

Targeting of IFN-γ signaling pathway components and inhibition of IFN-γ-induced phosphorylation of STAT1 by CP-SOCS1: It was next determined whether CP-SOCS1 delivered intracellularly could interact with components of the IFN-γ signaling pathway. CP-SOCS1-pulsed AMJ2.C8 macrophages were stimulated with IFN-γ and then Western blotting analysis of samples immunoprecipitated with antibodies against JAK2 (anti-JAK2) or STAT1 (anti-STAT1), which are interacting partners of SOCS1, was performed. These experiments revealed immunoreactive bands consistent with the size of CP-SOCS1 in samples immunoprecipitated with anti-JAK2 or anti-STAT1, indicating that recombinant CP-SOCS1 interacted with these components. Endogenous SOCS1 was not detectable under these experimental conditions.

SOCS1 serves as a cytoplasmic feedback inhibitor of the tyrosine phosphorylation of STAT1, the primary transcription factor thought that it also may inhibit the phosphorylation of STAT1. To assess this, the extent of phosphorylation of STAT1 after stimulation of CP-SOCS1-pulsed cells with IFN-g and LPS was analyzed. Concentration-dependent inhibition of STAT1 phosphorylation in AMJ2.C8 macrophages which contained CP-SOCS1 tagged with MTM at its N terminus was observed. The concentration of CP-SOCS1 that inhibited phosphorylation of STAT1 by 50% (IC₅₀) was <1.9 mM. CP-SOCS1 also attenuated IFN-γ-induced phosphorylation of STAT1 in BMDMs from C3H/HeJ mice. The inhibitory effect of CP-SOCS1 in both of these cell types was confirmed by Western blot analysis. To exclude the possibility that the MTM tag was responsible for the observed decreased phosphorylation of STAT1 in CP-SOCS1-pulsed cells, HEK 293F cells were transfected with plasmids encoding non-CP-SOCS1 or CP-SOCS1, incubated the cells overnight, and analyzed the extent of STAT1 phosphorylation in response to a 15-min stimulation with IFN-γ. It was observed at least a 50% reduction in the abundance of phosphorylated STAT1 in cells containing either non-CP-SOCS1 or CP-SOCS1 proteins compared to vector-transfected, control cells, confirming that the MTM in CP-SOCS1 was not responsible for inhibiting the phosphorylation of STAT1 in response to IFN-γ. Next, AMJ2.C8 cells were incubated with non-CP-SOCS1 or CPSOCS1 for 1 hour, after which the recombinant proteins were removed from the culture media (time 0) and the cells were subsequently stimulated with IFN-γ. As early as 20 min after the removal of CP-SOCS1 protein, the abundance of phosphorylated STAT1 in response to IFN-γ was similar in the cells pulsed with CP-SOCS1 and the control cells pulsed with non-CP-SOCS1. These results indicated that the inhibitory effect of CP-SOCS1 was both short-lived and reversible. Together, these data indicate that the functions of CP-SOCS1 recapitulated those of endogenous SOCS1.

CP-SOCS1 inhibits IFN-γ-induced production of proinflammatory chemokines and cytokines: Inhibition of IFN-γ-induced phosphorylation of STAT1 by CP-SOCS1 should result in attenuation of the production of cytokines and chemokines in IFN-γ-stimulated AMJ2.C8 macrophages. To test this hypothesis, the extent of the IFN-γ-stimulated production of cytokines and chemokines in cells incubated with CP-SOCS1, was analyzed. Pretreatment of AMJ2.C8 macrophages for 1 hour with CP-SOCS1 was effective in reducing the production of the chemokines CXCL10 (also known as IP-10) and RANTES (regulated on activation, normal T cell-expressed and secreted) and the cytokines interleukin-6 (IL-6) and granulocyte colony-stimulating factor (G-CSF) by 44, 71, 90, and 88%, respectively, when compared to that of cells pretreated with non-CP-SOCS. IP-10 and RANTES are encoded by genes that contain the IFN-γ activation sequence (GAS) promoter element, whereas IL-6 and G-CSF are increased in abundance in IFN-γ-stimulated cells deficient in SOCS1 relative to wild-type (WT) cells. No substantial induction of these cytokines or chemokines was observed when cells were treated with protein alone, indicating that the response was driven by IFN-γ.

Engineering of recombinant CP-SOCS1 in HEK 293 cells: Due to the persistent presence of residual LPS in E. coli-produced proteins (˜1 ng LPS per mg protein), a strategy was devised to produce CP-SOCS1 and non-CP-SOCS1 proteins in human embryonic kidney (HEK) 293-6E cells followed by their purification by metal-affinity fast protein liquid chromatography (FPLC). The yield of mammalian SOCS1 proteins from HEK cells was substantially lower than that from E. coli; however, the recombinant proteins produced in mammalian cells had the virtue of having undetectable LPS, as determined by the Limulus assay.

Intracellular delivery of mammalian CP-SOCS1 to AMJ2.C8 macrophages followed by their stimulation with IFN-g attenuated the expression of chemokines [IP-10, RANTES, and macrophage inflammatory protein 1β (MIP-1β)] and cytokines (IL-6 and G-CSF) compared to control cells treated with non-CP-SOCS1. Thus, these results obtained with LPS-free recombinant CP-SOCS1 and non-CP-SOCS1 proteins expressed in HEK 293-6E cells validated the results with E. coli-produced proteins. As before, treatment of macrophages with HEK 293-6E-produced CP-SOCS1 in the absence of IFN-γ did not have a measurable effect on the production of chemokines and cytokines. Moreover, to exclude the possibility that the attached MTM tag was responsible for the decreased production of chemokines and cytokines, HEK 293T cells were transfected with plasmids encoding non-CP-SOCS1 and CP-SOCS1 and measured the production of chemokines and cytokines in these cells after treatment with IFN-γ. A substantial reduction in the production of IP-10, RANTES, and MIG (monokine induced by IFN-γ) was observed in both non-CP-SOCS1- and CP-SOCS1-transfected cells in comparison to that of cells transfected with the vector control. These results indicated that the MTM tag was not responsible for CP-SOCS1-mediated suppression of the production of chemokines and cytokines. Thus, attenuation of IFN-γ-induced production of proinflammatory chemokines and cytokines depended on MTM-mediated delivery of its functionally active cargo, CP-SOCS1.

Intracellular delivery of CP-SOCS1 mutants to conduct structure-function analysis: N-terminal KIR (kinase inhibitory region) and SH2 domains of SOCS1 appear to be necessary for inhibition of JAK2-STAT1 signaling in vitro, whereas the SOCS box is less essential for inhibition of cytokine production in vivo. Moreover, the PEST domain in SOCS3 and SOCS1 may contribute to their intracellular instability. A mutational analysis of CP-SOCS1 was performed to establish whether deletion of these two domains, the PEST motif and the SOCS box, would change the inhibitory activity of CP-SOCS1 upon its intracellular delivery. This analysis could identify truncated versions of CP-SOCS1 of increased stability that would be sufficient to inhibit the phosphorylation of STAT1 and the production of proinflammatory cytokines and chemokines. An N-terminal truncated form of CP-SOCS1 (CP-SOC1ΔPEST) was constructed by deleting amino acid residues 1 to 50, a region that harbors the PEST motif. A double mutant (CP-SOCS1ΔPEST.SB) lacking both the N-terminal PEST region and the amino acid residues 168 to 212, which consists of the C-terminal SOCS box (SB) domain was engineered. Pretreatment of AMJ2.C8 macrophages for 1 hour with either of these cell-penetrating mutants of CP-SOCS1 showed their preserved ability to suppress production of IL-6 when compared with that of full-length CP-SOCS1. Thus, intracellular delivery of cell-penetrating mutants of SOCS1 indicated that the presence of the KIR and SH2 domains was sufficient to preserve the inhibitory activity of CP-SOCS1, whereas deletion of the PEST domain or the PEST domain and the SOCS box did not impair the inhibitory activity of mutated CP-SOCS1. These mutagenesis studies are consistent with in vivo studies of the SOCS1 transgene that does not contain the region encoding the SOCS box, which indicated that this mutant protein is capable of replacing functionally active full-length SOCS1 in terms of its anti-inflammatory activity.

Summary: It was shown that intracellular delivery of recombinant CP-SOCS1 produced either in bacterial or in mammalian cells inhibited the IFN-γ-evoked signal transduction required for the expression of genes encoding proinflammatory cytokines and chemokines in cultured macrophages. Our analysis of the mechanism of CP-SOCS1-induced attenuation of IFN-γ signaling documented its intracellular targeting of JAK2 and STAT1. Moreover, intracellular delivery of CP-SOCS1 mutant proteins that lacked the PEST and SOCS box domains suggested the central role of the KIR and SH2 domains in the attenuation of proinflammatory signaling in response to IFN-γ. Thus, these studies show the feasibility of suppressing proinflammatory signaling by the intracellular delivery of SOCS1, a key physiologic inhibitor of the IFN-γ signaling pathway. By establishing that the physiologic function of CP-SOCS1 is similar to that of endogenous SOCS1, a platform is provided for the facile study of the intracellular functions of SOCS1 because of the faster delivery, controlled input, and limited duration of CP-SOCS1 as contrasted with forced expression of the SOCS1 transgene. Moreover, by studying the mechanism of action of CP-SOCS1, a starting point is provided for the development of new therapeutics for inflammation-mediated acute syndromes, such as sepsis, the leading cause of morbidity and mortality in critical care medicine.

Evidence was presented that engineered CP-SOCS1, but not a non-CPSOCS1 control, was able to enter cells and was resistant to digestion by proteinase K. Consistent with the mechanism of action of endogenous SOCS1, it was also shown that CP-SOCS1 targeted the IFN-γ signaling pathway in AMJ2.C8 macrophages. It is noteworthy that the results from these immunoprecipitation experiments were not due to the induced production of endogenous SOCS1 (23.7 KD). An immunoreactive band was observed which was consistent with the size of recombinant CP-SOCS1, which has a molecular mass of 27 kD, because of the added MTM and 6× histidine tag. Moreover, the time frame over which endogenous SOCS1 protein is usually detected in response to IFN-γ stimulation is usually between 2 and 3 hours. These assays were performed with IFN-γ stimulation for 5 to 10 min. Thus, the application of CP-SOCS1 for mechanistic analysis of its intracellular targets recapitulates the known action of endogenous SOCS1.

Inhibition of the phosphorylation of STAT1 by SOCS1 is due to the ability of SOCS1 to bind to the phosphorylated tyrosine residue in the activation loop of JAK2 through its central SH2 domain and the N-terminal KIR domain. Intracellular delivery of CP-SOCS1 attenuated IFN-γ-induced phosphorylation of STAT1 and the production of proinflammatory chemokines and cytokines in primary and established macrophage cell lines. The extent of inhibition of the phosphorylation of STAT1 by CP-SOCS1 was dependent on its concentration. Thus, under steady-state conditions, CP-SOCS1 was effective even at low concentrations (<2.0 mM).

Intracellular delivery of CP-SOCS1 depended on the MTM, which did not influence the intrinsic inhibitory function of CP-SOCS1. As attested by experiments involving the transfection of HEK 293F or HEK 293T cells with the CP-SOCS1 and non-CP-SOCS1 constructs, SOCS1 proteins containing or lacking the MTM equally inhibited IFN-γ-induced phosphorylation of STAT1 and the production of chemokines and cytokines. Thus, these data are consistent with previous reports in which ectopic expression of SOCS1 was used to inhibit phosphorylation of STAT1 and production of cytokines. However, these results were accomplished by way of facile intracellular delivery of recombinant CP-SOCS1, which indicates the potential of the above approach for restoring the homeostatic balance between proinflammatory stimuli and anti-inflammatory regulators, as well as its therapeutic applicability.

Until now, the production of recombinant cell-penetrating protein therapeutics was based on bacterial expression systems. Intractable contamination of recombinant proteins with LPS inherently present in E. coli prompted us to embark on designing a system to express CP proteins in a mammalian system. We succeeded in producing CPSOCS1 and non-CP-SOCS1 in HEK 293-6E cells. Although these recombinant proteins were not as abundantly produced in these cells as they were in the bacterial expression system, they were free of LPS and displayed MTM-dependent inhibitory effects on the IFN-γ-induced production of proinflammatory chemokines and cytokines comparable to those of E. coli-produced proteins. It was shown that CP-SOCS1 inhibited the IFN-γ-dependent production of IP-10 and RANTES, which are encoded by genes that contain the GAS promoter element. In addition, CP-SOCS1 also inhibited the production of IL-6, G-CSF, and MIP-113, which are increased in abundance in IFN-γ-stimulated SOCS1-deficient cells compared to that in WT cells. The production of LPS-free recombinant CP-SOCS1 in the mammalian cell system points to the feasibility of testing this protein in animal models of inflammation, which is currently under way in our laboratory. It is noteworthy that SOCS1 proteins taken out of their intracellular milieu require protein stabilizers, such as L-arginine, a powerful suppressor of protein aggregation, to maintain protein solubility. Fortunately, CP-SOCS1 expressed in our mammalian cell system displayed increased protein solubility; nonetheless, addition of L-arginine was required, albeit at a reduced concentration compared to that required for CP-SOCS1 produced in bacteria. The technological challenges to producing recombinant SOCS1 proteins for intracellular delivery need to be overcome because of their potential use in treating multiple inflammatory disorders mediated by the uncontrolled production of proinflammatory chemokines and cytokines.

The full mechanism by which CP-SOCS1 acts likely extends beyond its inhibition of the JAK-STAT pathway. It is postulated herein, that CP-SOCS1 use might even extend beyond the inhibition of the JAK-STAT and TLR4 pathways. Work is currently under way in which we are combining our innovative approach of intracellular protein delivery with mass spectrometry to identify potentially new interacting partners for SOCS1. SOCS1 contains multiple domains that perform distinct roles. It inhibits the activity of JAK through its N-terminal KIR domain, a domain that is also present in SOCS3 but not in the other known members of the SOCS family. The centrally located SH2 domain in SOCS1 (and SOCS3) binds to phosphorylated tyrosine residues in JAK proteins and cytokine receptors. Finally, the C-terminal SOCS box serves as an E3 ubiquitin ligase that targets signaling proteins for proteasomal destruction. The latter domains, as well as the N-terminal PEST domain, contribute to the rapid turnover of SOCS proteins. A mutagenesis analysis of recombinant CP-SOCS1 proteins was performed to determine whether loss of the N-terminal PEST domain alone or with the SOCS box domains influenced the inhibitory activity of truncated SOCS1. It was shown that the loss of the PEST domain did not affect the inhibitory potency of CP-SOCS1, whereas loss of both PEST and SOCS box domains resulted in a mutant CP-SOCS1 (CP-SOCS1ΔPEST.SB) that displayed greater activity than the full-length protein. The increased activity of CP-SOCS1ΔPEST.SB might have been due to the loss of the PEST domain, which is responsible for increased protein turnover, thus leading to the increased intracellular stability of CP-SOCS1ΔPEST.SB compared to that of full-length CP-SOCS1. Alternatively, the increased activity of CP-SOCS1ΔPEST.SB might have been due to its smaller size compared to that of the full-length protein, which may facilitate more efficient transportation across the cell membrane than that of the full-length protein.

In conclusion, intracellular delivery of engineered, recombinant CPSOCS1 enabled its interaction with the IFN-γ signaling pathway to attenuate the IFN-γ-induced phosphorylation of STAT1 and the production of proinflammatory cytokines and chemokines. CP-SOCS1 recapitulated the functions of endogenous SOCS1 in both transformed and primary macrophages. The development of recombinant CP-SOCS1 establishes the proof of concept of its potential utility as a therapy for inflammatory disorders triggered by acute or chronic proinflammatory cues, such as IFN-γ and LPS, which are difficult to control by currently available measures. The work herein also evidences that controlled intracellular protein delivery, as a facile alternative to gene delivery, could be expanded through custom designing of recombinant CP proteins of interest to target other signaling pathways that are regulated by intracellular physiologic inhibitors.

### REFERENCES

Alexander et al., Annu. Rev. Immunol., 22:503-529, 2004.
Alexander et al., Nat. Rev. Immun., 2:1-7, 2002.
Alexander, W. S., and Hilton, D. J., Annu. Rev. Immunol. 22:503-529, 2004.
Babon et al., J. Mol. Biol., 381:928-940, 2008.
Babon et al., J. Mol. Biol., 387:162-174, 2009.
Babon et al., Mol. Cell., 22:205-216, 2006.
Coornaert et al., J. Biol. Chem., 284:8217-8221, 2009.
DiGiandomenico et al., Sci. Signal., 2:ra37, 2009.
Dimitriou et al., Immunol. Rev., 224:265-283, 2008.
Dinarello, C. A., Chest 118:503-508, 2000.
Hawiger, J., Cum Opin., Chem. Biol., 3:89-94, 1999.
Hawiger, J., Immunol. Res. 23:99-109, 2001.
Jo et al., Nat. Med., 11, 892-898, 2005.
Kamura et al., Genes Dev. 12:3872-3881, 1998.
Kiu et al., Growth Fact. 27:384-393, 2009.
Kuboet et al., Nat. Immunol., 4:1169-1176, 2003.
Liew et al., Nat. Rev. Immunol., 5:446-458, 2005.
Nicholson et al., EMBO J., 18:375-385, 1999.
Opal, S. M., and DePalo, V. A., Chest 117:1162-1172, 2000.
Rakesh, K., and Agrawal, D. K., Biochem. Pharmacol., 70:649-657, 2005.
Siewert et al., Eur. J. Biochem., 265:251-257, 1999.
Veach et al., J. Biol. Chem., 279:11425-11431, 2004.

1. A recombinant polypeptide comprising a suppressor of cytokine signaling (SOCS) polypeptide and a cell penetrating domain, wherein the suppressor of cytokine signaling (SOCS) polypeptide lacks: (i) a functional C-terminal SOCS box, (ii) PEST domain or motif, or combinations thereof.

2. The recombinant polypeptide of claim 1, wherein the SOCS box comprises one or more mutations, substitutions, deletions or combinations thereof.

3. The recombinant polypeptide of claim 1, wherein the C-terminal SOCS box is deleted.

4. The recombinant polypeptide of claim 1, wherein the PEST domain or motif comprises one or more mutations, substitutions, deletions, or combinations thereof.

5. The recombinant polypeptide of claim 1, wherein the PEST domain is deleted.

6. The recombinant polypeptide of claim 1, wherein the SOCS polypeptide is selected from the group consisting of SOCS 1, SOCS 2, SOCS 3, SOCS 4, SOCS 5, SOCS 6, SOCS 7, variants mutants, analogs, fragments, species or combinations thereof.

7. The recombinant polypeptide of claim 6, wherein the SOCS polypeptide is SOCS 3.

8. An isolated nucleic acid encoding a recombinant polypeptide comprising a suppressor of cytokine signaling (SOCS) polypeptide and a cell penetrating domain, wherein the suppressor of cytokine signaling (SOCS) polypeptide lacks: (i) a functional C-terminal SOCS box, (ii) PEST domain or motif, or combinations thereof.

9. The isolated nucleic acid of claim 8, wherein the SOCS box and/or the PEST domain or motif comprise one or more mutations, substitutions, deletions or combinations thereof.

10. The recombinant polypeptide of claim 8, wherein the C-terminal SOCS box is deleted and/or the PEST domain is deleted.

11. The recombinant polypeptide of claim 8, wherein the SOCS polypeptide is selected from the group consisting of SOCS 1, SOCS 2, SOCS 3, SOCS 4, SOCS 5, SOCS 6, SOCS 7, variants mutants, analogs, fragments, species or combinations thereof.

12. A pharmaceutical composition comprising a nucleic acid expressing a recombinant polypeptide or a recombinant polypeptide, the isolated nucleic acid or recombinant polypeptide comprising a suppressor of cytokine signaling (SOCS) polypeptide and a cell penetrating domain, wherein the suppressor of cytokine signaling (SOCS) polypeptide lacks: (i) a functional C-terminal SOCS box, (ii) PEST domain or motif, or combinations thereof.

13. A method of increasing half-life (t_{1/2}) of a suppressor of cytokine signaling (SOCS) polypeptides in vitro or in vivo, comprising:
engineering a recombinant polypeptide or an isolated nucleic acid encoding a polypeptide comprising a suppressor of cytokine signaling (SOCS) polypeptide and a cell penetrating domain, wherein the suppressor of cytokine signaling (SOCS) polypeptide lacks: (i) a functional C-terminal SOCS box, (ii) PEST domain, or combinations thereof;
administering the isolated nucleic acid or recombinant polypeptide to a cell or patient and,
increasing half-life (t_{1/2}) of a suppressor of cytokine signaling (SOCS) polypeptides in vitro or in vivo.

14. A method of modulating cytokine signaling in vitro or in vivo, comprising:
administering to a patient, an effective amount of a recombinant polypeptide or an isolated nucleic acid encoding a polypeptide comprising a suppressor of cytokine signaling (SOCS) polypeptide and a cell penetrating domain, wherein the suppressor of cytokine signaling (SOCS) polypeptide lacks: (i) a functional C-terminal SOCS box, (ii) PEST domain, or combinations thereof;
administering the isolated nucleic acid or recombinant polypeptide to a cell or patient; and,
modulating cytokine signaling in vitro or in vivo.

15. A method of treating a disease or disorder in a patient, associated with cytokine signaling, comprising:
administering to a patient in need thereof, a therapeutically effective amount of a cytokine modulator in a pharmaceutical composition; and,
treating the disease or disorder in the patient.

16. The method of claim 15, wherein a cytokine modulator comprises a recombinant polypeptide having a suppressor of cytokine signaling (SOCS) polypeptide and a cell penetrating domain, wherein the suppressor of cytokine signaling (SOCS) protein lacks: (i) a functional C-terminal SOCS box, (ii) PEST domain, or combinations thereof.

17. The method of claim 15, wherein a cytokine modulator comprises a nucleic acid encoding for a polypeptide having a suppressor of cytokine signaling (SOCS) polypeptide and a cell penetrating domain, wherein the suppressor of cytokine signaling (SOCS) polypeptide lacks: (i) a functional C-terminal SOCS box, (ii) PEST domain, or combinations thereof.

18. The method of claim 15, wherein a modulator comprises a cell expressing a polypeptide comprising a suppressor of cytokine signaling (SOCS) polypeptide and a cell penetrating domain, wherein the suppressor of cytokine signaling (SOCS) polypeptide lacks: (i) a functional C-terminal SOCS box, (ii) PEST domain, or combinations thereof.

19. The method of claim 15, wherein a disease associated with cytokine signaling comprises: autoimmune diseases or disorders, cardiovascular diseases or disorders, neurological diseases or disorders, neuroinflammatory diseases or disorders, inflammatory eye disorder, inflammatory skin disorders, cancer, neurodegenerative diseases or disorders, inflammatory diseases or disorders, liver, pancreas or kidney diseases or disorders, inflammatory disorders of placenta and amnion, diabetes, apoptosis, or aberrant cell proliferation.

20. A method of modulating an immune response comprising:
administering to a patient in need thereof, a therapeutically effective amount of a cytokine modulator in a pharmaceutical composition; and,
modulating an immune response.

21. The method of claim 20, wherein a cytokine modulator comprises a recombinant polypeptide having a suppressor of cytokine signaling (SOCS) polypeptide and a cell penetrating domain, wherein the suppressor of cytokine signaling (SOCS) polypeptide lacks: (i) a functional C-terminal SOCS box, (ii) PEST domain, or combinations thereof.

22. The method of claim 20, wherein a cytokine modulator comprises a nucleic acid encoding for a polypeptide having a suppressor of cytokine signaling (SOCS) polypeptide and a cell penetrating domain, wherein the suppressor of cytokine signaling (SOCS) polypeptide lacks: (i) a functional C-terminal SOCS box, (ii) PEST domain, or combinations thereof.

23. The method of claim 20, wherein a modulator comprises a cell expressing a polypeptide comprising a suppressor of cytokine signaling (SOCS) polypeptide and a cell penetrating domain, wherein the suppressor of cytokine signaling (SOCS) polypeptide lacks: (i) a functional C-terminal SOCS box, (ii) PEST domain, or combinations thereof.

24. A method of protecting a cell in vivo or in vitro from apoptosis, comprising:
contacting a cell in vitro or in vivo with a therapeutically effective amount of a cytokine modulator in a pharmaceutical composition; and,
of protecting the cell in vivo or ex vivo from apoptosis.

25. The method of claim 24, wherein a cytokine modulator comprises a recombinant polypeptide having a suppressor of cytokine signaling (SOCS) polypeptide and a cell penetrating domain, wherein the suppressor of cytokine signaling (SOCS) protein lacks: (i) a functional C-terminal SOCS box, (ii) PEST domain, or combinations thereof.

26. The method of claim 24, wherein a cytokine modulator comprises a nucleic acid encoding for a polypeptide having a suppressor of cytokine signaling (SOCS) polypeptide and a cell penetrating domain, wherein the suppressor of cytokine signaling (SOCS) polypeptide lacks: (i) a functional C-terminal SOCS box, (ii) PEST domain, or combinations thereof.

27. The method of claim 24, wherein a modulator comprises a cell expressing a polypeptide comprising a suppressor of cytokine signaling (SOCS) polypeptide and a cell penetrating domain, wherein the suppressor of cytokine signaling (SOCS) polypeptide lacks: (i) a functional C-terminal SOCS box, (ii) PEST domain, or combinations thereof.


SEQUENCE LISTING (23 sequences)

SEQ ID NO: 1 - protein, 15 aa, Homo sapiens
VTVLALGALAGVGVG

SEQ ID NO: 2 - protein, 12 aa, Homo sapiens
AAVLLPVLLAAP

SEQ ID NO: 3 - protein, 16 aa, Homo sapiens
AAVALLPAVLLALLAP

SEQ ID NO: 4 - DNA, 39 bp, Artificial Sequence
  Synthetic Nucleotide
gaggattcttaggaggagagaggtcggctcagtaccagc

SEQ ID NO: 5 - DNA, 72 bp, Artificial Sequence
  Synthetic Nucleotide
cgggatccgccatggcccatcatcaccatcaccataatgcccataccggtgcagctgtgcttctccctgtgc

SEQ ID NO: 6 - DNA, 36 bp, Artificial Sequence
  Synthetic Nucleotide
gagaattcttaaagtggagcatcatactgatccagg

SEQ ID NO: 7 - DNA, 72 bp, Artificial Sequence; identical to SEQ ID NO: 5
  Synthetic Nucleotide

SEQ ID NO: 8 - DNA, 36 bp, Artificial Sequence; identical to SEQ ID NO: 6
  Synthetic Nucleotide

SEQ ID NO: 9 - DNA, 75 bp, Artificial Sequence
  Synthetic Nucleotide
cgggatccgccatggcccatcatcaccatcaccataatgcccataccggtatggtcacccacagcaagtttcccg

SEQ ID NO: 10 - protein, 211 aa, Homo sapiens
MVAHNQVAADNAVSTAAEPRRRPEPSSSSSSSPAAPARPRPCPAVPAPAPGDTHFRTFRSHADYRRITRASALLDACGFYWGPLSVHGAHERLRAEPVGTFLVRDSRQRNCFFALSVKMASGPTSIRVHFQAGRFHLDGSRESFDCLFELLEHYVAAPRRMLGAPLRQRRVRPLQELCRQRIVATVGRENLARIPLNPVLRDYLSSFPFQI

SEQ ID NO: 11 - DNA, 1766 bp, Homo sapiens
ggcagctgcacggctcctggccccggagcatgcgcgagagccgccccggagcgccccggagccccccgccgtcccgcccgcggcgtcccgcgccccgccgccaggtgagccgggccctgggcgaggaggcgggagggaggagggaggggagtccagggcagccaggagtcgggcgagcctcgggggctgcagaatggggtcgcggccgcgatgcccctgaccctcgccggccccacccaggccgccccccgcgcgcggggctcccgcagcacagcctttctccggccctagcccaaatcgcccagaccaggcgcggatcccagcctggccagcaggcggcgggcgcggggcggcgagccggggccggacggctggagccagaaccggctgctctccacgcccccctctcggtgctgcccggaggccggactccgcctccaccgagcccccacccgccgggaagagctccgcggagtacagagcccattttctagctgtgtccactgaggctgaacggatccgcgcggacttggtgctccgtgctcgccccctagggccgggtccgccgggagcgccgccctccggagttgtccggccggcgcacacctgcccggccccgcagcgccccagctcacctctttgtctctcccgcagcgcacccccggacgctatggcccacccctccggctggccccttctgtaggatggtagcacacaaccaggtggcagccgacaatgcagtctccacagcagcagagccccgacggcggccagaaccttcctcctcttcctcctcctcgcccgcggcccccgcgcgcccgcggccgtgccccgcggtcccggccccggcccccggcgacacgcacttccgcacattccgttcgcacgccgattaccggcgcatcacgcgcgccagcgcgctcctggacgcctgcggattctactgggggcccctgagcgtgcacggggcgcacgagcggctgcgcgccgagcccgtgggcaccttcctggtgcgcgacagccgccagcggaactgctttttcgcccttagcgtgaagatggcctcgggacccacgagcatccgcgtgcactttcaggccggccgctttcacctggatggcagccgcgagagcttcgactgcctcttcgagctgctggagcactacgtggcggcgccgcgccgcatgctgggggccccgctgcgccagcgccgcgtgcggccgctgcaggagctgtgccgccagcgcatcgtggccaccgtgggccgcgagaacctggctcgcatccccctcaaccccgtcctccgcgactacctgagctccttccccttccagatttgaccggcagcgcccgccgtgcacgcagcattaactgggatgccgtgttattttgttattacttgcctggaaccatgtgggtaccctccccggcctgggttggagggagcggatgggtgtaggggcgaggcgcctcccgccctcggctggagacgaggccgcagaccccttctcacctcttgagggggtcctccccctcctggtgctccctctgggtccccctggttgttgtagcagcttaactgtatctggagccaggacctgaactcgcacctcctacctcttcatgtttacatatacccagtatctttgcacaaaccaggggttgggggagggtctctggctttatttttctgctgtgcagaatcctattttatattttttaaagtcagtttaggtaataaactttattatgaaagtttttttttt

SEQ ID NO: 12 - protein, 198 aa, Homo sapiens
MTLRCLEPSGNGGEGTRSQWGTAGSAEEPSPQAARLAKALRELGQTGWYWGSMTVNEAKEKLKEAPEGTFLIRDSSHSDYLLTISVKTSAGPTNLRIEYQDGKFRLDSIICVKSKLKQFDSVVHLIDYYVQMCKDKRTGPEAPRNGTVHLYLTKPLYTSAPSLQHLCRLTINKCTGAIWGLPLPTRLKDYLEEYKFQV

SEQ ID NO: 13 - DNA, 6381 bp, Homo sapiens
agccgcggcctcaactaaaagtggccattgacctttcaagctttcgagcagtgatgcaatagaatagtatttcaaagaaaaatgcttatcgaaattttggatccggttttcccgtgattgttaagggtttcttttaaaaagtaggtcacatttcaagtaggtcatatttcgggggcgggtgcgcagacaaggagatgagtttccactaaggccagggggcctccaacggggttggaggtgagaatcccaggtagggtagaggtgccgagatccttccgaatcccagccctggggcgtcagccctgcagggaatggcagagacactctccggactgagggaaccgaggccagtcaccaagccccttccgggcgcgcaggtaagggcgcccccttagcagccggcgcaggtgacccgggcgggccgccgggtctaccggagacgttggagcagaggggaggaggaagggaggagccgggtgggtgcgggtgacaaggagccggagcgccagggggaggggactaaggacggccggcgccgcttaaggaggcgctgctctcccgctcgctgccttccaggacctgatcaaggggaccgcctccggtccccggccgtgggcaccgggacgagcacggcgtccccacgccatcgatgtgtcttagagccggagagtctggtttccgaggacccacagtcgctcctgcacgcccaccccccgcaaaagtgcggccaggagggtcgcatcgagggggcgccgccgggatgtttagaggaacccacccccgtggcaggccaagggccaaggatcgctatccttccctgaacccgggcgctcagctggcccgggtagggggcaggctccggccgccgaaacggggttggctgtagccggtggccggggagatctctagcttgcgcccgagcaccccggggtgtggggccagaggcaggccgaccccggcgtgcacaccgcccgccctgcacccgagcgctctcacccggtcttccctgaagcctgtgtattgcgaccgagcctctttaaagcagtagcggggcccgcggtcacgtgaggccgattcctggaaagttcctggaaagccgcctccgcagcagccgggcggggcgcgagcggagcgctgactggggagggaggcggggagcaagggaggcgcgtcggtctgggaagtcgcgcgcactcgctgctcctgggaccgacgtttaactcttgccaagtctcgtcgcagccgccgcggctggcgggccttgggcttcccctgaagcatgagccctctcgcccgcagccaccctcaccgcgtggcccgcggacagtgcgcgccggggtcccgggtgcacagcctcaggataccccgtgcccgcagctcgggcgcccgcggcaggtaccggtagtgggggaagcccgaaggctccgccccgaggagagttaccggggagggcggcgaggcgcggccgcgtgcgccggggagcggcggacggcctgggcttccgcagctcggagcgccggggaagagagagtccgaaccgcggctctgccccgcggcctagcgttgctgcctgctttctccagccccttctcggcgtctggaagtgtctggagtttcttttttttatttcccctaaactgccattcaaattaataatcctcctaataacctgatctcccgctcctccccaccggcctgcctcccgccctcgctccttcctccctccctccctctctccttcccacctccagggtcgcagccggagggaaacccggcagcagtccgagagtggaggtgtcccagcccgtagggggcgtcgccgcgcggtgggggatggggtcgaggcaatgatcctcgaggcttttgtgtgccctctgcgcacggaactccgaccgccgcctccgagcgcggggctggttgcatccccgggcatctcgttcccaaattaaacgttaacgggggaaacaagggcagacgcccctcctctcccgggcccctcccacctccccttttccccccacccccccgccccatgtccgctgaggaggctgcctggtgcggaggcggcggcggcggccgcggccgaggtcgaggtaagagcgcggcgttggtggtttgcattttccgcagcgctcgcgaccgcccgctcggccccggcctcgcctcggagccccgcgagggctgcgcgctcgggccgattcctcgacagcgcccgcggcggcgagaaccaccgcagccatcctggtccgggagcgaccgccgcggcacccccaaccccgccagagcgggcaccaggaagcggggggaggggagcgaggagcctcccgggcggagatgcggcagctcccgcacctccgcaccccgggttggtgcctcggggccccgcagcgctttagggcggagaaccaagtttgtgtgggtgcttcttcctgggggccctcgggctgcgccaggtgtgagaccggaatgccaatttggggaatctctcctgttctttcctttttaattttttctacttgtccttgacaagggcctattcccactggatctgcaggtgactatttgctcttcctaggtcgccagtctttgatttcagctagtgaccctgggcgggaagacaccctgttcaccctcctcctgacctccgccccccacctcttcagccaactttatcttggatctctgctctctttatcttttctctagagctgggccagggcgctaactggaagcttggggttaaatggtgcaggaacgtagaggcggaggagtccctgggattttccacgtctatttccccacccccaccccagccgcaggggtccagtttggactgacccaacctcccgctttctctttgtaggcgatcagtgggtgaccgcggctgcgagggactttgtcatccgtcctccaggatctggggagaaagagccccatcccttctctctctgccaccatttcggacaccccgcagggactcgttttgggattcgcactgacttcaaggaaggacgcgaacccttctctgaccccagctcgggcggccacctgtctttgccgcggtgacccttctctcatgaccctgcggtgccttgagccctccgggaatggcggggaagggacgcggagccagtgggggaccgcggggtcggcggaggagccatccccgcaggcggcgcgtctggcgaaggccctgcgggagctcggtcagacaggtagggagccgatcggccgcgacgcgtgcgggagggagcgcctccccaaggaagcagctaggaagcggggtcgaggtgggaagcaaagaataagatggaaatacgtcccttgcttccaagggaccgcggagagcacgctcgcagggtcctgggtccttgggaatgcgtaaggaaagtggttctccagggactcaggcctggcggagcgcagagcgcgggaagagcttcttggaaatagcttcttagcacgctggaagatttactgttttccgcagctgctcagggttaggctcccgggctcgaaccccggccggggaaagcgtcgggcgcctccctttgcacggggtgcaatcagcaagtcggtgccgcgggccccgcaggcccagcagcatctggctcccgggcggagctgcggctgcagcccaggatcttggcggccaagttcagggactgacactgccgcgagggcggctgcccgggcgtcgagagtaggctgcgaagcccccgccgagcgcggcttccatggtcggcgcgcccagggtactgccgctttcaagtacagtcaggcaattcgcgcggcacgctgtctctaggcatctgaaaaaagaaaaaagaaaaaaagtaaagtatctggcaaatcattctggctttccaaatccgtatcctaaaagcttacagttgtggcacagttgaaattttggtagcaacagggggagggagagggaaaagttctctcgcttgcgattccggtctttccgtgtgggccacgggctttccctcccggggaggcaccggcagattgcaagtatcccggtaattgtgggggtggggggagcacaaatgttcagacttcttaaatacggacggtctctgagccttcttagaaagcagagtgggccggggagatggtgcaggcagatgaaatcaagaatgcaggggagatggatggttgtgattgggtttggattagggcaaagttcaaaagagtttagggaagattgtaaacttgaagtagtaattatcacttgggaattcccatttcttagtgactggtaaggtagtcataaagcagcgaacagaagctattgtactagagctgtgcttttacaacaagtacatagcagtgctttctacatagaaggctgttcatagcaggctttgcagattctttttttttttttttttttttttttggtggggggcacaccaaccatccaaaaaagttgcttctgtttttactgtagttcaattacaggagttgggttttccactttttaaagggcatggcttttatttctaacttctgatttttttctaatttacctaaatctttattttacttaaataatgttaaaagtgtcatgcttcatcagccagtgtgtcatgctgcagatgttcttttggttcattagaagaaaatcttaagaagagcttcagaaagttgattttttaaaaaagaaagaaaactggctcgccgtttgtgttcataaaatggacccaaatttttattaaattcctactgaaagtatttgtggtggcccggtaaactttcgctcacacaccaccttttttttttttcttttttagagctaaaaaaattattgccaattacttgctctgttctaagaattcttataataactgttttaccctcttttctttttctttttgaacaaaaaccccaggatggtactggggaagtatgactgttaatgaagccaaagagaaattaaaagaggcaccagaaggaactttcttgattagagatagctcgcattcagactacctactaacaatatctgttaaaacatcagctggaccaactaatcttcgaatcgaataccaagacggaaaattcagattggactctatcatatgtgtcaaatccaagcttaaacaatttgacagtgtggttcatctgatcgactactatgttcagatgtgcaaggataagcggacaggtccagaagccccccggaacggcactgttcacctttatctgaccaaaccgctctacacgtcagcaccatctctgcagcatctctgtaggctcaccattaacaaatgtaccggtgccatctggggactgcctttaccaacaagactaaaagattacttggaagaatataaattccaggtataaatgtttctctttttttaaacatgtctcacatagagtatctccgaatgcagctatgtaaaagagaaccaaaacttgagtgctctggataactatatggaatgctttctaagaacagctgaagctaatctaatttaaatttaacagcttgaagaggtagctaggtgtttaaagttcctccagatacttttacctgagtgatgcttcccttcctaaggctgaccaagacctgttgatccttttagattaaaaataaaatgtcgcatgtaaaggctgaagtcgcgttttatcagaatgccttgccttcttaggttcttttccattatgtcaaaggtccaggctccagtaggagagaaagaactcctcataggaatactgaagaagtgggaaggaaccaagctgacacaggcctcactgcaatttgatatgcctgctgatcagagtctcttgggcattttatattttgcattctgatgtacctaggagttttgttaaacagatgatgtatgtgagtatttatcccattttatgcaattaaccaaatcaaccaaaaaaagtgaccatgaagtcctgtatttgtctttttactacatgtaggaactctcatgtgaatgagtactgtagtaatccattctatgggagccttatttcagaaatatttcaaactggtgcaaatggaaaagactttctcttttcctttaaagctaaagacaagaatatcatgctatacaggtgcaactcaatccccgttaataaaaaccaatgtaggtataggcattctaccctttgaaatagctgtgtcccaacctgttgccattgattttttggaaatggctttagaaatatccaagttgtccttgaattgtctaaccatggacataaacagttgtctcccttctactgtgtagaatactttgacttaattttcttccagatacagggggatacctgcctgtttttcaaagtgtttatttactgctgttactatttgattagaatgtattaaataaaaaaaacctgatttct

SEQ ID NO: 14 - protein, 225 aa, Homo sapiens
MVTHSKFPAAGMSRPLDTSLRLKTFSSKSEYQLVVNAVRKLQESGFYWSAVTGGEANLLLSAEPAGTFLIRDSSDQRHFFTLSVKTQSGTKNLRIQCEGGSFSLQSDPRSTQPVPRFDCVLKLVHHYMPPPGAPSFPSPPTEPSSEVPEQPSAQPLPGSPPRRAYYIYSGGEKIPLVLSRPLSSNVATLQHLCRKTVNGHLDSYEKVTQLPGPIREFLDQYDAPL

SEQ ID NO: 15 - DNA, 3300 bp, Homo sapiens
ggctccgacttggactccctgctccgctgctgccgcttcggccccgcacgcagccagccgccagccgcccgcccggcccagctcccgccgcggccccttgccgcggtccctctcctggtcccctcccggttggtccgggggtgcgcagggggcagggcgggcgcccaggggaagctcgagggacgcgcgcgcgaaggctcctttgtggacttcacggccgccaacatctgggcgcagcgcgggccaccgctggccgtctcgccgccgcgtcgccttggggacccgagggggctcagccccaaggacggagacttcgattcgggaccaggtaggaaggaggagcgcggcgtggggaggggtctcgctcagtcccgggagcttttcccggtttcccctccccttcccgggtcattcccggcagggaggtgacgaggtaggggcagagcggatggaagccggagatcccaggttcccggaatactccggctggggccttcgggcttctcctgtcccctccctacccccgtgcctcgggtttctccctccgtccacaccgcccggggctactggactgagcggcgcccaggcagtcccgggggcccttctcctgctccccacccggccacactcctggagacctaacttccgcgcgcgagtttcccacgctgcgcccttgcagtgcgcgcctgggaaggggctgcccggggccaccctgccggcaggggcgggagccgtgcgggctccgtgaggcgcctggatcggagcgcgggcccaggagagggcccccggggcagtgggtgccccagtcgctcggcgaaggcaggggagccggggcgggccgggcgcgctggagggttccgggcactcaacgcgctcgcgccttcctctccgcagccccccgggatgcggtagcggccgctgtgcggaggccgcgaagcagctgcagccgccgccgcgcagatccacgctggctccgtgcgccatggtcacccacagcaagtttcccgccgccgggatgagccgccccctggacaccagcctgcgcctcaagaccttcagctccaagagcgagtaccagctggtggtgaacgcagtgcgcaagctgcaggagagcggcttctactggagcgcagtgaccggcggcgaggcgaacctgctgctcagtgccgagcccgccggcacctttctgatccgcgacagctcggaccagcgccacttcttcacgctcagcgtcaagacccagtctgggaccaagaacctgcgcatccagtgtgaggggggcagcttctctctgcagagcgatccccggagcacgcagcccgtgccccgcttcgactgcgtgctcaagctggtgcaccactacatgccgccccctggagccccctccttcccctcgccacctactgaaccctcctccgaggtgcccgagcagccgtctgcccagccactccctgggagtccccccagaagagcctattacatctactccgggggcgagaagatccccctggtgttgagccggcccctctcctccaacgtggccactcttcagcatctctgtcggaagaccgtcaacggccacctggactcctatgagaaagtcacccagctgccggggcccattcgggagttcctggaccagtacgatgccccgctttaaggggtaaagggcgcaaagggcatgggtcgggagaggggacgcaggcccctctcctccgtggcacatggcacaagcacaagaagccaaccaggagagagtcctgtagctctggggggaaagagggcggacaggcccctccctctgccctctccctgcagaatgtggcaggcggacctggaatgtgttggagggaagggggagtaccacctgagtctccagcttctccggaggagccagctgtcctggtgggacgatagcaaccacaagtggattctccttcaattcctcagcttcccctctgcctccaaacaggggacacttcgggaatgctgaactaatgagaactgccagggaatcttcaaactttccaacggaacttgtttgctctttgatttggtttaaacctgagctggttgtggagcctgggaaaggtggaagagagagaggtcctgagggccccagggctgcgggctggcgaaggaaatggtcacaccccccgcccaccccaggcgaggatcctggtgacatgctcctctccctggctccggggagaagggcttggggtgacctgaagggaaccatcctggtaccccacatcctctcctccgggacagtcaccgaaaacacaggttccaaagtctacctggtgcctgagagcccagggcccttcctccgttttaagggggaagcaacatttggaggggatggatgggctggtcagctggtctccttttcctactcatactataccttcctgtacctgggtggatggagcgggaggatggaggagacgggacatctttcacctcaggctcctggtagagaagacaggggattctactctgtgcctcctgactatgtctggctaagagattcgccttaaatgctccctgtcccatggagagggacccagcataggaaagccacatactcagcctggatgggtggagaggctgagggactcactggagggcaccaagccagcccacagccagggaagtggggagggggggcggaaacccatgcctcccagctgagcactgggaatgtcagcccagtaagtattggccagtcaggcgcctcgtggtcagagcagagccaccaggtcccactgccccgagccctgcacagccctccctcctgcctgggtgggggaggctggaggtcattggagaggctggactgctgccaccccgggtgctcccgctctgccatagcactgatcagtgacaatttacaggaatgtagcagcgatggaattacctggaacagttttttgtttttgtttttgtttttgtttttgtgggggggggcaactaaacaaacacaaagtattctgtgtcaggtattgggctggacagggcagttgtgtgttggggtggtttttttctctatttttttgtttgtttcttgttttttaataatgtttacaatctgcctcaatcactctgtcttttataaagattccacctccagtcctctctcctcccccctactcaggcccttgaggctattaggagatgcttgaagaactcaacaaaatcccaatccaagtcaaactttgcacatatttatatttatattcagaaaagaaacatttcagtaatttataataaagagcactattttttaatgaaaaa

SEQ ID NO: 16 - protein, 440 aa, Homo sapiens
MAENNENISKNVDVRPKTSRSRSADRKDGYVWSGKKLSWSKKSESYSDAETVNGIEKTEVSLRNQERKHSCSSIELDLDHSCGHRFLGRSLKQKLQDAVGQCFPIKNCSSRHSSGLPSKRKIHISELMLDKCPFPPRSDLAFRWHFIKRHTAPINSKSDEWVSTDLSQTELRDGQLKRRNMEENINCFSHTNVQPCVITTDNALCREGPMTGSVMNLVSNNSIEDSDMDSDDEILTLCTSSRKRNKPKWDLDDEILQLETPPKYHTQIDYVHCLVPDLLQINNNPCYWGVMDKYAAEALLEGKPEGTFLLRDSAQEDYLFSVSFRRYSRSLHARIEQWNHNFSFDAHDPCVFHSPDITGLLEHYKDPSACMFFEPLLSTPLIRTFPFSLQHICRTVICNCTTYDGIDALPIPSSMKLYLKEYHYKSKVRVLRIDAPEQQC

SEQ ID NO: 17 - DNA, 22364 bp, Homo sapiens
agcgcaggtcccgacagaggtcgccgggtggtggcgtcagctgcggcgccctccgcgtggaaattagccggttgcccgggcaaatcagggaggaagggagggagctgaacacggaagtggtggcggcgcccagggaaccggcggaggcgatgaccgtgacggctgggttgggaccggaacgccgaagcggggttgggggtggcagaaaagcatctgctttgtaagacctacacgaggtgcaggagtggttgggcctcccctctccacttaagcaagcgcccagactgatggcgatggtgatggcagcagttactcgcacaaccccagttaagctgcgctccgggaggtgagtgggggaagggtggccgctgcctggcctcgcaggccggggtcttgttcctgccctccaggctctcctcccaggaaacaatatccggatccctaaaaggatccggtctctttcttggggcccagttagcagacagatctttgagcatcttctctggaaaagaaggcgagatcttctagagcctctaatcttggcatccttcgtgccctcaccttgtcgcttctgtagagataaagaaggaatgacttaatttctgttccggttggttctcttggtacctgaaggtataattaattaatgctcaattaatgcttattctttgcgactctatatatccgttatgaagaaagtaacttgtgttctctattcagatcccacctaaagtttactgaagtgaatatttataaccaaaagaagagttataacttagtagccttgagcacttacatttgatccatgcaatatctgctaaagtcttggccactaagtttaagtttatgcttttcttgtatcttcattctccatattggtacagcagcctgtgatgaaaaccttcagaatgatattctttcatttgtacttatctttgagaatttttctgcgtttttgtgataccttccagtatcaatcacatttattttattacctgagacgtttttctgttgaagaggtttccctattgtttttttcaatctagttccattgtgaaaagttgcaaacacacaggcaaaccgtcgtgttcttaccgtgggtggcattgtgtgaggcgttgtttttagtactagagctccagatagccttgctagacgctgaggtttgtatatgaatgaagaggaagaatgattggcttaaaaacaacttgggatttgtggaaaaataacccactgttgatctcccgaaagagcagtggtaccagagatgatgctttctgagtgacctaattttttttttttggagatcatttgttttttggatagactggcttataaattttttcgttcaagtaacaatatcattggctagagtacatagagttcaagatgaactctcataatattcgtctctatgttgaataaagcgtggtgagatttctggttagtactattcagagctcagcataagaagaatctgcaaattggaacaagttatagctacaaaatgccttcactataatatctgtaatacccatgagactatgattgatgtccacacatctgtcattttctcttatcaatcaagccatttgattcataggttaaagaacaattttctttgcagattttgcatgtatggtaaaagcttgagcctccgagtttaacagtcctgggttttaattctaactgacacattgtgtgaccttgaatatgttcctttactgaaaatcgaattcctcatctctaaaatggagatgacacctacctccgtcgcattgttagagtatataggagatactcatggatgtataaaagtaattgttattaatatctcatgttactaaaggggtgctttgggtgggtatttagtgttttttgtttaaagtgggatttgtttaaataattatccagtaaatttttcaaatattaaaaaatgtattcttgtttttgctagtatcatgttttcacaattccatttgtttcagtcaaatggctacaaattgtaataccttcagtgagtttatggtcttgctgagggaaatagacattcaaacaatatgatcaagatgatagtgcagtaatgaaagatgtgtaacttaaatattagcaaaattgtctagttagttaaaataacatagttttttaaaaagaaatcagtgtagctgtgtcactgataggagtatcaaaaattttccaaatgtagttatttgtcattaacagtgcttatctcataggtgtgggtggctaaatgaactgactgaaaatgatgccaaagtcacagattgaattgcagtgatggttagttagctttgctgtgttgtaaccactcatactacccttagactcacaaatatttttgctcccaaggacaattgggtgagaaaacatgggtacattgccattctaccaatgatggtattacctatataaaatcatgtgatcttacaggatttatctaagaggatatatcagtattggcttatggaaggagtttaaaaaattattttctagtaaaatatgtacttggtgtaaaaatgattaatcttgtgaaaggtaaggctagaagagtgtaacaatagccaaggagaccaaagtattgaaaaaagaaacattgaaggcactttagaaatgtgaagatttttatatttccaaatgtgtcctgaaacttactagcctatttaataattttggggtaaatagtacttgtctgtatgtatacttaatatgatataaaaggcaatacagtaaaagtcgtcctcctactcatactcccagccatccagttcccctggctaaagacagccagtatttgattcttgtattacattaacactagatccttactacagcaagatgagataatcagtgttatatagatagaaaaacagaaaagttgtgatttatctagtctttatctagtcacacacctgcgaaatgtcagagctagaaatagaacccaggtctttcacctagtccaaggtagtctaccgcactgattctccaaatcatagctgttgccacaaaacttagaatttagtaaaacataaagtgtagagtcaccttttactaatttgtacgtaaaacttgttacccaaccttaaatgtaagcatatgtaattgttataaagcagtgaagaaaatataatacaatcttataaaatctttgcctgtttctacttaaatctatttactaagcttctaaaaaactacaattttttgttttaaagaatttaaaactttttaaaaataatatatttaacatcaggaataatataaagtgctttgtgatatttcagaaatggaagagcctccttgacttcagatttctgaggcacgtttttgtgcagattttctagccttacatatagcctttgtttcccctcccctttttcttaaaaaaatagtgttctttaaaaaaagtgttcccacctctcatttttagaaatgtacttcttctaacatgaaaagttctgagtaatactaggaagcagttaatatttcttttcattaagttcagtgtatgttgttttcataccttttactcctggcagtaaaaacatgaggtagctggcattatccagattttacagtgggagaacttaagctaaattagatttttttaaaccaggtaacatacctagtgagtggaaaagtctgaattgtagcccacatttgacagttcagcagctcatgctatttcatcttcaccatgtttattttcaaatatggtatagtgaaaaacagtgtaggttgttaattttttacagatctttaaaactttaaaattttctctttatgcttaattggtctcacaagaaaaaagaattcaaggaaataagccaaaatgtcaaaattgcttgcttttaaataatggaattatagcttttttccccctcctttttaaacttttctgaatggtccatgtattatacaatgagcatatattgtttttatcataacaaattacagattggaagaaaaaataaatctgttcaatctaatttattccataaaatggttcagtattatatcttaaatattctgtttcttaaattctgttaatcttccttctgcttttagaagttcaggtactgccagctcgtagtttctgctttcttcactctagattagccccttcagtgatgggttcctagacctctctccttatctttagctaataagcttagggaatgtacactgatgttaacagattagcccattaatgtttccccaaactttcttccacaaaaggatttatggcaaagtaaagacaggaagctatggtttatgcagatttctttactgaaggatctcctgggaacgtttgctaaatatgcactgtaatctttgagagaagaagcatttcccaaacttatttgacaagagaacctttttttctccagaatatgtgttaacatctttcagaactagtgtccacaagagtacagagtggggaaaagtgacataagcaaactataattaggaaagcatggaaaaagtggggagaaagggaaaggagggtgtagtgctactaaatcaatgattcttttttagaaaattgcaattaagtcccatgagccctggctcagtagcatgaaaaaaggagatactttaaaatatttactggaaaatgaacctgtattgcttctgtaatgagaaaacatttgttttttaggtcagatattttcccatacaaacaaacctgttttcagatttttttcttaacattccaaggagttcatctgttgtctgctctccagtgacttccgtttgtggagcctaagtgtttctacagaatccaactcaaatatcaaggactatgatcagaatgctttcaaggagtttttgggcacatgatggcaggtaaactttggaatcatgaatttcgtagggaaagtccccaggcttaattactaccagaataataatatatttgtgaattattaatgtagtatactgaattattctttataggatcaaatgtattccaatttagtatttacattagttgtggtgttagatgtatgtcagcatcatagacataagtagttcaatgaataattaacgtatgcaataatgctgttatcctacatcatagaggatactaattaatttccagataaatgaagcttatccttttaattattggatttgaattgtttcctgtagaaattttgaattaaaatcaccttctcagccttagtcagtattactaaatgttatttaaatattttgtaatgaaaattgacataatgggcactcattttgttattgttgtcattgtaatttgtggtagaacctgggaccaaatagtggcagaatctgggtacttgttttcttgagaaggcttttatataaacttgctttttaaacatttgctttttgctcattaagaaatatgacttaaaaaaaaaaagatgtcattctgtactccccagggcaatgtaacatagctgccttcgtaaaaaattaattattgaatcaagagccaaagcagccttttcccaagtgattctttcaccttctttttaaaaagcttttttgcagctacagtcaaagatctttgatttgtcaccatctttcaaaccatcatctttgatttctgtgtcacatgaaattgttggtaaagccaccttattattattctacctcatgtatacaaagtttttttttttcttttgagacggagtctcacccaggttagagtgcagaggcacgatctcagctcactgcaaggtccgcctcctgggttcatgccattctcctgcctcagcctcccgagtagctgggactagaggcgcctgccaccacacccggctaattttttgtatttttagtagagatggggtttcaccgtgttagtcaggatggtctcgatctcctgacctcgtgatccacccacctaggcctcccaaagtgctgggattacaggtgtgagccaccgcgcccggccctatacaaagtttttataaaattttaagaattaatgataggctatctggaacaacttgttcggattcagaaccctacactttttttttacattcagattttacatgtatggacaagatacgaaagcacaatttcaaaaaactaatctttagtcttcataacaataaaaataatttttctctcactaaaattaagttatttttattgaaagcaacatgattgatttttttatattttagaattttggttcaaagaagcttctggaagagattagaaaattctctttatatgtgtttaagtataaagatatgaaagtttgatagacttctctcatttttttgtaacagaatcaaataatggtgaaagaaattctaaatatcttttttctgataagatctctccatagcacacagttattttttaagcagttactttctcagtaattgttaagatgttacctatgccgaaaagtgaatgattaaatgtatgtgtttttaaaaaatatatctatctgctaagtagcttctgagagccacttgttatttcagtaatggttatgaaatttagaacacttgttattttgtaaaagaaaaatgcacaactcctcttaaaagttcaacaacttatgtaagtactttgtcttgagataattagcagaccttgcatgttataaaacgtctatggtcatgtctcattacaaagtattttaaagattctcttgtttaaaataatacaatccacgaaatattctagtaagatataaacagtaaagctgggcacggtggcgcatgcctgtaattccagcactttgggtggccgaggtgggtggatcatttgaggtcgaggaattcaacaccagcctggccaacatggtgaaaccctgtctctactaaaaatacaaaaattagctgggcgtggtggcacatgtcagtaatcccagctactggggaggctgaggcaggagaatcacttgaacctgggaggtggaggtgcagtgagccgagatcgcactgctgcactccaccctgggtgacagtgagaccctgtctcaaaaacaaacaaacaaaaagatataaacagtacactgaaagttaataaagaaatggagagggctggctgccataatttgtcagctttttggaattcccattcttctcttaggaacagcacagacaaatgaccatctgacaatgcatgatggtaacattatcaaccatatttagtactaatattagtttttgtatgttttagacaaatataaatagaaaaattttcttccaactttgaagactaaaatatctgatgacattctggatcatatattgatggggcaaaagataacattttataaaagtaggacttcttgagcatgaacttaacagttctctagcaaaagaatagctacgcaaaatttatataaagctctaattctgagatagatacaaaacagtaactaatcagaaagttttcaggccagtcgcggtgactcatgcctgtaatcttagcactttgggaggccgaggcaggtggatcacgaggtcaggagttcaagaccagcctggccaagatggtgaaaccccgtctctactaaaaaaaactacaaaaattagccaggcgcggtggcaggtgcctgtaatcccagctacttgggaggctgaggcaggagaatcacttgcacccaggcagcagaggttgcgccactgcactccagcctgggcgacagagtgagacttcacctcaaaaaaaaaaaaagttttcattgcattaactctctctctgtttttttttttttttccttgacatggaatcttgctctgtcgcccaggctggagtgcaatggtgcaatcttggctcactgcaaccgccgcctcccaggttcaagccattcttctgcctcagcctcccaagtagctgggactacaggtgcacgccaccacacccggctaatttttgtgtttttagtagagaggagattttgccatgttggcgaggctggtctcaaactcctgacctcagatgatccacccacctcggcctcccaaagtactgggattacagacatgagccaccgtgcccggcctgcattagctctttaagtacaggtgaacgtgtggaaaactcaactagggcagtgatatttttcactttattttatggacatctacagcattagcacacgtaaaaacagttaccctaaaatccaaacacacttgattgactggtacctgtaacatacaagttgggagtcttgcaaaagaaaacctgttttgttagcgtgtaaacttgttctttgggggaagatgtcaaatttttctgtaacttcatggtctatgagtttcacctgtatctgacatgtggatcctttttcactttgaaacattagttttcctcctgaaaattatatttgctcatgaaaatctctttaccttgtagtaacactgcactagagacagaggtattctgtgatttcttcattatgaagaaaactgtaaagccaataagactggcttaggtgctttaacaaaacaacatatgtaatctttgtttctttttaaaacaaaaagaaaatatgaacatatatgttaacatatattgagtaattaatacatttaggacctgagctgaatacatgcattattttatccccaacagcccagtgaaatagatagcattagtacctccattttacatgtgaatgcactgatgtttcaaggttatatggctagcacaaaacagaaactggacttaaaccatgtttcttttttttttttcttccagaaatattttaataaaaattggccatttccaagaaacacccactgcatgcccatgaataaaagagaaacataacaaaatagaaaatttaccatatggctatgatttaaagaaaaaaaaaatgcggacacgagcacattagcaaaatattaaagtcacatgtcttcttatggactcaaaccatgtttctaatgcctgccgccgctatgctgtgggaatgcatccttgccatccatatacatatatagagatactgagaatgttaaagtggttactgctgggtggtgggattatggatatatttactttttccttataactttatgcctagtttgggttttccatggtgagcatgaattttccttctcagaaagacttagtatctcctctttatctctaggcttctaaaattagaataatgtactttgatgttgagtgggatctttcaatatggaggtgcctgtcctttagtcctgggacattttctaaccttttttttttttttgagatgaagtctcactcttgtccctcagggtggagtgcgatggcatgatcttggctcactgcaacctctgcctccagggttcaagtgattctcctgcctcagccccccgagtagctgggattacaggtgcctgccaccacacccagctaatttttgtatttttagtagagacggggttttaccatgttggccaggctggtctagaactcctgacctcaggtgatccacccgtctcggcctcccaaagtgctgggattacaggcttgagccaccacacccggccattcctgggaaattttctaatatagtttctttaataatttctcccgtccattttgttgttgttttctgtgaaatgcctatattaatctctaattttcttgtcttttctgtctagttaaccattttttcctatttgctgtattagttcctaagcatacccacgaacttttctattgaatattttatttcaggccaggtgcagtggctcacacctgaaaatccaagcactttgggagccaaggtgggcagatcacttgagcccaggagttcaagaccagcctagataacatggcaggaccccatctctacaaaaaaatacaaaaattagccaggcatggtggcatgcacctgtagtcccagttatttgggaggctgaggcaggaggatcacttgagccctgggaaacagaggttgcagtgagctgagatcagtgccactgtactccagcctgggtgacaaagtgagaccctttctcaataaaaaaaaaaattattttagcaatcatattttttttttttttgagacagagtctcactctgtcgcccaggctagagtgcagtggcatgatctcggctcaccacagcctccaccacccgggttcaagcaattctcctgcctcagcctcccgagtagctgggactacaggtgcctaccactgtgcccagctaatttttgtagttttagtagagacagggtttcaccatcttggccaggctggtcttgaactcctgacctgatgatccacctgcctcccaaagtcctgggattacaggcgtgagccaccgtgcctggcctaacaatcatatttttaatttccaaaaaaattcttattctagttgtctccacctctttttttttattttttttttagaaggaatttcactcttgtcacccaggctggagtgcaggggtgcaatctcggctcactgcaacctccgcctcccggattcaagcgattctcctgcctcagccgcccaagtagctgggattacaggcgcctgccaccacacccagctaatttttatatttttagtagagacagggtttcactgtgttggccaagctggtcttgaactcctgatctcaggtgatcctcccgcctcggcctcccaaagtgctgggattacaggcatgagccactgtgcccggcccctacctcctttttataccatcttacttattcatagttgtaatatcttttcttgacttgaagtaagtcaattttagtttctactctttccatggttattgctttctccaaattctgtatattttgttatttcatatatgttagaagctttcctcaaaagtcttatattagtctgttctcatgctattaaagacatacccaagactgagtaatttataaagcaaagagatttaattgactcacagttccacatggctggggaggcctcataatcatggcagaaggtgaatgaggagcaaagtcacatcttacatggtggcaagcaagagagcttgtgcaggggaactcccatttataaaaccatcagatctcaagagacttattaccatgagaacagtatgggggaaactgccctcatgattcaattatctccacctggctgcacccttgacacatagggattattgcaattcaaagtgaaatttgggtgggacacaggcaaacaatatcaagtctgataacccaggagtgttctttcatagtttagagtgtacagctatgttgcctggaagctgtgtgtgagtagccagggcctgtggatatgtgtgcttcactgtatgcttgggtggtcttctagctttttattggaggattcccagctatcagctttgacagatcttttctctttggccattctctttctccaggaaagtatctgccactcttctgttagggaggtataaatctagctgctggagttctgggagctggatggcagaagagaacaaggaacttttactttgtttctgttttttaggcccagtcttcccctctaccctctgctgtgtctggtatttaaagtcctgagcccacctagttcagtttctccaaataataaatgtcccatctcctctaaatgtggataagaaactgagaaatctacctgctcttaggcagaatttcaacttatctccccatgttccaccctgttctccacccagactttctgtagcacccagtgcctgtgggtcctgaattgtacctgctctgttggcttgtgtcttatctgtgttacctctgtgcctgatgtctgtcacctgcatctctgctgctgagtcagttattgttcatttgttttctgtacttctattttatgactcaggttatagatatctgttcatttcattgaagaagaggtttgtgattttatttctcatattttcttgttgtttggactgacttctgagagaagggtggggcaaaaagatttttactcagccattttctgactggagtaatttttgttttataatgagaaaaaaccaaaaagctgtcttcatcttgaggggaaattaagttacactactcattgagaaatttagggctgggctcagtggctcatgcttgtaatcccagcactttcacttgagcctgggagttcaagaccagcctgggcaacctagggagactctgtctctacaaaaaaattttaaaaattagtctggcatggtggcacgtgcctgtattccctactactccagaggctgaggcaggagagtcacttgagcctgggaggtcgaggctgcagcaagccatgattgcaccactgcactccagcctgggcgatagagaccctgtcttaaaaaaataaaatgaatttagtagaaatgggaacagttacgaccaagaaagcattataataatcactgaaacattttataagcagacttagttgtttttgaaggtagcaagaagtttgagagagaaattgaagtagggccttggtaaaggtgagaatgtcatcaagaacacaggtagaggccaggcgcgatggcgcatgcctgtaatcccaccactttaggaggccgaggtgggcggaccacgaagtcaggagatcgagaccatcctggctaacatggtgaaaccttgtctctactaaaaatacaaaaaaaaattatccgggcatggtggtgggcacctgtagtcccagctactctggaggctgaggcaggagaatggagtgaacccaggaggtcgagcttgcagtgagccgagatcgtgccactgcactccagcccaggcaacagagcaagactccgtctcaaaaaagaaaaagaaaaaaagaacacagctagagagcttagctctcatatggaagtgaaggaagaaatgccacaatttagaggtagcaaggagacaagactagaggtcacactcataagtgaagttcatttagtaaataaaagcgccaagcatttcaaaccttattaataaatgactcttcatattcttcagcccatcttgttgcttctaatttttgttcaatggaatttagtcaatgaacagccaaagcaggcagttgaatacaattttggctgtgctaagtgatgaagtttctttcaaaattaaataagaaatacttttccttaaagatcttaaatggttatatatcattgagcacactaaatcaaaatacagttgtctctgcgtatccaagggagattggttctaggacctctgcagataccaaagtccatggatgttcaaatcccttaaaataaaatggagtgatattagcacataacctacacatgtcctcccaaacacttttaatcatttctagattacttataatactacaatgtaaatgctatataaagagttattataggccgggcgcactggctcatgcctgtaatcccagcactttgggaggtccaggcaggcagatcacaaggtcaggagatcgagaccatcctggctaacacagtgaaaccccgtctcttctaaaaatacaaaaaattagccgggcgtggtggtgggcgcctgtagtcccagctactcgggaggctgaggcaggagaatggcatgtacccgggaggcagagcttgcagtgagtcgagatcatgccactgcactccagcctgggcgacagagcgagactccatctcaaaaaaaaaagagagttattatactgtattgcttttaatttattggggattttttccttgaatattttcaatctacaattggttgaatctgaggatggagaacccatggatatggagggctgaccataataactataatcaccagatggttttttttttttttttggaactggggtctcactctgtcactcagactggagtgcagtgacacgatcatggctcactgcaacctctgcctcccaggctcatatgatcctcccaggctcctgtgtagccgagaccacaggcgtgcgccaccacacccagctaattttttgtatttttgttagagacagggttttgccatgttgcccaggctggtcccaaagtcagctcaggtgatccactcaccttggcctcccaaagtgctgagattacaggcatgagccactgcacctggccttagaaatggagttctttatatggcaagtctttattattagttcacacttttaaacacttgacagtcatattttctgatatagatacttgtttcaaatcccactactttgaaaatggattcaagtgtctgtagacatttacttgactgttgggatttttaaattttcctgtgacataatcagacaatcatatcagactgttgagtatgactgattttttttgttgcaaaatttatttttcatttgtaaaatatttgagattataggaccccttccccaagttcctggcgtaaatcttttttttttttcctccaagacagagtcttgctctgtcacccaggctggagcgcagaggtgcgatctcagcccactgcaacctctgcctcccgggttcaagcagttctcctcagcctccctagtagctgggattacaggcacccgtcactgtgcctggctaatttttgtatttttaatagagacagggtgtcaccatgttggccaggctggtctcgaactcctgacctcatgatccacccgcctcagcctcccaacgagctgggattacaggtgtgagccaccgtgcccagccaacccttaatcttttttttttttttttttttttttttgagctgaagtctcgctctgtcccccaggctggagtgccgtggcgcagtcttggttcaccacaacctccacctcctgggttcaagtgattctcctgcctcagcctcccgagtagctgggattacaggcgtgcgccgttgcacctggctaattttttgtatttttagtagagatggggtttcactgttttggccaggctgggctggtcatgaactcctgacctcaggtaatccgcctgcctcagcctctcaaagtgctagtattaccacctcccctggcaaccctaaatcttattctgcataagaatacttaagtaaattccatttacaaacaatcattatcattagtcttcatgaaactcggtattttacattgcaaattctagtatgttttaccaacttcagaggaaaggcagtgccttatagaaatttatctgcttgttttcaaatgatcaagaatacaaatgagtatgtgtttaaaatggctaatttttatcaattttgtattttctaaaaattgcctagactaatggtaaagttgactaattattgaagtgagtttaaaacactgtattgggaatgcttactgagcatttttgctgttcaatggtgattgtttatttaataaattcttacagctttacaacttaaaattgttcatattttaatcagtagggaaggaaatagggagaagaaggtgaaggaactatgtagggtagtagttctcaaataccagctcatagatttgtgtctgtcttctacaaataattcactggtctgctgtgaaataagaaaactatggtaaggagagatgagcataagatatacttcaagactagaaactattcacatttgagtatttattatggttgatagaggaggcttggctttttcttatccacactgaaaattagctatgagaaaggaactcactcttcactctatggcactattaagttatcatgtatattatataatatatagcatgttatttaagtgggaacaaagtgtcttgggactcccttccaacatctctttttcatgtactaactgtgcttttccctcttgtgtctaatagaagcttaatatgtaaactagacaaggtagatgacaaatggttaatgacttttttttgttttctttagatacatccagaaagtgcccagaagaaacttcctgctggaaaaaatgaaaaagcagtatttataacattagaatctggataatttgttaacatggcagaaaataatgaaaatattagtaaaaatgtagatgtaaggcccaaaactagtcggagcagaagtgccgacagaaaagacggttatgtgtggagtggaaagaagttatcttggtcaaaaaagagtgagagttattcagatgctgagacagtgaatggtatagagaaaaccgaagtgtctttaaggaaccaagaaaggaagcacagctgttcatccattgagttggacttagatcattcctgtgggcatcgatttttaggccgatctcttaaacagaaactgcaagatgccgtggggcagtgttttccaataaagaattgtagtagtcggcactcttcagggcttccgtctaaaaggaaaattcatatcagtgaactcatgttagataagtgtcctttcccacctcgatcagatttagcctttaggtggcattttattaaacgacacactgctcctataaattccaaatcagatgaatgggtaagcacagacttgtctcagactgaattgagggatggtcagctaaaacgaagaaatatggaagaaaatataaactgtttctcacataccaatgttcagccctgtgtcataaccaccgacaatgctttgtgtagagaaggtcctatgactggctctgtgatgaacctggtttcaaataacagtatagaagatagtgatatggattccgatgatgaaattctaacactttgcacaagttccagaaaaagaaacaaacccaaatgggatttggatgatgaaatcctgcagttggaaacacctcctaaataccacacgcagattgattatgtccactgtcttgtaccagacctccttcagatcaataacaacccatgttactggggagtgatggataaatacgcagccgaagcactactggaaggaaaaccagagggtacctttttacttcgagactcagcacaggaagactatttattctctgttagttttagacgctatagtcgttctcttcatgctagaattgaacagtggaatcacaactttagctttgatgcacatgacccctgtgtcttccattctcctgacattactgggctcctagaacattataaggacccaagcgcctgtatgttctttgaaccacttctatccactcccttaattcggactttccctttttccctgcagcatatatgcagaacagttatttgtaactgtacaacttatgatggcatcgatgcccttccaattccttcttctatgaaattatatctgaaggaatatcattataaatcaaaagttagagtactcaggattgatgcaccagaacagcaatgctagtaacaggatgggaacatgggaatgataatatatattttttcttttaatattttatttttctttttatgccactttggatttttctacaaaggcagtggtgtccaaaataaaatctctgccctaaattttactaataaatccatttttctagtgatacacaaattgtttaaggttatacactcgagcttaaatagatatttttaaccaggtgtttggtttttgtttttaccgtgtaggttgtatacttacattttttctttccttaatttatacatgatcctttttccttaatttatacatgatccaggcacatttgaaatttggtaggcattgcaaacacatttgaatattgtgtatttcatactattctttaaaagtaatcttatgtcctttcctacatgtaaaatattttgttaatctatgccattagtagtattatatataaaatatagttccccgtcccccttttcattgagtttgatattcttcagtaaagctgaatgttgtaattcaccattcatactaatgttattgacttttgtaacttactaatagggatgttaaaagtaacaaaaccaagtcaaacttggtgtatttttattttaaatattaactctaaagcaggattactaaatttgattaatctggtcaatgagaatcaaatggaaacatatcattctggaggtgcacttactcttctacaatgtggccggatcatttgtcattcctgaataggtctttctctcaacctgcaagagctaaaccatcttcaaaacataaatcttgttcctttctcattggaagctttgaaaataatgataacactattagtagctagtagttactaaagtgctaaagtaattgtggggtttttctgtattagcttatgaactgttgcttctacttagcccttttatagaaactctgagctgttactttggggaaattccacaatgtattagcagccacaaatttccactggtaaccaaagagtacctaagtagtttttcattattttaagttgaatttgaataaagattccagaaataaacatggagctcagtattcaggaagcttaatactgtttttgatctacaagatccaaataattatccctatcttcagaattaacttagtctgtaatgtggcttcaaaaaaagaaagccttttgacagctacatgacttacaccatacagaacagtactggacaaaaggggtgatattttaaatttactatcccagaaacagggcaacgtttttagaaacatctttagcccaaagtaactagtaaaaataaatggagtggtatcctatcttctttttttaaggaatcaatgaataataaatgtagatagacaattttcttctggtgagctgtctaaccctttgtacacattgacatttttaatatcgtaactgatttcttgatgctagtttagctatattaggaaactgcttctacctagattgaaagaaatttgactcataaacttccaagttagaacaaatatttcttcattattgttgcttttatgtgagtagcatttccctatcttgcagttctgttaacatgaaaatggcatttttccataatagctttaaaataattttttagtaattattacaaaattaaggaaaatctctattaccgttatcttttagcattttttttttcctcagtgatctcaagattgtcttagtctcaatgagatgtagctacaaaaatggcacctcttagcagtgatgaggctcaggatatcggatatcataacaccaatgtcaggataaatatctcttctaaagacgatatttaccttttacaaggttagaaaagtctcatactacctcatctttattgtggcgcttttgtagatcactgagaagcttatcttattaaccaatataccacttcctaaatatccatctttggtgaaagaaaatagtgtggtaagagtacccatgattatagttttttatcagaatttgataagattttctgtttctgtgaaacaattattttaaatatttttcttttaaaaataactttttatcagtacagaaaaacctaagggatgactagcttacaaatattctgttaaggggtagttttataaagaaaaatgatatatagttatgttgtcttttatagtgttaaaattagtatttatatgaaagtcaagatctaagtaccttttcatataattcaaactgattgcttgtgatatgttttctctggcttttttattttttcaacttcaaggtattaacagctgttaacacttaaaaagtactgccacatatgtagccttgaagtagttgatctaataaatgagagcagggaggagattcattgactctgaaatgatttttatttttcctttggtatttgctacagtgaaaagaagtttggaaagtagatacataaagacttgagaagggaattgtttaagggaaaagaaaagagtaataattttttttaaaacattaaggaattggaattcagaaaggttacccccttcctctgtgtctttgtaaggatacatttggaagaacatacagtattcagaaactaacctatataaaatgtaaacaatatattgattgcactatataaatgagtaaattttgagaggaacaatgtgggaatgtataaatttgtctaaaattttataactgttgaacaaagggaagatttgaacagtgtaaattccagagttgagaaagcatttagcatggtggaaatgtggagagcttattggtgagagaggatggagctaagagaaataagatcaaatattaaaattaggaatttaggcattacctcagggaaaagccttgacagtgaaaagaaactacttaatcgctaatagctagtctggaccagacacttaactgcgctttgggaatcatcctgggctaatcaggaaataaacttgacttttctatttttctctgaagctttacaggacatactcccactcccatttttttctaagcctacagcatcttgattcatagtataattcctgaagtatgctgaatggactttttaaggttcctttagcatttttgtatagagttatatgtgatctttctataaccatgccattcacttaaataattcatgaagggaatggtttaatttaaaggaagacctaataggaagaagattaaaggaagcttttagtcagcctgtggctagatttattgatttggtgatcaaacctgtgctatttcctgaatttcagctaatggccaactgtggtcaaagaatggctttcagttaaagttttgacttcttattataaataatataaagaatgtctcagtaattagaaatcccttaataagtagaacaaatgtggcaagtaggactcctatcggtatacaaattccatttctttttataagaaaaacacataaaccattatcatttattcatgagccaaagccattaagaagataaatccagttatcagcttcctgtctctgaagtttggatcattttatagatccacgcaatagagagcttccttcctatgaaacaaaaaccttgagatctctttttttttgagacagagtctcgctgttgtcagcccaggctggagtgcaatggcgcaatctcggttcactgcaacctctgccttccaggttccagcaattctcctacctcagctcccaagtagctgagatgacaggcgtgtgccaccacgcccagctaatttttgtatttttagtagcgatggtgtttcactacattggccaggctggtctcgaactcctgacctcaggtgaccacccgcctcagcctcccaaagtgctgggattacaggcatgagccactgtgcccggccaagatctaactcacgcttacttttcattaaaatatttcttgtcgcttttagctattgtcagaatccaataaacagcttacgcattcattttggtaaagcaaatttcacaggaaaatagacaaaattataagagattcctggagacaacacaaggaaaacaatgtttaaaagcaaaatagcctgtgtaatgttaggtaatgtaatgcccaagtgaataaaagatttttcgtcaagcaaactactgtttctactttgggggaaaaaagtcagttttacatttgtaatttaaggaaagaacagaaagcacaaaggtttttcttttctttattaatggcctactagaaatgagcagtgcaagagtctacctgtactattctaatacagtaagatattggacacaaaatggaggtaactttttaaaatagattggcttggaagttgaaatgtagattaatggatataacccaatagaaagggattttcaaataaaaccaaagtctatttttttatttactttctaattttgaacagatgactcagtcctaaatctttgcctttatttcctataaaatgtaggtgatacttgtaactcgacttcctggagttaattcttcagcaagaggtgaccctgggagcgtactttaaaataattgctgtacagccattgagtactaacatgatgataggttttcaaaatatctttgtagtggatgctgcataattacattcacttctcttagactgtaaaagactttcttgacttgttttaacagtagagatagcagtacaatttgaatttatggtttaggctctgcaattagaggaacaattgcagtttcctcctacccttcatatggtctgtgtaaaactgatgtttgcttaacttattttaaaagttgattacgttttcagaaaataaagataatcacttttgccatggttataatcaaatctaagctttcagacttgagagccatggtgtaaaactcaaggaggtttatttaaattatgctgactttgctagaattggataaattctgtataagccaagtatgagttcacatgtactcgaatatacagttttcacaaagctattactctcatcagtcaggcttgtatgatctattccttaccacaaaagaagtagacaattgccacttttatttctaatccttaagttgaatgtttcttcttggatgtaagttcaaataaattgatctggataaattttcatttctacttaattaaaacttcctatgtaaaatccaaa

SEQ ID NO: 18 - protein, 536 aa, Homo sapiens
MDKVGKMWNNFKYRCQNLFGHEGGSRSENVDMNSNRCLSVKEKNISIGDSTPQQQSSPLRENIALQLGLSPSKNSSRRNQNCATEIPQIVEISIEKDNDSCVTPGTRLARRDSYSRHAPWGGKKKHSCSTKTQSSLDADKKFGRTRSGLQRRERRYGVSSVHDMDSVSSRTVGSRSLRQRLQDTVGLCFPMRTYSKQSKPLFSNKRKIHLSELMLEKCPFPAGSDLAQKWHLIKQHTAPVSPHSTFFDTFDPSLVSTEDEEDRLRERRRLSIEEGVDPPPNAQIHTFEATAQVNPLYKLGPKLAPGMTEISGDSSAIPQANCDSEEDTTTLCLQSRRQKQRQISGDSHTHVSRQGAWKVHTQIDYIHCLVPDLLQITGNPCYWGVMDRYEAEALLEGKPEGTFLLRDSAQEDYLFSVSFRRYNRSLHARIEQWNHNFSFDAHDPCVFHSSTVTGLLEHYKDPSSCMFFEPLLTISLNMTFPFSLQYICRAVICRCTTYDGIDGLPLPSMLQDFLKEYHYKQKVRVRWLEREPVKAK

SEQ ID NO: 19 - DNA, 63829 bp, Homo sapiens
cgcagagcgcgcacccaagcggccggacctccccgactcccgggccgcccccgtctcgcgccctgcctccctccttcggccttcacctacccgcctccggattggccgctaggaatcccgcccctctaaagccctgcctgctattggtcacggtaggctgcccttcagagaggcgtgcccctgcccgccccccgccccccgccccgggaggtattttccattctggtgggggttggggggaggggggaggggaaacgggtgaagaaggggaggcggcagggaagggggtgggggcctggcgggggcatccggcggagctggggtccccgggctccgtccggaggaagcgacgctgcgctcgctgggcagtcggaggggacgggacgcaccggagggcaggcggactcgccctgtcggtgactgcgccgtccgggcccgtcctgcctggccgcaggtgccctggatgaggccgccccgcgcgccccaaacggtgagtgtccccgcggtcgcgcccggcccgccgcctgctccccggcccccgcgccgtcgtccgcggccgcctctcggtgccccagtgcccgcgcccggcgcattccgcccccggctgtcgcccccgcaccgcggcggaggcagcgccggcctctggctgggatgggctggccgggaaaaggactgctagcccgggccgcgagccccgtgcagaccacgccgctcacagtgggagcttgcgcttagtaggctctcgatgcatttctgtggaattgtttttctggttggaggaacgtggggttcctaaggggaagggggtcgtccggggccagtaggagggcatccgtgatcggggtcgtggtgggtcgcggggaggccaccggagactgaagcagttacacaggctgcagggaagggagcaccgaccaagtcacttggggctccaagagcccgatctgggggtcttcaaggtcgaggagaaaagatcctcttggggatgagggagggaaccaatgagtacatatgtgggaaatgaattgagtgccccgggaggtttaactaaccaaggtgggtaagggatgcggaggcgggagctggaagacccctctgcaactacttgggctcctaagagtctttgatgtggggtttgggcatccgagtagcagaagcggagtgatttttgtggagagggaattgcatttaaagaaggatgctaaaaaaacagtgtaaatacatcttggtttggttccgaactttgaaaggcttgagggattggagtcacaaggaaattttgacattttcttgtggcattccaactaaagggtgcctggctgctggactttgtcagttatcttttattgaaaaatatatgtgtatgtttgatggtattctatcagtcacaagtttttctggaaattatcataactacaccttgaaattcttgtagcaacctctctatccatagtggtggcttacttcttccctatttaacgtttagaatggattgtgctattatgtactactgacttttattctagaagactaataagtaattcacattctttcttttccttactttcgtttctctacaagatgatgcaaaattgaactacggattctatataagttttaaaatattttaacttgtgttgactgggaaggaggtggtaagctagcagctctattaagcaatactaaaatcaggttgagtttcttaacaattggtaatcttaatgtcagttgtttagtttaattttaataatttttcctttatatagcactcttgagaacatagaaaatgcacaccgggtttaggcagtggtggtctccagagtgcacctccatggcagtccgtggcggttcttcatttcactgagatctttaatcttatgaatattttcttaattttatatatgttagtaattttttacatttgtagcgtactttacggtttacaaaatgaaagctgattagtacttaaacatattttggaaaaatttgtatatcatattttggcccttttatccatcagagaaaacatttctgcaaaaaggtgttaatattttcagttggcaccttcttagttcttgaataactaatggataattatatatattttctccttattgtccttgaattttactattcgtcctcagggagaatgtactggcttacaatacagtagcctaaaatattgaaacctaacaccttttaacattataatagcttctacattattcaccttgagaatcatctatggcaaaccagtgattagggaagctttcccctatcacctttgtttagattgggctgttttctccttttgtagaactaaggaaaagtaagactcactggctgtgaagtagtagtaatagctaaacagaatgagaatgggaggcttggaaaaaagatgtatgtgtatgagtcacgtttcttctagagctggaagggtagaccatggagatgatattttgcagattaggaaaccaaggcccagagcagttgaagggattaaattgctatttaatggtggggccaggattggtgggcaggtctgtggactccagtccagagattgctttatcactttcacactgtttatttctaaagcattctcgtgccaactgttaatggtttttggattatttgtgatttgttcccttctaaggatgaagaagtttgagttgactacttttatccatgaaattttgtaaaactgggtgagcttttatttaaaagtttaaaacaatatgctgcattgttagcgttagtgctgatagttccctgtcattggaaagagacaaactgtcatttaggtgaaaatattaaatgaatgccctagagtagtttagcattctataatttttatgctaaaattaattgcttaaaagattttcaaaattcagaaggcttctatattttgaaagtaaattatgtgaacattattattttcagttagtgtggcggttttgttaaaatgggtttagtacctttaaaaagagagtagaattacatgatcattatgaatacatcattcctatcatagttataatagagaaagacaggatttgtatcccagccccttcacatgatcttaggcaagtgatttaaccactgtgtacctgagtttactcgtttgtaaagggagggtgttaaaagtacctacctcacagcatagtggtgaggtttaactgtgcctacctactacatagaaagggtacagcaaatattagacattattctctgtggttcgttagatgtaagcaggcagttgtgtgcccatttatttccttgctgtacacgcactttcaagttacaaaaattcctagaaaaatgcacgatcaaatcatactctttggtaagggggtacttggtacaaatgatgctctgatttttggtattgctctccttcctctgcttaccttggacctttaattaggtctgcatatcaaattagagtgaatgtgttactgtttctgaatgtcattgggagatccttgttgcagtttcattattctagaacaaaaatattaataattgatttgcagatattccagtgaaaatacagtactagagtactagagacctgctgggtacacttaggtgaggaagtcaggaatttataaggtctattattgatctcagctcttctctgccagtggaaccatgggaaaatcattgaaccctgttttctattaaacgaatattttgatgacttcattcattaagctgtgagattttagtgataaaatgtgtgtgactccaattttataacatatggttctttttaatttgctgtaacccaggtccaactagtgtcatgaccatgtgttttagcaaatgtttttgacggtgacagtttgagttataagttatctcagaatccttcttaatggtgtatcacttaaagaatggtgaatttctcttggtgttctgtgcttctgccttctcaggtttttctgttgcttttgtaacagcccttcttccatttgtggtcctttcaactttgattctcatgcatctttgtggtctttccttaagcatctttgccttctgtcacaatatgtaacttcttatctgtcatgcaagagctctattttaatgagtatgttttaacattcattttcattattttttcaatgtatctcacataatgtcttttaattgcccttatttgtagagtttcattgaagtgagtattttacaggtgagggattaaaattagaaacaagctatagctcgaaagcaatgttactgtactagtaatagtcttgctattgttttagatctcaggcaggcatgctgttggtggtacttcagatgataaatacctgggccagctattgagtccaaagtgatgctagcagggctaagaaagccacatgtagccatcacaagttttcacagttttttttttttcactgatgcagaattttagtgtttgaagagtgaccaatttctaatattcttttggttaactctcataataaggattatgaagttaccacaatttattggacatttattgcattcatgacatacttttaggaatttcataggtactctcttcaagtctattacataatctaatcatgtgaataaattagttaaatatggttttagaaggcatgtcatgttaaaatataaccaaccattgctcattttttcattaggttggagttttctttatacatttaaagataattatgccattgtgaatggactgttattcatatttaaaattcatggttatgtgaactgagtaggtgtagactgtacaaattactttcttagtttcagtgcagttgttagagaatgaaagagggaaaatatgaattatttgcattgtccatcttaacaaattcttcatctgagtcagccagctatcctactttcaaggggagtggggagggcttttgaatctggttagagcagaagtaatcttgaaagggtaattaggctggattgttgactcatgttaatattggataaatttgcatcttagtgacctgaaaattagttgcctgcctttacatgtaggtgaaaaattgtagggaatgatttcagagagaccatactgtattatgaaatataatatagcaagaatatattttcacagattcatatgctgcttaacaccaaaattaaatttaccttactctgatgagctttcatacagtaatctttttatcaaagctagtaagaggaagcagtgatgaagataatttttttaaaattgtatttgaaatgggcttatgtgctctgggattgtctttaaattgacttccttattttatttgaagatgaccataaacaggaacaatgtccagtcttcctattctgagcagtatccttgagttagaagtatttcaagcaattttaaagggtggaagcttatttaaataatagatatagggaaaatggaacaatgagaactccaatgatggaatggaaatgatacgggaactgcaggttagacctgcgtatatggtaggtagttggaggaaggggagttaggatgagaatcggaggctgggtagagaaaagtgttgaggtaaactgaattttgtaggaatttagccctagtgtaaatcctgaataccacaggttggcacgtctttgtatcaggatgtaatactattatgtactgtatacagtttgcttttcggtggtggttcctggctcaacacaggtcttgtaagcctcatcatttcttaagtgactagagcagaaagaatatcttttgttgaagtagttggcctcttatccttggttcctgaagcagctctgaaacagcttctgagcaataaaggtgaaagatagtcttttgttatttacaacaagccctttcaaacacacctgggcatatgttaatgaggctatttttggaaagtccctaggtaagcactgtttggaggtgttgatactccagggaaatcagcattgggctctgagggttggcactttcaactccacctcccaaccactggggagaagagaagggccagacatttaggggcttccaggaaggtgaacaagaacacatccatgtgctgggaagatggcacactccaacaccatggggacagaagttcctgcactccagaccttgctgtaattgtcactatttctgactgtttatttgtatccttaaaatatgttttgtaataaccagcaaaagtgttttaaaacacttttacttactttacagtgtttccctgagttctgtgagctgctctagcaaattaatcaaactcaaggagagggctgtaggagccccagtttatagccggttggtcagaagcacaggtaaaataattgagcttgggattggcatcagaagtggggctcagtcttttgggtctgggtcctcaacctgtgcaatctcacgctgtctctaggtgtagctggtgtcagaattggtttgaattagagggcagccagttggtagctgctttagaattgattgcttgcttttcggtagagagaaatccctacacatatgttttgaggtcacaaaagtcttctgtgttgattgttgaaggacagtgtgaagaaactgagtttgttttttcctagattaccagacaggaaagcgcctgaaatatgcttaacgctaaaggataaacccactgaatgttaaatataatctgttaaggagaagctcaagcactttcttatgggttaaaatatgcatgtggccaaaggggtgctcatttatctaattatttagtttttgacattggctatttacatgagaacagcagtccaaccccattatccttcaggtgatcagtactgggatgagttttatttaacatttttataaatgatttaaaagacggactggcagtgacagttgccagatttgcagctgactccatgtgctgttccaggtagttaaaagccaagccagcaaggagaagctacaggaatatcctctgatgagttttcatgtggcacagaacaagataatgcatttaggggaagatattccaaattatatttataggacctgaattgtgacccaggaaggggatccagaagttatgtagatgacttccttgaaggcatttattgtggttgttgccgggccagaaagtccaagaatatgttaaacattgttgggcatggttctgaaaacaaaaatgatgttatataaaaattataaaggggttatcatattttgactgtttattatgttccaggctaagtgatttattatgactgctctttcatttaattctcttagttcagaaaggtaggtggcattatcccattttatagaagagcaagtttgacacgggaaggttaagtgactttcccaagtcagcccatgtgtggtagagttgggactaatgtgctagtcttttgattgttaatcctagacatttcccaggatattaggaggcagcacttaaaacttgtaataatttaatttagggtttgtgaagtgctcttttactctttgctatggtcggctttggttgtcattttaccacaaggatggtatctaaatactttgagaggatttagagacattccttaaattcttgatttttaaggaaagctaggaatgtcttggtgtacagttttagtcacctctggtgacccctgacatttctccccttgactttataaaatgctgaatggttacattaataaaaggaacattaattaacaaggaaaatatcgtttaatagaaaagtcaccagaataggagtccagagacctggattgttctagtgctataaagtagtaatgtgaaaaacattaggatggcatggaaataagcataacattcttcttgccctggaggtagaggaaagcttgaactgtcttgagagatcagccggtgaagaggggttgtggattgcaggcagagggaatagtagtatattagatgagtcactgaacttctttgaacttcagtttcctcaaaattgagataaaatataagaaagtaccaacggcagcatttgattttggaatctcagagtgagattataaggatcacgtgagtaagtatatgtaaaaatgctttaaaaatttttaaatgctgtacaaatgaggatggaggaatactgatgttcctttgaagagtggacaaagaggatgcagtgaaaatttaggtggcaggactagcacagaaggtagaggaggagaagtggggaagggaggtgttaaagatgagaggaaaaggacaaatgtggagaaagttgaattatggttctctttgtgcctgtcagtcaaaaggtgggtattacatctataacggaagaattttaacatcaatttactgtaaagtggaagctcaggagatagttgaaacgttttaatcattttcatttagtctgtattgaccatgttaataaggctatgaaaataagctaattgagagttggtgtgttgtctgattgtttcttcactcattacttctgtataggtttagctaatgattgtaaatagtataagagcgctcataaaatgaaataagctggactgagttactatttattgtagatgattagaaatgaaaggacatagagtgtttaaggaggaagatgctatttgatttttctaaactagtgcaaaagaaaattaggaaatcagactaggaccgaggcaggaggggaacaaattgtgaagggttttgtatgtagccatataggagtttggatttttatttagttttatagatgggaagtaacacaaacacttttgtgttttagaaaattgatcatagcagtcataaagagaatagtctggaggagaaaagattggagacatttaccaaataattactgagcaaatacaaatgctcctcgacttacaatggggttagtcctgataaacccatcctaagttgaaaatatcattaggtcagaaatgcatttaatatgcctaacctaccaaacaacatagcttagcctagcctaccttaaatgtgctcagagcacttgcattagcctatagttgggcaaaatcatctaacacgaagtctgttttataataaagtgttgaatatcttatataatttaatgaatactgtactgaaaatgaaaaatagaatggttgtgagtttaaccaaattgtaagtcagagactgtttgtaatacacatggcaaggtgagtgaatgaagcaattctctttggcttgtggtagatagatgacagtcaaggatgacatttaaggctctgctctagttcaagaaagagactaattatatgatggtctgaattaaggaatttgtagtggggttggaaacaagagcactgctgatttcagagatacttaaaagtttgagtcctccagattttgttaatgttagaaggtgttagaagttgagtatgagtctaaggttcctggcttggcaatagaataatatatgcaagtttgggagagagagaatactgagtttaatttcagataagtaataatgctttttaggtaagtaatgataccacaatatcagttggaaatacgggtctggatcaggagcgtggtcaggattatagatatgattttgaaggcatatattttgtagctgaaaattatgaaaatagatgagtaatgcccataatatctaaatgataatgcgttcagaacaaaaagtaactgccattgctggtacttagttcaagagtttcataatatttacctattacccattgccaccccaaactctgttacaaaaaatgtagaaaaatatttgctgccagaaagccagaaatatttgagtgtccttgaaacactgtaccagttgggatctttcttcctgccttcggaatcctggctagccatccagtacaacacaggattcttaggcaaatgcacttccttatccctcaactagagtttctttttggaatccatgtttgcttctttgggctggtgaagtggtctctgaacagatactgaatcctgaagcctttttttttttttttttttttttttgagatagggtctcgctctgttgccaggctggtatgcagtggtgccatctcggctcactgcaacctccacctcccaggttcaacagattctcctgcctcagcctcccaagtagcggggactacaggcatgcaccaccacgcctggctaatttttgtatttttagtagagatgaggtttcaccgtgttggccagacggtctcaatttcttgacctcgcgatccgcccgccttggcctcccaaagtgctgcgattacaggctttcaccttctttactatctattccagagcatccctaagcaactagcactttgcctctttgcctctttacctttccagccccttagagcaactcaaagagggaaaaataaaaacagcttgagagaagaggctgtaatcttttcagttctcccccaaaggttctgctttctaagcaaaggatggagttaatttgtcataaagatgaggtgtccaatttcagtaaaataaatgtgtctgttaggaggaagatcttcaagtgctgaagggaatttggaaagaatctgacttaggtggtgtttagcacttagtggaaaagatgagttttgggtttggttttagaggaggtagaaagacaaggctgtgcagagaaaagtaggaataacactattaggagttgggaaaaacagtaccaagaaagtaatagaacattaagggtggagcagcagggggaagtgaaggacctggtgaacaagatttcctatctaaaatgcctattttgagttgggattaatgagaaatttgggtagttagatgattcaagactaaaatttggaattgcaactccctaaagcactgagtagaaaattgaacatgaacagaggatactataacataatttaaatagcaatttaggacttcagtgacaactgttatagtttatgatgtttgtagcactgattacttaatttacatactattttaaaatcccttgtatctttttgttttctccttacatacttctataacagcaattcttaaactttcctagacatgagaattacctggggctcttttttaaaatacagatttccagtctccaccccccatgcttgaattctggttcagtaaaagttaaggcaagggttcaagtgattctgatatatgtagtcctcagaccaaagtttaggagatactgttctagagggtgggcaaagagtaaggagatagaatttcagtttttggtggttgttttcctttattttatttatttatttattttttatgtagcatctcgctctgttgcccacgctggagtgcagtggcgcaatctcggctcactgcaacctgcacctcccaggttcaagcaattctcctgcctcagcctcctgagtaactgggattacaggtgtgtgccaccactcccagctaatttttgtatttttagtagagatggggtttcaccacgttggttaggctggtctcaaactgctgacctcgtgatccacccacctcagcctcccaaagtgttgggattacaggtgtgagccgcagtgcctggcctcctttaatttttttaaaaagtggaagtgtaatttacatatagtgaaatacacagatattaagtgtatgtacagttgcatacaccaaatatctttaataaccttaagaaatttaaatgtccacttgtgaaaaaaaactctaagcaaactaaatatagaagagaacttcttcagtctgataaagggcatctattaaaaacctatagctaacatcatattcagtggtgaaagactgagttctttctacactaaaaaagaaaatttaaagatctaaatagggatataccaggttcaagatgtacctaagttcaagattagttttgacaattaatacatctttgtaatttatgatttctgtcatccagaaagagcccctgtgctctcttcccattgagtccttgctgtttctccttttccgtggctgcagtcactgttctgatttatttcactgaagattaactttgttctagaattttttataaatggagtcatgcagtatgtactcttttgagtctggcttctttttatcagcatgtttttgaggttcatccatgttacatataagaattgttccttttagttgtagagtagtattccattatatgaatattattattgtgggaaagattgtttccagttttcaactgttaatgaataaagctaccatgaacattcttgtataagtctttttgtgacatgttttcttttctcttgggtcaatacatgaagtggaattgctgggttttaaaataggtctatgtttagcattatgagaaacttgcagaacaatttaatttctgagatagttttaccattttacattcccactagcaatctatgagagttctgtttcttattatcatcaatatttggtttagtcatcttactgggatatctcactgtggtttgaatttgcattttccttgtgagtatcgatgtcatgcattttgtcatgtgcttataggctattttgcatatcatccttagtgatatgtctgttcaaggcattaaccagttaccaattttatgtcattttactgttaagttttaggatttgtatattaattggatgcaacttctttgacacataaatgtgttagagatttttcttccagtctgtggcttgactgttcattgttttggtggcatctttcgatgaacaaaagttttgaatttagtggtctattttatcaatatttcctcttatgctagtgtttttctgggtcctgtctaagaatctttgccacccccacagttgtgaagatattatcctgttttcttccagatgctttatggtgataatttttgtgtattgaggtgataattttgtgtattgtgtattggttaagattagatttttgcccccatatagatagctaattgttccaacacagtatacttccctttccgcattaattgctttggcaccttgttaaagcgcagttaatggagtaagtgtggatctgtctctaaactctattctgttcatctgtttgtctttctgccactatgacactgtcttgatttaccatagcattaagtcttgaaataaggtaatagaacttctccagtttagttcttatttttcaagattgtttagactttttttaggtattgtacatttgcacataaattttgtaatcagcttatcaatatctacaaaaaagccttttggaatttttattagggttgcatttgaatctatgaattggagaaaattaacatcttaacatgaacataatatagctctatttagatcttcttttttagtgtagagagaactctttcaccattgtatatgttagctatacatttttcatagatgccctttatcagattgaagaagttctctttgcttagagcttttttttttcccacaagtggatgtttagctttttttttttttttttttttttttgagacacagtctcgctctgttgcccaggctggagtgcagtggcgctatcttggctcactgcaacccccgcctcctgggttcaagcgattcttctgcctcagcctcccaagtagctgggactataggcgcgtgccaccacgcccagataatttttgtatttttagtaaagacagggtttcaccatattggccaggctggtctcgaattcctgacctcgtgatccgcctgcctcggcctcccaaagtgctgggattacaggcgtgagccactgtgcccggcctgttcagctttcttaaaaaatgagtctgcactattgagacgcttatgtggtatttcttctttattctattaagatggtgaattacattgattggtatttgattattaaaccaaccttgcattcttgagataaacttggttgagatgtattgtcgtttttttgtttttgtttttgctttttgttttgtttcctctagattcaatgagggattattcttggtctatagttttttgtttgttttggtatcagagttatgctgactttttttatgaattaggaggtgtttcctcttattttgtggaagagtttggataaaattgatgttcttcgttaaatgtttgatggaatttatcaatgaagccatctggacctgtagttttctttgtggaaagttcttaattaaggaatttaatttataaaagaatgaatataggcccagcatggtggctcatgcctgtaatcccagcattttgggagactgaggtggaaggatcacttgagcccagaagtttgagaccagcttgggcaacataggcagaccccatctctaccaaaaatttaaaaactagccaggcctggtggcgcacacctgtagtttcagctactcaggaggttgaggcaggaagatcctttgagcctgggaggttgaggctccagtgagctgtgatcatgccactgcactccagggtgggtgacagagtgagtctctgggtctaaaacaaatgtaagactactcagattttctttttcttttttatgtctgttttggtaatttgcattttggagaatctgttgaatttatcagcatgaaaataaattataaattttatgaaacaatatttcctttttatgtcagtaggatctattccttcttttattcttgagattggatgtctttttccttttttccccttgattaatctaacttgagaaaggaatatcaatttcataaatcctttcaaagaaccaacttttgacattgctttttctctgtggtttatctgttttctgtttcatgatttgagctctttaattccttcccttctccttactttgggtttaatttgctcttttttccctagttttaagtgaaagcttagatcattgattttcgaaactccttttaaaaaacaattattaacatttaaagctataagtttccctgtaagcattctttagctgcatcccacagatattgatatgatttaattattattccattcaaaatatttttaaattcctcttggaatttatttgacccatgggttgtttgaaagaatgtttccaaataattttcaaatatttggtatttttcaggtatccttttgttactgtgttttcatttaatgccgtggtgatcagagaacatacatactcttaataacttcaaccaatataaactttttgattgttttatggcccaacatatggtatgtcttggtgaacgttcaatgtatacttgaaaagaatggttattttcctgttactaggtggggtgttctatagatgccaattaggttagtctgatcttcttatatcttacatctttactgattttctgtctgtggttctgtcaactattgtgagaggagggttgaagtttccaactgcagttttggatttgtctgtttcttctttcagttccaataagttttgtttcctatttcaagcatagtttacagcattgttgagtgcatgcacattggagattaagtgttgttgatgaattgacccctctgtcatcatgaaatggtcccctttatcccaggtagtttccgttgttctgaagtctctgtctgatacaaacttttcttttgattagtatttacatgatatatctttttcatccttttacttttaacctatttatatcattatacttgaagtggatttcttgtagagaatgtgtagttgggtctcactttttttatctagtctgataatctctcctttaattgatgtgtttagatcagggttggcaaactatggctggcaggttaaatctggcctgccacctgtttatgtaaataaaattttattggaacacagccatgcccccatgccaaccagtggtttagaccatttgcacttaatgtaattttgatatagttgaatgaaactgtgccatcttactatttgttttctgttggctggtgttgtttcttttttccttgattatctgcctgtttttggatggattattttttatgattctattttatctttcctgttggtttgtaacttagatctctttcaatttttttttattggcggccttaattagttaatcgtgatttgccttcaactaatattttaccacttaatgtgtagttcaagaatcataatagttacttccatttcttcttttccatatttcgtgcttttgttgtcatgcattttgagttgaagtgttctttctgctttgtcaggcagttaccttttcaagcaattaaaagtgacaaaaatactgtaatcccagcactttgggaggctgaggtgggtggattgcttgaacccaggagttcaagaccagcctgagcaacatagtaggacacagtatctataaaaaaataacaaaaattagccagacatggtggctcgtgcctgtaattccagctactctggaagcttaggtgggaggtattgcttgagcctgggctgtcgaggctgcagtgagcctagatcatgccactgtgcttgagcctgggtgacagagagtaacaccctgtcaaaaaaaaaaaaaagaaaagaaaaaagaaaaatgtctttaatatttaccttcatttaaccattttttagatctttatttctttgtatccatccaagtttgtgtctagtatattccctttgcttaaaaaacttcctgttacatttcctgtagttcaacattcctctcattttgttttcacttttgaaagttgttggtgtggatatggaattctaggtttttaggatcccctccgcccaccatcttaaagatattccactatcttcttgtttgtatagtttctgacaagaagtttgttgtaattcttgtatttgtttctctgtatataatatgtttttttcctttggctgcttttaagatttcgtttgtatcactgattgacagcaactttattatgttgagcctttgtgtggctttttggaagtttattgagtttcgtggatctgtgggtgtattgtattcatcacatttgtaaaatttttagccatctatcttgaagttttttttcttctcaggtgtctttctcctacagactctaatcccacatatgttcaaccacttaatattaacttgcagtttactgaggccctgtccatttttcttccaatctgtttctttttcctttagtttggaaattttctgctgccctgtcttcagggtaactgatttttttgttaatagcttctttgtctaatttgctgttatgcctgtccagtcaatttttcatgtcagacacccccccttttttttttttttagttctagaagttctcttacacatgtttggtttccacttctctatgaggtatttccttttttcttcattgtgttgatgtttttctttaggtctttaaactcttttctaatagctgttataaggtcttttactcaaatgtcctttacttaccccagccatttcttggtccttattgtagtttattatttatttttcataggcctggtaatttttaatggatttcatgatgttgagtgtcttcatttttttttttttttttttttttttttttgcatatagtgtttcttgattgttgagttttgtcctggcaggcagtttatttatggatcaacttttaaacatttttatttaattaactaattaattaattaagacagggcttcactttttcaccccagctagagtgcagtggtgcaaacatggcgcactgcagccttgaattcctgggctcaagacatcctcctgctttagcttgctgtgtagctggaaccataggtgcacaccaccacacccagctaatattttgacttttttgtgcagatagggcctctctatgttgctcaggctagtcttgaacttctatcaagcagtccttctgccccagcctccccaaatgctgggattacaggcatgagctactgtgcttggccaattttatgaatcaacttgaactttcaagttttgttttcagggttttttgtttggggaatttttgtttgtttccagacagatacggaatagcttttagtttagggcctgtttgggcccaccactgaggtgtgcacttttttgtgtctcctttaatttccccatgtattcatcaaggtttattcatcccagctggttattgatcctggccctgtgtgaactctaaatcgttggagagtgtcccagtatttgttattttcttggacgtttttctttgtgtagcctcatggagttacacaccacatgtatgcagattggtatttggccagaaactcgagaagaccccttttgcatgtttttggagcactttctctgtgtaacttcttcctttgtggttcgatgccctgcaaattctagctgtctcagactttacaaattctgagctctgtcacgtcagcttagtgagactactgactctatttggcttcctctccctgtgttttggttctaggcagaaagctggagcaatcacagggctccccttgcttgttttccttctttaggggatcatgttttcatattgcctcttatctcgtgtgaaaactattgtttggtattttgtctagttttctacttgtttgtaactgcaggacaattctagactttgcttctggctcatgactagaattcagaagtcttctgccttcattttttaaggatattcatgccagataaacaatttcgggttaacggtttttttttctttcaacattttaaggtcatcatttcgtaatcttccggtttatattatttctgatgagaagtcagtggtatttcttataattgctcacttaatgcagtatgtatttttttctcatcccactgctctaaagatttctctttttatctttggttttatgcaattttgtaaattttagggttaggttacctctaatatagaggcatgccttttcagagtctgtgttgaaagcatggggtattcaacaaggtcttttcactctagcttagctgctaggaattttaatgtctcccggacctgtgcttacaagcttaggtagttgtttggttcatactgtttagttgctctttccctagatgttattctttcactggtctgatggagtattgcgtgcacatgtgcagcttagtctctaggcaaagattccggagatctctctaaagatttctgaaactttgtctccacagctcccttcttctagttttgctgctcaaattccagcaaccttagtagcctcaaacttcaagctctatttcctcaactcagtgagattcttgtgttttgtgtgtgtgtgtgtgtgtgtgtttcccctgtatggggtataagcagaaagccagggcatttgtgagtctttgtttctcttttctcaggaatcacaatcctgtgctgcctcttgtccaatatctgaaaataatatttcatgtatattgccgagttttatagttgtttaccgaagatctagcccaatacagtttactccatcataattgaaagtggaagtttaggagtttactttttcttctttttaaaaactctttacagtttttggtatattatactttttagactatggctttatttttaaaaatcatgtcagctcatattttaattttcaagtgatacagagagttacagaagaaattttttaaaaaaggaaaaataactcaatatccccaaacccaaagtttaccagttatatgaacatcattttgggtatcttttattgcacatctactcagaaggataattattttatttttaaaatgccattcatactatttctgatataaggcatttaagtttaattaacagagctaaatttgttaaataaaagcaatatgggaaaaactgctcaaccacagataaatatttactaccagagataatactttctaaaagatttttctctaggcttaagaaataagattacagaaaaagccgaagagcttagagttatgatatcaataattgtgtgctccaataggtctctcacatagaaaagaataaagtattggcaaatatcttgctggtaggaacatacagtgatacagctttctaaatggcagtttggcaatattcagaatccttaaaatgttcataccctttgcccaagtttaatttttaggaatttattataaggaaataagagatgcacacaaatgtgtatgttacttattcattacaatggtatttataatgttgaaaaattggaagcctcaaatgattaggttatgctatagttgtgtaaccaaataccataaaactattacaaatcatgttctttaaaatttttatttagtgtactgttctgattataataaaaataactggcaggagatggaaaattaagagcttttggattttgagggttataagaaactgttcacaatatagtgaatgataaaagcaggatgatacatgtccaatccttttttccatttttgcagatactttagcttgagctttgttgagctttataactagatttaatgaaaggaacacttattcttacactgtacgtgtaatatgtatcctaaactttttccaaactaaggcagtctctcatgtttcatagtttaaaattatgagtttctatcaaacagttctttatgcacttgaataaatttaatattactatctttccttttttctttttttctagagctggttttaaagaagttttgatcttgctttctcagccagtgttcctgcccacttccttgcaggtgtggatgtccttatgttaaagaaagaatggacgtcatagtcttcgttttctcagtaaaatattatgtttgacgtctgtatccctggcatcttaagcatatggacctggtgctctttaggaactcactgtagtgactcagctccacatattttgctatatatagaacatgttggaaacccatagcagaaaattaataattaaaagtcaaagcattgttgtggtgaatgaacaaagaaataggtgtgtccttcagggaagcatcattcataccagacctgaaagaagaatgaccttaacctaagtggtctagaggctattcctcggcagtcgcagatccagaaactcagtttggtcagggcactgattctattctaaacagatttatggaaaactgataaagaaagcaaaggtaatttaaaaaatttatattaagaaacaatatcaagctatgatagtctgatggcccaccttttcattgtagggttatatctgctgaaaggatgtgagtgattgggttcactgatcagagcatgttgttctgaagtcttattatttaacctgagtctcaatgttctcatcattaaaactgtgataaaaacttgtacctcataggattattgtgagaactaaagataatccaagtaaagtgattagtacaattccttgcacatccttgctgttcagtaattaagattgttttcttgtacatctgaatttgaagtcttgaacttaaaggttcacttgatcttgattcctgcagacattgctgggaactgtactgctcttggtgtcataggatactaacattacttcatttacaagaatatttttgttctgaatggtaaaactttaataacaaactgtgatgagtttttgcatagattatgtcttactgtgagatctggctattttactactttgtgtctatggtaagaatctcttaaagaattcagcagtatttgcttttccaatttggcatttctaaaacagcttggtattttttataaaaccagaagtaaaatccaggtagcaaatttagtaagctgtttttactaataactgataaatttgtaagattagtgttctgtggttagtgtttgtaattcattgtatttgtagattgtggtattgtaagggatctggtccaattccctgtttttcagatgagtttacagagaaccagagtgaaactaagctagacagctgatgcatcatagggggtccctgggactgtgaggtaccagtgcagctgggtgcaggacagtgaggcccttaatataagtgtgtgtactctggaggaggcaaggagaggagccaagtcttccttttgggtgtcagcatcctttctttcacacggttctgagaaagcagtattgaattaagcagcaagcttgctgtcccgaggtgcagacatcctgagtgagtccgttctgctcctgcctgctcctgctttcttgattagtcaaatccaaatcagaggtgaatgaacaaatgtcctttttgtgtcaggtgtcatgtctactacctagacattacagcagttgagacttatctaagagcattttcatggcagagctgagactcctttgtattgcagtgcctctctactttaaatgaatttattttctcttccatggaagagatttatatgggagacttattaaagaatgttttgacatctatttagatgttattccctaactgtactccatagtactttattatgcacagtcttggtgtctaattatctcagatctgcattgtctcctttgccaagtctctgaggccctgtatcattttgatctctcttttgtatgacccttagggcatgggatattttaagcattcaataattaacttgactagtaggtaaatatttgttatcagttttttaattgcaccaaattttacattttagcataatggaaaacatgaagagctggatttgctacttaaagctcatatcctcagaaggtttaagatagagaagttatctagtgctgttgtcatttaaaaggagcctagtgcttccacattagacttatatttaatacgttaagaattaaaattaggaagagtgagataggtcacttacttaaatagtttttagtcatctggttagtctactaacttttatggcatcatcccaaaatatatgtagttacaaataatgaatagctgtcttctttgaattataatcctctgtgtgttttgttgtactgatttgctgtgggtttatcttggaggaaaaagaaatatttcatgccttcttaggatggttttacagaggggtgcaactgtgaccagtctaagtacttgatcccagtccgcattcaaagtgcatgtttttggtgtcagagggaattaggtgaaaaaaatatggatagattagtataaatctgtaactgctattagaaaaaacaggcacattccatattaataatggacagtattgtcatttctatttcaaggacccaatgaatatctagtatgaatatataatctttatttaaacttctatttgattgtatatactctgtttgctttgtcttaaattctcttttttacatggtgatattaaatatttgtgtttagttgatattaagtgttaatagcctctatgggtggttgaaagatatgcttgtttctcagtctaaagtttatacccatgtcatatctttgtaagtaattttttaaagtctccttccagctgtcttcattctcctgtttcactgatgcacacatgctcaaaggatttaagacattttgtatacatttgccagtcaagggggaaaaaaatctgtcaaccacgtctccagagtctgctttttcaattagtctgtcattcacatttaacttgaggtatatgtaggccagtaggttagattagtgagtatattcaagctggcttcttttattcatcgtaatgtctttgaaaatcagccaggttgttgtatgtatcaatggttgattccattttgttgctcaagagtattcctttgtatagctgcactaacagtttgtttatccattcacctgttgaaggacatatgggttatttccagtttggagcaattgtgaatagaactgttataaacatttatatatgggtttttgtgtgaacacagttttcatttctctaggataaataccaagggttgggattgctgggtcatatggcatatgtatgtttaactttatcaagagttgccaaactgttttcaagaatagctgaaaacagccccactcacaatgttatgagagttctagttgctttgcatccttgtcagcattttgtgttaccaccactttttattttatttgttcttatgtcattttggctttaatttggatttctctaatgtctaataacgttgaatatttttatgtactcagttgccttccatctgttcttttcagtgaactctgttcaagtcttttgcccatttttaatggctgttatatgctttcttaatgttgaattttgagagttccttacatatccaggatacagatcctgtgtcagatatgtcatttgccaatattttctcccagtctatactttgcttttcatactcttcacattgtctttcatggaacaaaagtttttaatttggtgaagtccagttcataatttttttttcttttatgggtggtactttcggtgtcatgtctaaaaattctttgcttcactccatgtcccaatgattttatctcatgttctgaaagttttataggttggcattctacaaaacattagatctgtgatccattttgagttattgtgtaaggtcttctgaagtttaggttaaggttcatgtattttgtcacaatgattttatctcatgttctgaaagttttataggtttgcattctacaaaacattagatctgtgatccattttgagttattgtataaggtgtgaagtttaggttaaggttcatgtattttggtatatgggtgtccaattgttacaacacctattgtttagggaaagactaccctttctcccttgagtcatcttgcactgttgtcagaaatcaattggtcacatttgtgtgggtctctttctcagctctattctgtttcgtgtatctgtcctttccctatattacagaatgttttaattactgttggtttttatgatgtttttaaaccttatgatttatgattctttcagttttattcctcatcaaaattatttggctattctagttcctttgcctttggtttcagaatcagtttgtctgtatcagtgcaaaatatcctggggaggggttgattagaattgtgttaaatctgtagatcagatgagaagaattgacatctttactgtgttgagtcttccaggccttgaatgtggtagctctctgcttatttacatcttttcttatttctttcattgtgttattagttgtactaaagaagctcttgtacatgttttgttagatttcatatttcattatttcagagctattatgattggtttttaaaatttttagtttctgactgttcattggtaatatataaaaatatgattgacttttgtgtgttgacctgtatcctgtgaccttgtaatcttactctttagttgtaagaggtttgcttggtagattctttgggatttctgtgtaaataaatatgtctgcaaatggagatagttttatttctttctaatttgtctgcttttttttttttcttcttgcttcctttcactggctaggatgtcaaatacaatgttgactagaagtgacaagagtaaatatcattgattccaattatagaggggaaagcatttttcccaactatagagtgaaagcattcaggctttcaccattaagcatgacgttagctgtaggcatgtccttttatgtgatttgggtttgttttgctctttttttcttctagcttcttaaggtggacagttagattactgatttgataagtttcttttttgttaatataagtatttaatgccatacattttcctctgagcattgcttaactacatcatacaaatgatgtatttttatttccttttcagttcgagatattttctaacttcatttgagacttcctttttgacttgtggattacttataagtacatcactaaatatctagatttttggagattatttctgttatttatttgtaatttaggttcattatggtcaaacaacatattttatgttgcttcagttcttttaaatttgttaagatttgttttatggcctaggatgtgatctgtcttggtgaacattttgcatgtacttgaaaagaaagtgtattttgtttgctcttcctgggtagaatgttttgtagatgtcaactagatacagttggttgatggtgttctatcttctggttgatatttttgtctattagatttttgttgcatagttttttgagaggagggtgttgaaatctccaactgtcattgtagatttgtctgtttctccttccagttctgtctgtttttgcttcatatattttgaagttctgttgttaggtgcatacacatttaggattatattttcttggtgaattggccttttcattattatgttgtggctctttttattcctgataattttctttgctttgaaatgtacctatttgataatgatatagccagttcaggtttcttcttattaatagttctatgttatatcttttttcatccttttacttctaacctacttatctcatcatatttgaaataaatttcttgtaggtggcatacaattgggtcatccttttcaaaaaaatctaataattttgtcttttattggtatttttaggttatttacatttaatataattactgatacgtttagatttggctctgctgttttattatttgctttctgtttgttctctttgtttattgttcctcttttcccccttcctgctgtcttttgagttatttaaacagtttttagtactctattttaacataagtatttttccacctaagtgagttgtagaaatcttaacaccatatgtatttctctgccttctttcttgtttgttgtattgtcttttatattacatctacatacatataaaaccccatcagacaattacatagtttctgctttgaacccaatgggtactgtttttttgttttttgtttttttaaacctcagcattcattggatggttaccatttctttgttcttccttaattatagataatacattttttcccttgatatcatttctcttctatcctttagtaactccttcagagtggatctgctggtaatgaattctcataattttacctttattcaaaagtgtcctcattttacttacttccctagagtctgagttgacaattattttctttcaaccctttaaaaatgtgctgtttcctttcgaactgcattgtttctggttagaaatttataggcagtcaaattactattcccctataagtaatgcgtcacttttttttttcttgctactgtcaaaatttttcgtttttattttttacctgtttgactatgatatgtctgggcatggatttctttgggtttatttaaaaatttatttatttatttttttgtagacggagtctcgctctgtcgcccaggctggagtgcagtggtgcaatctcagctcactgcaacctctgcctctccggttcaagcaattcttctgcctcagcctcctgagtagttgggattacaggcgtgcaccaccatgctcggctaattttttgtatttttagtagagacgtggtttcaccatactggccagactgatctaaaactcctaaccttgtgatccacccgcctcgacctcccaaagtgctggaattacaaggcatgagccactgtgcctggccctctttgggtttattctacttggtttctctgtgcttctagaatctgatatacactgtatcttttactaactttgttaaagttttcagccgttatttcctcaaatatttttctccattgctgtctttcactctcctttcaggataccagtgacacgaatgtcagaccttttgatatcccaaaggttcttatggctctctgttcactatttttctctttgttgttcagactagatcatttctaccagtctatcttcaagttcactgactttcctatgtcatctccattttgcttattaagcccatccagtgattaatttcagttacagtatttttcagttctaaattattattattattattattattattattattttgcaatggagtttcgctcttgttgcccaggctggagtgcaatggcgcgatcttggctcactgtaacctctgcctcctgggttcaagcgattctcctgcctcagcctcacgaatagctggaattacaggtgcctgccaccatgcccggctaattttatatttttattagagacggggtttctccatgttggtcaggctggtctcgaactcccgatctcaggtgatccgcccacctcagcctcccaaagttctgggattacaggcatgagccattgtgcccagcctaaaattttcattctgttctatttaatggcttctacttatttgttgagaccaatttgtttcaagagtgttttcccttgttggagccttcttttttttttttttttttttttgaaacggagtcttgctctgtagcccaggctggagtgcagtggcacgatctcagctcactgcaagctccgcctcccgggttcacgccattctcctgcctcagcctcccgagtagcggagcatttttataatagctgctttaaagtctttgtcagataattcaaatactgtgtcatctcagtgttggtgtctgtttattgtcccatgcaagttgatattttctgggtccttcatattctaagtaattttagattgcatcctgggcatcttcaatattacattataagactctggatcttacttaaattccatggagaatgttgctatttgtgttttagcagtcagttgagtctgttgggttcaaccttcaagttgcagccagctttctgtgggtggtgatttcaatgtcagtttgttttcaaagcctgtagtgcttcgctgtttgggtctgtcctatatgcagtatcagtctgatactgttccagtcagttcgagataggtggtaatctctctcttagttcagttctcatagtttttggtttgctgtttaggttgagatccatgccatgttcagctctgggatgaagccaggagtttataatcaactttatggattcactttgctgtgagttcctccctctcagcaatttcctttatactttctgctgccgacctcttttcagtccttgagaggaaaagctgggtctttttctactctgctcacttcctgtgattgcacccacatccagggctaagtggtaggagtacagagaggaaaaaaaagttgatggagagaggttgcctactctcttgaaacaacagcttctgggatagtggaggaatgtttccctccctcagagttttaggtacctgtgggcgcctaactgttaactgccaggatccttttcccagtttcttaagcctgaactggagggcttctcctggatctctgcattgatttccacttccaggtttcccccaagtccaggtggcaggatacaagaggagaaaaaagtaaacttaccactggttccatgctaccattgaattttgatattttgccatctacctcccaccatttacttttcagaatctttaaatagctgctccaagcattctggggttttatagctatattcagtaggagagacaggatggagtgtttttactctatcttggtttgcattgaagcaatgtgtactgttaagtatgatattcatttattcataagccagcacacgtagctatcatttgagtgcttactatgtattgggcacttattacatatattatttgtttaattctccaataattttcctcattttaggcatgaggaaatttaagggtaaaactgtttacctgtgtccatagatgttaaatacaagcagggatttttatttcttccttttcaagatagaatcttgctctgtcacctaggctggagtgcaatgctgctatctcagtcactgcgacctccacctcctgggttcaaaccattctcctgcctcagcctcccgaatagctgggactacaagtgtgtgccaccatgcccggctaatttttgtattcttagtagagatggggcttctccatgttggccagactggtcttgaactcctgacctcaaagtgattcgtccatcttggcctcccaaagtgctgggattataggcatgagccaccacgctggccagcgatttgtatttctgacccccaaagcttttccatttgcctctaagccagtggttctcaaagtgtggtaccagggctagcatcgtcagaatcaacctgggaacttgttagacacacaaaatttctggctctacctcagatctactgaatcagaaattctgggagtgagaccccataattcgttttgacaagccgtctgttatcctgatacatgcttaatttgagaaccatttatctgtgctgtataacttcacaagcactatgtgtggtttagtttcttagcaaaaccttaatataagaaactagtttttcaccattgtttgagacatatgctatatgctaaaatctaattttttactaacatcactgacatatagcttgaaattcacactgtgttactcaagataacagtagttaaaatataaatactgtgacttaaaaaatttaaatgtatgcatataataggggatccaaaagtagccatagttgcttaacaacagggatacgttctgggaagtatgtcattaagcgatttcattgttcgaccatcatagacaagactcacacaaacctagatggtatagcgtactatacacctaggccgtatagcgtagcctgttgctcgtaggctacaaacctgtatagcctgttactgtactgaatactctagacagttgtatcacaatggtaagtatttatgtatctaaacatctatacacaaagggtacagtaaaaatacagtataaaatatttttaaatggcacacctgtatagggcacttgttttgaatggagcttgcaggactggaagttgccgtgggtgagtgagtgagtgagtggtgtgtgaatgtgaaggcctagaacattactgtattgtagaccttataaacactgtacagttagtctacactaaatttacttaaaaatctctttcttcaataacaaattgacctttagcttcctttaacttttttactttgtagactttttaattctttataactttttgcctattttgtagtagtacttgttttaaaacacaaacatatacagctgtacaaaaatgttttttctttatatccttattctataagcttttttctgtttttaaatttttagcttttttgttaaagactaagatacaaacacacacattgtcctgagcatacacttggttaggatcatctagacatcattagaataggagtttttcagctctgttatgatcttaaaggaccaccatcatatatgccgtctctcattgacccaaacatcattatgcagcacatgactgtatatgtaaagaaatcaatcttagatacggattttggccaacaaatcacttcacatctggtaactactatgccatgtaatgctcggtattttaagtttcaggatattaatactgtttgtcctagatttcagggtaaaacttttgagattggcttagattaagattaggttggccaggcatggtggctcacgtctgtaatcccggcactttgggaggccgaggcgggcggattgcttgaggccaggagtttgagaccagcctggccaacatggtgaaaccccgtctctactaaaaatacaaaattagccaggcatggtggtgggcgcctgtaatcccagccactttggaggctgaggcaggcgaatcgcttgaactcaggaggcggaggttgcagtgagccaagattgtgccactgcagtccagcctgggcgacagagtgagactctgtctcaaaaagattgaatggcccatatttggagaatgggtcctatttcatacaattttttcccatgaaatattaaatgtataactaaatttaatttgcatattcctagaagacattttatatacacttgctaattgatgggaaaaaatctatcaatcacatttccggagtctgctttattaatcagtctataattctcatttaccttgaagtatatgtaggctaggatatcggattagtaggtatattcagatatgaaggttaattatggaccattgtctcttttgtgttacattttggggtcagaaacacttgtgtttaagtccgagctctccttttactaataaggttggtgggggtttggagcacagcaggtgttactaattctccattgtagaagtaggctttatagccattctttttatattcgattttaagtccattactccattgtgtctgcattagcaagatgcgtaccatctgctggatactcttcaaggagtatcagtactaaggtggaaagggctgctgtagactgaatgttcgtattccccccaaaattcatatgttgaaacctaatccccagtgtgattatataaggaggtggagcctttgagaggtgattaagttatgaggatgataccctcatgaatgggattcgtgcctttgtacggagctgaaaagatgagagttctccttccaccatgtgaggttacagtgagaagatggccatctctgaaccaggaagcaggtcctcaccagacaccaaatctgccagtgctgtgatgatcttagactttcagcctccagaactatgagaaatacatttcttttgtgtataaaccaccctgtttatggtgttttcgttatgacagccagaaaatactaatactaagacaaggaccctgccctcaaggagtttactgtctattgaaataaacaggaaaggacttacgcaaaaatgtgattcttatgacacagatatgtacaaaggactgtggatgtggtatgttggagccaggtttggccagtagaactgaactgttgttggtacatataccacagaaaccagcaaaatactgcgtccagtcagttctgctataatgagatgtatacattcttaaaaatcaccaagtgtagggagaaataagattgaggcacaacccataaaagtttcatcagtaacacatttttaaaagaaataggaacctaataaaaatgatagcaccattttaacagttttacataagagaaacataaaaactacaataaatatggcactttaccttaaaaaagacctgaagtttgcatgtgaaggtagtcatgggaaaagttgcctcttgtgagatactgtgaaatggtggaaggacagttgcctaaaatctgaggaaaagttaacaccagatgtggatggctgtggctggtaacttaagagatgtactgaggtagctaggagatgtttgaggtatatgtgttgtgtattcctatgttcaggctctgtggctggatgcagttttatttcattcatctagtgttaaatggcacataagaaaatgggattcatgtgttcatgttgtgccctagtgtaaccagcacattgaaacaaattgtagttttcaaaacaaacattatagtagaactgactgtatcagggcttttattccattccgagagcacaccactggaaggagctgtcagtcagcctggaggagtcttggatgcctttacagggaagatgatgagtgaactgagacttgccgaatgagtgtagtaatgtttttgctaggtatagtgtaagtagagggagggcattccaagcagagggaacagcagcttgtggaatagcatgcagaaggaagcctggtgtctccagaaaactcctatgtagtttgaacttggaaagaaaaagaaacactgttaatgaaacttagtggttaggcaggagccaaattcatcatggcccaatgtgccacttgaaagaatttacattttatccttcaaggggtagagaccactgaagaattttgaacagaaactatttcccagtgctgatttatgtaacactcctggagacagtatggaagatagtttgaagcaggcccagctgggctctgagaatctggaggtaaggaagaaccaatgtgtatctttaataggcttgacccctttgagaatattataaaagctatagatgctcactggcaactgtgtcctttctttgttcctagaaatcattatcactgtaatttcatctcttgatattaagggccagggccatgcgagttagtcttttatatggtgatgagttaggtgaatttataatcaactttcacaatagctggtttcactgttagctttattcttacaatgtttaaaaatcgtgtggtgaatcataattggagagattttaacatcttaatgtgtatccactggcaaataattacgtgggtcaaaggcctgtttacatactagattcctatttttgcccaaattatttttattttatttatttattttttgaggcagagtctcgttttattgctcagcctggagtgcagtggcgcgatctcagctcactgccatctccgcctcccgggttcaagtgatgctcctgtctcagcatcctgagtagctgggattacaggcgcatgccactatgcccagctaagttttatatttttagtagagatggggtttcaccatgttagccatcctggtcttgaactcctggcctcaggtgatccacccatctcagcctcccaaagtgttggtattataggcgtgagccaccatgcccagccagtcccagattattttataataaagaataactagttgggtgtggggcccatgcctgtaattccagcactttgggaaactgaagtggggggatcacatgagtccaggaggtcgaggctgcagtgatcctggattgcaccagcctaggtgacacagtgaaactgagaccctgtctcaaaaaaaaaaaagaaaaaaaaaaaaaaaggaataaccattgtaagggatgaataaataataagcaagtaaaattcaaggttaaaaaaagctataacattctttcaagcaaaagttacaaatagatacgaatgatttgaataacatctaaatgaagaaagccatgtatctcactctttaaaaaatcagtattttcccacagtgctccttagcaagtgttttggtgcctgttacagagtaaggtgctgagaaaactcataatctggttgaagatactaatgacacaccaagagtaattgaaagcaggaatatcaggttaaaacataaaaaccatgtcctacagttttctagttaataaactgtcccacactccatttaggatgtaatcagtatgcagtaactatttattaaaagactgaaatgtagtacttaacggagagttagaaagaaataatggaaacgttgtgggacttcttggaattttctgatctttgacactttggacagtataattaatggattatggattcacagaggccatataccctttcagtaatttgtctctgatacttctgtggagcaatagttggtgctgttttttattttcttaataggacactggtggttttttttaattagaaaactaaaatttacaaaaatattatacagtcattgcagaaaatgaatgccacttgtatgaataaaaattaaagctcttaataacctttaaaagcactgatttttctttataaccctttgagaggggtcagatcatcaaataccaagtttctggtactgttcagctatcacttactaacctatgacagccatttgtgaaaggaaacctgcatacaggtataggcatataataaaaaggaattttattattgatagatggttacagttatacaataattcattggccttagttcactttaataggcagatatttgaatccttacttaatctagccattaccgaagtatacatttatccatattcttctcccttaactctcagaatcataatcttttcttcatgtcatatttcttctagtcctcaatatttactaaaataataatgtacccttctcttacagaaataagattttttttttcctgaacccctgtcctcatcatacccaatccatactgctttgattgtcttccagctgagccatttggttcttctagcatatgcagagaaatgaagtcttagtcattgaataagaccacatccaggaataaaagcaccttttaaacatggcatggaagacgcttgagagtctctctcttccccacttttcaggttgctttgtcttttacttgacatcatccgccccatacatcagccatctgaatcattttccatcttttggagagccaggccctttccggagtccatgcctttgcggtatctgcctgaaattccattttccacttggcacatagctattcagtcattaaaacctagctcatgatcaccttttctgcgaagcctttcctgatcccctgcattagttaggattagttgggtacggtagcaagtaacagagacttcaaaataaggatggcctaaaaatgagaatttatttcctatcaaagttcagagtttgcagtgtagggctaaaatgacagctctgctccacgaagtaattttcagctcatggctttggaagctctcagatttgacccttgtccttatggttcaaaattgaggtgtctttgttttaagcagcagtatggaagaaaaaagagacaaagggcatacaccagctgtttctttaaaaagcttccctaaagcctcccacaccatgtatctggttgcatctcattggccagaacttagtccatggccacaaggaaggttatattattatggaagaaaagagaacaggtatggggacaattggcagtctgccacattactccaactagaggtagttgctttgcactttgtgttcccagagcactctaattatttcacatttaggctatattgtcatttgccttctctgcttgattctggacttaacagagactgtgtttttcatctttttatctctagcactttacataaagcttagcacatagtccatgttcatacttgttgaatgaatggataaagacatttgggaagagtacagcaaaaatatcagacataagcagatggcagtatttaaatacccatgtgtacaaagataattgcttatgtagacattgatgtcttttcttatacttaatttttttcctattttctccattatgtgaaaagaatagacacagtcaatagtagagggcatgcctcaaaattgaccacacagaacatagagttatttaaaaagtaatatgattctctgtgttatactttattattgtagtaaaatatacaaaacataaaatttgccattttaaccatttctctctctcccccattccccttcccttccctccccttctgtacctctctttccccctctctccctccctcttttgagacagtctctctctctggctcaggctgaagtgcggtggtgccatcatagctcactgcagcctcgactttctgggcccaattttgtcctcctgccttgacttcccaagtagctgggattgtaggtgtgtgccaccatgcccagctaatttttaactttttttgtaaaggtgaggcctcactatattacccaggctggccttgaactcctgggcccaagcgatcctcctgccttggcctcccaaagtgctgggatgacagatgtgagtcaccacacctggctcattttaaccatttttaagtgtatagttcagtggcattaagcacattcacattcttgtgcaaccatcatcaccatctatctgcagaactttttcatcttccccaactgagactctgtactcattaaaaactaacctcatttcctcctctccgcagcccctggaaactaccattctactgtctgtctctatgaatttattactctaggaacctcatgagaagaatcatcagtatttgcccttttatgactggtttattccacttagcacaatgtcttcaaggtacatccatgttgtagcatgtgttagaatatccttcctatttaaggctgaataatattccattacgtatgtagcacatttggtttgtccatgcatatgttgatggacgcttgggttgttgtgctacccctaaacacctattacccacaaacacaagccacatacaggttggtatcccttacctgaaatgcttgggaccagaagtgttttggatttcagatttttttttttttaatatttgcattatgtttactagttgggcatccctaatgtggaaattcgaaatgttccagtgaacatttcctttgagcattatgtaggcactcaaaaagtttcagagtttggaccattttgaatttcagatttttggatttagggatgctcaacctgtactcacatagcttcttgaaaggaaatttaagaaaagtacactcacctatattaagaacttatttttctagcaagtaatttatcacctctcccaatcttagcttctgaaatgtaaactaagaaagttatgaagaacattatccaggtgttaaatcttaaaagtagatccaagagtggaattagagaaatacagtggcctttcacaataaaagactagattttaagagtctcttaattatttggtgagagacacaggacagtacaatctgtgaatttctattcttttgaactatggagattttatcatgactggacaatgagaagaaatgttatagctgtagcttgatggttttgcttttataaaattcttctggtattgaagagaaaaagattattaatgggtaaagtaagaaaaattaactccattcctactgggtgacttttattatcagatagcaatctgataactgattattattagtttggtgacaataattatttttggatgcttggttgctaataatttgcctgatgcctagaaaattctgatttggggtctgtattctagtgggcatatagcccaaattagcaatcagattcttctactaaaattatctgatcaagctttcagataattcatgcatttacatagaatacatatcagtggtttaaacagaatttcacagacgctttcccattgaagctgattgtttggcaaaatggtgagtcatgctttactggccaactcacacaccagttgttgatataatcagttacctgagccaagatcaaagcagcagaatagaagatagtaccttggctctgatacctttctgtcctttctaaaaaaaaaaaaaattgggatgtaaatttccatccctctcctatgtgctttgggttttaagaagcaatataagaccattttttatctttagaatcttacagttgtagagataaaattgatatgcttgaaatgatctgaaaaggtgagaacaaattgtggtgttaactataaacccttttagagaaatcaacagggagacatttgaacttgatgtagatgttaaaattagaattgaatggtagagggccaactaagggaggtatttcagagtaaggagaaattagtttggataaagcagggcctgattatgggtggtaatgagtgaatgtgaggcgcaagatgctcaaactgttgaatgttcctaaaagaaacagaatctggcagtataatgagtccaggaagacaggagagagactaaggtgttgtagaaatctaggaatgagacaaaaatctggacaaagttactggcagtgaaagtagacagaaaaggtacactggagggacatcttgaaaaaagaaactgttgaactaggtaacattaaatatggtggtattaagtggaggaagagcaagatactctaagattttcccaagaaatcaagtacaatcctggtaaataacatttcaagtacaaacctggggagacatattgttaattttggatatagttaatttgaggtccgatggagaatagccagattgggacagacttttaggctgttagtggactgaagagtgtgtgttgaatgtgtggacccaagtgtgtttcccatacacatccagttgtatttgaggaggtgaaaattggtcctagagaatatttgtttataactgtaaaggtaggaacatatgttctcatagcttctatgtatgtgttaaaaaatctttaagtcaaataaaatttttttcccattagcatcatttcacttgaaggaactaggttggttaaatagatgaagttcatcaatcacatccaggaaactgaagttttttcccatacagtcaaataagagagctagtaagctgcattttgctagcagtaagtaagatgcagaaccaggataagaacgcagattctttaaggaacatcttggaataaacattgtggctgaataatgtcatatcaatgagcattatattcttaaatattgtaaattattgaactatttaacacgtttaatttttacaccatttgaagtaatgtaccagtagaaagtggatgcttcatgtttttctttggtagaatatttaattgggtcgactgtgccaaattttatttgtggctttctctctcagaatggctctttttgttaatagcaaaacatacataaccatatggagtcaaagagaaaaaaaacttggcagaagactatatccattgttattggattgtaaattgtcataagatgtaatatcttacaggaatgatagttcgaaaggtaatataattagagaaataatagtcttgtgtaggggtgaactattattggagtttcactgaaggatacctgaggtggttgttagtagcactttgcgagtgcctccaagtacaagtacagtactgctttctgctttctttgttgtctgtcttgcgtatgctgctttttggttattctccttttctgtagtaaaatgtaacccatatataaaagaataaatacattataaagatacagtttaatgagtaattataaagcatatttgctacccagattaagaaatagaatattgccagtgaaccagaagcctgctgtgtgccctttcccagtcatactccatctgtccccaccccatcgcatcccccagaagtaaccatattcttacttttgtgaacgtcattttttctttctctttaaacttttaccatctatatatgcatccctaaatggtatagtttagttttgcctcttttgtacacatgtctacacacatatgtatacatgtcagtattcgtgtgtatatttttatttctctaatccattgtgcatatactattccattgtatcaatatactataagatatttattctgctctactttataatattgaaaaacgcagctgtgaccagtttaggaaataatgcccaggaggatctcctctctcagagaactaaggtattaacgtattcaaacggaagcctttatctgtagaatacttgttaatatcatgtggtatactagaattctagggaacatgagttatgaaatactggataagaccttttgttattacattttgtcactgagtaatttcagtgcctgctattcctacaatcatgtctaaggagaacattcactgagggagtaaactaaggtagctgtgctttcaatcgtgaatgaccttaccttacctggccataggtgattggactggcatgggtatccaaattaaagtcaccagtgattggtcggccactgcccaaccaggtggaagcacagaaagttgtctccacatgggaatgagcttaaccaatcaggttctgacaggccttacaattggctatatttggttaaagaaatacagcttttgcttttaattttctgtctggatgtactccagatagacaaggtggagtactggttactgtaaggatacacatgatgtgtatcataaaactcacaaaaagaaatagagattggcagttaagtattggaagcagaatttgaaaaatcaagagagagataggaggggcctgtgatagatcaagaattagcaaaaggtatgagaaagtggcacctgtgaggaagaagaagaatacagagtctagaagcagagataccagctggtggctggaggtaaatcacatatctaaagctgatacccagggagttgctgggtcccagtaagaaccagagttctagaatgaggatctgccactgcctactgctcctcttgctgcttcctagggaagctctggaaccagacccttctcagtcccagtatccttgaggcccagacccacttgtgtcactgattttgttcttggacttctgacctgttttccttatcaaatccgtgcttattaatcaggatccttcctcagccatacacacccacatttgagtatgtcagaaccttgcaaccagaagagactcataaatgagttgaaattcattagtgatattacttaccactaattcctaatactgtttgagaaagtcacgctggcttgagtggagacctgtatttgcgattcctttctccattgattgtgttctctgcccctgtaacccaccatttatttgtgtctcttcccttcctcaccgttgcttcatctttaaaaagtttttaaaatatttcattattcatttcttctgcattttgcatatatctttgagtttataatatgaaattagattgatctttttcaagatcgacttcctttttcttttatttatttgaaacggagtctcgctctaccactcaggctggagtgcagtggtgcgatctcagctcgctgcaacctccacctcccaggttcaagtgattcttttacctcaatgtccaagtagctgggattacaagcgtgcgccaccatgccgggctaatttttgtatttttacaaatcccaaagtgttgggattacaggcgtgagccactgcacctggccccaagatctacttcctttttaaataacattttaaaaatattgaatctaatattttggaaagtacaacaaagaaaaaaaatgaaattacaataaatagaaactatccagaaataaccacagtgatatttcctaactatattttttaaaaaatgtttgtgatatagatgtgtgttgaggtctgtttagttgtactattttataaccatttttatttaatatcatgaacattaccagaatattaaatgccccagaaacatggttttttatgtctattatttcagcatgtaaaggcaccagaatttatttaaccattcttctattcactgagcaatatgattattgccaattttttcctaatataactaatgctatgatgaatatttcctgcatataaatctccatccttatctgattatgttcttggaataaatttctaattgggaagccttagatagaaagatgtgcatagttttcaagctttagtacataatatacaatttcctttctgaaacgtcatgtggatctatgttctcacattagtgtgtgacaatgctactttctcaatacccatgctagatagtaccattaaaatgtaaaagaaataaaaacaaactttgacaggttgataagtgataaatttgtatcacattttaatttggatgtctttgacaaattccaagaatgacctttattggccaagtatacatctgatttgtgattattctagagatatcctttgctgttttttaattgagttgttgtgttttaccattttctttgtcttcttttcttttttaaatagagacaggtcttgctttgttgcctaggctggactttgaggtcctgggctcaagcaagcatcctctccatacccccacccccacccccaccttcttagtagctggtctgtagacgcacaccactgtgtctggcatatctttttcttataaaatatatatattaattttcaacatatgacaacattgtcatgtatcatgtcgtttccatatgtcatatgttgaaaatgttttattcagcttatcatttacctttgtttatgacattttttgatatacagaaatcttaaattttatgtcatttagtcttttgatcattgccttttggtttattgcttcagcattttgcttaagtacaccttcccatcttttatttgagaagtcataagtttttttatgtttactacttctttttttttgagacagagtctcgctctgtcacccaggctggagtgcaatggcgcgatctcggctcactgcatcctctgcctcccaggtttaagcgatccttctgcctcagcctcccgagtagctcacattacaggcacataccactatacctggctaatttttgtatttttagtagagacggggtttcaccatgatggccaggctagtctagaactcctgaccttgtgacctgcccaccttggcctcccaaagtgctgggattacaggcatgaggttccacgcctggcctgtttactacttttttaatggaaggtttatgtttttacaatccgatgaaattcttacccattcatcttatgggaacatttgagattactcaattttaggccccaaagtatagttttttagtttcagaaaacttgactgttaactttttatgagtttttcatctacttcctccctccttccactgcatatatgtattatattcaacatgttacaggaaaggggtcccgatccagacccccaagagaaagttcttgagtcttgcgcaagaaagaattcagggtgagtccatagagtaaagtgaaagcaagtttattaataagttaaaggaataaaagaatggctcttccatagagcggccccgtgggctgctggttgcccatttttatggttatttcttaatgatatgctaaacaaggggtggattattcatgcctccctttttagaccatatggggtaacttcctgacattgccatggcatttgtaacctgtcatggcactggtgggagtgtaacagtgaggatgaccagagatcactcttgtggccttcttggttttggtgggttttggttggcttctgtcatgcagcctgttttctcagcaaggtcattatgacctgtattttgtgctgacctcctatctcatcctgtgacttagaatgccttaaccatttaggaatacagcccagtaggtctcagcctcattttacccaacccctactcagtatggttcacatgcctctggcaaacatatgtatacttgaacatttagtataatgataattgattaagcgtcatacaagggactggggggcatctccaaatatgtctgaaatttaaacaaaggccttccatttgtattttctgttggtaataggaaaaaaggagacacacaatgagttatatttttctcattttctacataaaggaagcatatcagttttttaaagagaaaccttggcagtaaagtcaaagaagaatttttttgtgtgggtctttatatcagggatgatgtcttttatactattttctttctttttttttttgagaaggcatctcactctgtccaccaggctagagtgcagtggcgcaatctcagctcactgcaacctccccctcctgggttcaagcgattctcctccctcagcctccctagtagctgggattacaggcatgtaccaccacacctggctaatttttgtattttcagtagagatggggtttcaccgtgttggtcaggcagattgtgaactcttgacctcaagtgatccgcctgcctcggcctcccaaagttctgggattacaggcatgagccaccacacccagccctttgataccatttttatagaacgtggaataatataagaccacttatttgataaactcttaagaagttgagagcataacattaagttgtaattctggtaaagtatttgtgtagggagccaaaataatacaaaccacatatatatctttgtggtgaagtaatttgattacagttagaacatagaaaatcagtgtgggtggtgagatatttccaaactagttctttcaaaataataaagactttttttttttttcttgagatggagttttgctcttgttgcccaagctggagtgcaatggcactatcttggctcactgcaacctctgcctcccaggttcaagtgattctcctgcctcagcctcctgagtagctgggattacaggcgcatgcccccatgcccgctaattttttgtatttttagtagaaacggggttttaccatgttagccaggctggtttcaaactcctgacctccggtgatccgcccacctcagcctcccaaagtgcagggctcacatgcgtgagccaccgcgcccggctgtaatcgagatttttgacccaattttattttatgtctatttgtggtttctatttagtgcttgggaaatagttgtggaatggttcatccatactagatctaagtattgcccctggaaagttttatattttattgtgaaaattagaaaatctgtataacaagatgtagaattatcccatttaagtggatttccaggattgacctgcaagcaataacaaaggatttttacccactatgcttcaaacaattatgatgcaaactgtttaatagtaagtcttaattcatttcagatttcagagaatgtcttaagaattttggggatattaatgtagcactacatccttatcaaagaatattgagaattctgctttaaactaccagttcatctttgagactgtttaaacctgcttttctttatctgcaaaagaagtctcaattttaagccatgtttatgggaaatgactataccttaaactgtgtagaagttcttttagggggagacaggaaagggaaagtttgtgttggaagaatagcatttctttgattacatatttttgtagctgacactacatttacattgtttaaattgaacataaaattgtacctgatcaaaaacatgtctagctatggagtatgagactatttaaataactcagtattatacattaggatttttcttttatttaattggagaggagtatgatcagaattagttgtaactgttgtgctatgttagaccttaaataactggattccatttgtggaatgctgtgcttattttaaaaattctaatattaaaagaaaaatcacagatctttgaggttagctaattgagttgcttgtagaggaaggactacctgggctggctagagacagagggttgtttatcctctttaacatttcccatgaaagactctgcacaaatctctttcgattgtttatttacctttttaaccatttgaaatttaattttttcctgagagtttctttgtcttggtctttagaaggattgaagagcggccagtcagaattcccatttttaaagaaagggtctctgtatatgtaaaaacaaacatttgatcaccttcattctccagatggcataattccaactatattatctcaatattcctaaattataataccgttaactgttgttttgctgtactttttcttcccagtttttaaagggtggaggaggtagccagacacggagccctttgagttttttttttttctctgatgcttgaaatcctgggatcaatattatgaaggatgctccagaagggagtgagggtggagaaaggtagactgtttaggcatctgattcatccatgcaaatgatgaagactgaacctgttagggggcagtggagatggagaagagggataggcccaatatggttgtgaagtaggctatagactcacattcttattattcaccactccccagagtaagtacaaaggcagtggtaactctagaatatctagcatgttttctctcctgccatttgaatcttctcctttggagtatagaaatattccatcagggccgggtggctcacgcctgtaatcccagcactttgggaggccgaggtgggtggattacaaggtcaggagattgagaccatcctggctaacacggtgaaaacccgtctctagtaaaaatacaaaaaattagccaggcgtggtggcggacatctgtagtcccagctactcgggaggctgaggcaggagaatggtgtgaacctgagaggtagaggttgcagtgagccgagattgcaccactgcactccagcctgggcgacagagactccatctcaaaaaaaaaaaaagatcccatcagcctgtctcctttaaggttatatatttttgttacatctcttttaattttggtttctacatgtttgttgttagtacggtatatagttatgtaattgatctttgtgtgttgatcttgtatcctgtgaccctgctaaactcacttagtttacttttctttcttttttaagattccttagggtttgctatgtagacaatcacatcatcagaaaatggagttttattttctcttttccaatttgcatgactttatttctttttttttttttttttttttgccttattgagctggctgttgcatctcttttgatctcttgttctggccatcagtgattacatatcaaggttgctagacagatttgtttcttctgcctagaacagtttgcatgggctggtgttttccttgagcaaagattttaattactgattcaaagtctttaacggctactgatttttaatttcttcttgagtcagttttggtaatttatgtttttctaaaaatctagtcatttcttcttagtttttaaatttatttgcttacagttgttcatagtggtctcttacttttttttgagacaggattttgctctgttggtcaggctggagtgtgatagtgcagtcacagctcactgcagcctttacctcctgggctcaagtgatcctcccacctcagcctccagagtaactggaaccacaggcgcacaccaccatacctggctaacttttgtatatagaggcagggttttgccttgttgcccaggctggtctcaaactcctgggctcaagcagtctgtctgccttggcctcccaaagtgctgagattacaggcatgagccactatgcccggctagtctcttttaaatctgttttatatctccatgtgttcccttttttatgtcttgcataatgtgtccatgtagtctgtggccctgggctgttacactttttaaggtttctttgttcttctcttcctgtggtcattgtacttacacgtagtactcctctataaccacatccccaacccttctaatttgcagtttctaagctgctatgcccttggtgtctagcaaataaagtttgcttaaattgaaggtcaatttagagtaaataggcatagagaaagagctttaaatatttacatctgagagcttgaactttatcgtcatattgtccacatgtggtactcagcaaatgttaaaatgaatatttaaaaaatcttttcagattctgagcttttgataggttaggtttaattttagttgttaataaactaaagatttcttgattgtcctatttctaaaaactaaatttgtatgttacatgctaattggtaaaaccaaaactaagctgtattacttggagtttctagttgatgattttgttcaacatgcttctctacatttgttttcttacgtgattaaaagttatccttatttgcctttacttctcccgatacccttctcaaatatcctgctaaagactaattttaagatgtttccattgtcttttctcttttctttttcttttttttttttttttgacacagtctcactctgtcacccaggctggagtgcagtggtacgaacttggctcactgcagcctcagcctcccaggttccagcaatcctcccacctcagcctcatgagtcgctgggaccacagatgcatgccacttggcctggctaattttagaaaatcttattgtagagacagggtctgtctgttgctcaggctggtctcaaactcctgggctcaagcgatcctcctgcctcagcctccaaaagtgttgggattataagcatgagccactgtgtctggacaaattttcctttttcttatggtgtcagggagggtagatccaaaaaggggcacgcattgtcctgatcagtttctttctttgtttctttcttggattcattacaagttgtcaaattattttccattagttttttttagagctaaaatttggcataggagacagtagtatcttttaagtttgttaaagattagtaaatgtattctaagcagttttagataaaaagtaaaaaagttgttattatcaaaacattattttatttattttagtccttagagtcttcatatttcttgtgatacttgatctaaactgctggggttataaagtggttatcctccctagagaaggaaatggtgcagattccatttgctcacttaccattggtaactgaatattatttgtaaattattagcagaatcataatgagctcactgttaagtagttcccctaatcagagactcttttctggattaagctgtctgctaaattcctcaactgagagttgttttactttgttggtatttttgtttatatttaaccaccttttatattaagggagatttgtgggaagcattgatgccctgtgctctagtaagcatgatcagtagaggcagtgctgacagtactagtcacatgtggttataatgctgagtaaaatgttatttactttactaagttggtttgcctaacggaagtagtctccctggagatgggatatcaatgcagaactgactcagcaataatctctccctagtcataaaacagccttactttttgttactgtcactaacatgaactaacatgctttggattggttgtatatcagaagctatgatttttgtcgccctattatatacttagttgtttttaccttatgtcatgttatcctggaaaggccgagacttaggtctctaacacagctagtgggagcttgaatttgattttaggtcctgctaaatctaaggcttatgttgtttgtgttataccacagtctgccaataatgctggccaaataagaaacattttgatgaatcgtgtatttgttagtatattacataagatattttccccataagaaaatgaagcataacctcttaggattttaggcttctctagtgtacagtatctatagagcaaaatttaaattgtggtagtgttttatagactaaaaaagaatcagaagcccctccaaatgaataaggactgtgttcccggaccttacccagtactgtacagattagctatccttagtttttgtagatgagctgtagagtcaatggcttaggaacattgaaatgtccatagtgtttcttaggatataatatggaagttggagtcatggatagaactttcactctggcagatactgtacataggaaagtgacatttgtggcatgacccagaagaactgaaagaaagaagtattttttaaaagagagaaaagctaaagatgctagataatactggtaacatttttaaaacctctctgtcttttgaatatctcctatgagccattttatgtatattatttctatcatcacatccaacccataaggtagatattatcgtcattgtaccaaggggctaatttaggttcagaaatatgaagaaagacattcaaggttataggattcttaaatggtaatgctggaattcatcctttctgctcctgcctcccaaatgtttgattgttttattgcctatccgcttttcattctggtgccctaagcaaagcataggcagtatctgtacgtgagatttgggcttaagaataagatttctgctaggctcggcgtgccctcctacgtgataccagaagttagaattgtgtggataaaacttaggaactggaaatgtaaaattaaatacttactttttttttaattagaaaaagaaatagccctaaaattgtcttttttggagagtaggattgcagggactgtatcacacatgactcttaatttggttctgtgtcttcttttttcttctttttaaaaatggaccactatattaatgtacagagaaaaatacaagaacttattaaactccatcccttgaaattaggcatctgattacaaaatacattcttaactgaccatataattaaaagtagtattcagagaagcagtagaacttactaatatagaaccatttgttacaataataccagaacatttctgagcggtatacaggcctacacaagggtcatgggtttaatagttgataggcatttaaagtacagataccgaaacattgttggtaggcccatgaatatacttgagattcttttggtcagagtattgtttcattacaattaaaatagaataggctagaaattttcttttgagtatgcatgtgctacctctgtcaacttttcatgttggagtgatgctgagtttgtataataatttgagatatattccatctttttatgttctccaataattcaaaatatggatatgatgtatcctctgaagttttaaagaacttaatgacactattgtacttggacttagaataattaatatactgttctaaaattgactcaaaaagaggtagagaagctgaatagaccaaataataatagaagaaattgaagtccaagttgttattaatctctcagatatatcggtttctacatctataaaattaagataattattactcgtagggttgttgggagaagtaaaatgcttatgtcagagcctggtacattgttagcccccaataatgtaaattgtcatcacagccattactgtcaagattattactcttttttcaagattcagcttttttccagagttttatttctttgattactcacttcaaagaatcttagatttactgttaaaaaatgtaattgccagaggtttttctcttttgctagtctgattccatctctgacatagtttacttaaaaaaaaatacactttataagaaataaaaaggaatgtagacacagacatgcaagtgtttttttttattataagagaagattatgtacaggttttgcataaaagaaaacatgtggaaagatactcaccagaatattaacagtgattatctctaggtggtgggacttcaagtgagggttagattttttaaaatatttctggagtattttaattattctgtgtgtatattatttttctaagaaagttttctattttataacttcatgctctagtgatcatccatttacttaatgcacttaagatgataatcttagagtattgatcttcaaactgttccatagaatgactgttagatttggtaaacataatcttaaatgttttatgtagtacatcaggggttggcaaactatggcgtatgggcaaaatctggcccaccactggttgttgtatagcccaccatgaactaagaatggattttatgtttttaaatggttaatgaaaatcagaagaataatattttgtgacaagggaaaattatatgaaatttaagtatcactgaccataaataaggttatattggcatgcagctatgcttatttacttgcatgttgcctatggctgcattcaggctacagtggcagagttcagtagttgccatagagactgtacggcccacaaaacctaaaacatttactgcctggccttttggagaaaatgtttgctgaaccctgtaccacattagagaaacctatttagttcagtgttttgttcttttttttttttttcggcgagtattaccacctgagctctacctcctgtctagtcagcggcagcattagatgctcataggattcagccctgttgtgaactgcgcacaggggatctaggttgcgtgttccttatgagactcttaactaatgcctgatgatataaggtggaacagtttcatcctaaaaccttccatgaaaccagtccctggtgccaaaaaggttggggactgctgttttagaagtctctgagtgcctcttctaattctctacccttccccataataggcaaccattatcctaactttgctgacatccccttgcttttgtatctttattatatatgtatgtatacctaaacaatatatttgggggtttgcttgtttaaaaggtaaattgtttcataatggatatattcatctcttataatttctttctctaagcattatgttttgagtgttacattttatattgatacaagtagctatagttgatcatttctgctatatagtgctccactgtatgaatgtatactaatggactaattatcatttacttacatatctccatgacagccacaacagcaacattttaaaaattcaatttgttgactgtcttcaaacccctacctgacaaaactgttttatttcgtgttttctctctgaaacagcatctctattcatgcatttgcctaagcaagactcctagtgtcattttcaataccactctctcctcatttcccattttcaattgatcttcaatcctatagatttgacatctgaacatctcatagccatctctcctatcaccatcctagtcaggtgcttgcttggactcctgcagtagtcttcagtgccatatcagcatccacccttgctctcttatcatggattatgaggcttacctctccaacttcatcttgacacacttcccttcactcactgggttgtgttaacttcagccattgttcatgtcatgctcatttctgcttcaggacttttgcaaatgctgttccctctgcctatagtacattgtcctgattcatttcttcttatccttcaggataaaattttgaaacatcacctcctcagaaaagtcttccttgctccccaaccccgaaactaggtcacgtttccctgttatctttaggactttgttacttgctactcaatcctggtactactacaaattttaattatgattgttaaggaaggcttatctgattcagtcagaaccagttgtctgtaaagtctaataaagcaatacatcatagaagggatacataccttaagtagtttattctaattttaagaatataactatgttcatttgccagtcttttgattaagaagctgttatcagaaaggggttcgatccagaccccaagagagggttcttggaccttgcgcaacaaagaattcagggtgagtcaatacagaaaagagaaagcaagtttattaagaaagcacaggaataaaataatggttactccataggcagagcagcagcatggactgcttgactgagtatacttatggtatttcttgattatatgctaaacaaggggtgggttattcatgagttttccgggaaaggggtgggcaattcctggaactgaaggttcctccccttttcagaccatgtaaggtaacttcctgacattgctgtggcatttataagctgtcatggtgctggtgggagtgtcttttaacatcctaatgcattataattaatgtataatgagcagtgaggacgaccagaggtcacttcggttgtcatctggttttggtgggttttaactggcttctttaacacaatctgtgttttatcaagggtctttgtgacctgtatcttgtgccaacctcctgtctcatcctgtgacttagaatgcctaaccccctaggaatgcagcccagtaggtctcagccttatttttcccagcccctattcaagatggggttgctctggttcaaacacctctgaaagagccaaggccttttctttcagagtaggtctactagttatagatccatattgtctgtattggggaagccatgcccataatgtatcatgtaagattccagtttgggttttttaagtagaacaagaatttaggctcttcagatgaattcttagcttaaaatccttatagatctttacaatattttttccatttctttgaactattttcttggcctctacctaattcagcagcagaagtatttcacttttattgagtatttcagagtcattcaactttataaaactccccactcttccatacttcattaggttagttgacatttaattaaatcacatagttacactaatataaatggcatgaacaggtttggaacctaacataactaaaagtgtaactcactgctgggaactggaagtgtctgggtatactagattagaggactgcacctctttttgagtgtacatggtctactgtattaatatatcatgtacactgtaatagtgtaactctaaggttgaattatatgtttattttttacccacaaaatagcaattttatatggttcaacctaatattaaataacagtataaaggataaatggccatccaattagataataatagatcctgtcacaatatgacgtgtaaataacaattaaaaataaatcatttttaaaaggccagaggattcttagattattttaagaagctgtagttatgacagccacacattcctgaaattctgggtcagttttactgtcaaacgtattatctttttctaaaaattattgaaatgccaccaaaatatcaatcaaaacttgtctcctaaactgcctcttgtgcctgaatttgatacagaaatacttttaatcagtctacctgtcccaatttttgttactgtaactgaaggactttctattggaaaggtgttacagctgagaagtctagatagactgaagaagaggaaggatttgagttgtcacattcttcgtctttatggcattcagcattaagaatataactgttatgttataactgtatattttaacaatataacataattaggtaagtataatagttctctctcaatgtattcttaaaacatgttaaacttaatttatttctcataaagcatgggttaagtcccattgaaataaactcctgggaattatttcaattattttgttttgtgtaaattttgtaatagatactagttggcattaagaaggaccattgactgtactatttcatttttatatatgtaaacttatgtagtgatagaatttgacctaattttccataattctaatttgtagcttatccagttaaatataatttttcaacaaaagctatttaacttccaaaagcattataaaatcagttttaaattattattccttcgaggaaagaataactttttagaaatttgctttggatagttttaagaacagctaagcatgtgtagaaatgtaagagattgtaatggatgataaacttttattctagaatcaatgcaattgaaatttaacgctttttttagccctgtataatttcatataaaaccataaagtagtagagttgtaatttagatctcaaagggtttcagctctatcttaaaaacatttctaaaaatttaacaaaacaaacaaaagaacttcgaatctgacattgttaataccaagatagttgaaatgccttcctgattgttatgtcgactagtctgtgaattactgtccgtattttttccttcctaatattaaagctgcccacatcacatttcacatgtcaagaaaaatcaactatctttctccatggagtaagatgcagtctgataacatgctccagaataagaatagagaaataactaaatttcatgctctgtgaaactgaaccagggggttcagtgttagaaactgcattttgtgccctggcaagtacaaatgttttctgcatatcacactaattcatacaacttataaagaactatataaggagatgagtggttttaggttaggtcactttttaaaagttattttttgggtataatttgcatgtcagagaagtatctttagatagctacaaaatttataaacacggagaagagagttaaattcttggacttcttgaatatggctcttctagtgtctagagtgatgtggtttatcttaaataggtggattagggtcagtaaactttttctgaaaggggctagatagaaaatattttaggctttgcagactacatgtggtctctgtcagatgttcttcgtgtgtgtatgtttaaatgtaaaaatttttctgagctgggatcccttcaaaaaacaggccacaggccagatttggccttcaggctttggtttgctggcccttgctctacatcagtaatgttcaatacaaatataacctgaatcacatatgttacttttaaattttctagtagccacatggtgggtggtgctgttttttgttaagagtcttgtagtgacatttgaccttgaaactacaaatatatgaattaggtaaaaataaaaactaattttaaaataaatgagaagaaactaggctagaaaggggctaggatattggaagagaacatctacaaagataatggagtcaccaagaatacggcaggaattcgactgtattaacaggactatgggcaaggaacggaaatcttaatttaagaagaagaataaatgataacaaggcacgtgaagttcaaatggcgtaaacctcataaaaaatgcttacgagagagatttttgcaagacagaggaggccgtgttctaaaatagactttactgagatttaacagttctgtctaatacattttattctaatatataccaactactgtacaaacttcctatgtgcctgacagtgtgcttttaataaagaattaaatcatctgaaagtacattttataatatcatggacattaattttctgtgttgtgtttccattagtccaagagccttttctgtgagaaactaatgggaagacctataattcattctaagaaagtttattctctgttattctcttattcctagttaacttcttgaaaatataagcgtttatgacaaaaataattagttgaagttatggcaagatcagctaaagttttctgctatcataatgtgacaaaaccaattgaattttaagtttaggtttttccatttcttctaatgttgacttttagctactgaactagacagtgtgcctgttgatactgtagtcttaaaagaatttatttgtaaagtatcatacactgctttagatttttttttaaggaacaagaataaagatgccaaattgtatgtttttggtttttgtttttgcgagtactacagtatcatagcctttctctattactttattacagtttcccaactcttccaggattaaagaattgggaagtttaaagacacctttcctggccgggcatggtggctcacacctgtaatcccagcactttgggaggccgaggccagtggatcacctgaggtcaggagtttgaaaccaacctgaccaacatggtgaaaccccgtctctactaaaaatacaaaaattagccgggcgtggtggtacacgcctgtaatcccagctactcagaggctgaggcaggaggatcatttgaacctgggatgtggaggttgcagtgagccaagatctcaccattgcactccagcatgaacaacagagcgagactccgtctcaaaaaaaaagaaaaaggaaaaaagacacctttcctaaatattacattctttttctttcttttctactcaaactgcttcttcaaaatcatggataaatttgatacattatagggtgggagggaagatatgggttttttataagtgatgaacacactgtaaaccttagaattgcaaggaaccagttaagtaaaattgggaagattgctactaatgaaaggtttttgaactaattttaatgtgttacacaaatgttctagttacaacttttgaaaagttgaatacaaatgtgtttcaacaatatggaacatattttgcctttgtaatattcaaattaaggaaacaggttgaagcacgggaaaaagaaaccagagtttaagagcgtttattttctttcacctgagttcagaatattctgcctgaaaggacttgggaagtaatgactggcttgaacaaggtcagtgcctaaaaggcccggtggccttcctgctgcccaacagaacacacggcctcatcacttcagcagggcagcgtaggtgaacgcttggccagtatcactgaaattctggacgggaaggggtgtgggcactgccttagctaccttcacatgctactttgattaatttatttttctctttttgctgttttgtctttagattttataatcaatggataaagtgggaaaaatgtggaataacttcaaatacaggtgtcagaatctcttcggtcatgagggaggaagccgtagtgaaaatgtggacatgaactccaacagatgtttgtctgtcaaagagaaaaacatcagcataggagactcaactcctcagcaacaaagcagtcccttaagagaaaatattgccttacaactgggattaagcccttcgaagaattcttcaaggagaaatcaaaattgtgccacagaaatccctcaaattgttgaaataagcatcgaaaaggataatgattcttgtgttaccccaggaacaagacttgcacgaagagattcctactctcgacatgctccatggggtgggaagaaaaaacattcctgttctacaaagacccagagttcattggatgctgataaaaagtttggtagaactcgaagtggacttcaaaggagagagaggcgctacggcgtaagttctgtacacgacatggacagtgtttccagcagaactgtaggaagtcgctctctaagacagaggttgcaggatactgtgggcttgtgttttcccatgagaacttacagcaagcagtcaaagcctctcttttccaataaaagaaaaatccatctctctgaattaatgcttgagaaatgcccttttcctgctggctcagatttagcccaaaaatggcatttgattaaacagcatacagctcctgtgagcccacattcaacattttttgatacatttgatccatctttggtttctacagaagatgaagaagataggcttagagagagaaggcggcttagtattgaagaaggggttgatccccctcccaatgcacaaatacatacatttgaagctactgcacaggttaatccattatataaactgggaccaaaattagctcctggaatgactgaaataagtggggacagttctgcaattccacaagctaattgtgactcggaagaggatacaaccaccctgtgtttgcagtcacggaggcagaagcagcgtcagatatctggagacagccatacccatgttagcagacagggagcttggaaagtccacacacagattgattacatacactgcctcgtgcctgatttgcttcaaattacagggaatccctgttactggggagtgatggaccgttatgaagcagaagcccttctcgaagggaaacctgaaggcacgtttttgctcagggactctgcgcaagaggactacctcttctctgtgagcttccgccgctacaacagatccctgcatgcccgaattgagcagtggaatcacaactttagtttcgacgcccatgacccgtgtgtatttcactcctccactgtaacgggacttttagaacattataaagatcccagttcgtgcatgttttttgaaccattgcttactatatcactaaataggactttcccttttagcctgcagtatatctgtcgcgcggtaatctgcaggtgcactacgtatgatggaattgatgggctccctctaccctcaatgttacaggattttttaaaagagtatcattataaacaaaaagttagagttcgctggttggaacgagaaccagtcaaggcaaagtaaactctccggtccccaaaggttgttaactaggtccgctttcatgtgcatcagacagtacacctatagcaagcacacgtagcagtgttaggctttttcatacagtatgtaagcttagtgttagtatctgtcagatgctacctgctgttacttattcagataaacatggtgcctattggaacaatagcggatagagctacaggtgttcagtaagactacaaaaacattttgcctatttcgctaacagtttggtttttaatggctgtggtatttgagtgaggcaactctggggcatttgttatgaagaattctatttcttactgaagaacaaattattaatattggatgagtatttcaacagtgtgactaatgtttgaaattattttttctaagagtttttctataaccttccaaaagtcgtgatgtttgtagttactataaatcaagctttggaagtccaaaaagaataaaagactgccttccttttagaaaaaaatgcaattttctggccacaagggcatagtgcagttcacttacgtgttgatgtagtttataatcagacgccttttctcttctgcaaaaggtactgttaagtaaaccagattttctaaataggcattcttaaaatttcagacttacaaagctagtagtagaattttattgaaaggcctaggtattaattttttaaatgagtgctttaacttaaaacaggcgtttggaatagctgctgcaatgtagtcttgtgtgtgatttttttttaagttgatgtgcagtctaattgttgtttcataaaagttggatctgttcctatgcccaggatgattttgtgaaccgtgaagtacgtgagactagaagacgcccaaacaagtcagataatagtaactacaatggttgctgatgttgagattattgttgaactataattaataatttggatggcagaatttatctcttttttgtaaactctcataactgaattgcttaagtataatttatagaatttcagtgcagttcattcttaatggaaaatctgaaacctaaattgcagatttaaaaggtactgtacaaccattatatctgtaaataacttagcacctttttgtcacttagaataatatgtactactacttgagtgagcgcttttggaagttatatcaagttctagtgtttgcttcttagtaactgaactgaatttacagttctgtcctagacattttgcactaaagtagccgaatccactctcatgtcttttcgttaatgtgctctgtaccactggtgagtgctccatagtttccttacctgctgctacagaatgttattttacatccctatggctattgccaaggctacaaaaaaggaaagctatatttgtatgcaacactaaccttttgactgctaatgtatgtttctgcttgctgtgccttgttatggctgctttttttgtgctaataaagtatgtttggtgttctccttgtatatctgctgttttatacatttgcaacaatttctcttgtaaatggaatggtttggggtttttaaataagcattaactaacaacctttctatagttaatgcagagttaatgaacagtctaatattgacttatcagaataagctaactctaaatttaatgctctacatcttatcagtcataattatatatactgtggaacagtatctgtagttactgcaaattactgtacagtttaggttataacagaaaactgacagagaagtaataaacctattgatttctctgcttataaatgaaagattgaaactatccaatgacatattatagtaaatgagtatctgtaacctcccactgcatcagaagcaggttaaatgaagtcttgtgaatttgtaatagatcagtaccatttattggtttggggaccatcttaattaaaaataaatgcccaaaatgtagaactttaaccaaagacttgtcccttttaaagcaaaatggggattgaagggacttataatttctgttgtttctaattaaagtccctgaagatcatataccaaagtgtttgagaacttcatccaaacctactttaaagcattatgtgcaattaagttgttatgacataattatattgcctaattgttgggtcttttttcttgagcttataatgtacctggaaaataaacctcttgagaaaaagaaaagttcatactgattattggaaaaggactatatatgtgagcaagattgtgttttagagaggaaacttgaaactccaagaaagcacttgatgtttttatatgcttgtagcaaattgatgttctaactgtagttttatagaaagtattaatgcttttatgtatttcaaaactttcatatgttaaatggaaattgttttaaatgtgtttgagtttatgtaagcatgtatacactgtgctaaaagtcacatgtttcagtttgtgtataatattaatatgcaatttttggtttaaatttttgtcttaaaatattagtggcttacattttaaaaaagaaaaatcaccagcatgaacttgcacctaagtctatattcactgtgtccttttctgaatcccattgtagcctgtcaactaaatttgagtgttaacggtctttttaaagtgcatttaaatacaaaccaggaatttctttagaagttga

SEQ ID NO: 20 - protein, 535 aa, Homo sapiens
MKKISLKTLRKSFNLNKSKEETDFMVVQQPSLASDFGKDDSLFGSCYGKDMASCDINGEDEKGGKNRSKSESLMGTLKRRLSAKQKSKGKAGTPSGSSADEDTFSSSSAPIVFKDVRAQRPIRSTSLRSHHYSPAPWPLRPTNSEETCIKMEVRVKALVHSSSPSPALNGVRKDFHDLQSETTCQEQANSLKSSASHNGDLHLHLDEHVPVVIGLMPQDYIQYTVPLDEGMYPLEGSRSYCLDSSSPMEVSAVPPQVGGRAFPEDESQVDQDLVVAPEIFVDQSVNGLLIGTTGVMLQSPRAGHDDVPPLSPLLPPMQNNQIQRNFSGLTGTEAHVAESMRCHLNFDPNSAPGVARVYDSVQSSGPMVVTSLTEELKKLAKQGWYWGPITRWEAEGKLANVPDGSFLVRDSSDDRYLLSLSFRSHGKTLHTRIEHSNGRFSFYEQPDVEGHTSIVDLIEHSIRDSENGAFCYSRSRLPGSATYPVRLTNPVSRFMQVRSLQYLCRFVIRQYTRIDLIQKLPLPNKMKDYLQEKHY

SEQ ID NO: 21 - DNA, 41298 bp, Homo sapiens
agtagtggacggggggcggcccgctcggctcctccgcacccgctccccgctcgggccgaggcgccgcggccgcgcgcccctcctggaggaggaggaggaaggaggcgggaggaggagttgccgagaggagaaggcgagcgcggcagtcgcgccggcgctgggcgaggaagcggagccgggccgcctccgggtaagcgtccggggagctcgggcgggaggcaggggcagggcttcgccgtccgcctccgggtcgcccgcggtccgggacacggctggttttcgcctcggagtgcagggggcggatccggcgcgggccgggccagggaggccgccgggcgcggggctgcagcgacgggtctgggtcagcgcgaggccctgcgccgtctcggcgggtgcgcgggcgggggccagggccggggaacgggggccggggccagtcccgggagcggggacgcggaaaccgggaagccggggccggggcgcggcggcccctgaagggccgaggcctgcgcggccgcggacggctcttctcgacccctcggcctcttcggccgcggcgcgccggcctgggccgccgcctttgttgcccgcagctcgccggggagaggccggggctccgcgaggcctgggcagcggcgcggggcctgcgcggctctgcgccccggccgggtccctcctcggacctccgcgggctcgggctcgggctcggggtcggggcgcggcgaggccgcaggcgcgagggcgggcctggggcgcggcgctgggactcggggacgcccccgccccaccgcgaggtcgcgccggcgctggtggaggtgcaggccggggctgctctgcggctgaaggtgccgccggccggctgcgcactcacccgggtgtagggaccggacgattgtggaatttaaaccgctcctgggaccgacacgggcctgaattttcagtttgcttcgggagttggcaacttcaggtacacccttgtggctcacggagcagaattatggacgtttctcaggggcagatcctggcacaaaacttgggatatgtgcagtagtgtgtgtgccaacttaacgttcagttggaatgtgaaattggggttcccatccccactgtttggaatgaagggccaggtgggaatatgtggcgggactcagtcttccggtagcaaaataaataatgttgaattcatttcaacaaatgtttctgcaacgtctgcccggataaggcagcatgcactgtgcatgaggacagagaacacagtaatgacagaaataacgtgaattacacactgaacgtctcccagagacattttaaatttactgtgttccttgggataaagatacatttggaatcaaacaaaagtaaacgttctacttcaagataaaatatctgacatcttctcaaccactgaaggtagcaatttatcttgacttgtttttaaccaactaccacaacattaaatccatttagcttacttgaatgaacgaatcttaattgtaatatttaatgctgtggacagccttacttaaaaacacgaatggcattgctcaccaggtttcacaaaaaccggaaaaaaagtctgtaatacttttccgtgggctttctaatttctcctcctagtgggttttgtgcacgcgtggatatggtaaggtctaggatccattcagctctaaattaaatatgtggccttaaccctttggttcaaacaccctccctaagcctgccttggtgccttggaggaacctggttcgtggacagtggatttgataaggattcctagaagaacttctatctggagtttggccagggctttccgcttgtgcctgggtaagggtcagccaggtattcacccgaatctatacttttttagaactccattcagcatctcttttgaaacactgtttctctcttacctttatcgttatcttctaaattaccctctaaattggtagtcttgcccctttctcttcagcagggcctcccacccaagcgcacaccctcgggtcatcccatgaccccctctcttggtgggccccccaccccgcagggagtggcagggaaggaatttgagagccattgactgggcgtagggtggaaaatggtgctgtactgactgggtttctagtcattttgccttccagtgtttttcttcattcttgcattagaactggaaaaggtgttcttttaatggcccccctttttttaggaaacagaaagcaagtatcttcttttttgtttttcctctgtcacccagggtggagtgcagtggtgcaatcatggctcactgcagccttgaactcctgggctcaagtgatccacctccctcagcctcctgagtagcttggaccacagacatgtgccaccacacctggctgattgttttttggagaggtggggtcttgctgtgttgccgaggctggtctcaaactctaggcctcaagatcctctcacctcagcctcccaaagccctgggattacaggcacgaaccaccacacccagcctgcaagtgtcctcttaagccttcttgtgtaactacactccttagagaaaaatcttgctagttttctccattatggccagttttcaggaaacttttgggttattgatcatatcttttattttgctcatagaggtgtgatcttatttaaaagtccaaacatatagatggtccccaattttggactttttgactttttgatggagcaaaaacgatacacatttagtatgcacttcgacttacatccagataaacccactgtgagttttgactttaagatattttcaatttatcatgggcttattgggactcaattattgttattctcttaaaaatatgcactcgtgacctcttccaattctggaaattatatttacatctgtggtgccgaagtacttttgtaaaattattcattttacaagggagaaattccaaatactagtatatgtcataaaaaggagaaactttcttatatggctagattgatagaaataattgttcagctgttaaaagaggaacaacgtaagatgtgtttgatattgttttgaagtgaaattgtcattaatgtttttatgggtaatttattatgctgtttggcgtgttggtttagctaataagtcgtgatggctttagggctgaggacacagtttacttaaactgatttcaggtctttacccacccatacaaaccttacattttacttcgttggggcagttgtttttaggtggccgttggtcttgatgtttcgagaacagacatacttagataaagtcacgtaagatttcagttctcaccttgcacactctggacatgttggggcatccatttacccgctctgtcacgttgaacacgttctgtatgccctctgagtcttatctggggctaaagtggggacaataatttatctcacagttataatgaatatgtatttatggtaacatccaaactcttgttttgttttgaaatggggatctcgctctggttgcccaggctggagtgcagtggcacggttaggactcactgcagcctcgacctcccaggctcaagcgatcttcttgcctcagcaccccctccgccagttgttgggaccacaagtgcatgccaccatgcccagctgattttttttaatttttggtagagatggggtttccttatgttgcccagttggtcttgaaatcacgggctcaagtgatcctcccacctttgcctcccaaagtgttgggattgcaggcacgagccactgtgcccagccaaaaatccaaactcttcctcatcgactataacatttctgcctagttctgaaatgtgttttcatttctccctaattctcatcttgagcaaccatctctgccttaccagggttctgccctagcctttccgttcctcaacaatctaagctgcttcctgctccgccacctttgacacagtagttctgtttgcctacagtgccttcggtggctcctgtggttggttcattctcagatcttaatgccagtggcaccacctcagagcatcaccacctcagagaagacttccttgactaccttatggctccttcagtaatcccaagtaattgacttgtgtggttccttcataacctctattattgtctgtaattactttgtttacttgtttctgctgttcccccgccaccccccccaaataagttctatgagggcagagtgtctactgtggtccttcttttctgtgtccctgacacatcccagggggcctggccactgagtaggaggggtgtgggtatacagaaagtagttgcggttttcatgttgttatctacaccttaaaggcagagatgtgctgtgtacatgttagaaagttgttggtatttcatgtcgtttgtccttttctgagctggtctgcttgcctggctgagtaaatcatagcactaataaaaccaaagtccttatcattacatggccccaacagccccacagagagacaactgtcactgcctgtagagctatgatgtgtgtctgagtgactcaagtaccagactgactggcagtgtagacgcccagatctctttcacttctggaagtatagtcatgtatagtgtatagaaagtcaccatcccaaaacccatttctcctgcttacatagcttagtgaagtgaaacctcagttatctgaaatctcctggtatcctcagttttctggtgtagaattaagccagaaggaaaatctcctttggaattagaaaagccagagacttagaagccctccatgctaactaaggaagacagtctctcatggggatgcagatctaggtttgaatttcagcacttcaattttatagggtagattaaaacaaaaaaaaaaacttaaggttttgttcctttacgtatataataatgatgatgtttctctcagagctggtgtgataatggaatgagatagccgggcgcggtggctcgcgcctgtaatcccaccactttgcggggctgaggcgggtggagcaggaggtcaggagttcgagaccagcctggccaacatagtgaaaccccgtctctactaaaaatacaaaaattagctgggcatggtggcacacacctgtagtcccagctactcgggaggctgagacaggagaattgcttgaacctgggaggtggaggttgtggtgagccgagattgtgccactgcactccagcctgggcaacagagcgagactccgtttaaaaaaaaaaaaaaagaaagaaaatggaatgagataatgcatgttagtgtattaagtgcctgaaatataacaagtgctaatactttaacactagtattctactcctttatctgactgcttttgactagttagattatgttttaaagtaatagtaataattcctatttaaagtaattattactattaattcttttctgtgtccctgacacatcccagggggcctggccactgagtaggagtccgtttttcttgtcagttttttttgttgttgttctttggaggggagttccctcaatttttcttttattggatgtttactttttggctgtgggagttttgttttctaactgttctcctcaccccctcttttataatatataataatggtaataattcctatttaaagtaattattactattaatttaaaacataatctgactagtcaaaagtagtattctgattaatcagccacacctgtgttagacttgaatgtttttgttaaggaagcctaatgtgggaaaagtgtttgagaccaaaagagcaagagaaatggaaatgaaagcagtaagtgaatgggcgccttcagaaggattgctagtgcaagctgctagactggtaaggattacaggtggtcacagtgttagcagcaattgcctgacctggctgtggggtcattgcagttcattggtgggagaccgagaacaaaaaagatgccgcacttgccttctctgcagagccttctgctctgattatgtttgttctgatactgttttatcactcctaacagcgaacacactaaaatactttctaaaattgttaaaagggcatcacacttggagactggggagtgatgattatcttattttccatagcatcggactcttgagtatagagaagaaaagaaagaagacaggatatatctgaaggcctggaaggcaggcagtaatctgaacatcagtaagtcctaatggtgtcattatgccatcctgaaattttccagcagataaatttggtcatctggagaggcatccatttatatacagcagtttgtgacagtctgagattatacttaatatacacacttgtgcttaaaatacctttttgaagacaggaatgtatggctcctattagataagatttccatcaaaaacaatttcaatagttcagtgttaatctttagacaaggtatttttacccagcgctctgggtagctgattatttggtgctgtttatggtggcacttacagtgatttttgtggtacacattgttctgaccatatcaataatatgctagatggttgaagcagctgggttagcatgctttgaaagtcagatgaattcttactggtagatttttactgtatctgtatgttacttgtttaaagtttgttgtgatgtgtataataatggtgtttgtttttaaagcttaattttggttttcagtaatgtattaccatccctcagacaatagtcatttttgtttgttgacattgcttttaaagttaaaaaagaaaagtgacatatttaggaaataacattattgccaaatatcacagactggcagaaaatgtaagcaaataccagtgcagagaatttcagtaggattttagtgattttatatatttgcagcagaattactgcattttagagctagaaggaactgctctgattatagattatcttcgtccttgcacagatgaggaaattgggtctaagcagggtgtcatctgcccagagtcacacaccacattagtagtcgatttcagagtggactctggtgtatgcagcccttcttgctgttgatgcttaaggttttaatttaatcctgggaactggggagtttggattcgtgtcttcctggaggatgttacagttgagctggctcttgcagggtgacgtgtgtttgattggagaaagatacagaaggcaggaacagtatctaaaacaggacgaatcacaggagcctctaccgtcaacttagaaattcttttctccttcccaagtggggttccttgttttcagttccttagtttaccgccttgatatgatgaagctcatcctcatagtgacttcctgtgaaaggtgatggggaagatgactttttttttgagccttgtgtgtcagaaaatgtctatgttctaagtatttggtgtatgcctggctgtgtataaaatttcaggttaatattttagttcagatcaaataagttatttggcagtgacaaattaactctgaaatctcagtgcatgaatagaggtctgtgtcttgctttcacaaagtctgctgtggacgccgcctcccttggcctcttcctttgggtccaatctgctttcacctacagttcctgtctgttgtccccatggcggatgtgggtgtatctgcaggtcgcacgctggtcaccataggccagaaccaggcctagtccctgcaagaattccagacgcatgcaagggcacaaggagagtcattgaactgtcagtgggggtcatagttgtaagtgtgtttcctcttggaatattgcaggcattgctccgttgtcgtccggttttcagtgttactgttaagtcacatgccgccagtttcccaggcagttgtaagccatttcttttccccctggaagcttttgggggacttttgtgttattgcctaaatactaacattttaccatggtgggtctggtgattgttcttttctactttggatgtgctgagcattccacagagcttttcagtctataaacttatgtttttcagttctagaaaaactattaaaatgacttttataatttcttccctttcattttctcattttctttcttttttttttttctgtcttaagctcttcttcattggacataggaactcctaaatccgtctacttattttcttagcttttttctccatttttcttgtctgttgttgttgttgttgttgttgttctttggaggggagttccctcaatttttcttttattgaatgtttactttttggctgtgggagttttgttttctaactgttctcctcaccccctcttttttaaacaaatagcatccgcctctcagttcagatttgtgaaaccttttctcaggctggtttttcgtcgtcttcctttccagcagtcttagattttggtttactccagtttgttttagccacttaacactgttgtcatctgccgtaattcgctttgtgattttcttcgttcctgtgtttttggggaatgatggagataaagaagtgtatgtttaatcaggagggtttttttttcccttcttttcaacagtttttgtttgttttgctagcatcagtgaatattaaacccttcgtttttgggaaaaccagatctatttttttctttaaataaaagtagtgttttcagttagtgctctcatgaagccacatctgtgtcactgagttcaattaacttagtcgatgaaatgcctgctgatctgtagtgggtggtaacataattattttaatttggactattttaaaagcatcaagagatcgcatctttgggaacccacaataaatggttacttttggcaatcagattattgttttataatacaagtaatcttttcattttgtaagatttttatattgtttgcttaatagggatatattattaaattgaataattattgctttccaagattcccttcagcgctaatgatcaatgaatgaagtttatgttggctcttaatgtacatttaataacaaatttcatgtggattactttttgttccccaaatctgctatttaaaactccattcttcatttggatgtactttagaaatggggaaaagattattttaactgaaaactcaaattctctactgcaaattgaatgacaaaatcttgtatgttctatgtgaaagcatttgaataatgggcaaattgctctttaaattagagcttttgtgttgatctgaaggctctgagagcagtcataacagttgtgttatttttcagttagttttacatgcatgttgcctttcacaagttagatatttttgctttagtagttcattacatttaagatattttaatatataaacaaattacttttaatcttaaagcaatgaagaaactattaaaacactttgctacagtacagaataaaaacaaaacatctttgttcataatttgtcaaattctgaaagaatcttacctacttggtagtttataattcatgaggtcattctttttcatagacaattaatgtggtgagatttttagggaatttgctatatttttagttaaaataattttgtttacattaaaatgttaaataattgttttttaatttgtcattgtcagctataatcatatataggtgaatggtattaaaagaactatatataagatagtattttacatttatcattctggaggtcaggatgtattatatatatatcagaaagtattacaatagctgtttctaagccaccaactgtaattataccttagccaataaggttgcagatgtggacatcattaaggatcttactagtagtaagttggggtgctgtcttttcagtgtgcaaatgatttccattatcaatgttgcccaggtctccactgatttagagttaagatacctatattaatttgttgaaaagtgtcatagttatttcataggagtctcaggcctgatacacacagtggtaaagataaaaggctgatttatttcttgtgatgtgtttccaacaccaacaaccacttcttcagtgtgaaaactgagtgtccaggctgggtgcggtggctcacacctgtaatcccaacactttgggaggccgaggtgggcggatcacttgaggtcaggagttcgagaccagcctgaccaacgtggcaaaaccccgtgtctactaaaaatacaaaaattagccaggcacggtggcacatgctgtaatcccagctacttgggaggctggaaggagaatcgcttgaacccgggaggtggagcttacagcgagccaagatcatgccattgcactccagcctgggcaacaagagcgaccctccgtcttaaaaacaaaaaaagaaaactgagtccaacatttcagttaattcagaccctacccagagtaagtacagtccccacaggcaacgggctgagtcccatgagactgccccgtcctcagatgccagtcacaagtcccaggcctctcatacttctgaccgactggctacaaatcaggggttcccactacctcctcagattagataatttgctggataaaactcaggaaaacattattattaagggcacaactcagcaacagcccagtagaagaggtgcacggagcaagcacggggggacgtggagtttctgtgccctcctagggtggcctcctgcccagctcacccttgtgtgtgcaaggtccccgaatcttgtagttagagtttctgtagaactcaatctctaatcctttccttttctcttcatttctcttcaggatagggacggggggtcggtgctgaaagttccacactctaggcactgggtctctgggtgaccagccccatccagaggccatctaggagggccgcttttaatcacagcgttagcattaacagttgtgattgaaaggggcttgttttgaacaataaaaaatatttctatctcaggaaatcccaaagatataggaactgtgccaggaactagagacaaagatgaaatatgtcttatatcacatttctttgaattggttaagtgcaaataagacaacaaaaaataatataaccatttatataacacttgtgttaggtgttataaataatctagagatgatttaaagtatggggaggatgtacataggttatatgcaaatactatgacgttttatataagggacttgagcattcatagcttctgttattcctggagccaatcccccatgaataccgagggatgaccgtatacgtataaagacagtgatgaattggcatatatatttactacttcttaaatgcaacttgttaaaatctgaactaaaaaattaaggagaggtgtgcagtcatttaatcaattctttatttttttttttccttaagacttagtctctctctcgcccaagctggagtgcagtggcatgatcttgactcactgcaacctctgtctcctgggttcaggtgattctcctgcctcagactcccaagtagctgggattacaggcacccaccaccacacctggttagtttttgtatttttttagtagagacaaagtttcaccacgtgggccaggctagtctcgaacacctgacctcaagtgatctgccagcctcagcctcccaaagtgctgggattacaggtgtgagccaccgcgcccagcctcattaatcaattcttataagccaacacaaaaagcatttttacatgattgacgttatgaatatttgtttcatattcagtttaataattgaataagaatgcattgaatacaattacagtgaaggatgcttagaaatgctcatgggaaagttgttcattgctggttagatatctaagtccgtacatatttttattttcattgagtcagaagcttttagaactggacatgatttagagattatcatgtattttatatggagattcacttagtatccccctgcaaatttaagctagtgctaccccatacttttgtgtcaatagcctataaagtaggagtgaatcacttgaaaatttattagaacttttgttgtaaatataaatttattcttaatttaagttaaaattatttgtcaactttaagttcatttttctcaatttggctatatttaacttttttaaatctacctttgtagccatgcagtgttcctgttgcatttcaattagaggttattgtggtaagagctaagggccctgaaagaagtcaaagcaactgtattccatttattatgcaaatgtaattctgtttttatgtagttgtaaatatttattatgtttgaatgcagttgccgagaagtgtgaaatgtctgagatttttgccttcaacacaagctcaaaggcaggctcaccagttagccagctaccagttggatgctggcagaagacataagactcttgggttagagataaaggaccttattacagtagtagccacagtgtcagcatctgtcccagtttgctgagacatgttgagaatagaatggggcaggggaaagaaagcaaggaaacttattaggaggtattgccataatgcaggagaaagaggatggtagcttggactagtatagtggcaaaaagtagttggattctgcatatgttttggaggtagctgaaattgggatttcatgacatactggatgaggaatgagagaaagaaaggagccaagggtgattctgaatgttctgatctgatcattggaagaatgtagagtagccggaaatggaagtatagagaagatttaaacctgcaagtggggctggggtgggggtggggtggggaacaacccgagtttagttttgaacttgtgaagtttcagatatctgcaagacatcaaagtggcagtgtcctgaagggagctgaatataactggtctgtaaatgaagagagagaatttgggagtgtagggcacatcgatgctatgcagtgtcatgagcatgagtgaaatcacaggtggagaagaggggaccaaagtcttgagtgctggggcactcctgttaagactccagagggagactagcaaaggagtcagactgagggaccgtgagagtgtggtgtgctgagcaaagaggtaaaatgatatgaaaggaaaaggagtggtcagtggtcacacacagtgcctctaagattaggactgtgaattcacgctaggcaaaggctgctctgtgcgatgattggtagagaaggctttggtgtgggtgtcagagaagattggagaggaatcgtgggcatcagtagactcagttctcctaaggcattttgctgcaaagggaatcagagagatggggctgcagctggtgaggaaggtggggtccccacaagtttgtttatatatgagaaataacattttcaggaatgagccagtggagtggggaaattgttgggagattaagtgtgccagctgacggtgggggcccttgagcaaggtagcagaatggagtagatggaaacatggtgctgagtgggtagatgggagccgagtccaaggtcttctgatgtcgttccttttcttggtgaaataggaagttgggccatctgctgcgatattaaatgagggagtgttccaggggtaatgagagtgttccaggggtggagaaaagagagggaaggtgtgaaatggtaggccgggaaggagaagaatggaccagggaaaagtagtttgatttccagacagcattaaggacccacctgcgggtcatggccttgactacaaagtgagacccatcagaccagttctctttttctccagccttatttagctctatgggtgcaggcatgacataagcggagttgaatttactaatatggttttaccaagagagtttttcaggagagtagtggaagggaatagaagtatacacaaggaaatgattacagtgatgaattgtggaatataagctgagtaggaagggaagagggtcttgaaaagctggtaggcttatcactcttgtcatttagactttgaatattcaaagccggttcctaactgtatatatatatacaccagtataaaaaaaaaatttaagtcagatttgcaaaaagttattttgagcaggtaaagaggaggaggctgtctaggttctaccaaagccaaagaataatagtaaaaacaaaaagctacaaaaaaacccaatagtctcatcttttgatttattttttgtttggcagtcatggtataactgaaagccaagaaaaggccctgcatttttaatggatttactcaaccatttcctttaaaaaaaagtaagaacaaacaagctcacaggttttattcttataaaaacactgcatgttcattataggatgttttgaaaataatgtttacaaaagttctgccatccagttaacagttctgctgtactttgtggtccagttttcctccctattctttctatgtaattgaaccacatacatataattttgtttcctgtgtttttgcacttaacaaacgaacaatatgaattttcccctcatgttatttataactctgtataaatatcaatttcactggttctctaaagttccattgtgtaaatgtgccaggattttctttactcatactgttggaaattaggatgtttccaatttttttaatttaaatttactttttgtcataaataatgatccagggaatatttttatgtttttttctttaatatttataaaaacagcattattgggtcaaggagtatcgttacttttcagttgcatattgccaaattaatttttatggctgtattaagatccttcagctgggtgcggtggctcacacctgtaatcccagcactttgggaggctgagtcaggtggatcatctgaggtcaggagtttgacaccagcctggctaacgtggtgaaacccatgtctattaaaaatacaaaaattagctgggagtggtggcatgtacttgtatttccagctacaggaggctgaggcaggagaatcgcttgaacccgggaagcagaggttgctgtgagctgagattgcactactgcactccagcctgggtgacagactgagactctcaaaaaactaataataataaattttttaaaaaagacactttcaggcaatcaatacaaccatttagaaaaatctttgctgatttgacttttggaaaatcatagtgttaatagtttaatttgtatatctctgattcttgtgagggttaatatttttccataaatgtgttgagtgaattggttgtatgtgtggaaaaatgtaaatctttacctacctcacacataaatcgtactagatttaatcaaggacttaaatatgaaaaccaaaacttctttacagcaaatacaggaggatatatgatctcaatgtcagatgaatttttttaagacaatagaaggggcagatagtaaaggtaaactgataaatttgactaaaataaaatggagaaacttctgatcctcaggtactagaaaaagaataaaaagtcaagatacaaggtcaatgcaacagtaaagaaaaagaaaagacacaggatgatctggatgaactgatacagaatgatttctaggggtctgaagtttacctgccgtgtgataaatttttttgttctctatatttagccacacacttcaatcatcgctttcctgcaatcggggaaaaaaaaaactttgcttaaaagccaacccatcttacattcccaccagcagtgtataaggtttctggtttctctgcatccctccctttgtcatgatattgtcatgatagtggttgtgcagtggtatctttttgtggctttaatttacattttgaatgactaatactattaagcaccttttcgtgtgcttattagccatttgtgtgttgtctttggtcaaaggtttgttcacatcttttgctcatttaaaaaattaggttttatttatatcttggtatcatctcttgtcagttacgatacacagatattctagcctgtggcttagaagatatgatacacagatattttcttctagcctgtggcttatctttcttttaatggtttgttttgaaacacaaacacttttaaattttagttattaaattttaatgatgtttaacttagcagagtttttctcttatatattgtgcttttggtattgtgtccaggaactctttgtccaaactagagtcacaaagtttttttaaagaagttttatagtttcatggccgagcgcggtggctcacgcctgtaatcccagcactttgggatgccgaggtgggcggatctgaaggtcaggagttcgagaccagtctggccaacatggtgaaacccagtctctactaagaaaatacaaaaatttgccgggcatggtggcatgcacctgtaatcccagctattggggaggccgacgcaggagaattgtttgaagccgggaggtgggaggttgcagtgagccgagatcgcgccactgcactccaacctgagcaacagagcaagacgccgtctcaaaaagaaaaaaaaaaaagttttatagttttagttcatacatttaggtatatgacccattttgacttaattcttacctatggtgtgaagtaagggtttacatttttgtttttagttattcatttgtcccagcaccatttgtcaagaagactttcctcattgactcgtcttgggacgtttcttgaaaatcagtcgactataaatgtaaggatttatttccagactcctttattctgtgatgtgtatgccagtctctgtgtcagcagcacactgtcttgattcctatagttttataagcactttcgaaattggatagtgtaactttgttctttttcaaaattgcatttcaatataaattttaggaatattgtatcaattaatataaaaaagagcttactaagcttttgatagggattgtttgaatctatagatcaattttggaagaattgccatattaataatattgaatcttccagttcaaaaacatggaatctctaattatttagaatctctatattctcttagcagtgttttgtagttttgactgtatgaatcttgtactacatgtgttaaatttattcctcagtcattttatattttgtgaacagaattgttttcttaattcacttttggtgtgctagtcgttttattgttgctaggtttgtaggcttgtagggtgggtgtgtgggggtgtgtgtgtgtgttgtgtgagttccttgggtttttctacgtataagattttgtcagctggccaggcgtgggggcttatgcctataatctcagcactttgggaggccaaggctggaggatcgcttgagcctaggagtttaagaccagcctgggaaacatagggagaccctgtctctattaaaagttagctaagtgtggtggcatgcatctgtagtccaagctccttggaaggctgaggcaagaagattggttgagctcaggaggtcgaggctgcagtgagctgtgatcatgccactgcaccccagcctggtgacagagtgacatcctgtcacaaaaaagcagccgaagtgggtggatcacgaggtcaggagatcaagaccatcctggccaacatggtgaaaccctgtctctactaagaatacaaaaaattagctgggtgtggtggcgcacgctactggggaggctactggggaggctactgggagctgaggcaggagaatctcttgaacctgggaggcggagattgcagtgagctgagatcgtgccattgcactccagcctggtgacagagactccatctcaaaaaaaaaaaaaaaaaaaaaagaaagaaagccaaaaactaagattgtgtcaattatgaataaagatggttttatttcttcctttccaatttgtatgcattttttttcttgttccactggctcaaaccttcagtacagtgttgagtagagggggtgagcgcaaatgtccttgtcttgcccccagtcttagtggcaaagcattcagcccttcagcattaagtatgatgttagctgtaggtttttcatagttgcccttaattagtttgagcaagtttccttctgttcttagcttataaaaagtttttatcaataggtgttgtcaatgcctttctgcaactattaagatgatcatgtgtgtttttttccatctttattagtatgctgtagtgcatcaattggttattgatgttaaaccaacctggataaatctcatttggtcaaggtatataatgctttttatatgttgctgaattcagtttgctgatgttttgttaagaagttttggccaagcatggtggcttacgcctgtaatcccaacactttgggaggtctaggcaggaggactctttgagcccaggagttctagacaagcttgggcaacatagggagaccccagctttacaaaaaataaaaaaattagccgggtgtgctggtgcgtgcctgtagtcccagctatttgggaggccatgatgggaggatcacttgagcctgaagatggaggttgcagcgagccatgattgcatcactgcactccagcctgggcaacagaatgagaccctgtctctaaaaataataaagaatttttctatcagtgttcatgatggatattgatttgtagttttcttgtgatgtctttgcctttgttatcagggtaatactggcctcacagaatggcttgtgaagtgttcctttcttctctgttagctgaaagagtttgtgaagatttgtattaattgtttaaatctttagtggaatttaccagtgaagccacccgagcctgacctttctttataggaaggtttttaattatcaattcattccttactcgttataggtctatttagattctctgtttccttttgagtcagatttggtaatttgtgtctttctaggattttgtccattttctcttaagttttctaatttgttggtaaaaggtgatttatagtattcccatatagtcctttaaatttccataggcttgatagtgatagcctttctttcatttctgattttgctaatttatgtcttctctcttaatttctttgtcagtctagcctaaagctttgtctgttttttatcttttcaaatgactcacttttggttttatttgttttttccctcttgtttttctgttttctattttattaatttctactctaacatttactattttattctactttcattgggtttagttattttctaatttcttaagggtgaaagcttaggttattgatttgagatataggcatttaaagctgtacattttttcctaagcactactttagccacatcccataaatttgtgtatgttttgtttttattctgttcaaaatatttactaacttttccttgtgattttttagtctttggcccattggttatttagaagtgttttgtttcatttagaaatattcggtgtttttccagatttatttctgttgtagatatatcattaatttacttccattgtggtcagagaacatacttggtataattatagtatcagtctttttaaatttattgaggcttttggagacagtcttgctctgtcacccagactggagtgcagtggcgtaatcatggacaagatgcagcctcagcactcctgggctcaagtgatcctcctgccccagcttcccatgtagctggggctacaggcatgcgccaccatacctggctaattttttttttaattttttgcaacaactaggtctcactatgctgtccaggctggttttgaattcctgggctcatgtgatcctcctgccttttcctcccaaaattctgggattacaggtgtgagtcaccacacctggctattgaggcttgttgtggtctatccggaaaaatgaagccttgaaaaaatattaattaggtcaagttagttgatagtgttgaagtattatataccttgctatttcatctttatttgttccatcatttatgaaattggctttttgaaatcttcaggtattattgttgaattgtctgtgtctcctttcagttctctcaggctttgctttatgtattttggggttttgttaagtgcatatgtgtttacaattgctgtgtcttcgttatgtattgacccttttatcattttgaaatgcccctttttgcccctggtaatactaaaagtctattttgtctgatattaatattgctattctagctctcatgattattatttacatggaaattttttcctgtcctttttaagtggtttgtgtctttctgtgctaggggagtctgttgtagccagcacatagttgtttcttttttaaaaaagatttagtctgacaatttctactttttgagtagagtgacatcagcaaactttttctgtaagggtcagattgtaaatgtgttaggctttgcatgctttatggcctcttaacaatgcaactttgttgcagtgagaaagcagccataaaagatatgtaacaaagtaggagtggctttattccaaacttttatttatgaacattgaaatttgaatttcatataatttccatttgtcacttttttgtttttttatcattaaataataaaaaaactttttagcttatgaaatacacaaaaaggatgatatgctgaattttgcctgtgaactaaagtttgctgactcctggagtaagggtttattagtccattcacacttaatgtgattattgattccattggatttatgtttgtcattttgctattctttttaaaatttatttttgtttatttgttcctctgttactgcattcttttgtgttaagtgcatattttttggtgtgccaatttaattcctctcttgacgttttattttttaaataatattgtctaagtaattttatttgtgattgctccacaaatcactccatgtatctttacttaccacaatctgcttcaaaataatgctaccttaatactgataaaatacagacacttggcttcatgtctccttttcctcccctaactttatgcctttatttttatttttatttttgagacaagagtcttgctctgttgcctaggctggagtgcagtggcatgatctcagctcactgcaacttccacctcccaggttcaagtgattctcctaccttggcctcccgattagctgggattacaggcatgtgccacctcacgcagctaattgttttgtgtttttagtagagacaggatttcaccatgttggccaggctggtctcgaactcctgacctcaagtgatcctcctgcctcagcctcccagaatgctgggtttacaggcgtgagccaccacgcccagcctgtgctattatttacataaattactatatgttgtatgttataacctcaacactactgttaacagttattaacttaaacagtctcatgccctttgaagaagaaatgaaaaaaatatatttatagtattttaaattgacgtacacacttaccatttccagttgtcaccaaatgtgatgaattcaaagtactgaaatggttgctattgacagtttagtccagcttcatagttacttttggggaaggagatttattggcctcctcattcctttttttaaaatatagttttcataaatggttttttttttttttgagacagggtctcactctgttgcccaggctggagtgcagtggtgcagtcacaggtcgctgcagccttgaccttcctttccaggcttgggtgatcctcccacctcagcctctttagtagctgggactataggtattcgtcaccatgcctggctaattttctaattttttgtggggttttttttttttttttttttgcagagacaggatcttcctgtgttgcccaggctggtcttgaactcctgggctcaagcagtctgcacacctcggcctcccaaagtgctaggattataggcgtgagccaccaagcccggccaaccacctcattccatcatgccagaagtgaatcccctaggtatatactgcttttatccccattttcacatgaggagactgagacatggatgtccaaggtcattaggaaatagtagttgcaatgtggactctcgtctcccgtctcctgtctatgctcttaatcatgaggttctgtgttgccatgcctgatgttaagtgagtgcacattcaagtaacatggcagtttctgaattatgcaccaagggttcagcacttgtatccattccttggtaacttcttttgtgactttgggcaaattattaaactaaggttcagtttgctcttctataaaacggagataaaaatagtgtcttccaagtagagctcttatgaggattcaagaagttaaacataacatttgttagtgcataacctccaagtgagtggcggttggagcagcagcagaagtagcagccttaatgttagaaagccttccctcattcctcctggtctgagttaggtgtggctctgagtgctgatggcattctgaaatatgtttctcttagagcacttctcacagtctcctgcagatgttccccaatgtattctacactagaatctaagatcctagaaggtaatgagttcatctttcatccttgtgtttttgctattgcttgaagagggaagcaaaaggctgaaatgaggtacttggaatgaggcaaggcatgaggctgatactttgcttcatgagattaaaagcgaggaacaattccaacagtttgggaaggcttatgggagatggattaaggaaagttgtttatacctttggccctgagagcatcacggacctgcttttcatgataccaagtaactgaaacacagagacagggtttttttacttagagtgccttattttaccttaaattgtacctgaacacttgatggggtggagtggtcattttgtagtaatgtaaaatcggctcgatggttacagaaaagtctaatacagaaaataaaagtcaacttcctgatcctttttactgttcactagagatgtaatcgctgttacttgtatatattttgaaacatttactatgcactttaggcatataaatatatatacaaatgagatgatctttgcatttagttttatagcttctttccccaataatgtgtagtgggcatctcgctttatctctacaacattcccttgtattgattatcatttattaaactagtctgatgatttttttttatggttattgttgcttcctgacatttaggttccacatttttgccaaaatgaattatgcagtaaatgtttcgtgtagggaagtgagctctaaaatcagttgtgtgactttaggcaagttccttaacctctctgtatttcagcttccccatctatacattgagaatattagtactactacttttgctagctgcaaacatctgtgtagcctaatataagccagacattgttttaaaaacatcacatatattaatgcatttaaacctcacaactagcctgagtgatgggtgctgatatgtctaagatcacaaaattattcagtggtggagccagaatatagtacgtgaacttacgcagtctggttccacagtgcacagtaccaaacttaacagggttataacaaagacttggcacatagtaggccaatatgtggtctgtatttaccaatattaggtcaatgatcaagtaatgtttgctttttgttttgtttttttttcaattaatgcaataaggaacattgttgtccatatgtctttgtttttggcatgagtatatatccaaagaacaaatcattagtagcataattacttggttaggtgtatatcaatttaaatttttgataaatattttcaaatcatcttttacaaagttttaccaatttacttcaatcatgagttatgagtgctcagttcaggatgcttttcccaacattaagtgttactaatttaagtttttcagactcagatgaagaggttatcttgttttggtttgaatttccttttcaatgaattttaaaaattgagatataatttgcatacagtgaaatcaccttttttagttgtattttgttgagttttgacaaatgcattcagttacgtaaacaccaccacaatcaagatatagaacaatttccattacgccaaaattctttggattttttttttttttttttttgagatggagtcttcctctgttgcccaggctggagtgcagtggcgtgatcttggctcactgcaaactccgcctcccgggttcaagcaattctcctgcctcagccctcctgagtagctgggattacaggcaagtgccacgcgccaccatgcctggctaatttttgtattcttagtagagacaggctttcaccatgttggccgggctggtctcgaactcttgacctcgtgatccacctgcttcagcctcccaaagtgctgggattacaggcgtgagccaccacaccgggtccatggattttttaatgatgaatgaattgagaatattttaggttattgattggccgcttgaatttgtgttctccctcttgcttttgtgatttctttcttattacctttcttcttccttttcatcattctttttgtgtgatgtttgtttctgtgctttgcccgtttgcttatagaattgtttgcctttctcttactgatttctaacagcacagtttatgttacaacattaatcctttgtcatatatatttgcagtttttctctattttttgctttctttatgccattttttggttatcaagaaatttgtacattttgcattgtcagatttattcttttatggattcaaggttttatgttatgtattagaaggccttctgcagtgcaagccttctaaaaaatatttttctccaacttttatattttttaaaaataaatatttatatatttgatgtttctggatttcattttgatacaagaaattaagtaagagccctgctggacccactagtcccgaaagtttctcccacctactcatcatcattcattgatagttcacctatccctggtggtcttaagatgtcttcttgatgacagactcttcccatatgtctcttggtctatttcttgactttctattctgtcatttccctcctctagcgttaagctgttgcagcatgttttaatatgtcatgtagctcattctccttcagttagtcttttagaatttttctcttttcgttgtttgcataacaaagttcactgtcattttgttgactttcttgaaaaagcattattgatgttttcttgggatctcgagtcaatattttgacaggattggattgttttacctgagaacaagatgtgtctatgtatatttgatctattatgattctctgaggagttttaaagtgtttcatgtagttttacatttgctattatttcaccttttttgaagctgttataaataggatctagtcttttattatgttttcttatctgcttcttaaaattatagaatccacaaaaattagccgggtgtggtggcacacacctgtaatgccagctactcgggaggctgaggcaggagaatcgcttgaacccgggagtcagaggttgcagtgagctgagatggcaccaccacactccagcctgggcgacagagtgagaccccatctcaaaactaaaaataaaaaagttatagaatttctagtgacttgtacgtatgaattttgtaattggtcacttgactgagttccttttttttccccctgatacagtcatgccctgcaaaatgacagtttagtctatgatgaaccgagtgtgtaatgtggtcctataggattataataccatatttttactctaccctttctttctttagatatgtttagatacacaaatacttaccactgtgttacattgcctacaatatttagaacagtaacatgctttacaggtttgtggcctagaagcaataggctataccataaagcctaggtaagctatacaatcacagtttgtgtaaatccactctatgtttgctcagtgacaaaatcacctaacgacatatttctcagaacatacctgttgttcagctgcatctattgaattttcagatgtttaatcatttcataaggataattttgtcctctcctttccaatatttatatttatttcttgtctaattcattggctaatatgttgggaataattctaattaataatacaattgatgtcctcgttgctggctttcagcatttcttcattacagctctgattttgtgtgtatatatatatatatatatatgtctattcctattttacaatgggtttggggtgtcaggaattgatgtttagtattttttcagatccttcttgtgactcatagctatgatcatattattttactcctttgatctaaggtaatatatattaataaacttccaaattagagccacgttagcaatcccaggatataccttatctagttgaattgttttctccctaccactcccaatgtttgctggtatttacggtttgtacttcaatgttcataagtgagatcagtttgatttttcttttttttggtcgtctttgtcaaattttggtatcagtatacttttgacctcaacaataaatttgggagctttctgttttctgtgtgctctgaagtgtgttgtgaattttggacctatgtctttattgtcctccccttttttctttctcctgaaggaaaaagagccatggtagctccttgatcactttctcagtttccttgacaattattagcttcttttggatttctgtgtcttctgctgcaaattttgatgatatatattttgctataaaattatccttttcatctgagtttcaaattaattttcatagaattttcaaagtagttttacacattttgattcctcatgcatgtgtggttattgctctatttctgattttgtataagcacattccctttcttgattatgccagctagttgtttaccaattgagttttctcttgcataatcaaagaatcaactcttgtaatgtttaatgctgttactttttattttctaattcattaatttgtgcttttatctttattttttctgatttttcttttgctggctgggggtttataattagcgacttcttgaattgaatgcataatttatttcttttaattccttaccaggtagttttgtaagtgtttaaatctttgaatttttctgtggacccagttttagggtcttgcagaatcacatatattttgatatgtttttttgtttttgtttttgtggttttttttgagatggagtttcgcttttgttgcccaggctggagtgcagtggcgcgatcttggctcactgcaacctccgtctcctgggtttaagcgattctcttgcctcagcctcactcctgccaccacgcccagcgaattttttatttttagtagagacagggtttcagcatgttggccaggctggtcttgagctcctaacctcaagtgatccgcccgcctcgacctcccaaagtgctgggattacaggcatgagccacctcgcccggctgatatgtagcattttcattatgttagtatacatttgtgttcactttgagattttgtctctgacttgagtcatttaaagatgtttgattttaatttaattaatttttttttgtttgtcctatttacttttattgccttgtggttaattaaggagttctctctgacttttaaaatttatgaaggttttcattggggcctttagtaactattccataagttcttgaaaaggagatgtgttctcagtttttagggtacagaatcccatatgtctaaaagataaattaattttgtaatttaaatttgctatattcttcatttttgtttacttcattttcctgggctgaaagagacattttaatgctgttccattttatgttcaaagatttttggcagatttttttcttcattatattatgaattatacttttcataatgtgccccttttcattttgtttgaccagtttatcttgaatgcagtgccatcttaccattgttactctagcttccttgttgccagcctatgcctttttccatttttttctgttagaggactcttttttttcttcaaaatttttcagaattgaatttttcagttaattgaaaaacttcactaaactagggactcgtgtactttaaacattttattttaaattaaaacaagtaaatacactctttcattaatttctaaatgtaggaccagtgtgttttctttaaatgtctattgactgaactctgtatcagtctaaatgatacactctatatccttacataatatgtctaggatttctagtttagcacttcttttaagtataattagaagtttaattgcctttgcaaagcaatctttatacaaaggcccagttatttttttcaaggtagcatagtggaatttacttattttttataatgtattttttatttcaataagtttttggggaacaggtggtttttggttacatggaagttctttagtggtgatttctgagatttgggttcagccatcacctgagcagtgtacattgtaccgagtgtgtagtcttttatccctcaccccctcccacactttcccctgagtctgcaaagtccattgtatcattcttatgcctttgcatcctcataacttagctcgcacttataagtgagaacatacgatgtttgattttccattccagagttagttacttcacttagaatggaagttagttacttccattcaggttgttgtgaatgccattctttcattcctttttatagctaagtagtattccatggtgtgtgtgtgtgtgtgtgtgtatacacatacatatatagatacatatatacacatatatacatacatatatacacatatatacatacatatatacatacatatatacgtatatatacacacttcatatatacatacacacacacacacacacacacacacaccacattttctttatccactcattggttgatggacgttgggctcattccatatttttgcagttgtgaatcgtgctgctatgtgtgtgccagtacaattttttttttttccctctgggtagatacctagtagtgggattgctggatgaaatggtagagctacttttagttctttacggaatttccagttttccgtagtggttctagtttacattcccgccaacagtgtagaactgttccctttttaccacattcacaccaacatcgttttgtttgtttgtttgagacagggccctacactcggtcacccaggttggtgtgtgggtgtgcatggtgtgatcttggctcactgcaacctctgcctccagggttcaagcagtcctcccaccttagcctcctgagtagcttgaactgcaagcacacactatcacacccagctaatttttgtaatttttggtagagatggagtttcagcatgttggccggactggccctcgaacgcctgacctcaagtgatccacctgcctcagcatcccaaagtgctgggatgacaggcgtgaaccactgtacctggcctattatttattatatagtttttttttaaaggaatgtttccctagtctttttgcctgtacgtgattctgcagaaaacatttttagccacctgcatttatttattatttctaaatcaatttgcactatttttatataaataattttcaaaaatattcttatttgttcatatgtttaaattgcataatgaatttaaatacaaaaaaaagtttgcatctgagcatcacccatgcccgggagctgtctttggtgttggagatacagtcatgaccactactgaaaacattcctgatctcaagtagaacacacttagtacaactagctggtagcataagaaaatcgcaggcacagtgaagaagagaatagttggaaagagtggaaatactggagagattttatgatattttgaaactttctgagataattttagtgtgtcctttccatatataacatattattgttgttttttttttttaatcagatcttaaaatgtttttcttctaggccggacgtggtggctcacgcctgtaatcccagcactttgggaggccaaagcgggtggatcacaaggtcaggagtttgagaccagcctggccaacatggcaaaaccctgtctctactaaaaatagaataattagccaggtgtggtggtgggcgcctataatcccagctactcaggaggctgaggcaggagaatcacttgaaactgagagacagaggtttcagtgagccgagatggtgccattgcactccagcctgggcgacgcgagcaagattccgtctcacaaaaaaaagaaaagaaaagaaaaaagagaagagaaaagaagaaagaaagaaaggaaattttgtctaatagttgaatttactccatttatatgtattggtataacaaacattttggagcctacatcttaccatatttcatgttgtatttgtattgtaattcttttttattatatggtctgttctattctttctttttctattctccctttctgggaagactttggtatctcttttttatttttcctttttttcctaagggaatggcaaatagtgttctcacaggtttaggagcggtggagtagaatctaagaaactgagatgaaagtacatgaaccatgcaggttcagaaattgacaatgcagcttatggtgtagagagagatttacaacgctattaactttttatgtggattctttgctttatgttaaagtctttctttttgttctcttaaattgccttcacagataatttaagcctgtggctctggtctgtggatcctctttcatcataatcctgaaacatcatttgtcttttttactgatggtacagaaagcaatgatggatgataaaactgatgatgccttaccatgagtcaaggcagcggcaccaaactgtaatagtaaatgtagtaatagtcgtatgttctttagtaagaaccaagcacttcagtaaaaaaaaaaaaaaaaaaaaaaaattggttatacttacggatgccattgatgaagctataaaaattaattttatgaaatcttcaccctgagcttacatctttctaaaattctttttatgaaatgtgaatatttcaaagacactttccagtacatactgaagaaggaagagtgtctccaggggaagcgcttgtttgaattgctggctacactagctaactagctcctttttttctgtgaaataaccacttttactaaaaacaacgatcgacagacacacatgattattccatgtgttttaacagatattttcccataaataatcaacatgagtctgtcacttcattaaaaacaaatgacagtattattgccagtgataaaattcaaagtttctttttttttttgagatggagtcttgcttgttgcccaggctggagtgcagtggtgtgatctcagctcactgcagtctctgcttcctgggctcaagtgattctccttcagcctccagagtagctgggattataggtgtgagccataatgcctggctaatgttgatatttttagtagagacggggtttcaccatgttggccaggctggtctcgaactcctggcctcaagtgttccacccacctcagcttcccagagtgctgggattataggcatgaccaatgggtttgaatgttatagagtaaaaaaattttgtgttatggttttagattttacagggcaatttatcattaagaaactattattcattgagttttgatatagtatcaaagaaaaatatccatagttatatgaaaaggttattaaataaaatatattgttcctttttccaactatgtatctggaggaggctgaattttcttcttacaggtagtcccctatgcacagtttcaattaccacagtcgatagtcatctgaagacagctaagtgcagtacagtaagatattttgcgagagagagagtccacattctcatagcttttattacaatattttgctataattgtttaattttattattagttatagttattaatctcttactgtacctaacttaaaaattaaactttatcataggtatgtgtgtataggaaaaaagcaaagtatacttgatatagggttctgtactatatgtggtttcaggaatccacaggggatcctgggatgtatcccttgcagataagagagggctactgtactgtaacagattcagtgcagaagtagatatgagagtctagctgtcttcctttagtcaggcagaagagatttacagtaatgtcatgtggtaccactttcactgatttttattatcattcatgaaaatgttatttatgttaacatgtaatgagtatattgtcattttaaaataaatataaaattaggctgggcatagtggctcatacctgtaatcccagcactttgggaagctgaggtaggaggattgcttgaggccaggagttcaagaccagcccgggcaacatagacctcatctctacaaaaagtagacaaaattagccaggcgtggtggtgcgtgcctatagtaccaactgctcaggaggctgaggtgggaggatctcttgaacccaggaggttgaggctgcagtgagccgagatcataccactgcactctagcctggccaacacagcgagactctgattctgtgtcttcaataaataaataaataacaaattttctcagttttttttttttttttttttttttttgacagagttttcgctcttgttgcccaggctggagtgcagtggcgcaatcttggctcactgcaacctctgcctcccagtctcaagcaattcttctgcctcagcctcctgagtagctgggattacaggcacacgccaccatgcctggctatttttttttttttttttttgtatttttagtagagatggggtttcaccacgttggccaggctggtctccaactcctgacctcaggtgatccgcctcggcctcccaaagtgctgggattacagaagtgagccatcatgccccgcctctcggttttattttctagtagggtacatttcaagagatgcaacccacataaacaatgcttcttcctgtcttccaataatttttaagagaataaaggggttctgagaccagaatgtttgagaaccactgatgtaagcagtagaatgacaggtaggacaggttggcataggggaagaatgtgagctttgcaaataaacagccaggttcagattctggatctgctgttgctagtagcaaattgtttaactttgttgggcaaattccagtctctctgataaccagtttcctcattttataaaatggggataataaagctgttttgtttattgaaggttaagtagttgtttgatctttttaattttttcattatgtgttatgatgactgacagacatgactacctcaagtcatttaagaaaaggttgaaattatttgaagaagttatcctttacccatatggataaagtttatgtctaaagctttttcccttgaaactttatatacattttcaagtatatgtataaacttaaaattttacaattagaaaagcagtaaaactaattcagaacatttacttaggcacgagacagatggtaatcctataaaaagatggcatatatttttggatgtgcaaacagaaaaacagatactcttaaaaattttagagtaatctttgcctagaatatgaagggaagagataaaagaccatatggcctaatagaacttttatagtttgtaatgtctaggattgaattttgtttctttaatattagtattgattaatagcttactaatatggattggtagttgaaaaaggaataattttcaagcaggtaattgccaagtacttttaccgtggttgctagtctctcgtgttttggggtatgggtgtgatagtgggaagcacaactatttgaatctcacatttagtagggttaatcagttactgattctggcagttataatagttcattatgcttgggtagaagacatgctactaagacactgagttcacctaatatacaacttcatttattatttaaaagtaactggtgcttcaaatattttaagttttttattttttacaacttattcagaaacatagtacatgacagtagacttttaaaaattgcttcttccttttggggacatgatagtactgatcccctgataggttgtctaatctgagtggtttttagtaagaatgattccatctgcacaccacccgaatcctatctgtaactaaaaggggacaattgtcaggttctctctctcctgtcagttcccaccttgtgtaccattatattactgaagggttgatactacagtttatctgttcatacatctgtctctcctactggattaggtgaccatcagttattcatcaatctgtgtattgccacacagggctgagccaagctgactcagcagaggccgagtaggggcttagtaaaggcagagtgaattcatgaatgggatcaaagctagagccatagtcttgtgccaagaaactgagtggcgctaaactgctgatttgggaggtcagaatcttgttctgggctttattagcatttcgctcaagtcaagcgaatcaccttgtcaagtcatatgcagagaaggcagaacatgacgttggaggaaagaaaacattctacacagtttaaacacagggcctttgtaaagtagtgttaggatcacttaacgtggtttatggaaccaagattttttgctgtgaaatgactttcagaaagtctttacagaattgaaatcctgtcatttcagtgtagaacaatgtacaaaaaaagtttagaaaatatatttctggatatagaagtaaaaatttgccatgggaaattgcaaaaaaagaagagtaagcaataataattctggaaatatgtttcataatcttccaaatagatcaaaactagacacatgggtttttatgggtgtacttcagagatgaagcttttcaacctgcgcgcattgagcttcttcctcctctgtgcattgggaacttgaaggtaaatgattcttgttctcaaggaattctcgagtatagaggaacatctctagatagtttattacagataatgaaattctttttgtttattttgacacaagcttaagtatttaaaatatgcatcagttgttgagttgactaggcttgttattgcagttctatggaacgcctttactgtcgtgccaggacacaggctgggacatggcaggcagcggaggagcagtcggagtcagtgctgaaggagtgcacaggtctagctagagtccagctgggaaggatgtggacactggcccacacgttagagtttataccaaaaagcaaggtgccaaaagaacaaaaggtagatgcaggcttgttctttataaacatcatcgggcggggcatggtagctcagcctgtaatcccagcactttgggaggccgaggcgggcggatcataaggtcaagagatcgagaccatcctggctaacacgatgaaaccccgtctctattaaaatacaaaaaacttagctgagtgtggtggtgggcgtctgtagtcccagctacttgggaggctgaggcaggagaatggcgtgaacccaggaggcagagcttgcagtgagccgagatcacgccactgcactccagcctgggcgaaagagtgagactcatctcaaaataataataataataataataaagtaaacatcatcaaatacttgccaaatgctcattgtgtagtaggcagtgatctgctctgctgggtgcaatagaaagaaagcccggtcctaacagagctgacattaagaaaagactggggtgttctcgttcagaagttaaaggattaaaactgactaaaacaaaacttttgtggaaatattttttaatatgactctttttatatttttattttttagaaaatggctccaaaggttaaatgaagcaggaaaaatacatagatgcagccttgcagcctctccagatgtttggggataatattccagatagaaatattgatcccttggattaggtaactagtcataatgaagaaaattagtcttaaaaccttacggaaatcttttaacttgaataaaagtaaagaagaaactgatttcatggtagtacaacaaccatcgctagccagtgactttggaaaagatgattccttatttggtagctgctatggtaaagatatggccagctgcgatatcaacggtgaagatgaaaaaggcggaaaaaacagatcaaaaagcgagagcctgatgggtacgctaaaaaggcggctttctgcaaaacagaagtcaaaaggcaaggcgggcacaccctctgggagctctgccgacgaggacaccttctcctcctcctcagcacccatagtctttaaagacgtgagagctcagaggccgataaggtccacgtcgctccgcagccatcactacagtcccgcgccgtggcctctgcggcccacaaactccgaggagacctgcatcaagatggaggtgagagtcaaggccttggttcactcttccagcccgagtccagccctgaatggcgtccggaaggatttccacgacctccagtctgagaccacgtgccaggagcaagccaattcactgaagagctcggcttctcataatggagacctgcatcttcacctggatgaacatgtgcctgtcgttattggacttatgcctcaggactacattcagtatactgtgcctttagatgaggggatgtatcctttggaaggatcacggagctattgtctggacagctcttctcccatggaagtctctgcggttcctcctcaagtgggagggcgcgctttccccgaggatgagagtcaggtagaccaggacctagttgtcgccccagagatcttcgtggatcagtccgtgaatggcttgttgattggcaccacgggagtcatgttgcagagcccgagagcgggtcacgatgatgtccctccactctcaccattgctacctccaatgcagaataatcaaatccaaaggaacttcagtggactcactggcacagaagcccacgtggctgaaagtatgcgctgtcatttgaattttgatccgaactctgctcctggggttgcaagagtttatgactcagtgcaaagtagtggtcccatggttgtgacaagccttacagaggagctgaaaaaacttgcaaagcaaggatggtactggggaccaatcacacgttgggaggcagaagggaagctagcaaacgtgccagatggttcttttcttgttcgggacagttctgacgaccgttaccttttaagcttgagctttcgctcccatggtaaaacacttcacactagaattgagcactcaaatggtaggtttagcttttatgaacagccagatgtggaaggacatacgtccatagttgatctaattgagcattcaatcagggactctgaaaatggagctttttgttattcaaggtctcggctgcctggatctgcaacttaccccgtcagactgaccaacccagtgtcccggttcatgcaggtgcgctcgttgcagtacctgtgtcgttttgttatacgtcagtataccagaatagacttaattcagaaactgcctttgccaaacaaaatgaaggattatttacaggagaagcactactgaaagattgagaaccctgcatcttgcactttgggaataagaacaagagattgaaatacagtttacaaactttcattgccatcaaaatcttttgctgccataactatttcagttttatgtgtaaaagagtcatcagtttgtttaggggtggggaagtgtcagcaaggtgtcttgggtttattttggttctttaaaaaagggaagtcttgaggttttagaggtgtgaattatgtttcatcaatgtgcagaataatcacaatgtgaattatcaaattctcctcaatgccccccccgcccagtcctttgctgctatccactgtgatttttatgcattaaaagcacatttcatgtgtattcaaccctaagtaaagttgaatgaaacttaacagaatggaaattgctatgtctttttaaatggtccattttcaaaagacagtgttgaataaacatacctgtgtgataaaacacagaatttacatatacactgaagatgagtttttaatctcttactttaaaaagatttatttagaatcgtgaattgacataatcttgggtaatggaacggagatctgcaacatatcttttaacaacacttttttctaaattatttctaaggttgtgctaattcttttggttgtgaaaagttgaatttttctgttgccttcgttttcatcttctagtttgtctattttaataaatggccttacattaaaaaattgtaaagaaatgtataccaccaatttagaaattgttgccttttctgtaattaaactcgggtacaaattggcataacatgaaaacctatggaactagaattattattaaagaaatattagatgatcatagcttcctgtgatagcatttttttgtgtgttacctatccttttggtaaaatgttttatctgtgatttctttagcttagtcaacattttcttgggtgaacttgattgtcaactaattttcataaatggactggattctcttgcaacattaatgtttataaaaagttttaaattgatttgaatagaaagaaaacattgttttaaagttggatttatatttttcttctatgtagttactataaaagtgtgctggatttgaccaatccttacccccactataaagagaacccgtgatgactttagtttaaaaattgtggaaattgtggagcaatttttctcacaatgtgagaaaaattctaaaccatattagataatgtggaagtcatattgtctatcatatatactgccatttaaaaataggtttttaaaatttagctaagtcttaagtaatttgccgttgctaataattttatctccttgagtcggttgttggggagagatgttatattcaataatttttagttattttgtaatgcagagtgtttattcatttcacagttctgcaatggatgtagtattttgggattgccctgtccagaaaattttcagctacacacctttaaaggaaaatgtttctatctcagatgaaacatgtaatttgggatggttcttcctttgtcacttaaaggaagagataggaaaagtctcttacccactttaaacatgagggtaaaggtttaggtcaaacttactggctttgtcattcaagcatatctgaatcctcacttttttctctttgctttttagggtcagaactgagatattaccaagaaaaggcacaatgccataatattatggtgttatggtattttgacttaaaggggaaaaggtacttaattttggtgggatgttgattgtaccttgttaaaaagactctcattttctcatatgttttctcctaataagatggaatatggagtatactgtaataatataagtgttcattataagctatttggattaagaactattgcagagttgtaagcttgttatcaaattaatgcaagacatttaaactatttttttgcaaaactatttatttttaaaacaacttaaagtatatctagggtgagttaaaagtccctgtgcatctatattagatggcaggttttgtcacagagtcactgtgtattaataataaatgttgaaatggcttctccgtgtctccagaagcatttacatgtcctccttgtgagatcatggtgcacagaggtctttggactgccctgaacccgtcttatgtggacataacctattcccttcgttttctcatcatgccatgtgtttaagatctacccgcttagtgtcaagattattgagattttcatctaaatgtttttaaaattgtattttgtgttttagtggaggacagtcttgatactgttctaattcagaaagccaaattttgatgcgcttttgtatattcataatatatactttaatatgccctattctaatctagtttgaaattgttagattctgatagtataatgataaaatcaaactatttgccaaaaagttaaatttacaagcagaaagatgttttgcgatattttctacttttgtgtagaaagataaccatttggggtaggcagcacaacagaatgtgaaagattggctttatatgatactaacacagtccagttaaaagtggaaacagggaaaagagagagagaaaactcatcatttagtttggtctagacacaatttggttaaattttaggattctattggcagttaacatagatgacatggacacagcctatgagtgctttttaaatacgtgcttctaaaaatctgagctcaaagcaagaaaaagaaaacaggaaagctgtatccatttgccataatagatgaatcagaattgttttctattaaatatttcatcacgaaatatgatggaggccagaagctatttttaggcttactataacactattatgcatttttataaataagaagaaggctgtagaaagtacttgaaaaatatctaattctctttctgtctaaggaaaactctttctacttggtgcaataatctgaaaatttagaaggtcaaaatatgtcacaggaaatgatcatagacctaaaaatagtttttaaggaaaccagctacacggggcaaccaaccatcttgctgttgaagaaaacagttccaaaaggctaaatcattggcataaaggaaacaaaacccaaagtatgcgttgtaattcttgagccagtattactgaaacaggataaaagttataaaatctaacataggttaatgcaatgtctctgctagtgctacaagtctaaaatatctttgtaacaatgctttcagaaatgccagttttaattacatttcacattgtatatgagagatactgctttatgaatgaatcggaataatacattttcatttaaatcttaattgcttttcattaaatgcgaagtcttaatttcaaaatgaaatcacactaccagcaatagacagttttcttgaaaactctaataaggaacaatagccagttccgtaaataactttaaaattctaaaagtgttggtacactagcaatcacaaatatggtttcttttaatacttttttaatcctgtaaagaagggtttttataaacattctttttattaatcagtcataacatggcgaagtgtgtagttgctgtttctgaaaagtgatccaaactctacatccagagattatgaaaaattcttatagaattttgtaacaagtatttacatgttgggtgaaagaaatttcatagtcgttggagtgccatgaaattaatacttgcaattcagattgcggtagtttacactttttctgtatgtttcaaatcaggtgtgtaccatttgtactgagaacaccacagaatgaattatccaaagtccattgattttaatacgtgttttggtttgtaaacaaattatagtaatttcttgcacatttttggaaattaattgtataagaatttatgtatctgcttctagatacagtgtgtaaataaaaaatttcgttcacaag

SEQ ID NO: 22 - protein, 440 aa, Homo sapiens
PWLWRGGRLLLQPPGPELPPVPFPLQDLVPLGRLSRGEQQQQQQQQPPPPPPPPGPLRPLAGPSRKGSFKIRLSRLFRTKSCNGGSGGGDGTGKRPSGELAASAASLTDMGGSAGRELDAGRKPKLTRTQSAFSPVSFSPLFTGETVSLVDVDISQRGLTSPHPPTPPPPPRRSLSLLDDISGTLPTSVLVAPMGSSLQSFPLPPPPPPHAPDAFSRIAPIRAAESLHSQPPQHLQCPLYRPDSSSFAASLRELEKCGWYWGPMNWEDAEMKLKGKPDGSFLVRDSSDPRYILSLSFRSQGITHHTRMEHYRGTFSLWCHPKFEDRCQSVVEFIKRAIMHSKNGKFLYFLRSRVPGLPPTPVQLLYPVSRFSNVKSLQHLCRFRIRQLVRIDHIPDLPLPKPLISYIRKFYYYDPQEEVYLSLKEAQLISKQKQEVEPST

SEQ ID NO: 23 - DNA, 48015 bp, Homo sapiens
ctccttggctatggagaggcggcccccgagccaggccctccgccaccgcccccgggccatggccccccgccgccacccttcctcgcgcggcccggcccgcggggctcccggccgccgcagctgatggtgttccgcaacgtgggtcggccgccggaggaggaggacgtggaggcggccccggagccgggaccctcggaactgctgtgtccccggcaccgctgtgccctggaccccaaggccctgccgccgggcttggcgctcgagcggacctggggcccggcggctggactagaggcgcagttggcggctctggggctcgggcagccggcggggccgggggtcaagacagtcggtgggggttgctgcccgtgtccgtgtcctcctcagccgccccctccgcagccccagccgcctgctgccgccccgcaggccggggaggaccccacggaaacgagcgacgcgctgctggtcctggagggcttggaatcggaggccgagagcctggagactaacagctgctcggaagaggagctcagcagcccgggtcgcggaggaggagggggcggccggcttctgctgcagcccccaggccctgaattacctccggtgcccttcccgctgcaggacttggtccctctggggcgcctgagtagaggggagcagcagcagcagcagcagcagcaacctcccccgcccccgcctcctcccgggcccctccggccactcgcgggtccttctcggaagggctccttcaaaatccgcctcagtcgcctctttcgcaccaagagctgcaacggtggctccggcggtggggatgggaccggcaagaggccttctggagagctggctgcttcagctgcgagcctgacagacatgggaggctctgcgggccgggagctggacgcggggaggtgagaccggccgggggctggccgacaaacttccttttcttgtttggtttccctttttatctattgctatcgcgatcctgacagttcttaagatagggtcttcaggaggaggcagagacttgggaccaaaggtggtaacatctcgcataaggtcagcgctaattgtggaaacagctttcactcttaaagagtgcacactccgtgacacttctcagctggtttgcatctgtcttgttggaagatggtttcagaccatcctcccatccccatcctggcatggctttttaagtttttccaaggcggctgtgtgccattgtccctggaagggagattgctggaagtgtttggcctttggggcaaaaagctggtgttgcaccttgaccgccttgcttattctgtgtaccagcaccgcacctcccttcacactcatgcatgcaaatgacaggagaatcttcaaatgcaattgtaggccagcaatcaattggacacctctattcaaagttttactgttttcctggttgttcccttagatgaacttggcatatcaaacttgggtgctgaccactgtctctgagaatagaaaatagcagaccgggcacagtggctcacacctgtaatcccagcactttgggaggccgaggagggcggatcacctgagctcaggagtttgagaccagcctgggcaacatgcgaaaccccatctctacaaaacaaacaaacaaaaaattagcccccatctctacgaaaaaaatacaaaaaattaggcgggagtggtcccagctactcgggaggctgaggcaggaggatcacttgagcccaggaggcggaggttgcagtgaatagtgatcgtgccactgcactccagcttgcgtgacagagtgagagcctgtctcgagaaaatagctttgcgctctctcaaagccttttgagaattctaagtaccagtaagatatagacaaggtagagacaggtacatgagttctaggccgattgatgaaaattccaaacatagacgttgagtttttttcctgcatttctgaggggtggactgatcttcggcatccagtgttacatctgtagcgttggtttacaaagctgtcaactttggtgtcagtgaaattagttaccttgtagtaactgccattgtggtttgtacttctttgggttctcagaaagattggaatgggttttgttgtggtttacgtgatggattcttgagacatactggcagccataaaccaaagctaaagaacaggtataaattgataaccttactgtgaactaagatcacttggggagctttttgaaaagcctaagagtttgaactttttaaaagctccccagttgatcctgaactgggaaccactcatccatagacttctgttttcaagggaacttgggtactccaacaagaaaatgttgggtggatttggctttcttgccctttctgaacagctgtgccatgggcttatcttggggatgaggccaaatccaaactttctaaagaaatcagtgtgttgtccctttaggtaggccagtgtttctgctttgcctcccagtgaattcatgctgggagttcagatcttgaaaagtgggtggctgctgaaacagctttttttgtggtaggctctgtattcgatgctccacttaccagcacagatgaaattttcatcagatgcaggggagggcgggattaaagctatgggtaatgacataggatagattgctaattttcttgtggtttgtcttgcctttgttccatattgaccaggagttcgtctgagattccagagacgccaaatacgaggtgtggcgtgaaattagctgctacgtctaatagattaggaagaggaaaaggcatatgacattatacggttttttgcctttggaagcagaatttaatggtcattggctgctctgtcaccaaccctcacactaagcacaatacagatttctgctgcagagcaaattgggctgtagctttgcaaagaagcttagttcagggcactggagaattacattttttggtccaagggctttttttgatgaattggagaagagctagttatttgtgtataaacgtttggttgactgctgtttggaggcagcatgctgtatagagaatgtgaaagtctagattccgtgtaaactccaacatcagctaggagatgaccaaatttgtcaccttcatgcttccctttttcccccatctgtaaaatgggtgagcgttgactgtcttgttcctgtccttataagagtagattgttgagaagagaaatctagatggaatattttgtgctttttagaagataagtaataggactgagctggtaaattttttaatgcttaggatttggtgctgcttgtgagcttttcgacagcttactatgtgccagtcttgctggcagcctcgcagctgtgagaaacttggtgagtctggccagggtagttgtgatttctgtggatctgcacctcctaagtccgcaatagtgggaggatgagtttggatttttagcttgttctcttgttttgattggtaggacctggaaatggtgagaaatttcagtcttttgccaaagggtatatgcctttgtcctcattgttgattggagttaattatgggcttctatagtctttgctgtataatttggggcttttgcagtaacctctgggcataattccctttgctattcccaacatctgccttcctacaatcattttagaaggtttaacaattcccacattttagcactaagcaaaacttccttttagttgcttttgagtctggtaagtggacctacctggatatattgtcttcagtgtggtatgagttagcttagagaagtctttttgttttgttttttgttttgaggtggagtcttgctctgtcgcccaggctggagtgcagtggtgcgatcttggcttactgcaacctctgcttcccgggttcaagcgattcttctgcttcagcctccctagtagctgggattacaggggcccaccaccacacccggctcatttttttattttttatttttagtagagctggggttttgccatgtttgccaggttggtcccgaactcctgacctcaggtgatctgcctgccttggcctcccaaagtgctgggattacagtcataagccaccaagtctggccaacttagataactgtttttaagtgaggtaagaaatgggcccaccagttgggtgtggtagtgtgcacctgtagtcccaatacttggaaggcagaggcaggaggattacttgtgcccaggaagttcaagggcacagtaagctatgatgtgccactgcattccagcctgggcaacagagccagactccatcttttttttggggccggggggatggagtctcactctgttgcccatgctggagtgcagtggcccgatcttggctcactacaacctccgcctcccaggttcaagccattctcccgaatagctgggactacaggtacacgccaccacgcctggctaattttttgtattttagtagagacggcgtttcactgtgttgcccaggctggtctcgaactcctgagctcaggcagtccgcccaccgcagtctcccaaagtgctgggattacaggcgtgagccaccacgcccagctgactccatctctttaaaaagaaagaaagaaagaaagaaagaaatgggcccagcaactaatcaggtttctgttctggggaactgggctagggcttataacacacatatgttggaattcaaagagctgggcttgatgcctgtggtcctcagtgtaatctagcattgctatttcaggaaaaccagtggagttcccatgtctgtttctcatatgttgagttgattgccttcctttggatttggtgagagatctaatttgatctgaccattttgaatcatcacgtaatacacacttggttggaaataggtaccttgttcacttttcaagatttgttttttaccaagttaacagatgaggctattactggctgtcttcatggtagtgaggtggagaaatggaggacggttctgtagtggtttcctatctatttatttgggggggaaaaaaacaaaatatagaattctggaaagagaggcaggctgctttggcatccatttggggccctgctgtcgtggacaggcctggcagacgtgagaaacttttttcactgtatccttgggtcttcaggtctaaagggatctttttgagtaatagcatgacatgctgagagcaaaatgattttatggtgtggcgaggaagttctgattttatgtttgggttttgggtccacacactgttgatctttttaactattttttccatgaagtggttgtggttggcagctctcctttagaacgaactcctgggcttaaaaaaaaatctctgtccatttgagtcctgtgcagtgcttctcactccttaacattctctctgccaacatcatttctaaggttttagaccttggaccatgcctcttggcttttccctggaagttccctctataggttctttagttggtttatgatggagttttggatctgaccttgctgcacagatgtctgtgtattggaactaaaaatgaacacatggtttttgtgcctgtagcacatgaacagacactggcttatgacgtgttggaaatggcacaggctggctgacttggcaccgtgtgctttagcaggggcagaggtacattggagccataaaatctggtatttggacatcctagtttggaagttactttagaaaaggacatgtgtaaatcttagggcatcagtacaggagatttatgggcaagataggttattattctttcattgtttttattttaggtttcaaggaatttgaaaaccagccaatggttacatctatgtgttgttgttctgtctagtctaatgcttttaactcctttatcccataaatctgttgcagtacagttaataatttgcagggtagaaatggacagaggcatcctgtttttttgttacctacagcataggcagtaatttagctgacacaaaggactgctggcctcaaagacctttgcctaaggcctgctgccctgtccttttctgtcacctgaagcacagcccaccaaatgctattctttcccaccctactcctccagtctttgttgctgctaagatgaaagtggtctactggcaaagttagaggggtataactttgcaccctaatgctttttgaggtgatctggggaagaataaataacatgggaatgtagtatagagggttagtttaaaatgttatctgaaattcctttattcatattgctctccaaaatatagtgtgtcagcttagaatggccttttatcattccaaatggttcatgtatacaaaaggggatatataaagtgtatgtctttgaataataataaaatacaaatgtaggtacccatcactaaacttaacaaatagaatgttatcagtacctttgaagagtgttgctttctaattacatcttacccctcccccaggtaaccaccatcctcaattttatgtattagtatcttgctttgttgttgtttttttttttttattgtcatctagattggttttttcatgaatgactgccattagaatgctctcaggatccttgagacagatactgctttgggtttgtagttaggcttgtctccccaaagagagaaagaagcatgcatatttattagtagccttttagcctcctagactttagaccttttacctttccaagcatccagccatgaggcccccttatttgccaggaaaaaccattctggcactctcgaacagctctccctggattcagatgtttttctttttctagatcctagaaactatgatttttcttcttcaaaatgccgggggtgggggagaagatgagctgaagtgagaaggcctccgcaagatgtgtgcatttgggtcctgccgttcttcagtgcttggcactgaagagactcgtccccagcatgggcagatggcagagtaatgcagcttcctttgaaaagaggcaaacggccaaatggctgaggtgtgaaccttttgaaaggccagtgcttcaggtttctacttgctgggtttaatgttgtacccacaagttatcaaataaaaatcagcttggaagctcttcagcagtcctaaagttcatgtccagtggaattaacattgcattccattcctctggatttttggaactaagctgcagtcatgagccacttttatgagcggagaaactgtgtggccatatcttatttttatgtagccacctctcctgtgggtgctgtgggtggaatattcatagtgagcgtgatcatcagaaccctcaagtatggaagaggttgccaagggaatgtaaagctaggtcaggagaaagacggaccagggtttcagctctttctgaataagatcttcatggtatggttttagaattttgtttgaggtgaattctgagaagctcagacatcagtgttaaaacataaatgcataaatatcttctgaagtcaaataatttaaaatagaatttcattttggattttctttgcttttgcccctctctattggtgcagtttatagttgaaaagagtttaagttctatttagacctttgggtaagagtatttttggaaggtgagccagctataatgctaggagacaggcactcaattctgcggtaggattttctttaagtctgttttactgttatatatatatatatattatttttttaaaaaaatttttaattaaaaaaaatttttttttgagacaaggtctctgtcacccaggctggaatgcagtggctcaatcatggctcactgcagccttgaccttcccaggttcaggtgaccctcccatctcacactcctgagtaactgggactagaggtgtgcaccaccacgcccagctaatttttatattttttgtagagacgaggtttcatcatgttgcccaggctggtcacgaactcctgggctcaagcaatctgcccacctcagcctctcaaagtgttgggattacaggtgtgagccatcgcaccgggcctgaggtataatttatataccgcaaaatttacccttttaagtgtccagctctattaattttgacaaatttatatatagtcatcataacttctaccataatcagcatgtaacatatttctttttctttttttttttttggggatggagtctcactctgtcacccaggctggagtgcagtggtgtgatcttggttcactgcaaccttcacctcccgggctcaggaaattctcctacctcagcctcctgagtagctgggattacaggtgcatgccaccatgcccagctaatttttgtttagtagagatggagtttcaccatgtttgccaggctggtctcgggctcctgaccttgtgatctgcccaccttggcctcccaaagtgttgggattacaggagtgagccactgtgcccagccagcatatggcatatttctttttttttatttttttatttttattttttattttattttactttattttgagacggagtctcgctctgtcgcccaggctggagtgcagtagtgcgatcttggctcactgcaagctccgcctcctgggttcacgccgttctcctgcctcagcctcccgagtaactgggactacaggcacccgccaccacacccggctaattttttgtatttttagcagagacagggtttcaccgtgttagccaggatggtcttgatctcctgaccttgtgatccgcctgccttggcctcccaaagtgctgggattacaggcatgagccaccgcacccggccagcatataacatatttctattacctcgacaagttcagtaattctgaggtgagtttttttggtttaacacacttctggccgtaatgtctttgttggagtcttttgagtttctttcatttcttgctgaaaagccacttgaaagtgtggtaagaagtgaagttatctttggtatattaaccaaaagatctgggttttcagactggttccctgaaacagcatcaagaagtcatggggagggctgtgtgagtggcagctcagagagacgaatgggccctcttggatgctaggcagagcccctaatctgaggaggcccacttagtagctgatggctttgacccatgccttggggcaattgtgcagaaatcagaggtcttcaaggttgaagactgcacatgcatcctgcagtgctattctcaggtcagaatctgttccttggttcactttgggatgacctatcctataggcaagcccagagggaaactttgctctgagctggtgcctgtccaggttaagagcccttttcttggctcccagatggaaattagctctgcatcagacttccttcctcttgaaatggacttgctaattaccactcaaggtgttttactcttttaatgtactgggtatggtgagctccaaagggatttctcattgctttgtaggttggtgaattctgtagccaaatctatttaaggaattgccttaattatttcattctccccctttattttatgctgtattacaagagtgttgctgacaacgactagggtttgctttatttctcagtgcatctggaactgagtgttgtggtgacttaaatgcatatgtgttcatttctcccctctattctctctctgctttctcactccctaggaaacccaagttgacaagaactcaaagtgccttttctccggtctccttcagccccctgttcacaggtaagggtaatatctttctctcttctgacatctgaaaacaggggcgttctgagacctgttgggaaacacttacagatgcatcacagggagctgtaggcgtgtctgcagttatttccttcattccatgcttagtgggtttggggtgaggctatttttatggtatggaatgtgttcttgactttggatgctttatagttggaggtaaaggctgcttgttggtaattaggtggcctgggtggccctagcagttatgcagtgcttagttcagggtcatcacatataaattaaaactaggaattacaaatttatgggctcacagtgcataaaagactctctagaagtcatacaatttctgtctctgcttccaagggagttcatgggttatctcataaaaatgatccatgattatctggtctatttttaaagacttttttgggcaaaggggattacatgagctcttgtctttttaggggcttgcagttcctgcagctgttagacttctttattgtatctaatttaagtctgccctcttgcagcagggaacacagttgctaaactaggaaggctgtagaatctcctttggatgatgggtaactgccttctcgtttcgtagggagctttttcagagagggagaccttctgttatgtcagtcacaggtgaagctcctgcatgaataagctggagaaaagtggggctttgcccatgtctcagaaactgagtacttctcattggttgtgactgagaagcctttaaaaaaaatcccaatctcaaactcctttggatatcatgtccctcaaaaaggtgattggaagacttacgatttgggaatggatgcttccaggaaaatgttaagcaggccaatgagacttgcagctgggtagactgctgtgaactgtcaaaacaagtgaattggtacctgtataggcatctactgtagtggagaaggaggtctctgttcttacccaagcccagaattaaaaaatcttatcttgcgataccatgtctttctagttaatagttgtttcctcattaaagtaaggaacatggcttggtcacggtcacggtggctcatgcctataatcccagcacttcgggaggttgaagtgagcagatcatctgaggtcaggtgtttcagaccagcctggccaacatggtgtgaaaccccgtctctactaaaaatacaaaaaattagctggggcatggtgccgcacgcctgtagtccagctactcgggaggccgaggcaggagaatcgcttgaacccgagaggctgaggttgcagtaagcagagattttgccactgcactccagcctgagcgactgagcctctgtctcaaaaaaaaaaaaggaacaccacctacatttgtagtttgaggttcaggtcagcctcagccctcttgaatgacgtcctttatgttaagtgctctaccaatagtctagactgctctggcctgtcaggaaatacacttgtctggctccatcgcattgctctgggcagccaggctttcagttaccaagctgtatggtgagtctggtaccagctttctagaacttccactgatggcttttgtttatttttgacacagggcctcactttgtctcccaggctggagtgcaatggtgcagtctcagcttactgcagcctcgacctcctaggcccaaacgatcctcccacctcagcctccagaatagctgggactataggcatgcaccaccatacctgaataatttttatatttttgtagagacggggtcttaacgtgttgcccaggctggtcttgaactcctgagtgcaagtgattcgcctaccttggcctcccaaagtgttggaattacaggtgtgacccactgcacctggtctcactgatgacttttagaagagcttttctgtttatcttaattagtattcaagagctttgtaaaaaaactgtgtatttaggttctgaaagcccagtttcctttggtagatattggtcacctcattttcttaacggggaaacatttaaaatactagctgaggggctcattcagccctgaattaaaaaaaaaggtgttggaaggaacaccgtaagggctaaattagagaaatcctgagttctgacttgtgcatcaggcctgccaaaataaccgtattcctggctatcagcttgtgttcatattaataaaatacctttcatcctctcgtgactcaagtggagagggaatagacagagttgggtggcaaggagtattgtatatggcagggaacagaactgcatgacatgctgggaagaagagaggccatgtgcctcctcccaccaagttcctatggagaagctgcctcttagattacctgacttttgtctgactgtgaaagtagttaatttttaggactataaattgtactttcagcctttgtagcctcagctgttaagaacagatatcttattcaggtagattctcttatgtgtgaatactctggaaaatcaagatatggactgccctctaggaagaagttaggactaacattgggaactttatctgcattcagcttaccgtacagttcttaggttttctggcccttgctttccatgtctgtttgggcatataacaagccacttggcttgcttcctaaggcagaaattgatacacaattctgaaaaaataaatggtatgggttctattggataaagaagaccccttgtttgagggtacgttgtaagtcagtggcggaaccagaattagatggtttttccactgtcagtcccatggttttgtcctaaagaatccttgacccttaacagggcaaaggaatgatagaatttaaaaattgacttcctggtgttggggtgatttcagccctgggggcattaggttttgagctttctgagagcagcagacccccagcctcgcctgcttgccctttgcttccctttgctttctgcatcagctttgggcaaggcgcttctgtaacttgcttgctccatgaatccctcaggtgaaactgtgtcgcttgtggatgtggacatttctcagcggggcctgacctctccacaccctccaactccccctcctcctccgagaagaagcctcagcctcctaggtatagtttcttcccctctccaccttcttgcctagtgggagggtgagctccagggacttgttctttgctgggggccgaccacgcttctgggtttccgatgatgactcaattatttcccagctctttctcagcaccccgtcatctccctaagcaattaaatgggtctcacccgcatgctgtaatacttactatacatatattaattcagtttacatttatcttgccttgaacccactgctattgggctgcattaggaatatccttactaccctgctttccaaaagcaaaacagagtaagatgattgttgccctctgtgcaaaaagactgggtgaaggttgggccatcctgccccagagggcagcctgatagtccttctccctctcctctgccttcagcgtgctcattctgtgctcacatttctgaacctcttgcttttgttctctcttctagatgatatcagtgggacgctgcctacatctgtccttgtggctccgatggggtcttccttgcagtctttccccctacctccgcctcctccaccccatgccccaggttagcttagtttagaggcaagacttgtaagagttgggagtacaaagtgatactttctaccatagaaggatgggagaaatctatttatggaaataacagcttagtagaaagtttaaatcttttccccttggctggaccagactccttagaaatgaggcggagaactggttttcctaacccgttttctttatggaggtcaatctgatttttgttagggatgaaagactactgaatttttcatcagtctttgctgctgagactgttttgcaatccttgagtagatatcacttaagaaactgccttaatttcaggatagtgaggtttaatatgttggtggttttcagcattctatggtttttctaagcttttagtatttactgcagccttgtaaataagtaccaagcatttagcagcccatacctgctttgccagcttacagtacccaaggaataaaatgaatgaaatcagcattcttttctacttgcctggcaaaatcattactcttcctgggcaagtaagcgagtcactatttccaaggctgccttatcggcacttcacttcgaggtgggagaagttggaatcgctctaggctggtgaagcccctgaagtagtcaccttagtaaggttagtgagcagctcccctccccctttcttggggtcaagtgcccccacccatccttcacagcttacacaagcccacacagctttttgttccagcccatccagctctttgccactgctttggcttctaccctttccagcttagcttctaatgccttgccctcttcagttagcatcctctgtttttgggggagggtctatttgtggttgggagcagtgagggtaaacaagtgaccaccactgtcttgccctgcagatgcatttccccggattgctcccatccgagcagctgaatccctgcacagccaacccccacagcacctccagtgtcccctctaccggcctgactcgagcagctttgcagccagccttcgagagttggagaaggtaggtggtacctaaggactggcaggtcacttctcctcccattagctaaattctgtattcggacacacctttatttatgtgtttatttatttttagagacagggtctggctctgttgcccaggctagggtgtggtggcataatcatagctcactgtaggctcaaactcctgggccaaaggatccttctgcctcagcctcctgagtagctaggactgcaggcatgcacgaccaagcccagctaaaatttatttatattttttgtagagactggtcttgcagtttcccaggctggtcttgaactcctgggctcaacgtgatcctcttgcctctgcctcccaaagctctaggattataggcgtaagccaccacacccagcccctgtttattgttttcagaggaagaatagtttacttgcttccatatttacagtgacaaaacagtgtgctcctgctattagttccatgagatgtaataaaattgaatttggggaaaaaatttggacatgataaaggttgttggagaaatgttttacatagtctaagctgattttgatagattaactgacccagttccctcttcttttcttggtagggtctttggtaacaaaattgtagaattattattctttgttttttgttgttgttgttgtcttgttttgtttttgagactgagtttcgctcttgttgcccaggctggagtgcagtggcgcaatctcggcttactgaaacctccgcctcctgggttcaagcaattctcctgcctcagtctcctgagtagctgggattacaggcgcccaccaccatgcccagctaattactgtatttttagtagaaatggggtttcaccatgttggccaggctggtcttgaactcctgacctcaggtgagccacctgccttggcctcccaaagtgctgggattacaggtgtgaaccactgcgcccagccattttgttttttgttttttgtttttttgacacagagtctcactgtgtaacccaggctggagtacagtggcgcaataccagctcactgcaacctccacctcctgggttcaagcgattcttctgcctcagcctcctgaatagctgggattacaggcatgtgccaccaaacccagctaatttttgtatttttagtagagatgggttttcaccatgttggccaggctggtctcgaactcctgacctcaagtgatccacccgcctcagcctcccaaagtgttgggattacaggcatgagccacagctcacggccagaattattattctgttactttggagggctaaaagtatagtgagcctgatgttcattgctaatggttgagacacttgtaagtggcttttacctattgaaatattttggagagaatgcagagggttctcccatgttaagatagattgcactgaaaactttgacatttctctgtcctgataactagtggactgcttcttgtctttgatagtgtggttggtattgggggccaatgaattgggaagatgcagagatgaagctgaaagggaaaccagatggttctttcctggtacgagacagttctgatcctcgttacatcctgagcctcagtttccgatcacagggtatcacccaccacactagaatggagcactacagaggtaagagatactggtaagagaggcttcttccccccttgatttgctctacctttgctgtctgacttttttgtggaagagattcataggaatggaactacaaataggggaaaaattatcttagactcagttgaatattgatatgagcgctgatttccgtcactagaagaaatggagggggtagggaagggagagtaagggttccatttaaaagtcttgtcagcagcatcttgtttcctaaataggagggcctgaacaaaactgttggagaggctcccgagaaatgagccggtaaaactctttaggtaaaaggaagaaagaacgtaggaccattaggcaaattctcacaagctttgtttgtagttaaacttatcatctagagatattttgttaaatcacgtatacaaagagggtgaatttagtgttgatcctgtaactgttctgttctgaatttctttcttttttttttttttgagacagagtctcgctctgttgcctaggctggagtgcagtggtgcgatctcagctcactgcaacctccgcctcctgggttcaagtgattcttctgcctcagcctcctatgtagctgggattacaggcatgtgccaccaggcctggctaatttttgtatttttagtagaaacagtgttttattctgttggccaggctggtctcaaactcctgacctcaggtgatccacccgcctcggcctcccaaagggcttggattacaggtgtgtctgaatttctttgtctgatagtcactttctcccaggcagcatggtgataatttcctctttttgtcaatgttgtagtctcttaggaaatattcattaaaatgttgagagtttgtgggatcaacatgctttagactatctgccattctcctagtggtacctcttcctttgtgatcagatggtacttcatgtgccagtcatgcctagaagccgacttgttaaacacagctctctggattgactttgtatcgagacattctgtatctaacttgggccagaaacttgtgatggtcattgtttctgaggctgggtaatacatcctttgtcaaaatacctgtgtatgctacaaattgaaaaagcatagagaactttggtgtgtttcagggacttgcttcccatccctcacagtcttaccatggtggtcaaccttaagtgaatgctctgagacattgcatctgaggtaagagcctctgtcctgcctcttctcaggccccacagcatcttccccactcagcccacactgcatcgggttctcttgtgaggcaggaagtagctgtttaggtcagcatagctttgtccatggtttcctctgtacccacttctaccttagagataacacgaagagaaaggaacttcaatccaatgacctgggaatgaattagataactaggtgggataggtgagggggactgcctcctgttctgctgtttgtgaaaacaaaaacacacaaggaaaaaaacaaaatgtgaagcggagcatttcagagtgaagtattttgttaaacctgttcaaatgactaggccctgggaattataaataaatacttgacctttaagttcttgataggtgagatgccaggaacaagaggtctttcaaatagttctccgattacttttttttttttttttttttgagttggagtctaactttgtcacccaggttggagtacagtggcacgatctcggctcattgcagcctccacctccttggttcaagcaattctttttttgtttgtttttttttttttgagacagagtttcgctctgtcaccaggctggagtgcagtggtgtgatcttggctcactgcaacctccgcttcctgggttcaagcaattctcctgcctcagcctcccatgtagctggtattacaggtgcccgccactatgcccagctaattttttttgtatttttagtagacacagggtttcaccatgttggtgaggctggtcttgaactcctgacctcgtgattcgcccgccttggtctcccaaagtgctgggattacaggcgtgagccaccatgcctggccgggttcaagcaattcttgtaactcaacctcccaagtagctgggattacaggtgtgcaccaccacgcccagctaatttttgcatttttagtagagacagggttttgccacgttggccaggctggtctcgaactcctagcctcacgtgatctgcctgcctcggcttcccaaagtgctgggatgacacgcgtgagccacctcacctggccaccagttactttttgttaaagggactttgcttccttgaataagtagaatttcttttttttgagacggggtctcaccctgctgcccaggttggagtgtagtggtgagatcatgcctcgtcgtagcctcaacctcccaggctcaagtgatcctcctatctcagccttgctagtaggtgggactagtgtgcatcaccatgcccagctaatttaaaaaaatttttttagtagagacagggtctcactatgttgcccaggctggtcttgaactcctggtctggagcaattctactgccttggcctcccaaagtgctgggattatagatataagccactgtacctcgcctataaccggaattttttttttttttttgagacagagtcttgctccatcgcccaggctagagtgcaatggcgcgatctcagctcagtgtcaacctctgccttccaggttcgagctattctcctgccttggcctcccaagttgctgggactataggcacccaccaccatgcccagctaatttttgtatttttagtagagacggggtttcaccatattggccaagcttgtctcgaactcctgaccttgtgatccgcctgcctcggcctcccaaagtgttgggattacaagcatgagccacggcgcccggccttataactggaatttttaatggaaaccttgcgggcctttccgttaccagtttgcagtggagcatttgtctttgtcttttttaagtgatactgtaagagtaggtagagagactcaaatctgcagttctcttgactgattagtatcccttttttgtgtttgtttgtttgttttgttttgttttttaaagacagggtctggctctgtgcccaggttggagggcagtgccacgatctcggctcactgcagtctccacctcctgagttaaagcgattctcctgcctcagcctcctgagtagctgggattacagatgggtgcctccatgcccaacttttttttttttttttgagacggagtctcgctttgttgcccaggctggagtgcagtggcgcgatctcagctcactgcaagctctgccttttgggttcaggcaattctcctgcctcagcctcctgagtagttcctggacgcgtgccaccacgcccagctaatttttgtatttttagtagagacggggtttcaccatgttggccaggttggccttgaactcctgacctcaggggatctgcccacctcagcctcccaaagtgttgggattactggcatgaaccaccatgcccggccgggacgggtatttttctgtgttgtccaggttggtcttgaactcctgagctcaagtgacctgctcacctctgcctcccaaagtgctgggattacaggtatgagccactgtgcctggcccttttgtgtttttttttttttttttttttttgtgggtgattcctttggaatctggtgcttcagttttttgagttctttcttgttttatttttttaagtcataggagttcagattcccttttccatttgggattttaaagtttgaggtgggatctttgtaatatgttggcgtggggttttaaagttaaagaaggaaagacttagatttctattttttgcactcaacacgcacttgaatgaatgagacccacaaaactgctgttcctgtctttcctcgactattccatatggtctctaagaagtatatatagttgacccttgaacaacctgggtttgaactgcgcaggtctgcttatacatggattttaaaaagtaaatatattggaaaattttttggagatttgtgacaatttgtaaaatcttacagataaactgtagcctagaaatatcaaaagaattaaaaaggtatgttatgaatgaataaaatatatgtggatactagtctattttaacatttgctaccataaaatatatacaaatctgttataaaaagttaaaactttatcaaaacttatgcacacaaaccgtacatggcaccattcactgttgagagaaatgtagacaaatataaaggtgcagtattaaatcataactgcataaaattaactgttacatactgggtgtactaccgtaatatttcatagccaactaactccctgttactattgtggtgagctcaagtgttgcaagtatccgcttaaaatgttgtgtgacgctagtcatcaccatgtgagtgtttatctctccagctaattgtgtatcacagtaaaaattgatctctcatggttcttgtatagttttcattgtgtttagtgcaatacagtaaaccttgagtaatactatgggacccactagtgccactagtgttgctggaagttcccctaagaggcagagaaaagttagaacattacaagaaaaattgctcagtttgtacatagtttgaggtccacagctgtggttgttcaccatttcaagataagtgaattcagcataaggactgttgtaataaaagaaaaaaggctggatgcggtggctcacacctgtaatcccggcactttgggaggccgaggcgggcggatcacatgaggtcgggagttcgagaccagcctgaccaacatggagaaaccccatctctactaaaaatacaaaattagccgtgtgtggtggcgcatgcctataatcccagctactcaggaggctgaggcaggagaatcgattgaacctgggaggcagaggttgtggtgagccaagatcgtgccattgcactccagcctgggcaacaagagcagaactctgtctcaaaaaaaaaaaagaaaagaaaagaaaagcatacctatagataaatatgatttaataaaagtaaagtcattatatgacaacttaaagcaagaggaaagtgaaaaaactagagctggagaagttaatgccggcaatggatggtttgataattttaggacagtttgacttgaaaaatgtcaagataacagaagacgtagcttctgctgaccaagaggcagcagacaggttcccagacaccatggggaaaatcactgaggagaaaggatatctgcctgaacaggtttttaatgaagatgaaagtgccctattctggggaaaagaaaatgccacaaaggatatttattagtaaggaaaagaagtaagaaccagaatttaaggcaggaaggggtaggctgactctctggttttgtgcaaatgcaattgggttaataatcaggactacctttatctatgaagctacagtcttttggttgtacagcaagaaggcatgcacaatgagaacctttttttctggattggtttcatcagtgctttgtctctggagtaaggaagtaccttgccagtaagggattgcctttaaagctcttttgatattggtcaatgcccctggccacccagaaccccatcagttcaacactgaaggtgtcaaagtggtctgcttgcccccagacgcgacgtcgctaattcagcctttagatcaggtgggtcataaggacctttaacactcattgcacttggtactctatgcaaaggattttaagtgctgtggaagaagacaccgatagaacatcatgaatgtctggaaagattcctccattgaagttgccatagttgaccgggtggagtggctcacgcctgtaatcccagcactttgagaggccaaggtgggtggatcacctgaggtcaggagtttgaggccagcctggccaacatggcgaaaccctgtctctactaaaaatacaaaaattagccaggcctggtggcacgcgcctgtgatcccagctacttgggaggctgagggaggagaatcgctccaacttgggaggtggaggttacagtgagccaagattgcttcgctgcactccagcctgggcgaccgagtgagactctgtctcaaaaaaaatgccatagttgttatagaagaaattgtgggccaggtgcagtggctcatgcctgtaatcccagcactttgggaggccaaggcgggcagatcacttgaacctggaagttagagaccagcctgggcaacgtggtgaaaccacatttccacaaaagatacaaaaattagctgggcactgtcatgcgtacctgtagtcccaggtactcgggagggtcaaggctgcagtgaaccgtgatcatgtcactgcactccagcctgggcaacagagtgagaccctgtctcaaaagaaaaagttgtgaaagccatcaagcccaaaacaataaattactgctgaagaaaactgtgtccagaaattgtatgtgacttcataggatctgtgacagagccaatcaaggaaatcatgaaagagatatggcaaaaaaagaaggtgaagggtgaaggatttcaagatagggatcttagagaaattcaagaactcatagataccacatcagaggaattaactgaaggtgacttgatggagatgagttcttctgagtcagtgccagatgacgaggaagaagacgtagaagaaacagtgccagaaaacattaatgtcaggcaatctggcagaagggtcgaggttatttgagactgcttttgagttattttagacttggacccttctatgatatgggcactaaaaccaaagcaaatggtagaagaagaattggtaccatatagaaacatttttagagaaatgaaaaagcaaacctgagtgtggaggctcaagcctgtaatcccagcacttgggaggccaaggcaggaggatcacttgaggccaggagttcaagaccagcctgggctggtagcttcagctattcaggaggctagggcaggaggatcacttaaacccagtagttcaaggatgcagtgagctgtgatcatgccactgcactctagcctgggtgatagagtgagaccctgtttctaaaaatagaaaaagcaaaaaaagtcagacaaattataacatttccataaatttataccacatgtgccttcctctcctacttccccttccaccttctctacctcttctacctctgccacccctgagacacccccctcttcctcttcctcttcctcctcagtctatttggtttgaaaacaatgaggatgaaaacctttatgaagatccactttatgatcactttatgatgatccacttccatttaatgaatagtaaatatattttctcttattttcttaatagtgttttcttaatagtattcctttttctagcttaattgtaagaatacagtatataatacatatcacatacaaaatatatgttaaccgactgtttatattattggtaaggctttcagtcaacagtaggttgttagtaaagttttgggggagtcaaatggatttttatatgtggattttttattttggtggggttgaggaggatgttgggccattgggatagggttcatgctcccccccacattattcaagggtcagtttatgtacgtaaattggtcattcctcacttaaaggtcattaataggttcttggaaactgactttaagcaaaatgacgtataacaaaaccaattttttttttttttgagacaaagtttcgctcttattgcccaggctggagtgcaatggtgtgatctcggcttaccgcaacctctacctcctgggttcaagcaattctcctgcctcagcctcccgagtagctgggattacaggcatgtaccaccatgcttggctaattttgtatttttaatagagacgggatttctccatgttggtcaggctggagtaaagaccaaacttctaaataaagaccaaaacacttctaaataaagaccagtattaaacagtgaaatatatgtaaactgtacacacatgtaagaaagattaatgaaaataagttaggtaatcatttacccaagtattccagctcaggatcacaaatggccggagcctatcctggcagttcaggatgcaaggcagaacaaccctggacagaacaccattccatcggggtgcatatacccacacacccacactcagactggcacagcttagcatgccatttcaccttacacgcacatctctgggatatgggaggaaaccagcatacccagagaaaacctgtgcagatgtgcaaactccacacagacagtggtcctggccggaaatcaatttttttttcttatcaacgttacaaggaaacaagcatggtggctcacgtttgtaatcccagcacttagggaggccgaggcgagtggatcacttgaggtcaggagttcgaggctagcctgaccaacgtggtgaaaccctgtctctactaaaaatacaaaaattagccaggcgtggtggcaggtgcctgtaatcccagctactcaggagtctgaggcgggagaatcacttgaacccagcaagtggaggttgcagtgagccaagatcccgccactacactccagcctgggtgccagagcaagaccctgtcgcaaaaaaaaaaaaaagttataaggtaacaacactgaacaaaatgttattcaaagacctgcttatatgtgtacacagtcatacactgtttagcaacattttggtcaatgatagactacatatatgacagtcgtcccataagattatattactgtatttttactgtagtttttctatgtttaaatgtgtttaggtacatgaatacttaccattctttagagttgcctccagtattcagcacagtaacatgctatacaggtctgtagcctagcagcaataggctgtaccacatagcctaggtatgtagtaggctacaccatctaggtttggataagtacactctgatgttcgcctaatgatgcatttctctgaacatatttctgtcattaagtaatgcatgactgtacctatacttaatatacataataagtacatctatatagagagaggaagcctgtgatttaaagattaatgttcatactcagtgaacaattttatatcattttttctgatttgaaattattaaaggttgagtatccctaatctgaaatccaaaatgcttaaaaatctgaaactttttgcacactgacatgatgctcaaaggaaatgctcattggagcactttggatttcagagttttggattagtaatgttcaactggtaagtataatgcaaatattccaaaaatatgaaaaaatccaaaatctgaaacacttctggtctcaagcatttcggataaggagtacttagcttgtatttacttaacaaaaaccttgtaagacctaatttatgtcttacaaaaggcaacatctttttacagttaactgtgagataagcaggaacaggtagcttcagagacctttgagccagcttgcccccaaactgccctcccaaaagtgagaatcatagttaaaatattcaaaccagcctctcatgtcttctgaagtaagttttaaaatttttacttcttaattttccatggcaggaaccttcagcctgtggtgtcatcccaagtttgaggaccgctgtcaatctgttgtagagtttattaagagagccattatgcactccaagaatggaaagtttctctatttcttaagatccagggttccaggtaaggctgtacttctgttaattatttaacttaagttgggtatgatccagtttagtgtcaaaaaacaccatccccacaaaaggccatggttaagctttttttccctggctgttggagccaccactgctcttacagagtcccatggttaatcccctgcctttgtacattaatctctcagcatactgaggaggaatctttgcttctctaaccacgtgaatgagaaaactgcttccacctctgatgtccaggcatcatgtgaacatttgacatttggaggtatccctgatctaggcatctcaacatttataagtgaagcagaaagatgagagctctcaggtcctacccataggaacggggaaggtaggagaatgtttaccagaaaggcaggggcgagcgtcagtggggctcagtatggctaggcgtggtggctcatgcctgtaatcccagcactttgagaggccgaggtgggaggattgcttgatcccaggaatttgagaccccatctcttaaaaaaataaaaaattagccaggcatagtggcacatgcctatagttccagctagttgggaggctgaggtgggaggatcacttgagcccaggaggtcgaggcttcagtgagctgtgatcatgccactgcactctagtctgggtgatagagcaaaaccctgtctcccccaaaaaagaaaaagtaagggccaaatccagttgcacattgcactctgttaggaagtatctgctgtgggtttgcccatgttgtactaaacttgctgccgaatagtaaatactttgttttgtatcttgccatccctaccattgatgaatacaagacctctctccatttgggagcactgggcagttgctatccttgctgatgtgattgtatagtcttcctgcccactaagttagggaggtgtctgtataatctgtggagggcaagaggggattgtttggagcaatctgcctttatagtgttgggtaagacaaaatgacacggacattaatatgaggaacttgtaagatgcaaacaacttaagccaggcatggtggctcacactgtaataatcccagtactttgggaggctgagacaggcagattgctcaagtccaggagttcaagaccaggctgggcaacatggtgaaaccatgtctctacaaaatacacaaaaattagccgggtgtgatgccaggcgtctgtagtcccagctacttgggaggctgaggtgggaggatcacttgagcctgggaggcagaggctgcaatgagccaagattacgtcactgcactccagcctgggtgacacagcgagaccctgtctcaaaaaaaaaaaacaaaaaaaaacaaaaaaaaaaaaacaaggcaggggttggtggctcacacctgtaattccagcactttgggaggctgaggcgggcagatcacctgaggtcaggagttcgagacagcctagccaacatagtgaaaccctgtctgtactaagaatacaaaaattagccaggtgtggtggcaggtgccttgtaatcccagctactcaggaggctgaggcgggagaattgcttgaacctaggaagcggaggttgcagtgagctgagattgcgccattgcactccagtctgggcgacagagccagactctttcaaaaaaacaaaaacaacaaaaaagaaacaacttaaaatgagaacttcatgctttcccattgaatcaaattaagattcttgaacagaacacctagaaattgctctaaactgtaggagctcaggggagcctcaggcagaagactaggaacaggttgagccaggtgacaggaatgtttcttgtaatcttggggatttgaataaggcatgacagggtgtgaactgtattaattctcacttcatttagttttgcctcagtgctgtcaacacagttgggtcttgtgcagttgacttgcttaaagggaaaagttagattccttccttataggagttgactctgcttagcagtttttaaagtttatggctttttggggggtttttatttaacaaacatttattttgtaacttaattatcaaaaataagctacctttctgccttcagagaagtcatagtctaaaagaagggacaggaaggaagacaaaggtcagagcacatgttcaagtcagtataaagcatgtggtggtaaccagcaagagtagggtagtcagctctgcctgtggacaagcccgggaaatctcaaagaagattctggactctggaacatcactctatttgtagaatattgagaaaacttcgtggaatattggactcctcagatacagcttatttcattgtacaaatgtaccctgtctgcagatattttaaccaagattcttagcattgtattaagggctagagtgtgtagtgtttgcaactgttaatattgagtgcctgttaaatgccaggtacttggccaggctctttatttaaatatattgacatttttacaacaacctcaaagttaggtaatatagatcaatctattaaaataatatggctgggctcggtggctcacgcctgtaatcccatcactttgggagtccgaggtgggtggatcacctgaggtcaggagtttgagaccagcctgaccaacgtggtgaaacctcgcctctactaaaaatacaaaattagccaggtgtggtggtgcacgcctgtaatcctagctactctggaggctgaggcaggagaatcgcttaaaacccaggaggctgaggttgcagtgagctgagattgtgccactacactctagcctgggcccccaagcaagactctgtctcaaaaaaaaaaaaataataataataatgataatgatgtgtccaacatggtgaaaccacacctctactaaaaatacaaaaattagccaggcatggtggcgggcacctgtagtcccagctacctgggaggctgaggcaggagaatcacttgaacctgggaggtggaggttgcagtcagccaagattaagccactgcacttcagtctgggtgacagagggagactctgtctccaaaaaataaaaataaaataaatttaaaaataaaataatacatgagacttcattttgccagtgaggaaactgaggctgaagctcaaaagttaagttacttactctaaaggtaactcggttgatcagtgctgaagccagaattcaaacctgggcttgactacaaatccttgactttatttcccaccggtctgttattggaagaggtagtagtaatcagattgaaagtttgctggcatttgtggaaactacctaaagaaggtagaaccatgtttactgggggcaggagggtgcacatgctccagttgaatatctgtgtatacttaaaattgtaatcaagttgagagggcaaagcagagctattgcccctcgaccctaagtgacctctctttgaaggatagggccaaagaagacaaagcctttaccactgtttgctgccctcttttgcttataaaggggaaggcattatataagcttgattttattagaggagactattagggtgaggaagaggtaggtattagcatttaggttgagctctgtcagtctgcgtcagggattttaacttgatcgattcagaaaggaggctgtgtcttcagactttggaatgatgacaactgaacacctacaaggagccaggtgttattccatttaaccaagtgttaccctttgaggggatagtaccgtctcctttttattttttttctttttgaaaaagcaaaagaaactgaggtccagtgagttgcagaaccaggatgagatctcaggtttttctggctcagatcccagagctggatgcagtaggagggtgttaagactggtatcaggcttggcgcagtggctcacgcctgtaatcccagcactttgggaggccaaggtgggcggattgcctgagctcaggagttcaagaccaccctgggcaatatggtgaaaccccatctctactaaaatacaaaaaattagttgggcatggtggtgcacatctgtagtcctagctatttaggaggctgaggcatgagaattgcttgagccccagaattggagattgcagtgagccaagtcataccactgcactccaaagactctgtctcaaaaaaaaaaaaaaaaaaaaaaaaaaactaaaaaaactaaacccctaacaccagacagcagtgagagtgtgactgatgtcacacaaaacctctgatcctattagagccaggctaaaagcaaacataccctttttaattgagagaatcttcatctgtaaagttcgtgaagaaaaaaatatctttaaaaaatcatgctttgatcaggcttggtggctcacatctgtaatcccagcactttgggaggtagaggtggaaggattgtttgagcccaagagttctaactagcctggataatgtagagagacctcatctctacaaaaacaaaacaaaacaaaaaagcctagccacatgtgatggtgtgtgcctttggtcccagctgcttgggaggctgaggtaggaggatctcttaggccgggtaggtcaaggctgcagtgagctgtgattgcaccaccacactccagcctgagtaatagagcgagaccctatctcaaaaaaaaaaaaaaaaaaaaaaaaatcatgctttgaattggacagctcatttttgtttgtttgtttcaagatggagtctaccccagtcacccaggctggagtgcaatggcacgatctcggctcactgcaacctcctccgcctcccgagttcaaacgattctccttcctcagcctcccgagtagctgggattacaggcgcccgccaccacgcccagctaatttttgtatttttagtagagacggggtttcaccatgttggccaggctgcccttaaactcttgacctcatggtccacctgcctcaacctcccaaagtgctgagattacaggcgtgagccaccacgcttggcggaattggacagctctttaaggaaattggttacttcctcttttgtaacccttgttaagtttataattacttgttcagtgacatgtcttccctgtttaccagggtttctcagctctgcattattgacattttaggctggacagttttttgttgtggggtctgtcccgtgtattggaggaagccctagcttttgcccactagatgccaggagcactcacccccaccccaagctgtgacaatcacattatgtctccatatgtccctgggggccaaaatcatccccagttgggaaacattgttatataccataggggttaaaaagtacaagttctggatccagagcaccgagtgctaccccaggtcctgccagttactagctctgtgatcttaggcatgaactgaacctctcttgcctcagtttcctcaacagtaaaatggggatgattatagtacctcacttatagggttgttttgaggaatgcaagagttaactcatgtaaaatatttaagacaatgcctgacacagagtaagcactcaacagatgttacttttctcagtgctctctaagatgtaggtgcataggaagtgtttaaggtggtatctctggtgttctcctggccctataaaggtggtacccaagataatatgtgttgaatgatactctgggtggcccctcagatatccttgtgatatcctttgagatgccctttgtcgttattttatcttacctgtctgattgccaggtcatccagagcaagaactgtctttccttagctctatattattttgagatgaagtctcactttgttatgcccaggctggagtgcagtggcgcaatctcggctcatcgcaacctctgcctcctgggttcaagcaattctcctgagtagctgggattataggcgcccaccatcacgcccggctaaatttttgtattttttagagacggggtttcaccatattggtcaggctggtctcgaactcctgaccttaggtgatccacccgcctcggcctcccaaagtgctgggattacaggggtgagccaccgcgcccagcctgctctatattattttgtatattagaacactcaaacatttgctgatgagatattagacatactcagaagaggttatttcacttacgcttgtgaaagaagccaggagtcctttcctggggatttgtgaccagtgtctctgagcatctccccagtaagcggttcgtccttgttttataggtacagagtcatgccactgatggggctcccctccttgctctggctgcatgaccccctttagcaacaccgtaagactcccttgaggatatttgcaaactagacttattcctggcttattacctgctttatgtttatttgtatggtgtgcagaatggtgtaataaaccctgtgtatccatcacccagccccagcaaccatcagcccatggccactcctgccctgtttgtatacctgtccacttcttcattgcatattagtgtttagtgtttaaatatacttttgttttgttttgttttgttttgagacagggtctcactctgtcactcaggctggagtacagtagtgcaatcatgccttgacctcccaggctcaagtgatcctcccacctcagcctcccaagtagctaggactacaggcatgggccaccatgctcggctaatttttcaatttttttgttgagacaaggtgtttctatgttgcacaggctggtcatgaactcctgggctcaagtgatcttcccttcttagcctcccaaagtgttgagattacaggtgtgaaccaccatgcccagcttcttttttttttctttgagatggagtctccctctgtcacccaggctggagtgcagtggtgcgatctcggctcactgccaccatccacatcccgggttcaagtgattctcctgcctcagcctcctgagtagctgggattacaggcacacaccaccacacctggctaattttttaatttttagtagaggcagggttttaccatgttggtcaggcttgtcttgaactcctgactttgtgatccacccgcctcagcctcccaaagtgctgggattacaggcgtgagccactgcacccagctctttttttttttttttttttttttgacggagttttgctcttattgtccaggctagagtgcagtggcgcaatctcagctcactgcaacctccgccttccggttttgagcaattctcctgcctcagcctcccaagttgctgggattacaggcatccgccaccacgcccagctaattttttgtatttttagtagagacggggtttcatcatgttggtcaggttggtcttgaactgctgacctcgtgatccacccgcctcagcctcccaaagtactgggattacaggtgtgagccaccgtgcccagcagcacttcttttttaatttgtagttttcttcctccatttctttttccctttttttcttgcaatttatatatttaaaatattgttatgtttggagtttacctattgcttctccatgacgttgtttaatatgttcctctgtactctgaatttcctgtaaaatggtaattgaattcaaaggcttacccaatttggattcgttggggagggggagcagcgtgacagcattacttcatctgaattttttttcttttttttttttttttaaatagaaaaccacaaaaatcatatgcatggtttaatattattataaggtgaacaccactcaggcaaaggaaccaaaacttttccagccactccagaaatccttccgtgtgccccatactaatctctacacctgtctttataataatcacttcctttcatttttccatgaatgcttttatcaccccagtgtatatccctgaacactatagtttgatattgctcatttgaaaaaaatttcatgtcttttaaatatcttaatctgtggctttttctttcatttccttacgatttacttattgaataattgggctgtttgacctggaaaatttccttggattttactaattacgtactcttgttggagtttagcatgttgtcctgtcttctgtatttcttacaaattagcaggagtctgattcaaactcaggcttgactgaacatataatagtagctcatgcctaaaatcccagcactttgggaggtgaggcaggaggatcgcttatacccaggagttcaagacctgcctgggcagcatggtaagaccctgtctctaccaaaaaaataaattaaaaactagccgaggccaggcgcggtggctcaggcctgtaatcccagcactttgggaggccgaggcaggtggctcacctgaggtcaagagtttgagaccagcctgaccaacatggggaaaccctgtctcactaaaaatataaaaattagccaggcatggtggcccagacctgtaatcccagctacttgggaggctgaggcaggagaatggcctgaacccaggaggcggaggttgcaatgagccaagattgtgccactgcacttcagcctgggcaaaagagggaggctgtgtctcaaaaaaaaaaaaaaaaaaaaaaaaaaaaaaaattagccaggtggccgaggcagggtaatcgcttgagtctgtgatgtcaaggccacagtgagctgtgttcatgccactgcactccagcctgggtgacagagtgagactgtatctcagaaacaaacaaaaaaactgaggcttgatccttttagcaagattatataggtagtatttggcatgcccataggaaacacatgatgtctggttgtgtctctttttgtgatcttagcagctgttcattgggagttgcaaaatgatgatactctaattttatcactttttatttattagatggaatagttttatagagacatttctcctcatttattatttaccaaattcatatagaaaaagcaggataaatgcttgactctttttctttatttactaattttcaagataacgaattggttccctatcatcctttgaaggtgaccatttgaaaaatataattaatggccgggcacagtggctcatgcctgtaatcccagcactttgggaggctgaggtgggcggatcatgaggtcaggagattgagaccatcctggctaacacggtgaaaccccgtctctactaaaaaatagaaaaaattagctgggcgtggtggtgggcacctatagtcccagctactcgggcggctgaggcaggagaatggtgtgaatcgggaggcagagcttgcagtgagctatcgcgccactgcatttcagcctgggcgacagagcgagactcttatcttaaaaaaaaaaaaaaaaaaaaaatatatatatatatatatatatatgtgtgtatatatatatatatgtgtgtatatatatatatatgtgtgtatatatatatatgtgtgtatatatatatatatgtgtgtatatatatatatatgtgtatatatatatatatatgtgtgtatatatatatatatgtgtgtatatatatatatatatgattaattcagccaggtgcagtgactcacgcctgtaatcccagcattttgggaggccgaggcagttggatcacctgaggtcaggagttaaacaccagcctggccaacatagctaaaccccatcctctactaaaaatacaaaaaaaatagccgggcgtggtggtgggcacctgtaatcccagctacttgggaggctggggcaggagaatcacttgaacctgggaagtggagattacagtgagccgagatcaccccactgcactccagcctgggtgacagagagactctgtctcaaaaaaatatatacatatatacacaatatatagtatatatatacacaatacataatatatatacacaatatattgtatatattatatacacaatatatagtatatatatacacaatacataatatatatacacaatatattgtatatattatatacacaatatattgtatattatatatacaatatattgtatattatatacactatatattgtatattatatatactatatattgtatatatactatatattgtatattatatatactatatagtataatatacagtatatagtatattatatactatatagtataatatactatatacagtatattatatactatatacagtataatatatatatactatatatagtatatatacaatatactatatatgtttatactgtatattgtatatattatactatatattttatatatttatatatttatattatacatattatatatatactatatattatatacatataaaattcatggaattaaacatacttaatggctttcttttttttttttttgagacagagtctcactctgtcacccaggctagagtgcagtggcgtgatcttggctcactgcaacctccgactcccgggttcaagcgattctcctgccccagcctcccatgtagctgggattacaggcacgtgccactacacctggctaatttttttgtatttttagtagagacgggatttcgccatgttggccaggctggtctcgaactcctgacctcaggtgatccaactgcattggcctcccaaagtgctaggattataggcataggtgtgaaccaccgtgcctggcccacttaatggcttttaattcattacagttataattattgaagctcgtattgtctcacctttaaccagcagaaggctcttcatgtcggggtatttttccctctctccctcccttccttccttgcttttatctaacagctttattgagatataattcacataacatgaaattcacccatttaaaatgtgtgattcaaggccaaagaaggtagattacttgagcctaggggttcaagtccagcctggacaacatggcatgaaaccccatcttgataaaaaaaaaaaatacaaaaattagctaggagcggtggtgagcacgtgtagtcccagctactcaggaggctgaggcaggagaattgcttgaacccaggaggcagaggttgcagtaagctgagatcacaccactgtacttcatcctgggtgaagacagaacaagactgtgtctcaagaaaaaaaaatactgtgcaattcagtggtttttagtatattcacagagttgtacaacagtcaccacaatcaattttagaatgttttcatcacaccaaaatgaaaacctcagactattagcagttactccgtattccctctacttcctggcccctaccaaccactaatctgttttccgtttctatggatttgactgtttggtacatttcacataaatggaatcatacaatatttttatgtcttgtttcttttacttagcataatgttttcaaagttcaccagtgttgtagcatctgtcagcacagcattcttttttatgactgaataacattccactctggatgtaccacattttgtttctccattctttaggttgtttccagtttttggctattatgaaataaataatgcttctttgaacattcatgtacagctgtttatgtgaatatcttttcaattctcttggatatattcctaggcgtgaaattgctgggtcatatggtaatcccatatttaaccccatgccagactcccaaaacaattgcacccaagattttgttattgtccatctttttgaatatagccatcctagtgagtatgaagtggtagctcattgtggttttgatttgcatttccctaatgactaatgatgttaacatcttttcatctgcttattggctattgtatctctattcacaacctttgctcattttataattgagctatttgtcttcttattgagttatgagttctttacaggctgggtatggtgcctataagcccagtacttagggaggccagggcaggaggattgcttgagcccaggagctcaagacctgcctgggcaatacagggagacactgtctctacaaaaaatttaaaaattagcccagtgtggtggtgcatgtctgtagtcctagctacttggaagactgagatgggaggattgcttgagtccaggaggtggaggttgcagtgaaccgagatcacgccactgcactccagcttttattttatctcaaaaaataaagttctttatatattctggatacaagtcccttgtcagatataaattacaaatattttctcccattctgtgggttgtctttttactttcttgatggtattctttgaagcatgaaggattttgatgagttcctattccaacttttttttttttttttgcttgttatatttagaaaagtttgcctaaccaaaggtgatggagcttaatgcctatattttcttctaggagacttaaacttttagcttttacattaggtctactatccgttttgagttaatttttgttttttaatttttttgtttggtgtgtatgtgtgtgtatatatatatatatgtacatatatatatgtacatatatatatatatgtacatatatatatatgtacatatatatgtacatatatatatatgtacatatatatatatatatgtacatatatatatatatagagagagagagagagagagagtgagagagagagagacagagtctctctctgtcaccgaggctggagtgcagtggcacgatcgtggctcactgcaacctccgcctcctggccttaagtgatcctcccacctcagcctctcaaagttctgggattacaggtgtgagccaccgtacccagccttgaatttttgtgtatggcatgagaaaggagtccaactttattctttttcatgtgaatattcagttgttctagcgccatttgttgaaaagatactctttccccattgaattgtcttggcatctttgttgaaatcagttgattgtaactgtgaaggttaatttccggactcagttctattccattgagttgtatgtccatccttatgccattactgtcttgattcctatagttttgtagtactttttgagatggaaaagtgtgagtctccaactttgttgtttttcaagttggttttgactattttgggtctcttgcatttccatatgaattttaggatcagattgtcaatttctgcaaagaagcctactgagattttaatacccccatgttggcaagagtgtggagttgagtcagtacatcaatttggggagggaatattggcatgtcaacaatattatgtcttctgatccatggacataggctgtctttctgcttatttaggtctttttaaaaactctttgaacaatgttttatattttttcttttgttcgttttccttccttccttccttccttccttccttccttccttccttccttccttctctttctttctgtctttttttttttttttgtcccactgtcacccagtctggagtgcagtggtgcctggctcactaactacaacctccacctccctggcccagtgtgtcctccacttcagcctcctgtgtagctgggactataggcacacacaaccatgcccagctgatctttatatttttagtagagatggggtctcaccgcattgcccaggctggtttctaaccctttggctcaagccatccacctcgcctcagcctcccaaagtgctgggattataggtatgagccactgtgcctggcccatattttttcataatataagttttgtatttcttttgttaaatttattttttagtatttgtttttgatcctattgtaaattgttttcttaattttaattatttttggattgtttatagctagagtatagaaatttaattgctttttgcacattcatgttatatcctgtaaacttgcataactcatttattagttctattatgtatttctttttaaattgtgaaataacatgtatgtggtaaaaaggtcaaatagtgcgcaagggaatataaggaaaagtgaatcttctgccctctcacccctgctcccactaagctaaactattgtttagtttttcttaagctgtttcaagctgaagaaaggagtgggatattaaaaaacagtgcttcactgttagctgaatatcttttttttagacagggtcttaccctatcactcaggctggagtgcagtggtacaatcatagctcactgcagcctcagccttctgggcttaagcagtcctcccacttaagcctcctgagtagctgggaccactgttgtgcaccacaatgcctggctaatttttttaattttaatttttgtagaggtaaggtctccctccctatgttgcccaggccaatcttgaactcctgggctcaagcagtcctcccacctcagcctcccaaggtgctgggattacaggcgtgagccaccgcactgggccttcttttacttttgaccttgtgcttgtttttttcttcttggaggattacatataagtcaactagactccctagtttattctctttttacttacactgttaagaaaatgagatctggaatgtgaaacagatgtgattccttatctgattctgtcttcaggggtcagaaaaacctttggtgtggcagaaagacatgggtatttgggtgtttatacctggaagagatttcttacccctaacatggtctttgttgggagaaacgaaaaatagtgcatggctagaaactaggtttgtgcccaggacctctggcatgaggactggaagaaagttactgacgattaatgttcgacttcacccttactcttttagtcccagttgggtcttatcctggattcttcacattttgaactgcttctgttagcatgctctaaatgtgagagttctaattgtttgttggcgtttgtgggcaaacagtaaatggtcagattaataccagggaatatcagaaaattaacaggtttcaaattgctatttgtaaacctaaaggctaaaagtaatacaattaaataggcaagtatatgtggtcattaaaattcaagtaggcaaacaatcagataattgtatgttgcatattatcctgatctctttaagcacagtgttatctttgggaataaattattagatgtataaattacttttatctctgaaacatcatgttgagtttaccagtattacagccaatcccccaaagctttctgtttgccacagtttaatttactaaagaatatataaataccagtgagggacaggacaaattcaaagaggcatttgaacccaaaattatttcttaaaaccacttgagttaaatatattcaggtgagaaattcacgtgtgagttctttttcttttcttttaatggagaggaccagccatgtgggtagaggagagaagtagggcttcaaaatacaagacttagttttgaaggggtgggagtgccagggaggggactaggagaggctggtcatgaaccagagtgctgtgactgtcctggacaacctccttgttagatgggcagaggtgcaggcactctagagaccctcctggagaaccctgctgagaggttcctcggctatggcagagctaccgataattcttccttagggcaggtgttcaaggcaggccggctttagtggctccatgccacagcaagttctctaagctcagtgggcctcagtttctttatatgtgaaatggggatggtggtgtttgcctcaaagtatagttttaaagactaaataagttatcattattatttttgttttttttttagagacagggtcatactttgtcacctaggctggagtgcagcggcacaatcttggctcactgtagcctcgagttcctggcctcaagtgatcttatctccttggtctcccaaagtactaggattataggcgtgtgccactgcacccagcctgagttaaatgtttttaagatgagcttaacttgttcagtaacaataaaataatgtattttgattttttttttcccccggaaagtatgttgtcagtgatcatggaaaggccttgggatgcggtgtacatgctttcatttgttttgttttcatgccttcaaataaaattttaaaactatctgggccgggcatggtggctcatgcctataatcccagcactttgggaggctgaggcaggtggatcacctgaggtcaggagttcgaaactagactagccaacatggtgaaaccccgtctctactaaaaatacaaatattagctgggtgtggtggcgggcgcctgtaatcccagctactcgagaggctaaggcaggagaatcgcttgaacctaggagttggagcttgcagtgagtcaaaatcgcgccactgcactccagcctgggtgacaagagcaagactctgtcttaaaaaaacaaacaaacactttgggaggctgaggtgggcggatcacaaggtcaggagatcaagaccatcctggttaacacagtgaaaccccgtccctactgaaaatacaaaaaattagcccggcgtggtggtgggtgcctgtaatcccagctactcgggaggctgaggcaggagaatggcatgaacccgggaggcagagcttgtagtgagccgagatcgcgccactgcaccccagcctgggcgacagagcaagactccgcctcaaaaaacaaacaaacaaacaaaaaaactatttgaacttaaaccccaataatagtagatattttattttccaatcagttgtgtctatagaatgtcttttgatatgacaatggctgagaattctttgacaggttttgcaactcagattttctcaaagaatcttgtttaccttccatagcccctttgtttattgaaagcaaaatattcttagttcccttgaatcccagtgttcctgccattctggatgtctccatgctgtggcgtggggccctttttggggtgggattccccagcacagagcctgctaatacataagggtgtggtactcccattgagtctatttggcatcaatcagcaggcgggtttgcactgagcggtactaatcctagcttgcccaagcgattgtagttgttcttttaaaagcatcatctttttttggtctcctggcctaaaatttcagaagcaggaactgggggttatgggggttgggtataaaaggtagctaaacctgattatcctggcttgaaataatggcaggaataataggcttgttaactttgatcctagctgttaccttctacttaccagcctccctgctgaaccagcctctctgctgaaccagcctctgctgaaccaattggaatttggtggggactcctctgaacggcttttctgggtcctcaccttcaggatactgctagatgtgcccaacaagtatctcccattcccagcagggatacttttgattcttggccttcccgagagcctcaggcctgggattggccacttgggtccaagcagtgtctctagtttggcataattgggctactccgaggagcaggactctcagctgttatgagtgatgctaggagccaggcacagtggctcacgcctgtaatcctagcactttgggaggctgaggcgagtggattgcttgagcccaggagttcgagactagcctgggcaacatggggaaaccccgtctctactaaaaatacaaaaattatctgggtacgatggcatgcatctgtggtcccagctactcaggaggctgaggcgggaggattgcttgagtctgggaggtcaaggctgcagtgagctgagactgggccactgcactccagcctgggtgacagaacgagaccctgtctcaaaaaacaaaacaaaagacacacaaaaaagtgatgctagggaggcctttggcagagaatggatttccttaattgtgaatgtggtggtggtgcttctggaaatggaaatcaacagcactttgtggaggggcttttgcacagaatacatatatgaaccataaagaagtcccatttcccctccctaggttctcttcatacaagaaatggtagttagctccattgtttcctctgaatactttcctttgttttctttcttgaaggactgccaccaactcctgtccagctgctctatccagtgtcccgattcagcaatgtcaaatccctccagcacctttgcagattccggatacgacagctcgtcaggatagatcacatcccagatctcccactgcctaagtacaatggggttgtcaggtttgggacaggaatgagtaagggggttgtggggaggtaacaatgtcagtgaggcctgcaagctaccttcacaggcagagtctggcataaaatatttgcagatgtcattcttacacatttcctgtgtcttagcacctaactctagaactgtcatattgcagccagagggaggaaggtgtgtgtgtcagccttccttctgcagtctctaggaagaatagagtagaagtggtaagagtaaggaagacagaactataataactgagaaaatggggattgtgttaggtctcaaccatgtagggagtgaagaagagttgaacaaaggagttacttcttttatattttgtataaaaaccttttgtgtatatccgtcatttgtttttcacagacctctgatctcttatatccgaaagttctactactatgatcctcaggaagaggtatacctgtctctaaaggaagcgcagctcatttccaaacagaagcaagaggtggaaccctccacgtagcgaggggctccctgctggtcaccaccaagggtatgagctctctgcctcactgcccagccacatttcttcctgggcagtcatcatcatgcattgagccttgggccccctccccacaactcccaacatggatagccccgatccaggcatttgcccatagaatgacaagatctgaccgtggagtcctcaaggggcacacaccccatttgggagaaaagcagacagacgagaaagacatgaaagtagattgccaggaaacgtgcaagctaatcactgaatgtgagcaaggagggggacacccaagtgtagctaagagtgcggcctctggcaaggagagggtaattgaaaaaaataagaagaagagtgcagcctcctctcatacagacttgggctgatctccctgatactgcacaaatggcatataaagcaggaagctaaagggaaaggaaacttcatcaaagagaatgcaaacatgcctttaattgcaacaggcatgcacttcagtgagactccaccatgtcccacagcttggaatatggggatgaggaaaaacagcccactcttctatttagcgttccatttgggccattacaaagtaaattgttgaaactaaggaattcatttatctacgaggctcttctcccatcattacacagttctacccatttattttcccataaaacatcttgcaatactggtttgtgcagatgaattccaattagtcttttaaattgcactaaagtgaacattgtggggccaagttcctgagtagggaacacaattgccagggagccatgactttggtggtggtggtgacaagcaagattgactgccagtgtgcagtgactgctttcttctggtgtggatcctggagtagatgtacagtctcatcatgtcttcaattatatgcaaattgtatgcctttggaaatgactaaatatctaatgtcttgtgtctaggtggatttctctgcatgatggctttgcagttgagattagggtgagaggtagagcactagggaggtccacctacctgactacccttgaagagtaattaggccattcagttagaggaccacgtctgtgggtgcagtgctgtagaagaacccatataaacagggagcatgttcagagaaaggccttgtggcattggagagctcaaacttcatcatatgggaataaatggcctagaaagaaatgtgattaacccagagcacggaggttttagggagggatagctgaaagatgcatttgatgggttttcctaaggaagagagtgattaacctcctctctgtggttccagtggggagcaagttcagcattacgcttattctttgccacaggcaacaatcctgatgtcacagcaaaaagagatcctgagagttcagttcaaacctttgccagtgcgtgactgctcccaacactcaagacaagtggctccctgccggcctgttcctgatcatgccacacctttcccagttgaggcagggcctttatacctctaggtccccttgcccagaatgctctgccccaaggccctcccatgtcacagcaaaaagagacatggataaggaccaaaatgaatgtaagttacaaggaactagatttcagtccaactttacaagaacctttttaacagagacgtccaaaactgggatggttttcctcaagagcttccattcagcagatagctgtatatttgtgtttatttggcacccagtatgaattgagaatagagtgctgagcaaagcaaaggccctgttttcactcagcttcccttctcctaaggaaagacaggcagcaaacatgaacataaatgaatgggctaatttcaggtgactgtacagtgttgtgaggaaagtaaaactgggtaaagtttagtgattagtggtgggatactcctcaagtttagatggtcagggagagacttgtgaggaggtgccatttcagccgatacccaaatatcaaggagccagccatgggagtgccttggagcagagcattctgggcaaggggacctagaggtataaaggccctgcctcagctgggaaggacgtggcatgatcaggaacaggccagtgggtagccatttgtcttgagtgttgggggcagtcatatattggcgaaggtttgaactgaactcccaggatccctttttgctgtgacatcagtattgatgcctgtgacaaagagtaagcgtaatgctgaacttgctcctggtgtagaccctaggagccctggaaagggaggaagtaacacttcccaagggaaaggtcttttttttttttttttttgagatggagtttcgctcttgttgcccgggctggagtgcaatggcgtgatcttggctcactgcaacctccatctcccgggttcaagcaattctcccacctcagcctcccagtgggaggctgggattacaggtgtgcaccaccacgcctggctaattttgtatttttagtagactgggtttcaccacgtttgtcagttgggtctcaaactcctgacctcactcaagtgatccacccaccttggcctcccaaagttctgggattacaggcatgagccactgtgccccattgggaaaggtctttagggtatgaaatttggcctttatgacttcttgttgttcaggctgattttaatttgggagttctgaatggcctgcttggtctatgtgtgctgttacctggggaatttcatgccagggggtttggagtcagactatagagtaatggctagaacagtgcctgtctcaagtcggaaggtggaagcaacagtggcctttgtgtttctgtgtcaaccctgtccagttgacagaagctgcagaaatacagagctctctggccaggtgtgatggttcacgtctgtaatcccagcagtttgggaggccaaggcgggctcatcacgaggtcaggagtttgagaccagcctggccaacatggtgaaaccctgtctctactaaaaatacaaaattagccaggcatggtggcgcatgcctgtaatcccagctacttgggaggctgagacaggagaatcttgaacccaggaagtggagattgcagtgagccaagatcatgcctttgcactccagcctgggtgacagagcgagactccgtctcaaaaaaaaaaaaaaaaaagaaagaaagaaatatagagctctttcttgccaggttcaaaaggggttactccaggggcttacttcccttttcagtccctagagcaattgagcctcagggaaaaaatgggtaggaaatgaaggggtaagggaaagggaaagagagaatttatgcaatgtggtaggatgtatttattagttatttcaggaccttgttttgtcatccaagtcataggctgctcaggtccccctgacaaaggccactgctgcatccctagtcacatgtggccctcatggaagcgttcagctctccaggctccgttctccttttgaggtgtgctgctccacctccagtggctggctgttatttcactccattttggttctattctgttgaactgttttaattttttttccttttctacctaggcatttggttgccaagctccagctttgaagaaccaaattaagctaccatgaaaagaagaggaaaagtgagggaacaggaaggttgggattctctgtgcagagactttggttccccacgcagccctggggcttggaagaagcacatgaccgtactctgcgtggggctccacctcacacccacccctgggcatcttaggactggaggggctccttggaaaactggaagaagtctcaacactgtttctttttcagaa